(19) United States
(12) Patent Application Publication
(10) Pub. No.: US 2026/0310076 A1
(43) Pub. Date: Oct. 8, 2026

(54) PHOTOSENSITIVE RESIN COMPOSITION, CURED SUBSTANCE, LAMINATE, MANUFACTURING METHOD FOR CURED SUBSTANCE, MANUFACTURING METHOD FOR LAMINATE, MANUFACTURING METHOD FOR SEMICONDUCTOR DEVICE, SEMICONDUCTOR DEVICE, POLYIMIDE, MANUFACTURING METHOD FOR POLYIMIDE, AND COMPOUND

(71) Applicant: FUJIFILM Corporation, Tokyo (JP)

(72) Inventor: Atsuyasu Nozaki, Shizuoka (JP)

(73) Assignee: FUJIFILM Corporation, Tokyo (JP)

(21) Appl. No.: 19/559,138

(22) Filed: Mar. 6, 2026

Related U.S. Application Data
(63) Continuation of application No. PCT/JP2024/034429, filed on Sep. 26, 2024.

(30) Foreign Application Priority Data
Sep. 29, 2023 (JP) 2023-169927

(51) Int. Cl. G03F 7/038; C08G 73/12; G03F 7/004; G03F 7/039; H10P 76/20

(52) U.S. Cl. CPC G03F 7/0387; C08G 73/124; C08G 73/127; G03F 7/0042; G03F 7/039; H10P 76/2041

(57) ABSTRACT

Provided are a photosensitive resin composition containing a polyimide including a repeating unit represented by Formula (1-1) and a photosensitizer, a cured substance obtained by curing the composition, a manufacturing method for the cured substance, a laminate including the cured substance, a manufacturing method for the laminate, a semiconductor device, a manufacturing method for the semiconductor device, a novel polyimide, a manufacturing method for the polyimide, and a compound; in Formula (1-1), X¹ represents an organic group, Y¹ represents an organic group, R¹ represents a specific structure including an acetal group, n and m each represent an integer of 0 to 4, and n+m represents an integer of 1 or more.

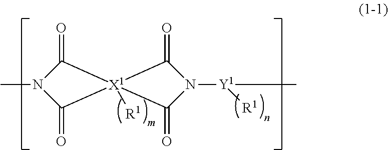

### CROSS-REFERENCE TO RELATED APPLICATIONS

This application is a Continuation of PCT International Application No. PCT/JP2024/034429 filed on Sep. 26, 2024, which claims priority under 35 U.S.C § 119(a) to Japanese Patent Application No. 2023-169927 filed on Sep. 29, 2023. Each of the above application(s) is hereby expressly incorporated by reference, in its entirety, into the present application.

### BACKGROUND OF THE INVENTION

### 1. Field of the Invention

The present invention relates to a photosensitive resin composition, a cured substance, a laminate, a manufacturing method for a cured substance, a manufacturing method for a laminate, a manufacturing method for a semiconductor device, a semiconductor device, a polyimide, a manufacturing method for a polyimide, and a compound.

### 2. Description of the Related Art

In recent years, a resin material that has been produced from a photosensitive resin composition containing a resin has been utilized in various fields.

For example, a heterocyclic ring-containing polymer such as a polyimide is applied to various applications because of excellent heat resistance, insulating properties, and the like. The above-described use applications are not particularly limited, and examples thereof in a semiconductor device for mounting include the use as a material of an insulating film or sealing material, or as a protective film. In addition, such a resin can also be used as a base film, a cover lay, or the like for a flexible substrate.

For example, in the above-described applications, the heterocyclic ring-containing polymer such as the polyimide is used in the form of a photosensitive resin composition containing the heterocyclic ring-containing polymer or a precursor thereof.

Such a photosensitive resin composition is applied onto a base material by coating or the like to form a photosensitive film, and then, as necessary, subjected to exposure, development, or heating, whereby a cured substance can be formed on the base material.

Since the photosensitive resin composition can be applied by a known coating method or the like, it can be said that the photosensitive resin composition is excellent in the manufacturing adaptability, for example, a high degree of freedom in a case of application in designing the shape, size, application position of the photosensitive resin composition to be applied. From the viewpoint of excellent adaptability in manufacturing in addition to the high performance of the heterocyclic ring-containing polymer such as the polyimide, the industrial application of the above-described photosensitive resin composition is expected to be further expanded.

For example, WO2022/270541A describes a negative-tone photosensitive resin composition containing (A) a polyimide, (B) a crosslinking agent including a polyfunctional (meth)acrylate, and (C) a photopolymerization initiator, in which the polyimide (A) contains a structural unit having a specific structure.

### SUMMARY OF THE INVENTION

In the photosensitive resin composition containing a polyimide or the like, it is required to achieve both resolution and breaking elongation.

An object of the present invention is to provide a photosensitive resin composition from which a cured substance having excellent resolution and excellent breaking elongation is obtained, a cured substance obtained by curing the photosensitive resin composition, a laminate including the cured substance, a manufacturing method for the cured substance, a manufacturing method for the laminate, a manufacturing method for a semiconductor device including the manufacturing method for the cured substance, and a semiconductor device including the cured substance.

Another object of the present invention is to provide a novel polyimide, a manufacturing method for a polyimide, and a compound.

Examples of representative embodiments according to the present invention are described below.

<1> A photosensitive resin composition comprising:
a polyimide including a repeating unit represented by Formula (1-1); and
a photosensitizer,

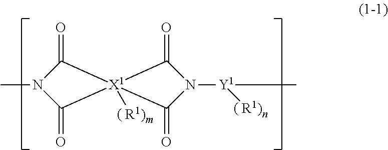

in Formula (1-1), X¹ represents an organic group having 4 or more carbon atoms, Y¹ represents an organic group having 4 or more carbon atoms, R¹'s each independently represent a structure represented by Formula (R-1), n and m each represent an integer of 0 to 4, and n+m represents an integer of 1 or more,

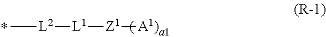

in Formula (R-1), L¹ represents an acetal group, L² represents a single bond or a divalent linking group, Z¹ represents a (1+a1)-valent linking group, A¹ represents a polymerizable group, a1 represents an integer of 0 or more and a maximum substituent number of Z¹ or less, and * represents a bonding site to X¹ or Y¹ in Formula (1-1).

<2> The photosensitive resin composition according to <1>,
in which L¹ in Formula (R-1) is thermally decomposable.

<3> The photosensitive resin composition according to <1> or <2>
in which a1 in Formula (R-1) is an integer of 1 or more, and
A¹ is a radically polymerizable group.

<4> The photosensitive resin composition according to any one of <1> to <3>,
in which X¹ and Y¹ in Formula (1-1) each include a structure obtained by removing two or more hydrogen atoms from a structure represented by any of Formulae (V-1) to (V-10),

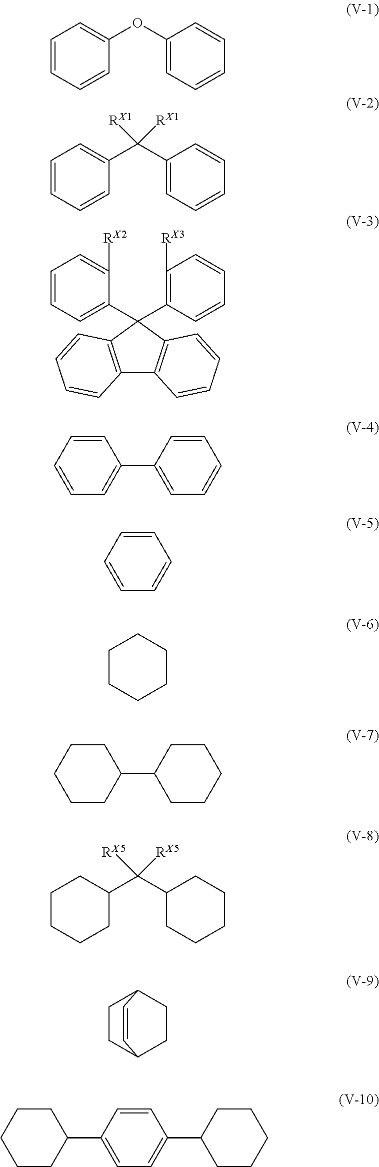

in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure,
in Formula (V-8), R^{X5}'s each independently represent a hydrogen atom, an alkyl group, or a halogenated alkyl group.

<5> A photosensitive resin composition comprising:
a polyimide from which a structure having 2 or more carbon atoms is eliminated upon heating at 230° C. for 3 hours; and
a photosensitizer.

<6> The photosensitive resin composition according to <5>,
in which the eliminated structure includes a polymerizable group.

<7> The photosensitive resin composition according to any one of <1> to <6>,
in which a polymerizable group equivalent of the polyimide is 0.2 to 5.0 mmol/g.

<8> The photosensitive resin composition according to any one of <1> to <7>,
in which in a case where a film-like cured substance having a film thickness of 10 m is formed using the photosensitive resin composition, a transmittance of the cured substance at a wavelength of 365 nm is 15% or more.

<9> The photosensitive resin composition according to any one of <1> to <8>,
in which the polyimide has a chain-like alkyl group having 6 or more carbon atoms.

<10> The photosensitive resin composition according to any one of <1> to <9>, further comprising:
an azole compound.

<11> The photosensitive resin composition according to any one of <1> to <10>, further comprising:
a silane coupling agent.

<12> The photosensitive resin composition according to any one of <1> to <11>, further comprising:
an organic titanium compound.

<13> The photosensitive resin composition according to any one of <1> to <12>, further comprising:
a solvent having a boiling point of 100° C. to 260° C. at 1 atm.

<14> The photosensitive resin composition according to <13>,
in which a content of the solvent having the boiling point of 100° C. to 260° C. is 40% by mass or more with respect to a total mass of the composition.

<15> The photosensitive resin composition according to <13> or <14>, further comprising:
two or more kinds of the solvents having the boiling point of 100° C. to 260° C.

<16> The photosensitive resin composition according to any one of <1> to <15>,
in which the photosensitive resin composition is used for forming an interlayer insulating film for a re-distribution layer.

<17> A cured substance obtained by curing the photosensitive resin composition according to any one of <1> to <16>.

<18> A laminate comprising:
two or more layers consisting of the cured substance according to <17>; and
a metal layer provided between any layers consisting of the cured substance.

<19> A manufacturing method for a cured substance, comprising:
a film forming step of applying the photosensitive resin composition according to any one of <1> to <16> onto a substrate to form a film;
an exposure step of selectively exposing the film; and
a development step of developing the film using a developer to form a pattern.

<20> The manufacturing method for a cured substance according to <19>, further comprising:
a heating step of heating the film at 50° C. to 450° C.

<21> A manufacturing method for a laminate, comprising:
the manufacturing method for a cured substance according to <19> or <20>.

<22> A manufacturing method for a semiconductor device, comprising:
the manufacturing method for a cured substance according to <19> or <20>.

<23> A semiconductor device comprising:
the cured substance according to <17>.

<24> A polyimide comprising:
a repeating unit represented by Formula (1-1),

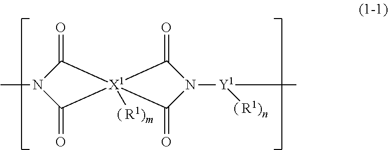

in Formula (1-1), X¹ represents an organic group having 4 or more carbon atoms, Y¹ represents an organic group having 4 or more carbon atoms, R¹'s each independently represent a structure represented by Formula (R-1), n and m each represent an integer of 0 to 4, and n+m represents an integer of 1 or more,

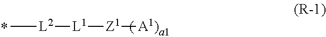

in Formula (R-1), L¹ represents an acetal group, L² represents a single bond or a divalent linking group, Z¹ represents a (1+a1)-valent linking group, A¹ represents a polymerizable group, a1 represents an integer of 0 or more and a maximum substituent number of Z¹ or less, and * represents a bonding site to X¹ or Y¹ in Formula (1-1).

<25> The polyimide according to <24>,
in which a1 in Formula (R-1) is an integer of 1 or more, and
A¹ is a radically polymerizable group.

<26> The polyimide according to <24> or <25>,
in which a polymerizable group equivalent is 0.2 to 5 mmol/g.

<27> The polyimide according to any one of <24> to <26>,
in which the polyimide has a chain-like alkyl group having 6 or more carbon atoms.

<28> The polyimide according to any one of <24> to <27>,
in which both of X¹ and Y¹ in Formula (1-1) include a ring structure.

<29> The polyimide according to any one of <24> to <28>,
in which X¹ and Y¹ in Formula (1-1) each include a structure obtained by removing two or more hydrogen atoms from a structure represented by any of Formulae (V-1) to (V-10),

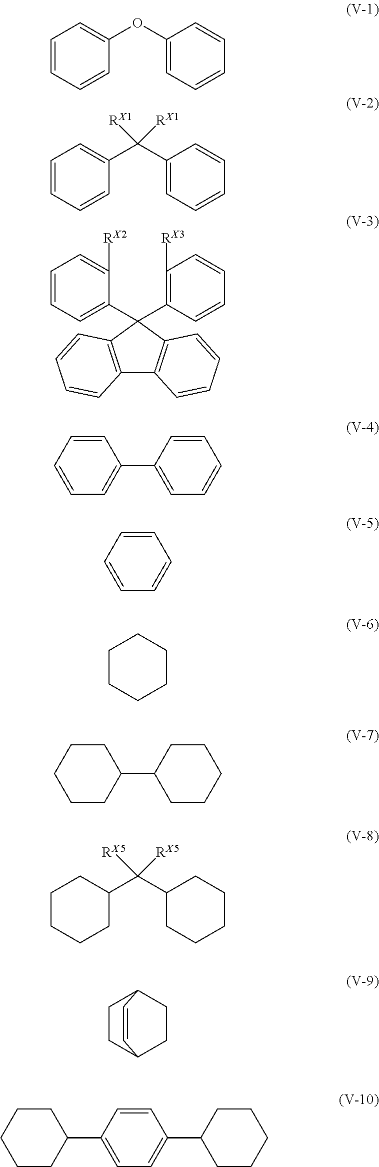

in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure,
in Formula (V-8), R^{X5}'s each independently represent a hydrogen atom, an alkyl group, or a halogenated alkyl group.

<30> A manufacturing method for the polyimide according to any one of <24> to <29>, the method comprising:
a step of reacting a polyimide having an acid group or a precursor of the polyimide with a compound represented by Formula (B-1),

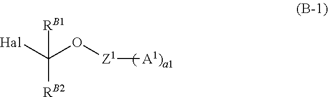

in Formula (B-1), Z¹ represents a (1+a1)-valent linking group, A¹ represents a polymerizable group, a1 represents an integer of 1 or more and a maximum substituent number of Z¹ or less, R^{B1} and R^{B2} each independently represent a hydrogen atom or a monovalent organic group, and Hal represents a halogen atom.

<31> The manufacturing method for a polyimide according to <30>, further comprising:
a step of obtaining the polyimide having the acid group from an acid dianhydride and a diamine.

<32> A compound represented by Formula (A-1).

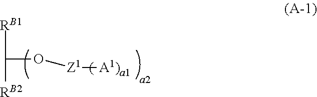

in Formula (A-1), Z¹'s each independently represent a (1+a1)-valent linking group, A¹'s each independently represent a polymerizable group, a1's each independently represent an integer of 1 or more and a maximum substituent number of Z¹ or less, R^{B1} and R^{B2} each independently represent a hydrogen atom or a monovalent organic group, and a2 represents 2.

<33> The compound represented by Formula (B-1),

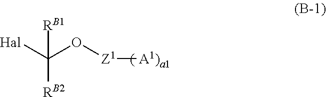

in Formula (B-1), Z¹ represents a (1+a1)-valent linking group, A¹ represents a polymerizable group, a1 represents an integer of 1 or more and a maximum substituent number of Z¹ or less, R^{B1} and R^{B2} each independently represent a hydrogen atom or a monovalent organic group, and Hal represents a halogen atom.

<34> The compound according to <33>,
in which the polymerizable group in A¹ in Formula (B-1) is a radically polymerizable group.

According to the present invention, there are provided a photosensitive resin composition from which a cured substance having excellent resolution and excellent breaking elongation is obtained, a cured substance obtained by curing the photosensitive resin composition, a laminate including the cured substance, a manufacturing method for the cured substance, a manufacturing method for the laminate, a manufacturing method for a semiconductor device including the manufacturing method for the cured substance, and a semiconductor device including the cured substance.

In addition, according to the present invention, there are provided a novel polyimide, a manufacturing method for a polyimide, and a compound.

### DESCRIPTION OF THE PREFERRED EMBODIMENTS

Hereinafter, the main embodiments according to the present invention will be described. However, the present invention is not limited to the specified embodiments.

In the present specification, a numerical value range described by using “to” means a range including numerical values described before and after the preposition “to” as a lower limit value and an upper limit value, respectively.

In the present specification, the term “step” means not only an independent step but also a step that cannot be clearly distinguished from other steps as long as the desired action of the step can be achieved.

In describing a group (an atomic group) in the present specification, in a case where a description of substitution and unsubstitution is not provided, the description means the group includes a group (an atomic group) having a substituent as well as a group (an atomic group) having no substituent. For example, the “alkyl group” includes not only an alkyl group that does not have a substituent (an unsubstituted alkyl group) but also an alkyl group that has a substituent (a substituted alkyl group).

In the present specification, the “exposure” includes not only exposure using light but also exposure using corpuscular beams such as an electron beam and an ion beam, unless otherwise specified. In addition, examples of the light that is used for exposure include an actinic ray such as a bright line spectrum of a mercury lamp, a far ultraviolet ray represented by an excimer laser, an extreme ultraviolet ray (EUV light), an X-ray, or an electron beam, and a radioactive ray.

In the present specification, “(meth)acrylate” means one or both of “acrylate” and “methacrylate”, “(meth)acryl” means one or both of “acryl” and “methacryl”, and “(meth)acryloyl” means one or both of “acryloyl” and “methacryloyl”.

In the structural formulae of the present specification, Me represents a methyl group, Et represents an ethyl group, Bu represents a butyl group, and Ph represents a phenyl group.

In the present specification, the total solid content refers to the total mass of components excluding a solvent from the entire components of the composition. In addition, in the present specification, the concentration of solid contents is a mass percentage of other components excluding a solvent with respect to the total mass of the composition.

In the present specification, weight-average molecular weight (Mw) and number-average molecular weight (Mn) are each a value measured using gel permeation chromatography (GPC) unless otherwise specified, which are defined as a polystyrene equivalent value. In the present specification, the weight-average molecular weight (Mw) and the number-average molecular weight (Mn) can be determined, for example, by using HLC-8220 GPC (manufactured by Tosoh Corporation) and using GUARD COLUMN HZ-L, TSKgel Super HZM-M, TSKgel Super HZ4000, TSKgel Super HZ3000, and TSKgel Super HZ2000 (all of which are manufactured by Tosoh Corporation) as a column connected in series. The molecular weights thereof are measured using N-methyl-2-pyrrolidone (NMP) as an eluant unless otherwise specified. However, in a case where NMP is not suitable as an eluent, such as in a case of low solubility, tetrahydrofuran (THF) can also be used. In addition, the detection in GPC measurement is carried out using a detector with an ultraviolet ray (a UV ray) of a wavelength of 254 nm unless otherwise specified.

In the present specification, in a case where the positional relationship of respective layers constituting the laminate is described as “upper” or “lower”, it suffices that another layer is on the upper side or the lower side of the reference layer among the plurality of layers of interest. That is, a third layer or element may be further interposed between the reference layer and the other layer, and the reference layer and the other layer need not be in contact with each other. Unless otherwise specified, the direction in which the layers are laminated on the base material is referred to as “upward”, or in a case where a resin composition layer is present, the direction from the base material to the resin composition layer is referred to as “upper”. The opposite direction thereof is referred to as “downward”. Furthermore, such a setting of upward and downward directions is for convenience in the present specification, and in a practical aspect, the “upward” direction in the present specification may be different from a vertically upward direction.

In the present specification, a composition may contain, as each component contained in the composition, two or more compounds corresponding to the component unless otherwise specified. The content of each component in the composition means the total content of all the compounds corresponding to the component unless otherwise specified.

In the present specification, unless otherwise specified, the temperature is 23° C., the atmospheric pressure is 101,325 Pa (1 atm), and the relative humidity is 50% RH.

In addition, in the present specification, a combination of preferred aspects is a more preferred aspect.

### (Photosensitive Resin Composition)

A photosensitive resin composition according to a first aspect of the present invention (hereinafter, also simply referred to as a “first resin composition”) contains a polyimide including a repeating unit represented by Formula (1-1) and a photosensitizer.

A photosensitive resin composition according to a second aspect of the present invention (hereinafter, also simply referred to as a “second resin composition”) contains a polyimide from which a structure having 2 or more carbon atoms is eliminated by heating at 230° C. for 3 hours, and a photosensitizer.

Hereinafter, the first resin composition and the second resin composition are also simply referred to as “resin composition”.

Hereinafter, the polyimide including the repeating unit represented by Formula (1-1) is also referred to as a “first specific resin”, the polyimide from which a structure having 2 or more carbon atoms is eliminated by heating at 230° C. for 3 hours is also referred to as a “second specific resin”, and these are also collectively referred to as “specific resin”.

The resin composition according to the embodiment of the present invention is preferably used for forming a photosensitive film that is subjected to exposure and development, and is more preferably used for forming a film that is subjected to exposure and development using a developer containing an organic solvent.

The resin composition according to the embodiment of the present invention can be used, for example, for forming an insulating film of a semiconductor device, an interlayer insulating film for a re-distribution layer, a stress buffer film, or the like, and it is preferably used for forming an interlayer insulating film for a re-distribution layer.

In particular, one of the preferred aspects of the present invention is that the resin composition according to the embodiment of the present invention is used for forming an interlayer insulating film for a re-distribution layer.

In addition, the resin composition according to the embodiment of the present invention is preferably used for forming a photosensitive film that is subjected to negative-tone development.

In the present invention, the negative-tone development refers to development in which, in exposure and development, a non-exposed portion is removed by the development, and the positive-tone development means a development in which an exposed portion is removed by the development.

As the exposure method, the developer, and the development method, for example, the following ones in the description of the manufacturing method for a cured substance, which will be described later, are used: an exposure method to be described in the exposure step, and a developer and a development method to be described in the development step.

According to the resin composition according to the embodiment of the present invention, a cured substance having excellent resolution and excellent breaking elongation can be obtained.

Although the mechanism by which the above effect is obtained is not revealed, it is presumed as follows.

As one method for improving the resolution, there is a method for increasing a development residual film rate. The development residual film rate refers to a residual rate of a film thickness of a film remaining after development with respect to a film thickness of a film formed from a photosensitive resin composition before development in a step of exposing and developing the film, and a residual film rate of a pattern portion remaining after development is the development residual film rate.

As the development residual film rate is higher, the film thickness during coating can be made thinner with respect to the required film thickness, and as the film is thinner, the non-pattern portion is easily removed by development, and the pattern formation is easily performed, so that the resolution can be improved.

For example, in order to increase the development residual film rate, for example, it is considered to improve the crosslinking density of the exposed portion by increasing the amount of polymerizable groups bonded to the molecular chain of the polyimide.

However, the present inventors have found that, in a case where the amount of polymerizable groups in the molecular chain of the polyimide is increased, the alignment of the polyimide resin in the cured substance (particularly, in a case where the polyimide resin includes a ring structure such as an aromatic ring structure, the alignment of the ring structure) is decreased, and the crosslinking density of the resin is high, the structure is rigid, and thus the breaking elongation may be poor.

Therefore, the present inventors have considered that, in order to improve the breaking elongation, it is preferable to add as few polymerizable groups or the like as possible to the molecular chain, and to set the state in which the interaction between resins such as the packing of the resin is likely to occur or the state in which the crosslinking density of the resin is low.

However, in a case where the molecular chain having a polymerizable group in the resin is simply reduced, the crosslinking density is insufficient, the development residual film rate is decreased, and the film thickness needs to be increased, so that the resolution is deteriorated. For the above reasons, it is difficult to achieve both the resolution and the breaking elongation.

Therefore, as a result of intensive studies, the present inventors have found that, by using the polyimide having the repeating unit represented by Formula (1-1) in the resin composition, it is possible to achieve both the resolution and the breaking elongation.

It is presumed that, in the repeating unit represented by Formula (1-1), since the development residual film rate can be improved by the polymerization of the resins with each other or the polymerization of the resin and the polymerizable compound, a photosensitive film having excellent resolution can be formed, and the acetal structure portion is decomposed in the cured substance by the effect of heating or the like, so that the molecular chain is released from the resin, and thus the breaking elongation is excellent.

Here, WO2022/270541A does not describe a resin composition containing a resin corresponding to the specific resin.

Hereinafter, the components contained in the resin composition according to the embodiment of the present invention will be described in detail.

### <Specific Resin>

### [First Specific Resin]

The first resin composition contains a polyimide (first specific resin) including a repeating unit represented by Formula (1-1).

It is preferable that the second specific resin includes a repeating unit represented by Formula (1-1).

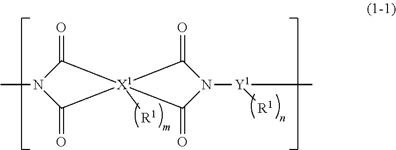

In Formula (1-1), X¹ represents an organic group having 4 or more carbon atoms, Y¹ represents an organic group having 4 or more carbon atoms, R¹'s each independently represent a structure represented by Formula (R-1), n and m each represent an integer of 0 to 4, and n+m represents an integer of 1 or more.

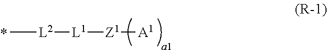

In Formula (R-1), L¹ represents an acetal group, L² represents a single bond or a divalent linking group, Z¹ represents a (1+a1)-valent linking group, A¹ represents a polymerizable group, a1 represents an integer of 0 or more and a maximum substituent number of Z¹ or less, and * represents a bonding site to X¹ or Y¹ in Formula (1-1).

In the present specification, the polyimide refers to a resin having a repeating unit including an imide group in a molecular chain, and it is preferably a resin having a repeating unit including an imide ring structure in a molecular chain.

In addition, in a case where the polyimide is a linear resin, the polyimide is preferably a resin having a repeating unit including an imide group in a main chain, and more preferably a resin having a repeating unit including an imide ring structure in a main chain.

In the present specification, the term “main chain” represents the relatively longest bonding chain in a resin molecule, and the term “side chain” refers to other bonding chains other than the main chain.

In the present specification, the imide group refers to a structure represented by *—C(═O)N(—*)C(═O)—*, in which * represents a bonding site to another structure, and it is preferably a bonding site to a carbon atom and more preferably a bonding site to a quaternary carbon atom.

In the present specification, the imide ring structure refers to a ring structure including all of two carbon atoms and a nitrogen atom in the imide as ring members. The imide ring structure is preferably a 5-membered ring.

The first specific resin includes an imide ring structure which is a 5-membered ring in the repeating unit represented by Formula (1-1).

The polyimide may be a so-called polyamideimide having an amide group in addition to the imide group in the molecular chain. In the present specification, the amide group refers to a structure represented by *—C(═O)N(— #)-*, in which * represents a bonding site to another structure, and it is preferably a bonding site to a carbon atom and more preferably a bonding site to a quaternary carbon atom. In addition, # represents a bonding site to another structure, and it is preferably a bonding site to a hydrogen atom or a carbon atom and more preferably a bonding site to a hydrogen atom.

In the present invention, the polyimide precursor refers to a resin which is polyimide due to a change in chemical structure caused by an external stimulus, and a resin which is polyimide due to a change in chemical structure caused by heat is preferable, and a resin which is polyimide due to a ring closure reaction caused by heat to form a ring structure is more preferable.

A preferred aspect of the polyimide to be formed is as described above.

A content of the polymerizable group (polymerizable group equivalent) with respect to the total mass of the specific resin is preferably 0.2 to 5.0 mmol/g, more preferably 0.25 to 4.0 mmol/g, and still more preferably 0.3 to 3.0 mmol/g. In the present specification, the polymerizable group equivalent is defined as a molar amount of polymerizable groups contained in 1 mol of the compound/number-average molecular weight of the compound.

A preferred aspect of the polymerizable group contained in the specific resin will be described later.

The polymerizable group equivalent of the specific resin can be calculated by measuring the resin taken out from the composition by ¹H-NMR.

For example, in a case where the polymerizable group is a vinylphenyl group, the amount of the vinylphenyl group can be measured as follows. In a case where the polymerizable group is another structure, an integral intensity may be calculated for a peak specific to each polymerizable group instead of the peak derived from the vinylphenyl group.

For example, using benzenesulfonyl anilide as a reference substance, the polymerizable group equivalent in the specific resin can be calculated from the ratio of the integral intensity of a peak near 4.9 to 7.0 ppm derived from the vinylphenyl group to the integral intensity of a peak derived from the reference substance, the amount of the reference substance, and the amount of the above-described resin A in the ¹H-NMR chart.

1.00 g of the resin and 0.0233 g (0.1 millimole) of benzenesulfonyl anilide are dissolved in 9.0 g of deuterated DMSO, and ¹H-NMR (accumulated 640 times) is performed. The integral value of the peak near 10.3 ppm, which is the NH peak of benzenesulfonyl anilide, is set to 0.1, and the accumulated value of any one of 4.9 to 5.5 ppm, 5.5 to 6.2 ppm, or 6.5 to 7.0 ppm derived from the vinylphenyl group is obtained.

The unit of the above-described accumulated value is mmol/g, and this value is defined as the vinylphenyl amount. The molar amount of the vinylphenyl group in the specific resin can be calculated from the amount of the specific resin.

The same applies to the composition.

### —R¹—

In Formula (R-1), L¹ represents an acetal group. In the present invention, the acetal group is preferably a group represented by Formula (AC-1).

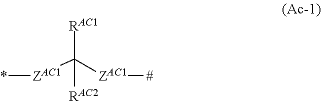

In Formula (Ac-1), R^{AC1} and R^{AC2} each independently represent a hydrogen atom or a monovalent organic group, one of R^{AC1} or R^{AC2} may be bonded to Z¹ in Formula (R-1) at a position other than # to form a ring structure, Z^{AC1} and Z^{AC2} each independently represent —O— or —S—, * represents a bonding site to L² in Formula (R-1), and # represents a bonding site to Z¹ in Formula (R-1).

In Formula (Ac-1), it is preferable that one of R^{AC1} or R^{AC2} is a hydrogen atom and the other is a monovalent organic group.

The monovalent organic group in R^{AC1} or R^{AC2} is preferably a hydrocarbon group, more preferably an alkyl group or an aromatic hydrocarbon group, still more preferably an alkyl group having 3 to 6 carbon atoms or a phenyl group, and even more preferably an isopropyl group, a t-butyl group, or a phenyl group.

In addition, in a case where one of R^{AC1} or R^{AC2} is bonded to Z¹ in Formula (R-1) at a position other than # to form a ring structure, examples of the ring structure to be formed include a tetrahydrofuranyl ring structure and a tetrahydropyranyl ring structure.

It is preferable that at least one of Z^{AC1} or Z^{AC2} is —O—, and it is more preferable that both of Z^{AC1} and Z^{AC2} are —O—.

In Formula (R-1), L¹ is preferably thermally decomposable.

Specifically, in a case where 60% by mole or more of the structure is decomposed in a case where the specific resin is heated under the conditions of 1 atm, 230° C., and 3 hours, the structure is thermally decomposable.

The temperature at which 60% by mole or more is decomposed is preferably 220° C., more preferably 200° C., and still more preferably 170° C.

in Formula (R-1), L² represents a single bond or a divalent linking group, and it is preferably a single bond, or a group represented by a bond of two or more of a hydrocarbon group, —C(═O)—, —S(═O)₂—, or —NRN—, and it is more preferably a single bond or —C(═O)—.

In Formula (R-1), Z¹ represents a (1+a1)-valent linking group, and it is preferably a hydrocarbon group or a group in which a hydrocarbon group and at least one group selected from the group consisting of —O—, —C(═O)—, —S—, —S(═O)₂—, and —NRN— are bonded to each other, more preferably a hydrocarbon group or a group in which a hydrocarbon group and —NR^{N}—C(═O)—NR^{N}— are bonded to each other, and still more preferably a hydrocarbon group or a group in which two hydrocarbon groups are linked to each other through —NR^{N}—C(═O)—NR^{N}.

R^{N} represents a hydrogen atom or a monovalent organic group, and it is preferably a hydrogen atom, an alkyl group, or a phenyl group, and more preferably a hydrogen atom.

The hydrocarbon group is preferably a saturated aliphatic hydrocarbon group having 2 to 20 carbon atoms, an aromatic hydrocarbon group having 6 to 20 carbon atoms, or a group represented by a bond of these groups, more preferably a saturated aliphatic hydrocarbon group having 2 to 10 carbon atoms, an aromatic hydrocarbon group having 6 to 10 carbon atoms, or a group represented by a bond of these groups, and still more preferably a saturated aliphatic hydrocarbon group having 2 to 10 carbon atoms.

In the present specification, in a case of being described as a saturated aliphatic hydrocarbon group, an aromatic hydrocarbon group, or the like, unless otherwise specified (for example, as being unsubstituted), the hydrogen atom in these groups may be substituted with a substituent within a range in which the effect of the present invention can be obtained.

in Formula (R-1), A¹ represents a polymerizable group, and it is preferably a group having an ethylenically unsaturated bond, an epoxy group, an oxetanyl group, or a benzoxazolyl group, and more preferably a group having an ethylenically unsaturated bond.

In addition, A¹ is preferably a radically polymerizable group.

Examples of the group having an ethylenically unsaturated bond include a vinyl group, an allyl group, a vinylphenyl group, a (meth)acryloyl group, a maleimide group, and a (meth)acrylamide group.

Among these, a (meth)acryloyl group, a (meth)acrylamide group, a vinylphenyl group, or a maleimide group is preferable, and from the viewpoint of reactivity, a (meth)acryloyl group is more preferable. In addition, from the viewpoint of reducing the dielectric loss tangent or the like, a vinylphenyl group or a maleimide group is preferable.

In Formula (R-1), a1 is preferably an integer of 1 or more and a maximum substituent number of Z¹ or less, preferably an integer of 1 to 4, and more preferably 1 or 2.

In the present invention, an aspect in which a1 in Formula (R-1) is an integer of 1 or more and A¹ is a radically polymerizable group is also one of the preferred aspects of the present invention. In the above-described aspect, preferred aspects of a1 and A¹ are as described above.

In Formula (1-1), in a case where m+n is an integer of 2 or more, m+n pieces of R's may have the same structure or different structures.

Specific examples of the group represented by R¹ are shown below, but the present invention is not limited thereto. In the following formulae, * represents a bonding site to X¹ or Y¹ in Formula (1-1).

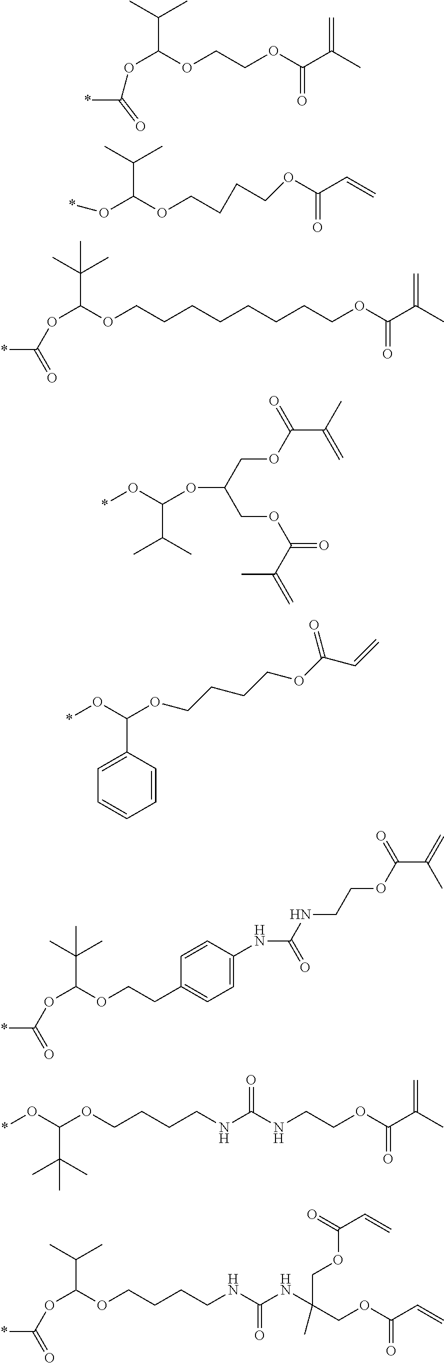

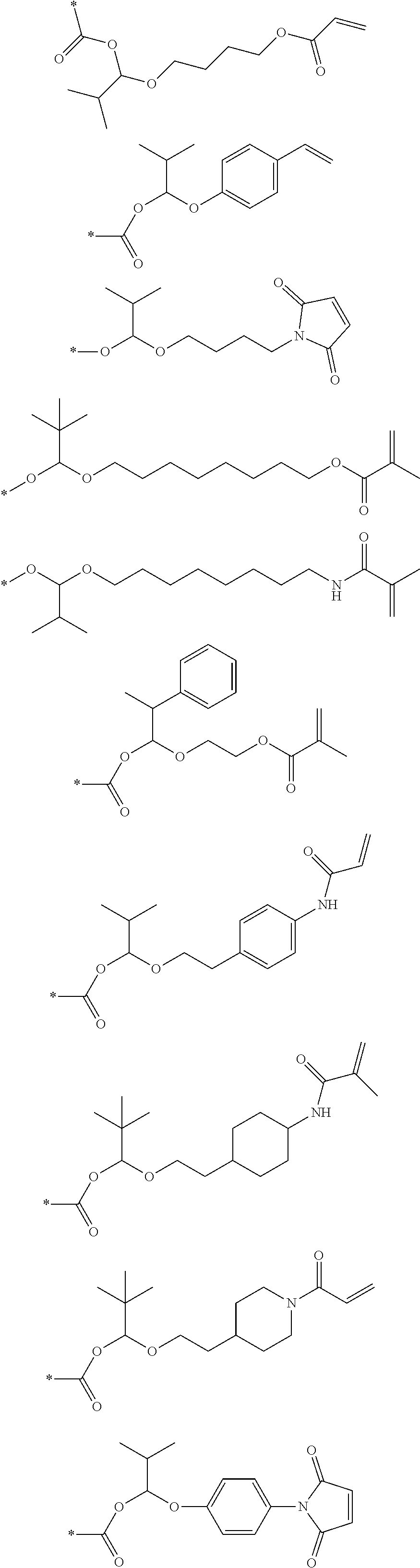

In Formula (1-1), X¹ preferably represents an organic group including a structure obtained by removing two or more hydrogen atoms from a structure represented by any of Formulae (V-1) to (V-10).

By being an organic group including a structure obtained by removing two or more hydrogen atoms from a structure represented by any of Formula (V-1) to Formula (V-10), chemical resistance and flatness of the cured substance are improved.

By the fact that X¹ is an organic group including a structure in which two or more hydrogen atoms are removed from a structure represented by any one of Formulae (V-1) to (V-5), the chemical resistance and the flatness of the cured substance are improved.

In addition, by the fact that X¹ is an organic group including a structure in which two or more hydrogen atoms are removed from a structure represented by any one of Formulae (V-1) to (V-5), the effect of suppressing the generation of development residues, the effect of reducing the dielectric constant of the cured substance, the effect of reducing the coefficient of thermal expansion, and the like are also obtained.

Since the organic group is an organic group including a structure obtained by removing two or more hydrogen atoms from a structure represented by any of Formula (V-6) to Formula (V-10), effects such as that the pattern of the cured substance is less likely to be tapered due to improvement in the transmittance of ultraviolet light and that the tolerance to the exposure amount is wide can also be obtained.

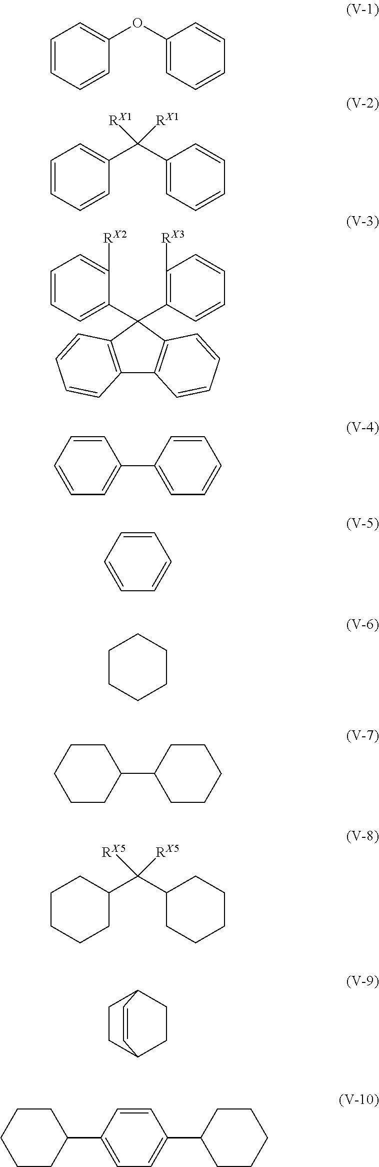

In Formula (V-2), R^{X1}'s each independently represent a hydrogen atom, an alkyl group, or a halogenated alkyl group.

In Formula (V-3), R^{X2} and R^{X3} each independently represent a hydrogen atom or a substituent, and R^{X2} and R^{X3} may be bonded to each other to form a ring structure.

In Formula (V-8), R^{X5}'s each independently represent a hydrogen atom, an alkyl group, or a halogenated alkyl group.

In Formula (V-2), R^{X1}'s are each independently preferably an alkyl group or a halogenated alkyl group, more preferably an alkyl group having 1 to 4 carbon atoms or a halogenated alkyl group having 1 to 4 carbon atoms, and still more preferably a methyl group or a trifluoromethyl group. The halogenated alkyl group refers to a group in which at least one hydrogen atom of an alkyl group is substituted with a halogen atom. As the halogen atom, F or Cl is preferable, and F is more preferable.

In Formula (V-3), R^{X2} and R^{X3} are each independently preferably a hydrogen atom.

In a case where R^{X2} and R^{X3} are bonded to each other to form a ring structure, the structure formed by the bonding of R^{X2} and R^{X3} is preferably a single bond, —O—, or —C(R)₂—, more preferably —O— or —C(R)₂—, and still more preferably —O—. R represents a hydrogen atom or a monovalent organic group, preferably a hydrogen atom, an alkyl group, or an aryl group, and more preferably a hydrogen atom.

In Formula (V-8), R^{X5}'s are each independently preferably an alkyl group or a halogenated alkyl group, more preferably an alkyl group having 1 to 4 carbon atoms or a halogenated alkyl group having 1 to 4 carbon atoms, and still more preferably a methyl group or a trifluoromethyl group. The halogenated alkyl group refers to a group in which at least one hydrogen atom of an alkyl group is substituted with a halogen atom. As the halogen atom, F or Cl is preferable, and F is more preferable.

In a case where X¹ is a group including a structure obtained by removing two or more hydrogen atoms from a structure represented by Formula (V-1), X¹ is preferably a group represented by Formula (V-1-1). In the following Formula, * represents a bonding site to four carbonyl groups to which X¹ in Formula (1-1) is bonded, and n1 represents an integer of 0 to 5, and it is also preferable that n1 is an integer of 1 to 5. In addition, the hydrogen atom in the following structure may be further substituted with a known substituent such as R¹ in Formula (1-1) or a hydrocarbon group.

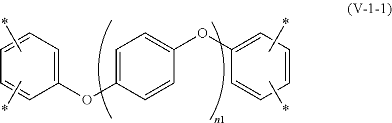

In a case where X¹ is a group including a structure obtained by removing two or more hydrogen atoms from a structure represented by Formula (V-2), X¹ is preferably a group represented by Formula (V-2-1) or Formula (V-2-2), and from the viewpoint of reducing the amine value in the resin, X¹ is preferably a group represented by Formula (V-2-2). In the present specification, a bond that intersects with a side of a ring structure means that any hydrogen atom in the ring structure is substituted. In the following Formula, L^{X1} represents a single bond or —O—, and * represents a bonding site to four carbonyl groups to which X¹ in Formula (1-1) is bonded. In addition, the definition and preferred aspect of R^{X1} are as described above. In addition, the hydrogen atom in these structures may be further substituted with a known substituent such as R¹ in Formula (1-1) or a hydrocarbon group.

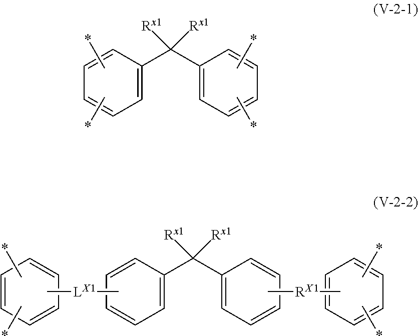

In a case where X¹ is a group including a structure obtained by removing two or more hydrogen atoms from a structure represented by Formula (V-3), X¹ is preferably a group represented by Formula (V-3-1) or Formula (V-3-2), and from the viewpoint of reducing the dielectric constant of the cured substance or the like, X¹ is preferably a group represented by Formula (V-3-2). In the following Formula, * represents a bonding site to four carbonyl groups to which X¹ in Formula (1-1) is bonded. In addition, the definitions and preferred aspects of R^{X2} and R^{X3} are as described above. In addition, the hydrogen atom in these structures may be further substituted with a known substituent such as R¹ in Formula (1-1) or a hydrocarbon group.

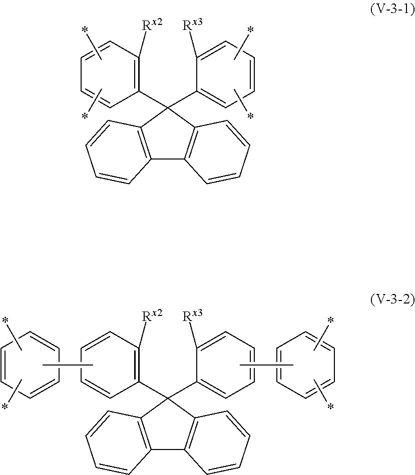

In a case where X¹ is a group including a structure obtained by removing two or more hydrogen atoms from a structure represented by Formula (V-4), X¹ is preferably a group represented by Formula (V-4-1).

In Formula (V-4-1), * represents a bonding site to four carbonyl groups to which X¹ in Formula (1-1) is bonded, and n1 represents an integer of 0 to 5. In addition, the hydrogen atom in Formula (V-4-1) may be further substituted with a known substituent such as R¹ in Formula (1-1) or a hydrocarbon group. Examples of the known substituent include an alkyl group, a halogenated alkyl group, and a halogen atom. However, it is also preferable that none of the hydrogen atoms in the structure represented by Formula (V-4-1) are substituted.

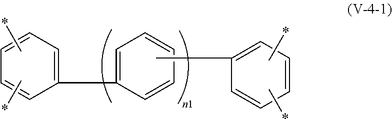

In a case where X¹ is a group including a structure obtained by removing two or more hydrogen atoms from a structure represented by Formula (V-5), X¹ is preferably a group represented by Formula (V-5-1). In the following Formula, * represents a bonding site to four carbonyl groups to which X¹ in Formula (1-1) is bonded. In addition, the hydrogen atom in Formula (V-5-1) may be further substituted with a known substituent such as R¹ in Formula (1-1) or a hydrocarbon group. Examples of the known substituent include an alkyl group, a halogenated alkyl group, and a halogen atom. However, it is also preferable that none of the hydrogen atoms in the structure represented by (V-5-1) are substituted.

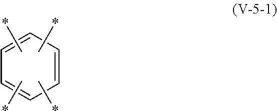

In a case where X¹ is a group including a structure obtained by removing two or more hydrogen atoms from a structure represented by Formula (V-6), X¹ is preferably a group represented by Formula (V-6-1). In the following Formula, * represents a bonding site to four carbonyl groups to which X¹ in Formula (1-1) is bonded. In addition, the hydrogen atom in the following structure may be further substituted with a known substituent such as R¹ in Formula (1-1) or a hydrocarbon group.

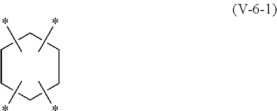

In a case where X¹ is a group including a structure obtained by removing two or more hydrogen atoms from a structure represented by Formula (V-7), X¹ is preferably a group represented by Formula (V-7-1). In the following Formula, * represents a bonding site to four carbonyl groups to which X¹ in Formula (1-1) is bonded. In addition, the hydrogen atom in the following structure may be further substituted with a known substituent such as R¹ in Formula (1-1) or a hydrocarbon group.

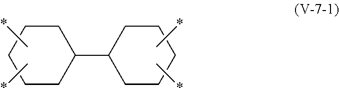

In a case where X¹ is a group including a structure obtained by removing two or more hydrogen atoms from a structure represented by Formula (V-8), X¹ is preferably a group represented by Formula (V-8-1). In the following Formula, * represents a bonding site to four carbonyl groups to which X¹ in Formula (1-1) is bonded. The definition and the preferred aspect of R^{X5} are as described above. In addition, the hydrogen atom in the following structure may be further substituted with a known substituent such as R¹ in Formula (1-1) or a hydrocarbon group.

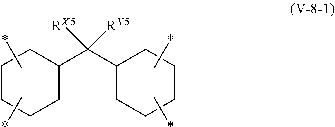

In a case where X¹ is a group including a structure obtained by removing two or more hydrogen atoms from a structure represented by Formula (V-9), X¹ is preferably a group represented by Formula (V-9-1). In the following Formula, * represents a bonding site to four carbonyl groups to which X¹ in Formula (1-1) is bonded. In addition, the hydrogen atom in the following structure may be further substituted with a known substituent such as R¹ in Formula (1-1) or a hydrocarbon group.

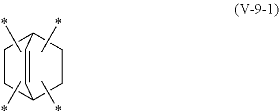

In a case where X¹ is a group including a structure obtained by removing two or more hydrogen atoms from a structure represented by Formula (V-10), X¹ is preferably a group represented by Formula (V-10-1). In the following Formula, * represents a bonding site to four carbonyl groups to which X¹ in Formula (1-1) is bonded. In addition, the hydrogen atom in the following structure may be further substituted with a known substituent such as R¹ in Formula (1-1) or a hydrocarbon group.

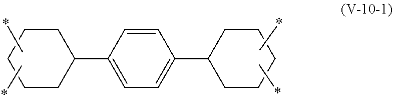

In addition, X¹ may be a tetracarboxylic acid residue remaining after the removal of the anhydride group from the tetracarboxylic acid dianhydride described in paragraphs 0055 to 0057 of JP2023-003421A.

In addition, it is preferable that X¹ does not contain an imide bond in the structure.

In addition, it is preferable that X¹ does not contain a urethane bond, a urea bond, and an amide bond in the structure.

In the present invention, the urethane bond is a bond represented by *—O—C(═O)—NR^{N}—*, where R^{N} represents a hydrogen atom or a monovalent organic group, and *'s each represent a bonding site to a carbon atom. R^{N} is preferably a hydrogen atom or a hydrocarbon group, more preferably a hydrogen atom or an alkyl group, and still more preferably a hydrogen atom.

In the present invention, the urea bond is a bond represented by *—NR^{N}—C(═O)—NR^{N}—*, where R^{N}'s each independently represent a hydrogen atom or a monovalent organic group, and *'s each represent a bonding site to a carbon atom. The preferred aspect of R^{N} is as described above.

Furthermore, it is preferable that X¹ does not contain an ester bond in the structure.

In the present invention, the ester bond is a bond represented by *—O—C(═O)—*.

Among these, it is preferable that X¹ does not contain an imide bond, a urethane bond, a urea bond, and an amide bond, and it is preferable that X¹ does not contain an imide bond, a urethane bond, a urea bond, an amide bond, and an ester bond.

In addition, X¹ may be a structure represented by Formula (X-2) or a structure in which a hydrogen atom of a group represented by X² or a hydrogen atom of a group represented by L³ in the structure represented by Formula (X-2) is substituted with a group represented by R¹ in Formula (1-1).

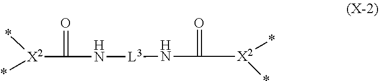

In Formula (X-2), X²'s each independently represent a trivalent linking group, L³ represents a divalent linking group, and * represents a bonding site to another structure.

In Formula (X-2), examples of X² include a linear or branched aliphatic group, a cyclic aliphatic group, an aromatic group, and a group obtained by linking two or more of these groups through a single bond or a linking group, where X² is preferably a linear aliphatic group having 2 to 20 carbon atoms, a branched aliphatic group having 3 to 20 carbon atoms, a cyclic aliphatic group having 3 to 20 carbon atoms, an aromatic group having 6 to 20 carbon atoms, or a group obtained by combining two or more of these groups through a single bond or a linking group, and X² is more preferably an aromatic group having 6 to 20 carbon atoms or a group obtained by combining two or more aromatic groups having 6 to 20 carbon atoms through a single bond or a linking group.

The linking group is preferably —O—, —S—, —C(═O)—, —S(═O)₂—, an alkylene group, a halogenated alkylene group, an arylene group, or a linking group obtained by bonding two or more these, and it is more preferably —O—, —S—, an alkylene group, a halogenated alkylene group, an arylene group, or a linking group obtained by bonding two or more these.

The alkylene group is preferably an alkylene group having 1 to 20 carbon atoms, more preferably an alkylene group having 1 to 10 carbon atoms, and still more preferably an alkylene group having 1 to 4 carbon atoms.

The halogenated alkylene group is preferably a halogenated alkylene group having 1 to 20 carbon atoms, more preferably a halogenated alkylene group having 1 to 10 carbon atoms, and still more preferably a halogenated alkylene group having 1 to 4 carbon atoms. In addition, examples of the halogen atom in the halogenated alkylene group include a fluorine atom, a chlorine atom, a bromine atom, and an iodine atom, where a fluorine atom is preferable. The halogenated alkylene group may have a hydrogen atom, or all hydrogen atoms in the halogenated alkylene group may be substituted with a halogen atom. However, it is preferable that all hydrogen atoms are substituted with a halogen atom. Examples of the preferred halogenated alkylene group include a di(trifluoromethyl)methylene group.

The arylene group is preferably a phenylene group or a naphthylene group, more preferably a phenylene group, and still more preferably a 1,3-phenylene group or a 1,4-phenylene group.

In addition, X² is preferably derived from a tricarboxylic acid compound in which at least one carboxy group may be subjected to halogenation. Here, the halogenation is preferably chlorination.

In the present invention, a compound having three carboxy groups is referred to as a tricarboxylic acid compound.

Two carboxy groups of the three carboxy groups of the tricarboxylic acid compound may be subjected to acid anhydrization.

Examples of the tricarboxylic acid compound which may be halogenated include a branched aliphatic, cyclic aliphatic, or aromatic tricarboxylic acid compound.

One kind of these tricarboxylic acid compounds may be used alone, or two or more kinds thereof may be used.

In addition, it is preferable that X² does not contain an imide structure in the structure.

In addition, it is preferable that X² does not contain a urethane bond, a urea bond, and an amide bond in the structure.

Furthermore, it is preferable that X² does not contain an ester bond in the structure.

Among these, it is preferable that X² does not contain an imide structure, a urethane bond, a urea bond, and an amide bond, and it is preferable that X² does not contain an imide structure, a urethane bond, a urea bond, an amide bond, and an ester bond.

Specifically, the tricarboxylic acid compound is preferably a tricarboxylic acid compound containing a linear aliphatic group having 2 to 20 carbon atoms, a branched aliphatic group having 3 to 20 carbon atoms, a cyclic aliphatic group having 3 to 20 carbon atoms, an aromatic group having 6 to 20 carbon atoms, or a group obtained by combining two or more of these groups through a single bond or a linking group, and more preferably a tricarboxylic acid compound containing an aromatic group having 6 to 20 carbon atoms or a group obtained by combining two or more aromatic groups having 6 to 20 carbon atoms through a single bond or a linking group.

Specific examples of the tricarboxylic acid compound include 1,2,3-propanetricarboxylic acid, 1,3,5-pentanetricarboxylic acid, citric acid, trimellitic acid, 2,3,6-naphthalenetricarboxylic acid, and a compound in which phthalic acid (or phthalic anhydride) and benzoic acid are linked through a single bond, —O—, —CH₂—, —C(CH₃)₂—, —C(CF₃)₂—, —SO₂—, or a phenylene group.

These compounds may be a compound (for example, a trimellitic acid anhydride) in which two carboxy groups have been subjected to anhydrization or may be a compound (for example, trimellitic anhydride chloride) in which at least one carboxy group has been halogenated.

In Formula (X-2), examples of L³ include a linear or branched aliphatic group, a cyclic aliphatic group, an aromatic group, and a group obtained by linking two or more of these groups through a single bond or a linking group, where L³ is preferably a linear aliphatic group having 2 to 20 carbon atoms, a branched aliphatic group having 3 to 20 carbon atoms, a cyclic aliphatic group having 3 to 20 carbon atoms, an aromatic group having 6 to 20 carbon atoms, or a group obtained by combining two or more of these groups through a single bond or a linking group, and L³ is more preferably an aromatic group having 6 to 20 carbon atoms or a group obtained by combining two or more aromatic groups having 6 to 20 carbon atoms through a single bond or a linking group.

The linking group is preferably —O—, —S—, —C(═O)—, —S(═O)₂—, an alkylene group, a halogenated alkylene group, an arylene group, or a linking group obtained by bonding two or more these, and it is more preferably —O—, —S—, an alkylene group, a halogenated alkylene group, an arylene group, or a linking group obtained by bonding two or more these.

The alkylene group is preferably an alkylene group having 1 to 20 carbon atoms, more preferably an alkylene group having 1 to 10 carbon atoms, and still more preferably an alkylene group having 1 to 4 carbon atoms.

The halogenated alkylene group is preferably a halogenated alkylene group having 1 to 20 carbon atoms, more preferably a halogenated alkylene group having 1 to 10 carbon atoms, and still more preferably a halogenated alkylene group having 1 to 4 carbon atoms. In addition, examples of the halogen atom in the halogenated alkylene group include a fluorine atom, a chlorine atom, a bromine atom, and an iodine atom, where a fluorine atom is preferable. The halogenated alkylene group may have a hydrogen atom, or all hydrogen atoms in the halogenated alkylene group may be substituted with a halogen atom. However, it is preferable that all hydrogen atoms are substituted with a halogen atom. Examples of the preferred halogenated alkylene group include a di(trifluoromethyl)methylene group.

The arylene group is preferably a phenylene group or a naphthylene group, more preferably a phenylene group, and still more preferably a 1,3-phenylene group or a 1,4-phenylene group.

In addition, X¹ may be a structure represented by Formula (X-3) or a structure in which a hydrogen atom of a group represented by X² or a hydrogen atom of a group represented by L³ in the structure represented by (X-3) is substituted with a group represented by R¹ in Formula (1-1).

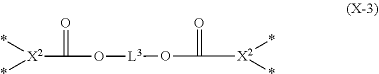

In Formula (X-3), X²'s each independently represent a trivalent linking group, L³ represents a divalent linking group, and * represents a bonding site to another structure.

In Formula (X-3), preferred aspects of X² and L³ are the same as the preferred aspects of X² and L³ in Formula (X-2).

### —Y¹—

The number of carbon atoms in Y¹ is 4 or more, and is preferably 4 to 50 and more preferably 4 to 40.

In Formula (1-1), Y¹ may be a group including a structure obtained by removing two or more hydrogen atoms from the structure represented by any of Formula (V-1) to Formula (V-10) described above.

By being an organic group including a structure obtained by removing two or more hydrogen atoms from a structure represented by any of Formula (V-1) to Formula (V-10), chemical resistance and flatness of the cured substance are improved.

In a case where Y¹ is a group including a structure obtained by removing two or more hydrogen atoms from a structure represented by Formula (V-1), Y¹ is preferably a group represented by Formula (V-1-2). In the following Formula, * represents a bonding site to two nitrogen atoms to which Y¹ in Formula (1-1) is bonded, and n1 represents an integer of 1 to 5.

In addition, the hydrogen atom in the following structure may be further substituted with a known substituent such as R¹ in Formula (1-1) or a hydrocarbon group.

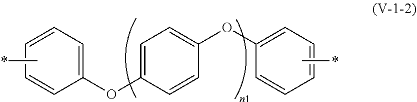

In a case where Y¹ is a group including a structure obtained by removing two or more hydrogen atoms from a structure represented by Formula (V-2), Y¹ is preferably a group represented by Formula (V-2-3) or Formula (V-2-4), and from the viewpoint of reducing the dielectric constant of the cured substance or the like, Y¹ is preferably a group represented by Formula (V-2-4). In the following Formula, L^{X1} represents a single bond or —O—, and * represents a bonding site to two nitrogen atoms to which Y¹ in Formula (1-1) is bonded. In addition, the preferred aspect of R^{X1} is as described above. In addition, the hydrogen atom in these structures may be further substituted with a known substituent such as R¹ in Formula (1-1) or a hydrocarbon group.

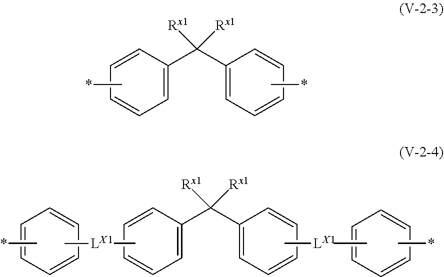

In a case where Y¹ is a group including a structure obtained by removing two or more hydrogen atoms from a structure represented by Formula (V-3), Y¹ is preferably a group represented by Formula (V-3-3) or Formula (V-3-4), and from the viewpoint of reducing the dielectric constant of the cured substance or the like, Y¹ is preferably a group represented by Formula (V-3-3). In the following Formula, * represents a bonding site to two nitrogen atoms to which Y¹ in Formula (1-1) is bonded. In addition, the hydrogen atom in these structures may be further substituted with a known substituent such as R¹ in Formula (1-1) or a hydrocarbon group.

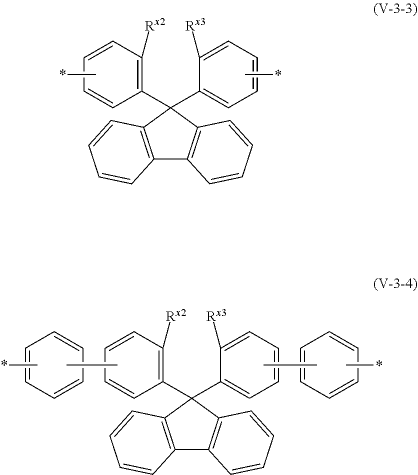

In a case where Y¹ is a group including a structure in which two or more hydrogen atoms are removed from a structure represented by Formula (V-4), Y¹ is preferably a group represented by Formula (V-4-2) or (V-4-3). In the following Formula, * represents a bonding site to two nitrogen atoms to which Y¹ in Formula (1-1) is bonded, and n1 represents an integer of 0 to 5. In addition, an aspect in which n1 is 0 is also one of the preferred aspects of the present invention. In addition, the hydrogen atom in the following structure may be further substituted with a known substituent such as R¹ in Formula (1-1) or a hydrocarbon group. Examples of the known substituent include an alkyl group, a halogenated alkyl group, and a halogen atom.

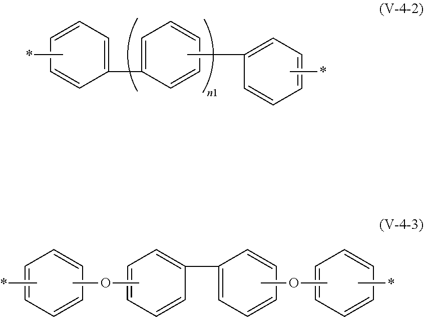

In a case where Y¹ is a group including a structure obtained by removing two or more hydrogen atoms from a structure represented by Formula (V-5), Y¹ is preferably a group represented by Formula (V-5-2). In the following Formula, * represents a bonding site to two nitrogen atoms to which Y¹ in Formula (1-1) is bonded. In addition, the hydrogen atom in Formula (V-5-2) may be further substituted with a known substituent such as R¹ in Formula (1-1) or a hydrocarbon group. Examples of the known substituent include an alkyl group, a halogenated alkyl group, and a halogen atom. However, it is also preferable that none of the hydrogen atoms in the structure represented by (V-5-1) are substituted.

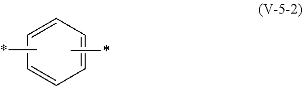

In a case where Y¹ is a group including a structure obtained by removing two or more hydrogen atoms from a structure represented by Formula (V-6), Y¹ is preferably a group represented by Formula (V-6-2). In the following Formula, * represents a bonding site to two nitrogen atoms to which Y¹ in Formula (1-1) is bonded. In addition, the hydrogen atom in the following structure may be further substituted with a known substituent such as R¹ in Formula (1-1) or a hydrocarbon group.

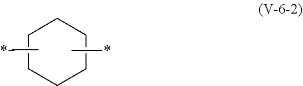

In a case where Y¹ is a group including a structure obtained by removing two or more hydrogen atoms from a structure represented by Formula (V-7), Y¹ is preferably a group represented by Formula (V-7-2). In the following Formula, * represents a bonding site to two nitrogen atoms to which Y¹ in Formula (1-1) is bonded. In addition, the hydrogen atom in the following structure may be further substituted with a known substituent such as R¹ in Formula (1-1) or a hydrocarbon group.

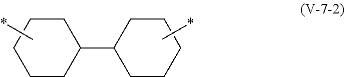

In a case where Y¹ is a group including a structure obtained by removing two or more hydrogen atoms from a structure represented by Formula (V-8), Y¹ is preferably a group represented by Formula (V-8-2). In the following Formula, * represents a bonding site to two nitrogen atoms to which Y¹ in Formula (1-1) is bonded. In addition, the hydrogen atom in the following structure may be further substituted with a known substituent such as R¹ in Formula (1-1) or a hydrocarbon group.

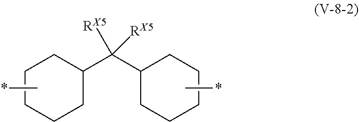

In a case where Y¹ is a group including a structure obtained by removing two or more hydrogen atoms from a structure represented by Formula (V-9), Y¹ is preferably a group represented by Formula (V-9-2). In the following Formula, * represents a bonding site to two nitrogen atoms to which Y¹ in Formula (1-1) is bonded. In addition, the hydrogen atom in the following structure may be further substituted with a known substituent such as R¹ in Formula (1-1) or a hydrocarbon group.

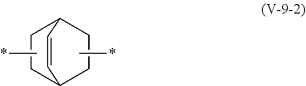

In a case where Y¹ is a group including a structure obtained by removing two or more hydrogen atoms from a structure represented by Formula (V-10), Y¹ is preferably a group represented by Formula (V-10-2). In the following Formula, * represents a bonding site to two nitrogen atoms to which Y¹ in Formula (1-1) is bonded. In addition, the hydrogen atom in the following structure may be further substituted with a known substituent such as R¹ in Formula (1-1) or a hydrocarbon group.

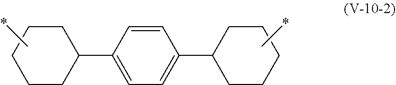

In addition, Y¹ may be a group described in paragraphs 0042 to 0053 of JP2023-003421A.

In addition, it is preferable that Y¹ does not contain an imide bond in the structure.

In addition, it is preferable that Y¹ does not contain a urethane bond, a urea bond, and an amide bond in the structure.

Furthermore, it is preferable that Y¹ does not contain an ester bond in the structure.

Among these, it is preferable that Y¹ does not contain an imide bond, a urethane bond, a urea bond, and an amide bond, and it is preferable that Y¹ does not contain an imide bond, a urethane bond, a urea bond, an amide bond, and an ester bond.

Among these, it is preferable that both X¹ and Y¹ in Formula (1-1) include a ring structure, it is more preferable that both X¹ and Y¹ are organic groups including a structure in which two or more hydrogen atoms are removed from a structure represented by any of Formulae (V-1) to (V-10), and it is still more preferable that both X¹ and Y¹ are organic groups including a structure in which two or more hydrogen atoms are removed from a structure represented by any of Formulae (V-1) to (V-5). Preferred aspects of these groups are as described above.

—n and m—

In Formula (1-1), m is preferably an integer of 0 to 2, and more preferably 0 or 1. In addition, an aspect in which m is 0 is also one of the preferred aspects of the present invention.

In Formula (1-1), n is preferably 1 or 2, and more preferably 2.

### [Repeating Unit Represented by Formula (4)]

The first specific resin may further include a repeating unit represented by Formula (4).

The repeating unit corresponding to the repeating unit represented by Formula (1-1) does not correspond to the repeating unit represented by Formula (4).

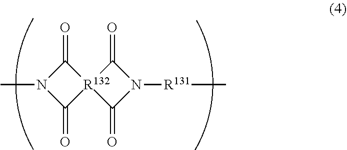

in Formula (4), R¹³¹ represents a divalent organic group, and R¹³² represents a tetravalent organic group.

R¹³¹ represents a divalent organic group. Examples of R¹³¹ include groups described in paragraphs 0042 to 0053 of JP2023-003421A. These descriptions are incorporated into the present specification.

R¹³² represents a tetravalent organic group. Examples of R¹³² include the compounds described in paragraphs 0055 to 0057 of JP2023-003421A. These descriptions are incorporated into the present specification.

In addition, it is preferable that R¹³¹ and R¹³² are organic groups including a structure in which two or more hydrogen atoms are removed from a structure represented by any of Formulae (V-1) to (V-10), and it is more preferable that R¹³¹ and R¹³² are organic groups including a structure in which two or more hydrogen atoms are removed from a structure represented by any of Formulae (V-1) to (V-5). Preferred aspects of these groups are as described above. However, the description that R¹ in Formula (1-1) may be a substituent is to be read as a description that a structure represented by Formula (R-2) may be a substituent.

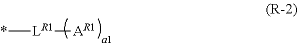

In Formula (R-2), L^{R1} represents a (a1+1)-valent linking group, A^{R1} represents a polymerizable group, a1 represents an integer of 1 or more, and * represents a bonding site to another structure. However, the structure corresponding to Formula (R-1) does not correspond to the structure represented by Formula (R-2).

In Formula (R-2), L^{R1} is preferably a group represented by Formula (L-2).

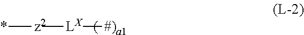

In Formula (L-2), Z² represents —O—, —NR^{N}—, —C(═O)O—, or —C(═O)NR^{N}—, R^{N} represents a hydrogen atom or a monovalent organic group, in a case where a1 is 1, L^{x} represents a single bond or a divalent linking group, in a case where a1 is 2 or more, L^{x} represents a (a1+1)-valent linking group, a1 represents an integer of 1 or more, * represents a bonding site to Y¹ in Formula (2-1), and # represents a bonding site to A^{R1} in Formula (R-2).

In Formula (L-2), Z² is preferably —O— or —C(═O)O—. In addition, in a case where Z² is —NR^{N}—, R^{N} is preferably a hydrogen atom or a hydrocarbon group, more preferably a hydrogen atom, an alkyl group, or a phenyl group, and still more preferably a hydrogen atom.

In Formula (L-2), in a case where a1 is 1, L^{x} is preferably an alkylene group, more preferably an alkylene group having 1 to 10 carbon atoms, still more preferably an alkylene group having 1 to 4 carbon atoms, and particularly preferably a methylene group.

In Formula (L-2), in a case where a1 is 2 or more, L^{x} is preferably a group represented by a hydrocarbon group, a heterocyclic group, or a combination thereof, more preferably a saturated aliphatic hydrocarbon group having 2 to 20 carbon atoms, and still more preferably a saturated aliphatic hydrocarbon group having 3 to 15 carbon atoms.

In Formula (L-2), a1 has the same meaning as a1 in Formula (R-2).

In Formula (R-2), a preferred aspect of the polymerizable group in A^{R1} is the same as the preferred aspect of the polymerizable group in A¹ in Formula (R-1).

Here, A^{R1} is preferably a maleimide group, a (meth)acryloxy group, a (meth)acrylamide group, or a vinylphenyl group, and from the viewpoint of reactivity, it is preferably a (meth)acryloxy group. In addition, from the viewpoint of reducing the dielectric loss tangent, a maleimide group or a vinylphenyl group is preferable.

Among these, it is preferable that A^{R1} in Formula (R-2) is a vinylphenyl group and L^{R1} is a group represented by Formula (L-2-1).

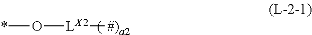

In Formula (L-2-1), L^{X2} represents a hydrocarbon group, and a1 represents an integer of 1 or more.

In Formula (L-2-1), L^{X2} represents a hydrocarbon group, and it is preferably an aliphatic saturated hydrocarbon group.

In a case where a1 is 1, L^{X2} is preferably an alkylene group, more preferably an alkylene group having 1 to 10 carbon atoms, still more preferably an alkylene group having 1 to 4 carbon atoms, and particularly preferably a methylene group.

In Formula (L-2-1), a1 has the same meaning as a1 in Formula (R-2).

In addition, it is preferable that A^{R1} in Formula (R-2) is a maleimide group, L^{R1} is a group represented by Formula (L-2), and L^{X} is an aromatic group or an aliphatic saturated hydrocarbon group having 4 or more carbon atoms.

The aromatic group may be any of an aromatic hydrocarbon group or an aromatic heterocyclic group, but is preferably an aromatic hydrocarbon group.

The aromatic hydrocarbon group is preferably an aromatic hydrocarbon group having 6 to 10 carbon atoms, and more preferably an aromatic hydrocarbon group having 6 carbon atoms.

Examples of the heteroatom in the aromatic heterocyclic group include an oxygen atom, a nitrogen atom, and a sulfur atom. The number of heteroatoms in the aromatic heterocyclic group is preferably 1 or 2. In addition, as the aromatic heterocyclic group, a 5-membered ring or a 6-membered ring including the above-described heteroatom is preferable. Furthermore, the aromatic heterocyclic group may be fused with another aromatic heterocyclic group or another aromatic hydrocarbon ring group.

The aliphatic saturated hydrocarbon group having 4 or more carbon atoms may have any of a linear structure, a branched structure, a cyclic structure, or a structure represented by a combination thereof.

The number of carbon atoms in the aliphatic saturated hydrocarbon group having 4 or more carbon atoms is preferably 4 to 20 and more preferably 5 to 10.

In Formula (R-2), a1 is preferably an integer of 1 to 4 and more preferably an integer of 1 or 2. In addition, an aspect in which a1 is 1 is also one of the preferred aspects of the present invention.

In addition, the number of ester groups included in Formula (R-2) is preferably 1 or 0.

The content of the repeating unit represented by Formula (1-1) is preferably 30% by mass or more, more preferably 50% by mass or more, still more preferably 70% by mass or more, and particularly preferably 80% by mass or more with respect to the total mass of the specific resin. The upper limit of the content is not particularly limited, and may be 100% by mass.

In addition, a total content of the repeating unit represented by Formula (1-1) and the repeating unit represented by Formula (4) with respect to the total mass of the specific resin is preferably 50% by mass or more, more preferably 70% by mass or more, still more preferably 80% by mass or more, and particularly preferably 90% by mass or more. The upper limit of the content is not particularly limited, and may be 100% by mass.

In addition, the specific resin may contain two or more kinds of repeating units represented by Formula (1-1) having different structures. In that case, it is preferable that the total amount is within the above-described range.

In a case where the specific resin includes a repeating unit represented by Formula (4), the third specific resin may include two or more kinds of repeating units represented by Formula (4) having different structures. In that case, it is preferable that the total amount is within the above-described range.

### [Second Specific Resin]

The second resin composition contains a polyimide (second specific resin) from which a structure having 2 or more carbon atoms is eliminated by heating at 230° C. for 3 hours.

The first specific resin is preferably a polyimide from which a structure having 2 or more carbon atoms is eliminated by heating at 230° C. for 3 hours.

The temperature at which the above-described decomposition occurs is preferably 220° C., more preferably 200° C., and still more preferably 170° C.

Here, it is preferable that the structure of Formula (R-1) is eliminated from the specific resin by 10 mol % or more, more preferably 40 mol % or more, and still more preferably 60 mol % or more by heating at 230° C. for 3 hours. The upper limit of the above-described molar amount is not particularly limited, and may be, for example, 100 mol % or less.

The number of carbon atoms in the structure to be eliminated is preferably 5 or more, more preferably 8 or more. The upper limit thereof is not particularly limited, but is preferably 50 or less and more preferably 30 or less.

The structure to be eliminated preferably includes a polymerizable group.

A preferred aspect of the polymerizable group is the same as the preferred aspect of the polymerizable group in A¹ in Formula (R-1).

The structure to be eliminated is preferably the structure represented by Formula (R-1).

A preferred aspect of the structure represented by Formula (R-1) is as described above.

The structure formed in the specific resin after the above-described structure is eliminated is preferably a carboxy group or a hydroxy group.

The weight-average molecular weight (Mw) of the specific resin is preferably 3,000 to 100,000.

The lower limit of the above-described Mw is preferably 5,000 or more, more preferably 8,000 or more, and still more preferably 10,000 or more.

The upper limit of the Mw is preferably 100,000 or less, more preferably 50,000 or less, and still more preferably 30,000 or less.

In a case where the weight-average molecular weight is set to 3,000 or more, it is possible to improve the breakage resistance of the film after curing. In order to obtain an organic film having excellent mechanical properties (for example, breaking elongation), the weight-average molecular weight is particularly preferably 5,000 or more.

A number-average molecular weight (Mn) of the specific resin is preferably 1,000 to 40,000, more preferably 2,000 to 30,000, and still more preferably 5,000 to 20,000.

The dispersity of the molecular weight of the specific resin is preferably 1.5 or more, more preferably 1.8 or more, and still more preferably 2.0 or more. The upper limit value of the dispersity of the molecular weight of the polyimide is not particularly specified, but is, for example, preferably 7.0 or less, more preferably 6.5 or less, still more preferably 6.0 or less, even more preferably 4.5 or less, and particularly preferably 3.0 or less.

In the present specification, the dispersity of the molecular weight is a value obtained by calculating “weight-average molecular weight/number-average molecular weight”.

In a case where the resin composition contains a plurality of kinds of polyimides as the specific resin, it is preferable that the weight-average molecular weight, the number-average molecular weight, and the dispersity of at least one kind of resin are in the above ranges. It is also preferable that the weight-average molecular weight, the number-average molecular weight, and the dispersity, calculated by using the plurality of kinds of resins as one resin, are within the above ranges.

The imidization rate (also referred to as the “ring closure rate”) of the specific resin is preferably 70% or more, more preferably 80% or more, and still more preferably 90% or more, from the viewpoint of the film hardness, the insulating properties, or the like of the organic film to be obtained. The upper limit of the imidization rate is not particularly limited, and it may be any imidization rate of 100% or less.

In addition, a content of the imide structure in the specific resin is preferably 3 mmol/g or less, and more preferably 2.5 mmol/g or less. The lower limit of the above-described content is not particularly limited, but can be, for example, 0.5 mmol/g or more.

The imidization rate is measured by, for example, the following method.

The infrared absorption spectrum of the specific resin is measured, and a peak intensity P1 in the vicinity of 1,377 cm⁻¹, which is the absorption peak derived from the imide structure, is obtained. Next, the specific resin is subjected to a heat treatment at 350° C. for 1 hour, and then the infrared absorption spectrum is measured again to obtain a peak intensity P2 in the vicinity of 1,377 cm⁻¹. Using the obtained peak intensities P1 and P2, the imidization rate of the specific resin can be determined based on the following expression.

Imidization rate (%)=(peak intensity P1/peak intensity P2)×100

### [Aliphatic Hydrocarbon Group]

From the viewpoint of reducing the moisture content of the cured substance and reducing the dielectric loss tangent, the specific resin preferably has a chain-like alkyl group having 6 or more carbon atoms (hereinafter, also referred to as a specific substituent A).

It is preferable that the specific resin has the specific substituent A at the main chain terminal.

It is preferable that the hydrogen atom in the specific substituent A is unsubstituted or substituted with a halogen atom. As the halogen atom, a fluorine atom is preferable. In addition, an aspect in which the hydrogen atom in the specific substituent A is unsubstituted is also one of the preferred aspects of the present invention.

The number of carbon atoms in the specific substituent A is preferably 6 to 30 and more preferably 6 to 20.

### [Manufacturing Method for Specific Resin]

The manufacturing method for a specific resin according to the embodiment of the present invention includes a step of reacting a polyimide having an acid group or a precursor thereof with a compound represented by Formula (B-1).

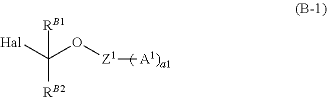

In Formula (B-1), Z¹ represents a (1+a1)-valent linking group, A¹ represents a polymerizable group, a1 represents an integer of 1 or more and a maximum substituent number of Z¹ or less, R^{B1} and R^{B2} each independently represent a hydrogen atom or a monovalent organic group, and Hal represents a halogen atom.

As the acid group in the polyimide having an acid group or a precursor thereof, a carboxy group or a phenolic hydroxyl group is preferable.

In Formula (B-1), preferred aspects of Z¹, A¹, and a1 are the same as the preferred aspects of Z¹, A¹, and a1 in Formula (R-1) described above.

In Formula (B-1), preferred aspects of R^{B1} and R^{B2} are the same as the preferred aspects of R^{AC1} and R^{AC2} in Formula (Ac-1).

In Formula (B-1), Hal represents a halogen atom, and it is preferably a fluorine atom, a chlorine atom, a bromine atom, or an iodine atom, more preferably a chlorine atom or a bromine atom, and still more preferably a bromine atom.

Specific examples of the compound represented by Formula (B-1) include BC-1 to BC-17 in Examples described later, but the present invention is not limited thereto.

The compound represented by Formula (B-1) can be obtained, for example, by reacting the compound represented by Formula (A-1) with an acid halide.

The acid halide is not particularly limited, but a carboxylic acid halide is preferable, and examples thereof include acetyl chloride.

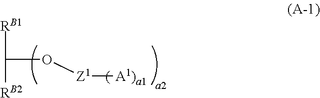

In Formula (A-1), Z¹'s each independently represent a (1+a1)-valent linking group, A¹'s each independently represent a polymerizable group, a1's each independently represent an integer of 1 or more and a maximum substituent number of Z¹ or less, R^{B1} and R^{B2} each independently represent a hydrogen atom or a monovalent organic group, and a2 represents 2.

In Formula (A-1), preferred aspects of Z¹, A¹, a1, R^{B1}, and R^{B2} are the same as the preferred aspects of Z¹, A¹, a1, R^{B1}, and R^{B2} in Formula (B-1) described above.

Specific examples of the compound represented by Formula (A-1) include AC-1 to AC-17 in Examples described later, but the present invention is not limited thereto.

The compound represented by Formula (A-1) can be obtained, for example, by reacting an aldehyde or a ketone with an alcohol having a polymerizable group in the presence of an acid catalyst.

The acid catalyst is not particularly limited, and examples thereof include methanesulfonic acid.

The manufacturing method for a specific resin preferably further includes a step of obtaining the polyimide having an acid group from an acid dianhydride and a diamine.

For example, the method for reacting a tetracarboxylic acid dianhydride and a diamine at a low temperature, the method for reacting a tetracarboxylic acid dianhydride and a diamine at a low temperature to obtain a polyamic acid and esterifying the polyamic acid using a condensing agent or an alkylating agent, the method for obtaining an ester compound thereof (for example, a diester) by reacting a tetracarboxylic acid dianhydride and an alcohol and then reacting the ester compound with a diamine in the presence of a condensing agent, the method for obtaining an ester compound thereof (for example, a diester) by reacting a tetracarboxylic acid dianhydride and an alcohol and then acid-halogenating the remaining dicarboxylic acid using a halogenating agent and reacting the acid-halogenated dicarboxylic acid with a diamine, and the like can be used. Among the above-described manufacturing methods, the method for obtaining an ester compound thereof (for example, a diester) by reacting a tetracarboxylic acid dianhydride and an alcohol and then acid-halogenating the remaining dicarboxylic acid using a halogenating agent and reacting the acid-halogenated dicarboxylic acid with a diamine is more preferable.

Examples of the condensing agent include dicyclohexylcarbodiimide, diisopropylcarbodiimide, 1-ethoxycarbonyl-2-ethoxy-1,2-dihydroquinoline, 1,1-carbonyldioxy-di-1,2,3-benzotriazole, N,N′-disuccinimidyl carbonate, and trifluoroacetic anhydride.

Examples of the alkylating agent include N,N-dimethylformamide dimethyl acetal, N,N-dimethylformamide diethyl acetal, N,N-dialkylformamide dialkyl acetal, trimethyl orthoformate, triethyl orthoformate.

Examples of the halogenating agent include thionyl chloride, oxalyl chloride, and phosphorus oxychloride.

In addition, in a case where a polyimide is desired to be obtained as the specific resin, the polyimide can be synthesized by a method for completely imidizing the resin obtained by the above method using a known imidization reaction method, a method for introducing a partial imide group by stopping the imidization reaction in the middle of the reaction, or a method for introducing a partial imide group by blending a completely imidized polymer and a polyimide precursor thereof. In addition, another known method for synthesizing polyimide can also be applied.

In the manufacturing method for the specific resin, it is preferable to use an organic solvent at the time of reaction. One kind of organic solvent may be used, or two or more kinds thereof may be used.

The organic solvent can be appropriately determined depending on the raw material; however, examples thereof include pyridine, diethylene glycol dimethyl ether (diglyme), N-methylpyrrolidone, N-ethylpyrrolidone, ethyl propionate, dimethylacetamide, dimethylformamide, tetrahydrofuran, and γ-butyrolactone.

In the manufacturing method for the specific resin, it is preferable to add a basic compound in a case of carrying out the reaction. One kind of basic compound may be used, or two or more kinds thereof may be used.

The basic compound can be appropriately determined depending on the raw material; however, examples thereof include triethylamine, diisopropylethylamine, pyridine, 1,8-diazabicyclo[5.4.0]undec-7-ene, and N,N-dimethyl-4-aminopyridine.

### —Terminal Blocking Agent—

In the manufacturing method for the specific resin, in order to further improve the storage stability, it is preferable to seal a carboxylic acid anhydride, an acid anhydride derivative, or an amino group, which remains at the resin terminal of the specific resin. In a case of blocking the carboxylic acid anhydride and the acid anhydride derivative, remaining at the resin terminal, examples of the terminal blocking agent include a monoalcohol, phenol, thiol, thiophenol, and a monoamine. It is more preferable to use a monoalcohol, phenols, or a monoamine from the viewpoint of reactivity and film stability. Examples of the preferred monoalcohol compound include primary alcohol such as methanol, ethanol, propanol, butanol, hexanol, octanol, dodecanol, benzyl alcohol, 2-phenylethanol, 2-methoxyethanol, 2-chloromethanol, and furfuryl alcohol; secondary alcohol such as isopropanol, 2-butanol, cyclohexyl alcohol, cyclopentanol, and 1-methoxy-2-propanol; and tertiary alcohol such as t-butyl alcohol and adamantane alcohol. Examples of the preferred compounds of phenols include phenols such as phenol, methoxyphenol, methylphenol, naphthalene-1-ol, naphthalene-2-ol, and hydroxystyrene. In addition, examples of the preferred monoamine compound include aniline, 2-ethynyl aniline, 3-ethynyl aniline, 4-ethynyl aniline, 5-amino-8-hydroxyquinoline, 1-hydroxy-7-aminonaphthalene, 1-hydroxy-6-aminonaphthalene, 1-hydroxy-5-aminonaphthalene, 1-hydroxy-4-aminonaphthalene, 2-hydroxy-7-aminonaphthalene, 2-hydroxy-6-aminonaphthalene, 2-hydroxy-5-aminonaphthalene, 1-carboxy-7-aminonaphthalene, 1-carboxy-6-aminonaphthalene, 1-carboxy-5-aminonaphthalene, 2-carboxy-7-aminonaphthalene, 2-carboxy-6-aminonaphthalene, 2-carboxy-5-aminonaphthalene, 2-aminobenzoic acid, 3-aminobenzoic acid, 4-aminobenzoic acid, 4-aminosalicylic acid, 5-aminosalicylic acid, 6-aminosalicylic acid, 2-aminobenzenesulfonic acid, 3-aminobenzenesulfonic acid, 4-aminobenzenesulfonic acid, 3-amino-4,6-dihydroxypyrimidine, 2-aminophenol, 3-aminophenol, 4-aminophenol, 2-aminothiophenol, 3-aminothiophenol, and 4-aminothiophenol. Two or more of these may be used, and a plurality of different terminal groups may be introduced by reacting a plurality of terminal blocking agents.

In addition, in a case of blocking the amino group at the terminal of the resin, it is possible to carry out blocking with a compound having a functional group capable of reacting with the amino group. The preferred blocking agent for the amino group is preferably a carboxylic acid anhydride, a carboxylic acid chloride, a carboxylic acid bromide, a sulfonic acid chloride, sulfonic acid anhydride, or a sulfonic acid carboxylic acid anhydride, and more preferably a carboxylic acid anhydride or a carboxylic acid chloride. Examples of the preferred carboxylic acid anhydride compound include acetic anhydride, propionic anhydride, oxalic anhydride, succinic anhydride, maleic acid anhydride, phthalic anhydride, benzoic anhydride, and 5-norbornene-2,3-dicarboxylic acid anhydride. Examples of the preferred carboxylic acid chloride compound include acetyl chloride, acrylic acid chloride, propionyl chloride, methacrylic acid chloride, pivaloyl chloride, cyclohexanecarbonyl chloride, 2-ethylhexanoyl chloride, cinnamoyl chloride, 1-adamantanecarbonyl chloride, heptafluorobutyryl chloride, stearic acid chloride, and benzoyl chloride.

### —Solid Precipitation—

In the manufacturing method for a specific resin, a step of precipitating a solid may be included. Specifically, it is possible to obtain a specific resin by filtering out a water-absorbing by-product of the dehydration condensing agent that is present together in the reaction solution as necessary, subsequently putting the obtained polymer component in a poor solvent such as water, an aliphatic lower alcohol, or a mixed solution thereof, precipitating the polymer component to be precipitated as a solid, and then carrying out drying. In order to improve the degree of purification, operations such as redissolution, reprecipitation, and drying of the specific resin may be repeated. Further, a step of removing ionic impurities using an ion exchange resin may be included.

### Specific Example

Specific examples of the specific resin include SE-1 to SE-24 in Examples described later, but the present invention is not limited thereto.

### [Content]

The content of the specific resin in the resin composition according to the embodiment of the present invention is preferably 20% by mass or more, more preferably 30% by mass or more, still more preferably 40% by mass or more, even still more preferably 50% by mass or more, and most preferably 60% by mass or more, with respect to the total solid content of the resin composition. In addition, the content of the specific resin in the resin composition according to the embodiment of the present invention is preferably 99.5% by mass or less, more preferably 99% by mass or less, still more preferably 98% by mass or less, even still more preferably 97% by mass or less, and even further still more preferably 95% by mass or less, with respect to the total solid content of the resin composition.

### <Another Resin>

The resin composition according to the embodiment of the present invention may include another resin (hereinafter, also simply referred to as “another resin”) different from the above-described specific resin.

Examples of the other resins include resins different from the specific resin and corresponding to polyimide precursors, polyimides, polybenzoxazole precursors, polybenzoxazole, polyamideimide precursors, polyamideimide, phenol resin, polyamide, epoxy resin, polysiloxane, resin including a siloxane structure, (meth)acrylic resin, (meth)acrylamide resin, urethane resin, butyral resin, styryl resin, polyether resin, and polyester resin.

Examples of the other polyimide precursors, the other polyimides, the polybenzoxazole precursors, the polybenzoxazoles, the polyamideimide precursors, and the polyamideimides include the compounds described in paragraphs 0017 to 0138 of WO2022/145355A. The above description is incorporated in the present specification.

In a case where the resin composition according to the embodiment of the present invention contains the other resin, the content of the other resin is preferably 0.01% by mass or more, more preferably 0.05% by mass or more, still more preferably 1% by mass or more, even still more preferably 2% by mass or more, even still more preferably 5% by mass or more, and even further still more preferably 10% by mass or more, with respect to the total solid content of the resin composition.

The content of the other resin in the resin composition according to the embodiment of the present invention is preferably 80% by mass or less, more preferably 75% by mass or less, still more preferably 70% by mass or less, even still more preferably 60% by mass or less, and even further still more preferably 50% by mass or less, with respect to the total solid content of the resin composition.

As one preferred aspect of the resin composition according to the embodiment of the present invention, an aspect in which the content of the other resin is a low content can be adopted. In the above aspect, the content of the other resin is preferably 20% by mass or less, more preferably 15% by mass or less, still more preferably 10% by mass or less, even still more preferably 5% by mass or less, and even further still more preferably 1% by mass or less, with respect to the total solid content of the resin composition. The lower limit of the content is not particularly limited, and it may be any content of 0% by mass or more.

The resin composition according to the embodiment of the present invention may contain only one kind of the other resin or may contain two or more kinds thereof. In a case where two or more kinds thereof are contained, the total amount thereof is preferably within the above range.

### <Polymerizable Compound>

The resin composition according to the embodiment of the present invention preferably contains a polymerizable compound.

The melting point of the polymerizable compound is preferably 25° C. or lower.

By setting the melting point to 25° C. or lower, the coating film is likely to flow during drying and heating, and the flatness of the cured substance can be improved.

Examples of the polymerizable compound include a polymerizable compound (radical crosslinking agent) having a radically polymerizable group or another crosslinking agent.

### [Radical Crosslinking Agent]

The resin composition according to the embodiment of the present invention preferably contains a radical crosslinking agent.

The radical crosslinking agent is a compound having a radically polymerizable group. The radically polymerizable group is preferably a group containing an ethylenically unsaturated bond. Examples of the group containing an ethylenically unsaturated bond include a vinyl group, an allyl group, a vinylphenyl group, a (meth)acryloyl group, a maleimide group, and a (meth)acrylamide group.

Among these, a (meth)acryloyl group, a (meth)acrylamide group, or a vinylphenyl group is preferable, and from the viewpoint of reactivity, a (meth)acryloyl group is more preferable.

The radical crosslinking agent is preferably a compound having one or more ethylenically unsaturated bonds; however, a compound having two or more ethylenically unsaturated bonds is more preferable. The radical crosslinking agent may have three or more ethylenically unsaturated bonds.

The compound having 2 or more ethylenically unsaturated bonds is preferably a compound having 2 to 15 ethylenically unsaturated bonds, more preferably a compound having 2 to 10 ethylenically unsaturated bonds, and still more preferably a compound having 2 to 6 ethylenically unsaturated bonds.

From the viewpoint of the film hardness of the pattern (cured substance) to be obtained, the resin composition according to the embodiment of the present invention preferably contains a compound having two ethylenically unsaturated bonds and a compound having three or more ethylenically unsaturated bonds.

The molecular weight of the radical crosslinking agent is preferably 2,000 or less, more preferably 1,500 or less, and still more preferably 900 or less. The lower limit of the molecular weight of the radical crosslinking agent is preferably 100 or more.

Specific examples of the radical crosslinking agent include unsaturated carboxylic acids (for example, acrylic acid, methacrylic acid, itaconic acid, crotonic acid, isocrotonic acid, and maleic acid), and esters and amides thereof, where esters between unsaturated carboxylic acids and polyhydric alcohol compounds or amides between unsaturated carboxylic acids and polyvalent amine compounds are preferable. In addition, addition reaction products produced by reacting unsaturated carboxylic acid esters or amides, having a nucleophilic substituent such as a hydroxy group, an amino group, or a sulfanyl group, with monofunctional or polyfunctional isocyanates or epoxies, dehydration condensation reaction products produced by reacting the above esters or amides with a monofunctional or polyfunctional carboxylic acid, or the like are also suitably used. In addition, an addition reaction product produced by reacting unsaturated carboxylic acid esters or amides, having an electrophilic substituent such as an isocyanate group or an epoxy group, with monofunctional or polyfunctional alcohols, amines, or thiols, and further, a substitution reaction product produced by reacting unsaturated carboxylic acid esters or amides, having an eliminable substituent such as a halogeno group or a tosyloxy group, with monofunctional or polyfunctional alcohols, amines, or thiols is also suitable. In addition, as other examples, it is also possible to use a group of compounds in which the unsaturated carboxylic acid described above is replaced with an unsaturated phosphonic acid, a vinylbenzene derivative such as styrene, a vinyl ether, an allyl ether, or the like. Regarding the specific examples thereof, reference can be made to the description of paragraphs 0113 to 0122 of JP2016-027357A, the content of which is incorporated in the present specification.

The radical crosslinking agent is also preferably a compound having a boiling point of 100° C. or higher under normal pressure. Examples of the compound having a boiling point of 100° C. or higher under normal pressure include the compounds described in paragraph 0203 of WO2021/112189A. The content thereof is incorporated in the present specification.

Examples of the preferred radical crosslinking agent other than those described above include the radically polymerizable compounds described in paragraphs 0204 to 0208 of WO2021/112189A. The content thereof is incorporated in the present specification.

As the radical crosslinking agent, dipentaerythritol triacrylate (as a commercially available product, KAYARAD D-330 (manufactured by Nippon Kayaku Co., Ltd.)), dipentaerythritol tetraacrylate (as a commercially available product, KAYARAD D-320 (manufactured by Nippon Kayaku Co., Ltd.)), A-TMMT (manufactured by Shin-Nakamura Chemical Co., Ltd.), dipentaerythritol penta(meth)acrylate (as a commercially available product, KAYARAD D-310 (manufactured by Nippon Kayaku Co., Ltd.)), dipentaerythritol hexa(meth)acrylate (as a commercially available product, KAYARAD DPHA (manufactured by Nippon Kayaku Co., Ltd.)), A-DPH (manufactured by Shin-Nakamura Chemical Co., Ltd.), or a structure in which a (meth)acryloyl group thereof is bonded through an ethylene glycol residue or a propylene glycol residue is preferable. Oligomer types thereof can also be used.

Examples of the commercially available product of the radical crosslinking agent include SR-494 which is a tetrafunctional acrylate having four ethyleneoxy chains, SR-209, 231, and 239 which are a difunctional methacrylate having four ethyleneoxy chains (all of which are manufactured by Sartomer Company Inc.), DPCA-60 which is a hexafunctional acrylate having six pentyleneoxy chains and TPA-330 which is a trifunctional acrylate having three isobutylene oxy chains (all of which are manufactured by Nippon Kayaku Co., Ltd.), UAS-10 and UAB-140 which are a urethane oligomer (all of which are manufactured by Nippon Paper Industries Co., Ltd.), NK Ester M-40G, NK Ester 4G, NK Ester M-9300, NK Ester A-9300, and UA-7200 (all of which are manufactured by Shin-Nakamura Chemical Co., Ltd.), DPHA-40H (manufactured by Nippon Kayaku Co., Ltd.), UA-306H, UA-306T, UA-306I, AH-600, T-600, and AI-600 (all of which are manufactured by KYOEISHA CHEMICAL Co., Ltd.), and Brenmer PME400 (manufactured by NOF Corporation).

As the radical crosslinking agent, the urethane acrylates as described in JP1973-041708B (JP-S48-041708B), JP1976-037193A (JP-S51-037193A), JP1990-032293B (JP-H02-032293B), and JP1990-016765B (JP-H02-016765B), and the urethane compounds having an ethylene oxide-based skeleton described in JP1983-049860B (JP-S58-049860B), JP1981-017654B (JP-S56-017654B), JP1987-039417B (JP-S62-039417B), and JP1987-039418B (JP-S62-039418B) are also suitable. As the radical crosslinking agent, the compounds having an amino structure or a sulfide structure in the molecule as described in JP1988-277653A (JP-S63-277653A), JP1988-260909A (JP-S63-260909A), and JP1989-105238A (JP-HO1-105238A) can also be used.

The radical crosslinking agent may be a radical crosslinking agent having an acid group such as a carboxy group or a phosphoric acid group. The radical crosslinking agent having an acid group is preferably an ester between an aliphatic polyhydroxy compound and an unsaturated carboxylic acid and more preferably a radical crosslinking agent obtained by reacting an unreacted hydroxy group of an aliphatic polyhydroxy compound with a non-aromatic carboxylic acid anhydride to have an acid group. The radical crosslinking agent is particularly preferably a compound in which an aliphatic polyhydroxy compound is pentaerythritol or dipentaerythritol in a radical crosslinking agent having an acid group obtained by reacting an unreacted hydroxy group of the aliphatic polyhydroxy compound with a non-aromatic carboxylic acid anhydride. Examples of the commercially available product thereof include M-510 and M-520 as polybasic acid-modified acrylic oligomers which are manufactured by Toagosei Co., Ltd.

An acid value of the radical crosslinking agent having an acid group is preferably 0.1 to 300 mgKOH/g and more preferably 1 to 100 mgKOH/g. In a case where the acid value of the radical crosslinking agent is within the above-described range, excellent manufacturing handleability is exhibited, and excellent developability is exhibited. In addition, good polymerization properties are exhibited. The acid value is measured in accordance with the description of JIS K 0070: 1992.

The radical crosslinking agent is also preferably a radical crosslinking agent having at least one selected from the group consisting of a urea bond and a urethane bond (hereinafter, also referred to as a “crosslinking agent U”).

In addition, as the radical crosslinking agent, the crosslinking agent U or a radical crosslinking agent having an isocyanuric ring structure is preferable, and a radical crosslinking agent having a urea bond or an isocyanuric ring structure is more preferable.

In a case where the resin composition contains a crosslinking agent U, chemical resistance, resolution, and the like may be improved.

The mechanism by which the above-described effect is obtained is not revealed; however, it is considered that, for example, a part of the crosslinking agent U undergoes thermal decomposition during curing by heating or the like to generate an amine or the like, and the amine or the like accelerates the cyclization of the precursor of the cyclization resin such as the polyimide precursor.

The crosslinking agent U may have only one urea bond or urethane bond, may have one or more urea bonds and one or more urethane bonds, may have two or more urea bonds without having a urethane bond, or may have two or more urethane bonds without having a urea bond.

The total number of urea bonds and urethane bonds in the crosslinking agent U is 1 or more, and it is preferably 1 to 10, more preferably 1 to 4, and still more preferably 1 or 2.

In a case where the crosslinking agent U does not have a urethane bond, the number of urea bonds in the crosslinking agent U is 1 or more, and it is preferably 1 to 10, more preferably 1 to 4, and still more preferably 1 or 2.

In a case where the crosslinking agent U does not have a urea bond, the number of urethane bonds in the crosslinking agent U is 1 or more, and it is preferably 1 to 10, more preferably 1 to 4, and still more preferably 1 or 2.

The radically polymerizable group in the crosslinking agent U is not particularly limited; however, examples thereof include a vinyl group, an allyl group, a (meth)acryloyl group, a (meth)acryloxy group, a (meth)acrylamide group, a vinylphenyl group, and a maleimide group, where a (meth)acryloxy group, a (meth)acrylamide group, a vinylphenyl group, or a maleimide group is preferable, and a (meth)acryloxy group is more preferable.

In a case where the crosslinking agent U has two or more radically polymerizable groups, the structures of the respective radically polymerizable groups may be the same or different from each other.

The number of radically polymerizable groups in the crosslinking agent U may be only one or may be two or more, and it is preferably 1 to 10, more preferably 1 to 6, and particularly preferably 1 to 4.

The radically polymerizable group equivalent (the mass of the compound per 1 mol of the radically polymerizable group) in the crosslinking agent U is preferably 150 to 400 g/mol.

From the viewpoint of the chemical resistance of the cured substance, the lower limit of the above-described radically polymerizable group equivalent is more preferably 200 g/mol or more, still more preferably 210 g/mol or more, even more preferably 220 g/mol or more, even still more preferably 230 g/mol or more, even still more preferably 240 g/mol or more, and particularly preferably 250 g/mol or more.

From the viewpoint of developability, the upper limit of the above-described radically polymerizable group equivalent is more preferably 350 g/mol or less, still more preferably 330 g/mol or less, and particularly preferably 300 g/mol or less.

Among the above, the polymerizable group equivalent of the crosslinking agent U is preferably 210 to 400 g/mol and more preferably 220 to 400 g/mol.

The crosslinking agent U has preferably, for example, a structure represented by Formula (U-1).

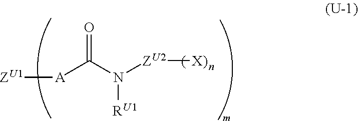

In Formula (U-1), R^{U1} is a hydrogen atom or a monovalent organic group, A is —O—, or —NR^{N}—, R^{N} is a hydrogen atom or a monovalent organic group, Z^{U1} is an m-valent organic group, Z^{U2} is an (n+1)-valent organic group, X is a radically polymerizable group, n is an integer of 1 or more, and m is an integer of 1 or more.

R^{U1} is preferably a hydrogen atom, an alkyl group, or an aromatic hydrocarbon group, and more preferably a hydrogen atom.

R^{N} is preferably a hydrogen atom, an alkyl group, or an aromatic hydrocarbon group, and more preferably a hydrogen atom.

Z^{U1} is preferably a hydrocarbon group, —O—, —C(═O)—, —S—, —S(═O)₂—, —NR^{N}—, or a group in which two or more of these are bonded, and more preferably a hydrocarbon group or a group in which a hydrocarbon group is bonded to at least one group selected from the group consisting of —O—, —C(═O)—, —S—, —S(═O)₂—, and —NR^{N}—.

As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. Examples of the above-described hydrocarbon group include a saturated aliphatic hydrocarbon group, an aromatic hydrocarbon group, and a group represented by bonding of these groups. R^{N} represents a hydrogen atom or a monovalent organic group, and it is preferably a hydrogen atom or a hydrocarbon group, more preferably a hydrogen atom or an alkyl group, and still more preferably a hydrogen atom or a methyl group.

Z^{U2} is preferably a hydrocarbon group, —O—, —C(═O)—, —S—, —S(═O)₂—, —NR^{N}—, or a group in which two or more of these are bonded, and more preferably a hydrocarbon group or a group in which a hydrocarbon group is bonded to at least one group selected from the group consisting of —O—, —C(═O)—, —S—, —S(═O)₂—, and —NR^{N}—.

Examples of the hydrocarbon group include the same ones as those exemplified as Z^{U1}, and the same applies to the preferred aspect thereof.

X is not particularly limited; however, examples thereof include a vinyl group, an allyl group, a (meth)acryloyl group, a (meth)acryloxy group, a (meth)acrylamide group, a vinylphenyl group, and a maleimide group, where a (meth)acryloxy group, a (meth)acrylamide group, a vinylphenyl group, or a maleimide group is preferable, and a (meth)acryloxy group is more preferable.

n is preferably an integer of 1 to 10, more preferably an integer of 1 to 4, still more preferably 1 or 2, and particularly preferably 1.

m is preferably an integer of 1 to 10, more preferably an integer of 1 to 4, and still more preferably 1 or 2.

It is also preferable that the crosslinking agent U has at least one of a hydroxy group, an alkyleneoxy group, an amide group, or a cyano group.

From the viewpoint of the chemical resistance of the cured film to be obtained, the hydroxy group may be an alcoholic hydroxy group or a phenolic hydroxy group; however, it is preferably an alcoholic hydroxy group.

From the viewpoint of the chemical resistance of the cured film to be obtained, the alkyleneoxy group is preferably an alkyleneoxy group having 2 to 20 carbon atoms, more preferably an alkyleneoxy group having 2 to 10 carbon atoms, still more preferably an alkyleneoxy group having 2 to 4 carbon atoms, even still more preferably an ethylene group or a propylene group, and particularly preferably an ethylene group.

The alkyleneoxy group may be contained in the crosslinking agent U as a polyalkyleneoxy group. The number of repetitions of the alkyleneoxy group in this case is preferably 2 to 10 and more preferably 2 to 6.

The amide group refers to a bond represented by —C(═O)—NR^{N}—. R^{N} is as described above. In a case where the crosslinking agent U has an amide group, the crosslinking agent U can include, for example, a group represented by R—C(═O)—NR^{N}—* or a group represented by *—C(═O)—NR^{N}—R. R represents a hydrogen atom or a monovalent substituent, and it is preferably a hydrogen atom or a hydrocarbon group and more preferably a hydrogen atom, an alkyl group, or an aromatic hydrocarbon group.

The crosslinking agent U may have, in the molecule, two or more structures selected from the group consisting of a hydroxy group, an alkyleneoxy group (however, a polyalkyleneoxy group in a case of constituting a polyalkyleneoxy group), an amide group, and a cyano group; however, an aspect in which only one structure is contained in the molecule is also preferable.

The hydroxy group, the alkyleneoxy group, the amide group, and the cyano group may be present at any position of the crosslinking agent U. However, from the viewpoint of chemical resistance, it is also preferable that the crosslinking agent U is such that at least one selected from the group consisting of the hydroxy group, the alkyleneoxy group, the amide group, and the cyano group, and at least one radically polymerizable group contained in the crosslinking agent U are linked by a linking group (hereinafter, also referred to as a “linking group L2-1”) containing a urea bond or a urethane bond.

In particular, in a case where the crosslinking agent U contains only one radically polymerizable group, it is preferable that the radically polymerizable group contained in the crosslinking agent U and at least one selected from the group consisting of a hydroxy group, an alkyleneoxy group, an amide group, and a cyano group are linked by a linking group including a urea bond or a urethane bond (hereinafter, also referred to as a “linking group L2-2”).

In a case where the crosslinking agent U contains an alkyleneoxy group (however, a polyalkyleneoxy group in a case of constituting a polyalkyleneoxy group) and has the above-described linking group L2-1 or the above-described linking group L2-2, a structure that is bonded to a side of the alkyleneoxy group (however, a polyalkyleneoxy group in a case of constituting a polyalkyleneoxy group) opposite to the linking group L2-1 or the linking group L2-2 is not particularly limited; however, it is preferably a hydrocarbon group, a radically polymerizable group, or a group represented by a combination thereof. As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. Examples of the above-described hydrocarbon group include a saturated aliphatic hydrocarbon group, an aromatic hydrocarbon group, and a group represented by bonding of these groups. In addition, the preferred aspect of the radically polymerizable group is the same as the preferred aspect of the radically polymerizable group in the above-described crosslinking agent U.

In a case where the crosslinking agent U contains an amide group and has the linking group L2-1 or the linking group L2-2, a structure that is bonded to a side of the amide group opposite to the linking group L2-1 or the linking group L2-2 is not particularly limited; however, it is preferably a hydrocarbon group, a radically polymerizable group, or a group represented by a combination thereof. As the above-described hydrocarbon group, a hydrocarbon group having 20 or less carbon atoms is preferable, a hydrocarbon group having 18 or less carbon atoms is more preferable, and a hydrocarbon group having 16 or less carbon atoms is still more preferable. In addition, examples of the above-described hydrocarbon group include a saturated aliphatic hydrocarbon group, an aromatic hydrocarbon group, and a group represented by bonding of these groups. The preferred aspect of the radically polymerizable group is the same as the preferred aspect of the radically polymerizable group in the above-described crosslinking agent U. In addition, in the above aspect, a carbon atom side of the amide group may be bonded to the linking group L2-1 or the linking group L2-2, or a nitrogen atom side of the amide group may be bonded to the linking group L2-1 or the linking group L2-2.

Among these, from the viewpoint of the adhesiveness to the base material, the chemical resistance, and the Cu void suppression, the crosslinking agent U preferably has a hydroxy group.

From the viewpoint of the compatibility with the specific resin and the like, the crosslinking agent U preferably contains an aromatic group.

The aromatic group is preferably directly bonded to a urea bond or a urethane bond contained in the crosslinking agent U. In a case where the crosslinking agent U contains two or more urea bonds or two or more urethane bonds, it is preferable that one of the urea bonds or urethane bonds is directly bonded to the aromatic group.

The aromatic group may be an aromatic hydrocarbon group or an aromatic heterocyclic group and may have a structure in which these groups form a fused ring; however, an aromatic hydrocarbon group is preferable.

The aromatic hydrocarbon group is preferably an aromatic hydrocarbon group having 6 to 30 carbon atoms, more preferably an aromatic hydrocarbon group having 6 to 20 carbon atoms, and still more preferably a group obtained by removing two or more hydrogen atoms from a benzene ring structure.

The aromatic heterocyclic group is preferably a 5-membered or 6-membered aromatic heterocyclic group. Examples of the aromatic heterocyclic ring in such an aromatic heterocyclic group include pyrrole, imidazole, triazole, tetrazole, pyrazole, furan, thiophene, oxazole, isoxazole, thiazole, pyridine, pyrazine, pyrimidine, pyridazine, and triazine. These rings may be further fused with another ring, for example, as in the case of indole or benzimidazole.

The heteroatom contained in the aromatic heterocyclic group is preferably a nitrogen atom, an oxygen atom, or a sulfur atom.

It is preferable that the aromatic group is included in, for example, a linking group that links two or more radically polymerizable groups and links a linking group having a urea bond or a urethane bond, or at least one selected from the group consisting of a hydroxy group, an alkyleneoxy group, an amide group, and a cyano group, to at least one radically polymerizable group contained in the crosslinking agent U.

The number of atoms (linking chain length) between the urea bond or urethane bond and the radically polymerizable group in the crosslinking agent U is not particularly limited; however, it is preferably 30 or less, more preferably 2 to 20, and still more preferably 2 to 10.

In a case where the crosslinking agent U contains two or more urea bonds or urethane bonds in total, the minimum number of atoms (linking chain length) between the urea bond or urethane bond and the radically polymerizable group is sufficient to be within the above-described range in a case where the crosslinking agent U contains two or more radically polymerizable groups, or in a case where the crosslinking agent U contains two or more urea bonds or two or more urethane bonds and two or more radically polymerizable groups.

In the present specification, the phrase “number of atoms (linking chain length) between the urea bond or urethane bond and the radically polymerizable group” refers to the smallest number of atoms (minimum number of atoms) for linking targets among the number of atoms for atomic chains on a path that links two atoms or atomic groups between the linking targets. For example, in a structure represented by the following Formula, the number of atoms (linking chain length) between the urea bond and the radically polymerizable group (methacryloyloxy group) is 2.

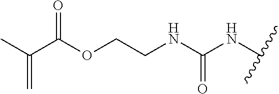 [SMILES: *NC(=O)NCCOC(=O)C(=C)C]

### [Symmetry Axis]

It is also preferable that the crosslinking agent U is a compound having a structure which does not have a symmetry axis.

The fact that the crosslinking agent U does not have a symmetry axis refers to that the crosslinking agent U is a left-right asymmetric compound which does not have an axis that generates the same molecule as the original molecule in a case where the entire compound is rotated. In addition, in a case where the structural formula of the crosslinking agent U is shown on the paper surface, the fact that the crosslinking agent U does not have a symmetry axis refers to that the structural formula of the crosslinking agent U cannot be shown in a form having a symmetry axis.

It is considered that the aggregation of the crosslinking agents U is suppressed in the composition film in a case where the crosslinking agent U does not have a symmetry axis.

### [Molecular Weight]

The molecular weight of the crosslinking agent U is preferably 100 to 2,000, more preferably 150 to 1,500, and still more preferably 200 to 900.

A manufacturing method for the crosslinking agent U is not particularly limited; however, the crosslinking agent U can be obtained, for example, by reacting a radically polymerizable compound with a compound having an isocyanate group and a compound having at least one of a hydroxy group or an amino group.

Specific examples of the crosslinking agent U are shown below; however, the crosslinking agent U is not limited thereto.

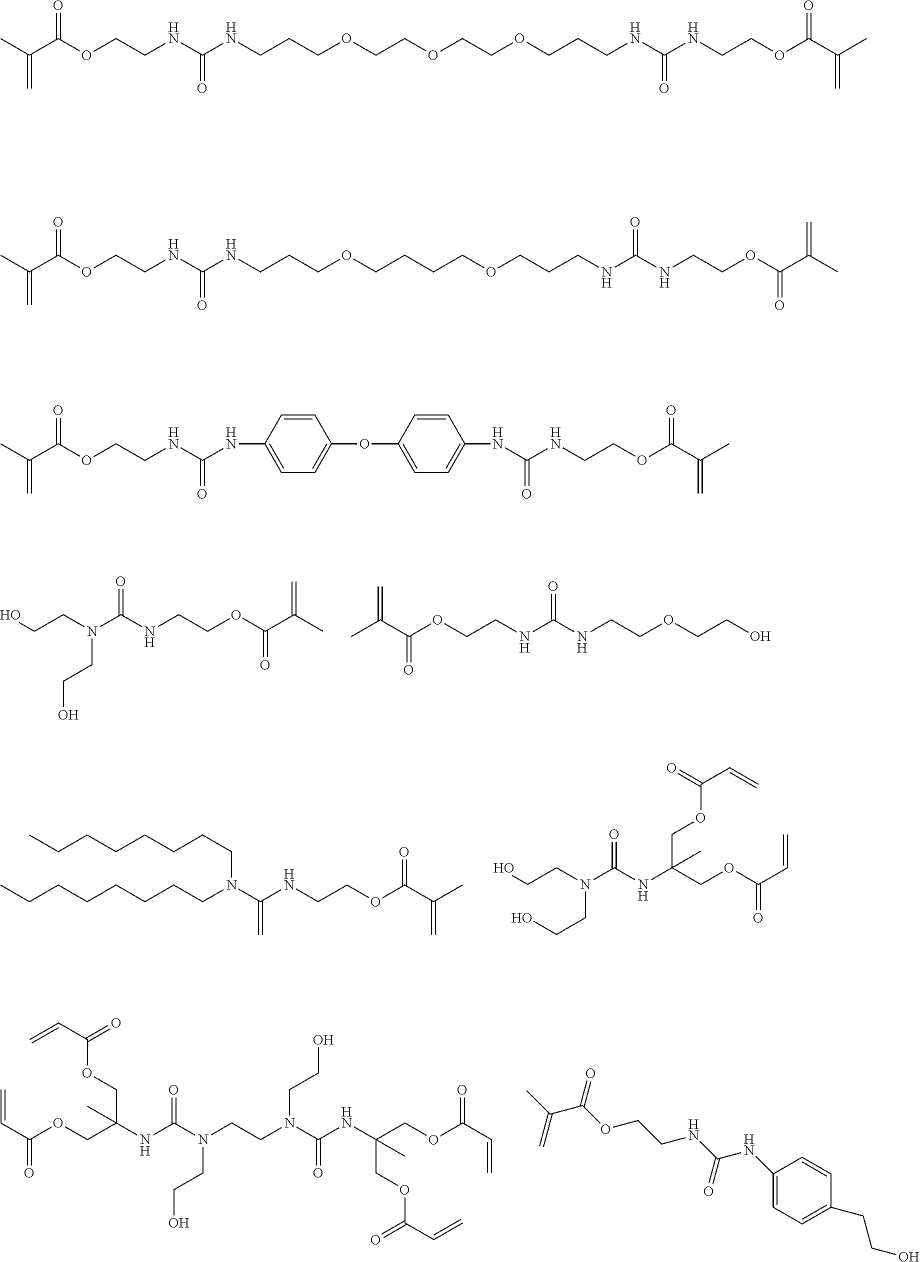

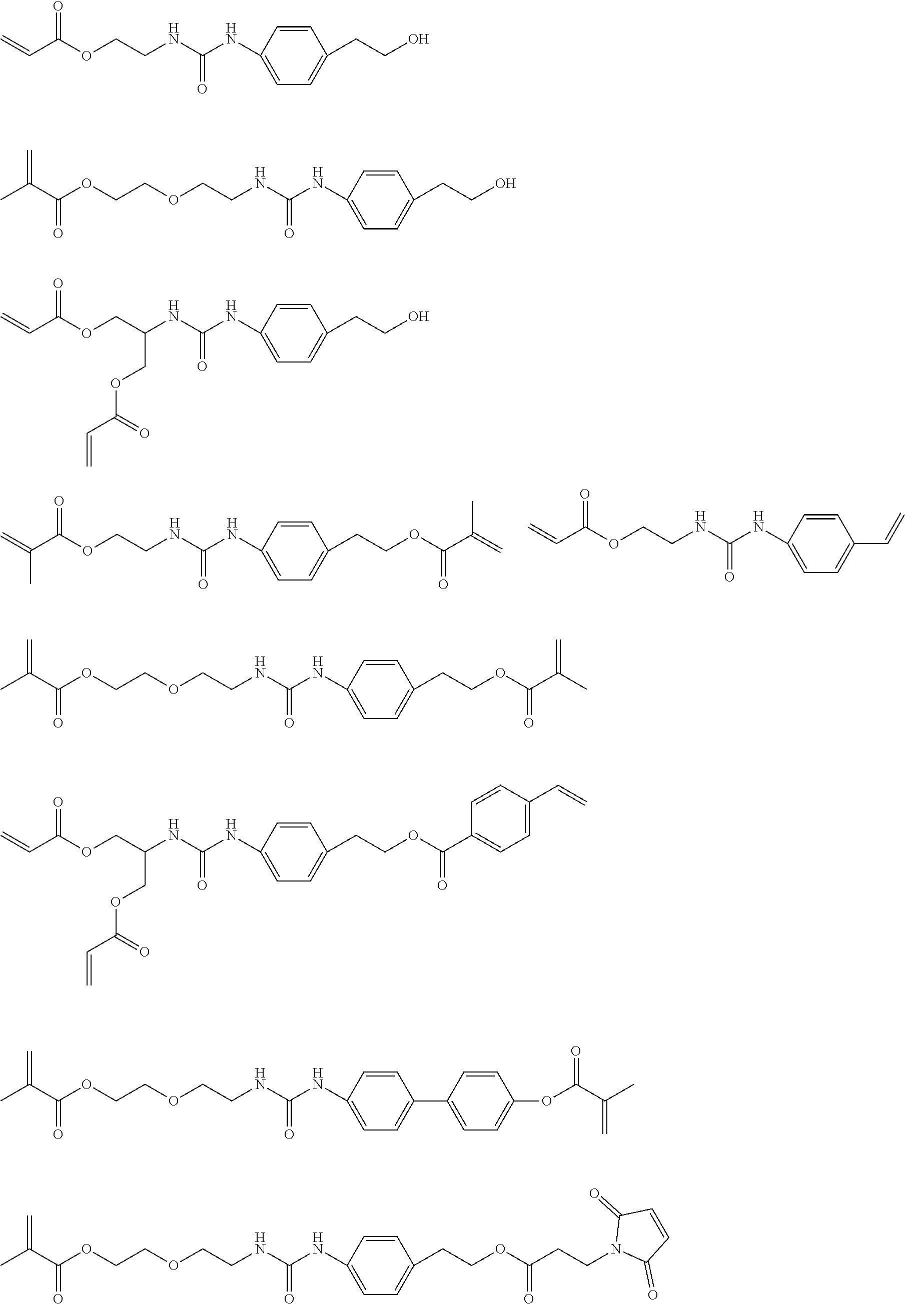

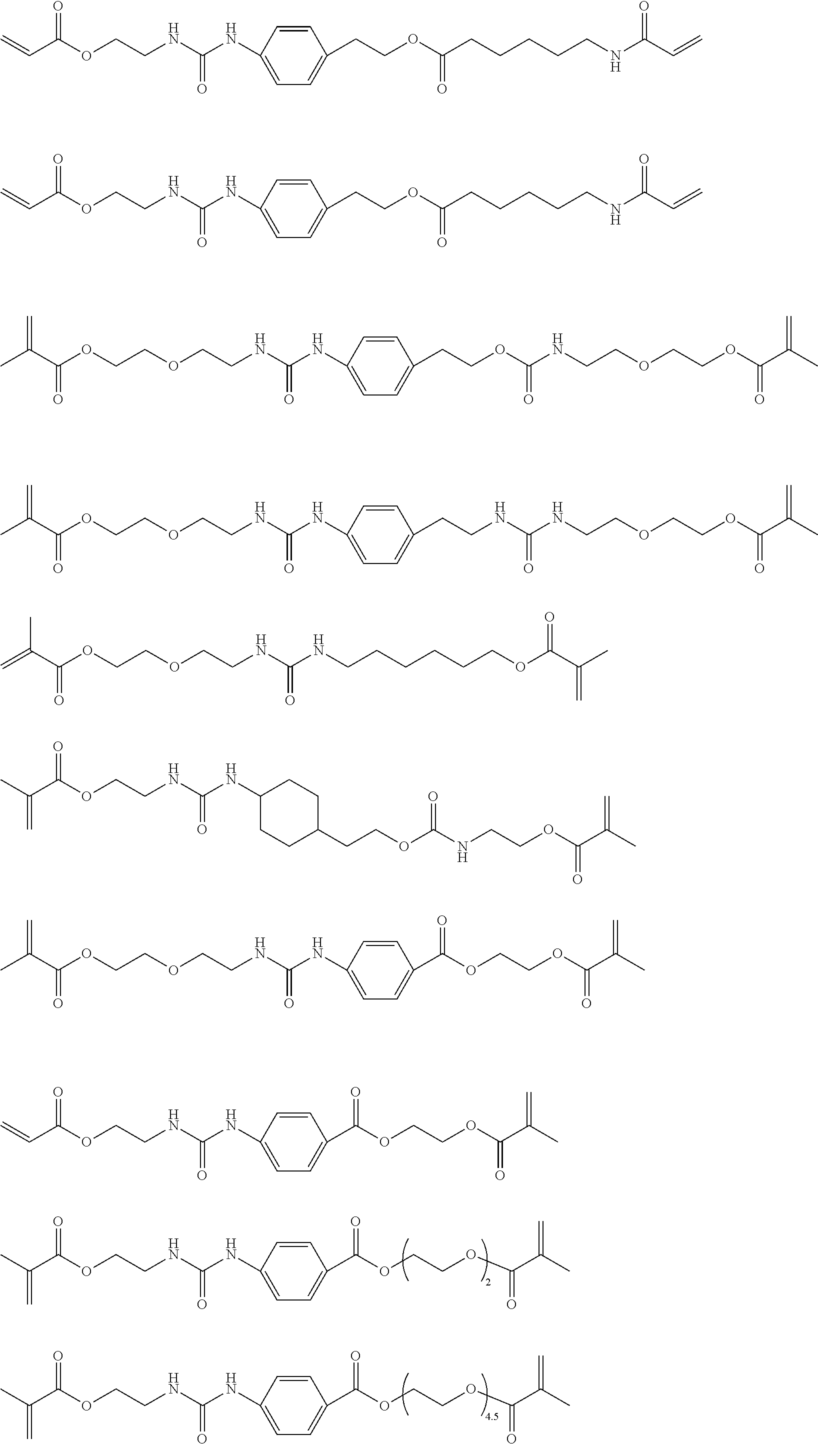

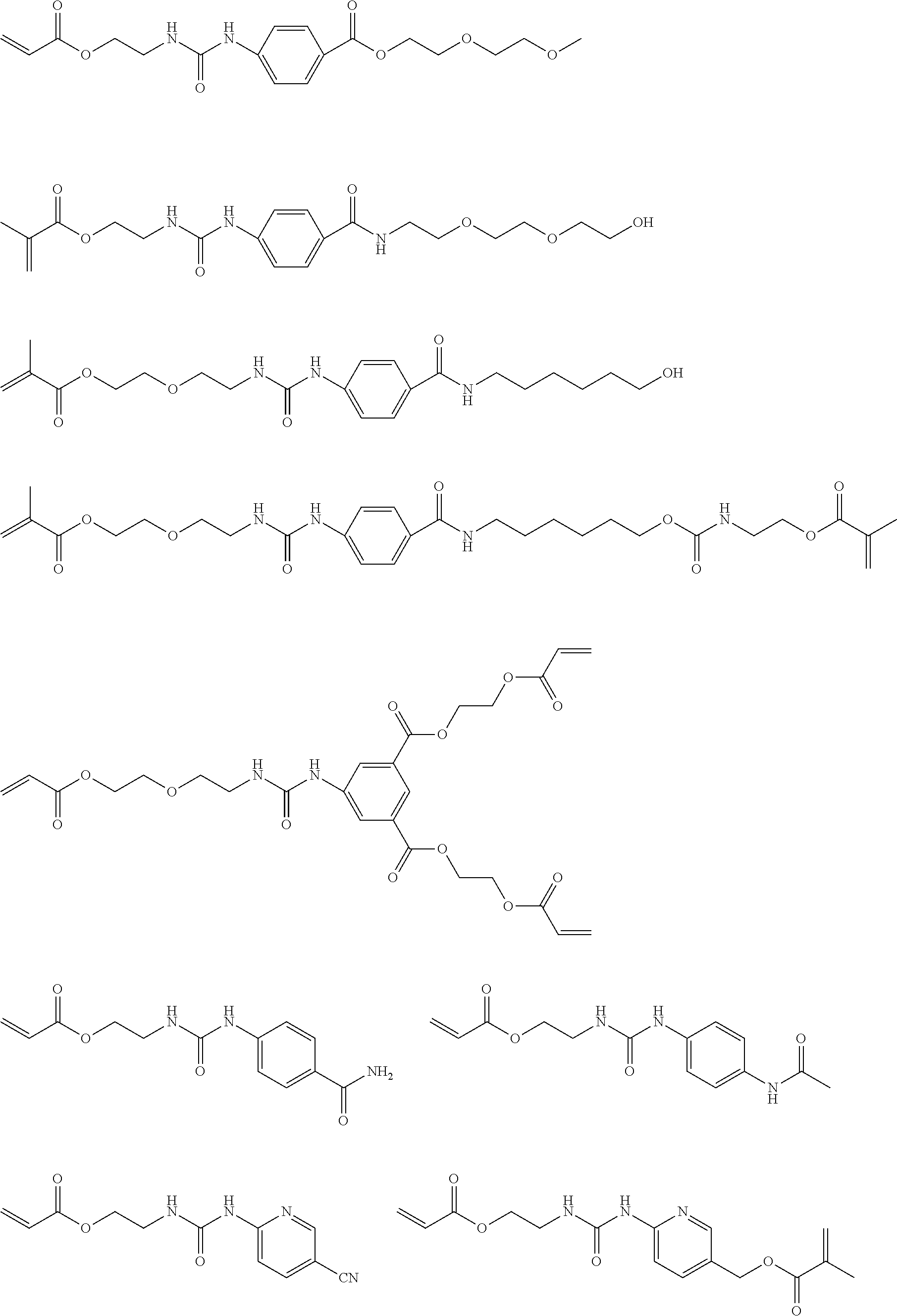

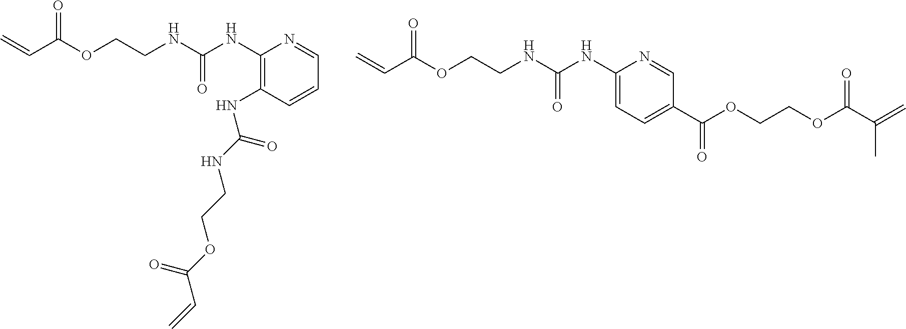

As the radical crosslinking agent having an isocyanuric ring structure, a compound having two or three radically polymerizable groups is preferable, and a compound having three radically polymerizable groups is more preferable.

In addition, examples of the radical crosslinking agent having an isocyanuric ring structure include tris(2-acryloyloxyethyl) isocyanuric acid, tris(2-methacryloyloxyethyl) isocyanuric acid, ethylene oxide (EO)-modified diacrylate of isocyanuric acid, EO-modified triacrylate of isocyanuric acid, and a compound having the following structure, but the present invention is not limited thereto. In the following structure, n's each independently represent an integer of 1 to 20, and R represents a divalent linking group.

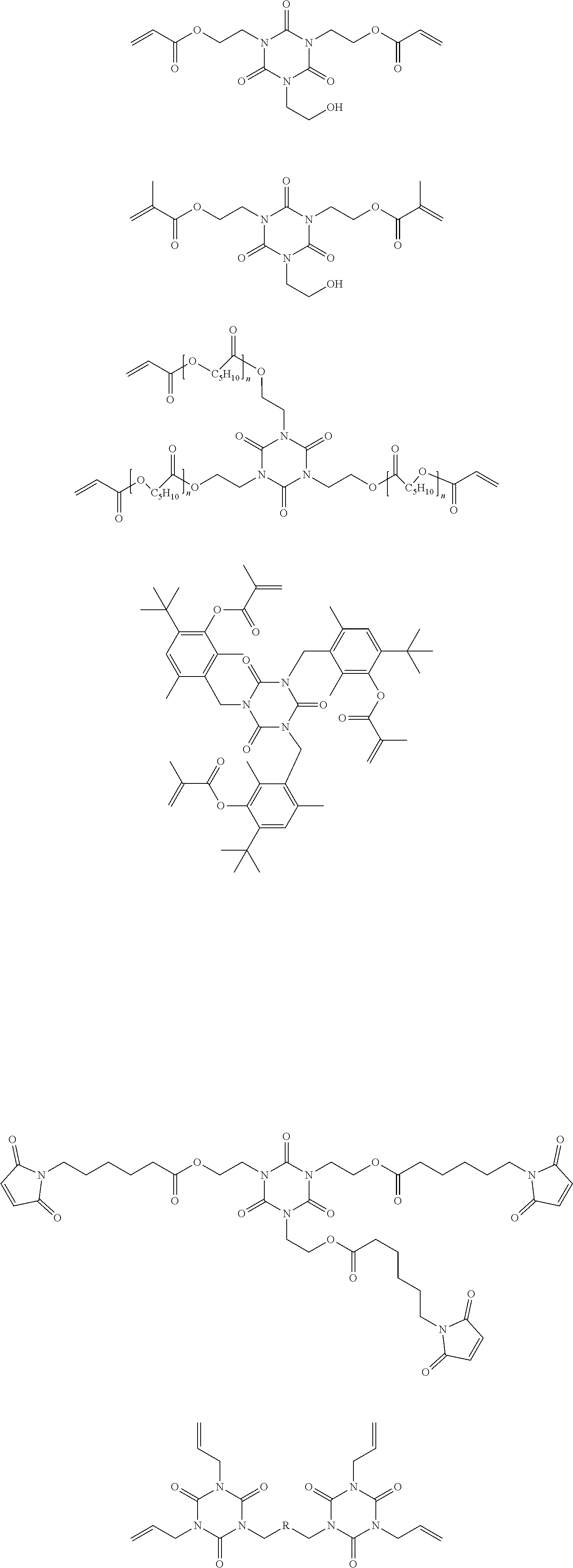

In the resin composition, it is preferable to use difunctional methacrylate or acrylate from the viewpoint of pattern resolution and film elasticity.

As the specific compound, the following compound can be used; triethylene glycol diacrylate, triethylene glycol dimethacrylate, tetraethylene glycol dimethacrylate, tetraethylene glycol diacrylate, polyethylene glycol (PEG) 200 diacrylate, PEG 200 dimethacrylate, PEG 600 diacrylate, PEG 600 dimethacrylate, polytetraethylene glycol diacrylate, polytetraethylene glycol dimethacrylate, dipropylene glycol diacrylate, tripropylene glycol diacrylate, neopentyl glycol diacrylate, neopentyl glycol dimethacrylate, 3-methyl-1, 5-pentanediol diacrylate, 1,6-hexanediol diacrylate, 1,6-hexanediol dimethacrylate, dimethylol-tricyclodecanediacrylate, dimethylol-tricyclodecanedimethacrylate, a diacrylate of an ethylene oxide (EO) adduct of bisphenol A, a dimethacrylate of an EO adduct of bisphenol A, a diacrylate of a propylene oxide (PO) adduct of bisphenol A, a dimethacrylate of a PO adduct of bisphenol A, 2-hydroxy-3-acryloyloxypropyl methacrylate, another difunctional acrylate having a urethane bond, and difunctional methacrylate having a urethane bond. As necessary, two or more of these can be mixedly used.

It is noted that, for example, the PEG 200 diacrylate refers to a polyethylene glycol diacrylate having a polyethylene glycol chain Formula weight of about 200.

In the photocurable resin composition according to the embodiment of the present invention, a monofunctional radical crosslinking agent can be preferably used as the radical crosslinking agent from the viewpoint of suppressing the warping of the pattern (cured substance). As the monofunctional radical crosslinking agent, (meth)acrylic acid derivatives such as n-butyl (meth)acrylate, 2-ethylhexyl (meth)acrylate, 2-hydroxyethyl (meth)acrylate; butoxyethyl (meth)acrylate, carbitol (meth)acrylate, cyclohexyl (meth)acrylate, benzyl (meth)acrylate, phenoxyethyl (meth)acrylate, N-methylol (meth)acrylamide, glycidyl (meth)acrylate, polyethylene glycol mono(meth)acrylate, and polypropylene glycol mono(meth)acrylate, N-vinyl compounds such as N-vinylpyrrolidone and N-vinylcaprolactam, allyl glycidyl ethers, and the like are preferably used. As the monofunctional radical crosslinking agent, a compound having a boiling point of 100° C. or higher under normal pressure is also preferable in order to suppress volatilization before exposure.

In addition, examples of the bi- or higher functional radical crosslinking agent include allyl compounds such as diallyl phthalate and triallyl trimellitate.

In addition, the polymerizable compound may be a compound having an allyl group and at least one group selected from the group consisting of a (meth)acryloxy group, a (meth)acrylamide group, a vinylphenyl group, and a maleimide group.

The allyl group has a low dielectric loss tangent of the structure after polymerization among the polymerizable groups, and the dielectric loss tangent of the cured substance can be reduced by including such a compound.

In addition, from the viewpoint of increasing the compatibility with the resin and easily forming a structure having a low dielectric loss tangent uniformly in the film, the compound preferably has an aromatic ring structure. The aromatic ring structure may be an aromatic hydrocarbon ring structure or an aromatic heterocyclic ring structure, but it is preferably an aromatic hydrocarbon ring structure and more preferably a benzene ring structure. The aromatic ring structure may have a known substituent or a fused ring structure.

In addition, from the viewpoint of suppressing the occurrence of residual film during development, the compound preferably has an aliphatic saturated hydrocarbon group having 4 or more carbon atoms.

The number of carbon atoms in the aliphatic saturated hydrocarbon group having 4 or more carbon atoms is preferably 4 to 20 and more preferably 4 to 8. The above-described aliphatic saturated hydrocarbon group is preferably a chain-like aliphatic saturated hydrocarbon group. The above-described aliphatic saturated hydrocarbon group may have a known substituent.

Specific examples of such a compound include BS-1, BS-2, and the like in Examples described later.

In a case where a radical crosslinking agent is contained, the content of the radical crosslinking agent is preferably more than 0% by mass and 60% by mass or less with respect to the total solid content of the resin composition. The lower limit thereof is more preferably 5% by mass or more. The upper limit thereof is more preferably 50% by mass or less and still more preferably 30% by mass or less.

One kind of radical crosslinking agent may be used alone, or two or more kinds thereof may be mixedly used. In a case where two or more kinds thereof are used in combination, the total amount thereof is preferably in the above range.

### [Another Crosslinking Agent]

The resin composition according to the embodiment of the present invention also preferably contains another crosslinking agent different from the radical crosslinking agent described above.

The other crosslinking agent refers to a crosslinking agent other than the above-described radical crosslinking agent, where it is preferably a compound having a plurality of groups, in the molecule, which accelerates a reaction of forming a covalent bond between other compounds in the composition or reaction products thereof, by the photosensitization of the above-described photoacid generator or photobase generator, and is more preferably a compound having a plurality of groups, in the molecule, which accelerates a reaction of forming a covalent bond between other compounds in the composition or reaction products thereof, by the action of the acid or the base.

The acid or the base is preferably an acid or a base, which is generated from a photoacid generator or a photobase generator in the exposure step.

Examples of the other crosslinking agent include the compounds described in paragraphs 0179 to 0207 of WO2022/145355A. The above description is incorporated in the present specification.

### [Photosensitizer]

The resin composition according to the embodiment of the present invention contains a photosensitizing agent.

Examples of the photosensitizing agent include a photopolymerization initiator and a photoacid generator, where a photopolymerization initiator is preferable.

### [Polymerization Initiator]

The resin composition according to the embodiment of the present invention preferably includes a photopolymerization initiator. The photopolymerization initiator is preferably a photoradical polymerization initiator. The photoradical polymerization initiator is not particularly limited and can be appropriately selected from publicly known photoradical polymerization initiators. For example, a photoradical polymerization initiator having photosensitivity to rays ranging from the ultraviolet ray range to the visible light range is preferable. In addition, it may be an activator that acts with a sensitizing agent to generate an active radical.

The photoradical polymerization initiator preferably contains at least one compound having a molar absorption coefficient of at least about 50 L mol-1 cm⁻¹ within a range of a wavelength of about 240 to 800 nm (preferably 330 to 500 nm). The molar absorption coefficient of a compound can be measured using a well-publicly known method. For example, it is preferable to carry out a measurement at a concentration of 0.01 g/L using an ethyl acetate solvent with an ultraviolet-visible spectrophotometer (Cary-5 spectrophotometer manufactured by Varian Medical Systems, Inc.).

As a photoradical polymerization initiator, well-known compounds can be optionally used. Examples thereof include a halogenated hydrocarbon derivative (for example, a compound having a triazine skeleton, a compound having an oxadiazole skeleton, or a compound having a trihalomethyl group), an acylphosphine compound such as an acylphosphine oxide, hexaarylbiimidazole, an oxime compound such as an oxime derivative, an organic peroxide, a thio compound, a ketone compound, an aromatic onium salt, a keto oxime ether, an α-amino ketone compound such as aminoacetophenone, an α-hydroxy ketone compound such as hydroxyacetophenone, an azo-based compound, an azide compound, a metallocene compound, an organic boron compound, and an iron arene complex. With regard to details thereof, reference can be made to the description of paragraphs 0165 to 0182 of JP2016-027357A and paragraphs 0138 to 0151 of WO2015/199219A, the contents of which are incorporated in the present specification. In addition, examples thereof include the compounds described in paragraphs 0065 to 0111 of JP2014-130173A and JP6301489B, the peroxide-based photopolymerization initiator described in MATERIAL STAGE 37 to 60 p, vol. 19, No. 3, 2019, the photopolymerization initiator described in WO2018/221177A, the photopolymerization initiator described in WO2018/110179A, the photopolymerization initiator described in JP2019-043864A, the photopolymerization initiator described in JP2019-044030A, and the peroxide-based initiator described in JP2019-167313A, the contents of which are incorporated in the present specification.

Examples of the ketone compound include compounds described in paragraph 0087 of JP2015-087611A, the content of which is incorporated in the present specification. As a commercially available product thereof, KAYACURE DETX-S (manufactured by Nippon Kayaku Co., Ltd.) is also suitably used.

In one embodiment of the present invention, a hydroxyacetophenone compound, an aminoacetophenone compound, and an acylphosphine compound can be suitably used as the photoradical polymerization initiator. More specifically, for example, the aminoacetophenone-based initiator described in JP1998-291969A (JP-H10-291969A) and the acylphosphine oxide-based initiator described in JP4225898B can be used, the contents of which are incorporated in the present specification.

As the α-hydroxy ketone-based initiator, Omnirad 184, Omnirad 1173, Omnirad 2959, Omnirad 127 (all of which are manufactured by IGM Resins B.V.), IRGACURE 184 (IRGACURE is a registered trademark), DAROCUR 1173, IRGACURE 500, IRGACURE-2959, and IRGACURE 127 (all of which are manufactured by BASF SE) can be used.

As the α-amino ketone-based initiator, Omnirad 907, Omnirad 369, Omnirad 369E, Omnirad 379EG (all of which are manufactured by IGM Resins B.V.), IRGACURE 907, IRGACURE 369, and IRGACURE 379 (all of which are manufactured by BASF SE) can be used.

As the aminoacetophenone-based initiator, the acylphosphine oxide-based initiator, and the metallocene compound, it is also possible to suitably use, for example, the compounds described in paragraphs 0161 to 0163 of WO2021/112189A. The content thereof is incorporated in the present specification.

Examples of the more preferred photoradical polymerization initiator include an oxime compound. In a case where an oxime compound is used, exposure latitude can be more effectively improved. The oxime compound is particularly preferable since the oxime compound has a wide exposure latitude (a wide exposure margin) and also works as a photocuring accelerator.

Specific examples of the oxime compound include the compounds described in JP2001-233842A, the compounds described in JP2000-080068A, the compounds described in JP2006-342166A, the compounds described in J. C. S. Perkin II (1979, pp. 1653-1660), the compounds described in J. C. S. Perkin II (1979, pp. 156-162), the compounds described in Journal of Photopolymer Science and Technology (1995, pp. 202-232), the compounds described in JP2000-066385A, the compounds described in JP2004-534797A, the compounds described in JP2017-019766A, the compounds described in JP6065596B, the compounds described in WO2015/152153A, the compounds described in WO2017/051680A, the compounds described in JP2017-198865A, the compounds described in paragraph Nos. 0025 to 0038 of WO2017/164127A, and the compounds described in WO2013/167515A, the content of which is incorporated in the present specification.

Examples of the preferred oxime compound include compounds having the following structures, 3-(benzoyloxy(imino))butan-2-one, 3-(acetoxy(imino))butan-2-one, 3-(propionyloxy(imino))butan-2-one, 2-(acetoxy(imino))pentan-3-one, 2-(acetoxy(imino))-1-phenylpropan-1-one, 2-(benzoyloxy(imino))-1-phenylpropan-1-one, 3-((4-toluenesulfonyloxy)imino)butan-2-one, and 2-(ethoxycarbonyloxy(imino))-1-phenylpropan-1-one. In the resin composition, it is preferable to use an oxime compound, particularly as a photoradical polymerization initiator.

The oxime compound as a photoradical polymerization initiator has a linking group of >C═N—O—C(═O)— in the molecule.

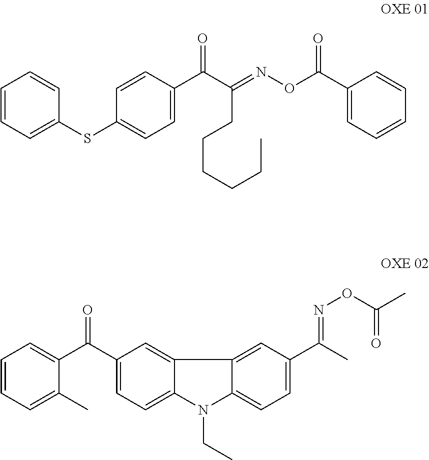 [SMILES: CCCCCC/C(=N\OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21]

Examples of the commercially available product of the oxime compound include IRGACURE OXE 01, IRGACURE OXE 02, IRGACURE OXE 03, and IRGACURE OXE 04 (all of which are manufactured by BASF SE), ADEKA OPTOMER N-1919 (manufactured by ADEKA Corporation, the photoradical polymerization initiator 2 described in JP2012-014052A), TR-PBG-304 and TR-PBG-305 (all of which are manufactured by Changzhou Tronly New Electronic Materials Co., Ltd.), ADEKA ARKLS NCI-730, NCI-831, and ADEKA ARKLS NCI-930 (all of which are manufactured by ADEKA Corporation), DFI-091 (manufactured by DAITO CHEMIX Co., Ltd.), and SpeedCure PDO (manufactured by SARTOMER ARKEMA). In addition, oxime compounds having the following structures can also be used.

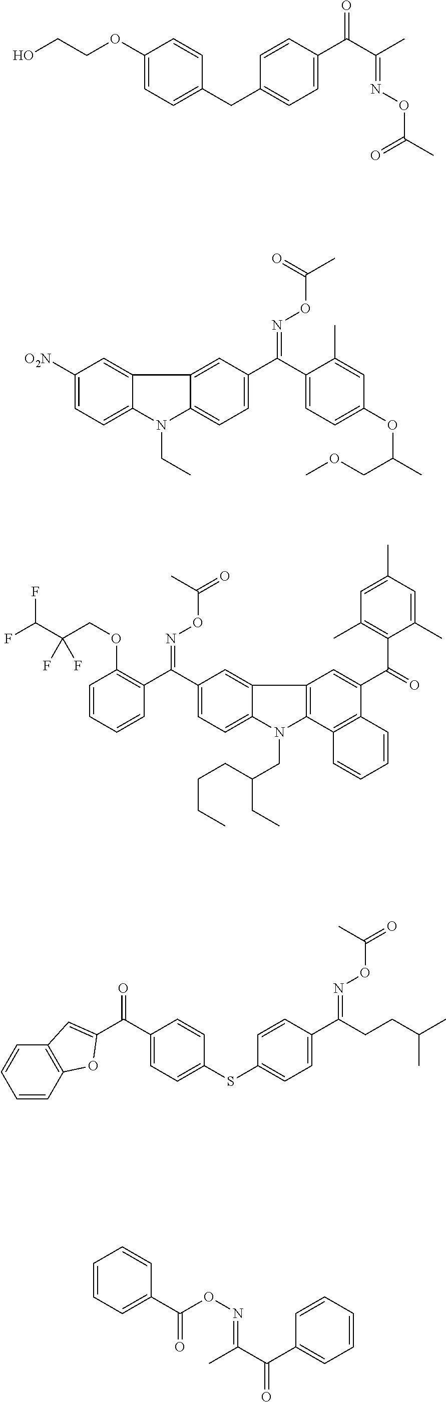 [SMILES: CC(=O)O/N=C(\C)C(=O)c1ccc(Cc2ccc(OCCO)cc2)cc1 ; CCn1c2ccc(/C(=N/OC(C)=O)c3ccc(OC(C)COC)cc3C)cc2c2cc([N+](=O)[O-])ccc21 ; CCCCC(CC)Cn1c2ccc(/C(=N\OC(C)=O)c3ccccc3OCC(F)(F)C(F)F)cc2c2cc(C(=O)c3c(C)cc(C)cc3C)c3ccccc3c21 ; CC(=O)O/N=C(\CCC(C)C)c1ccc(Sc2ccc(C(=O)c3cc4ccccc4o3)cc2)cc1 ; C/C(=N\OC(=O)c1ccccc1)C(=O)c1ccccc1]

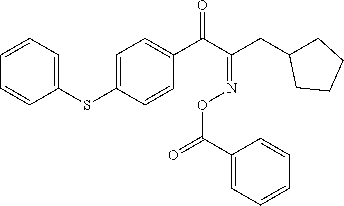 [SMILES: O=C(O/N=C(/CC1CCCC1)C(=O)c1ccc(Sc2ccccc2)cc1)c1ccccc1]

As the photoradical polymerization initiator, it is also possible to use, for example, the oxime compounds having a fluorene ring, which are described in paragraphs 0169 to 0171 of WO2021/112189A, an oxime compound having a skeleton in which at least one benzene ring of a carbazole ring is a naphthalene ring, or an oxime compound having a fluorine atom.

In addition, it is also possible to use the oxime compounds having a nitro group, which are described in paragraphs 0208 to 0210 of WO2021/020359A, an oxime compound having a benzofuran skeleton, or an oxime compound in which a substituent having a hydroxy group is bonded to a carbazole skeleton. The content thereof is incorporated in the present specification.

In addition, as the photopolymerization initiator, compounds described in paragraphs 0113 to 0117 of JP2023-058585A can also be used. This description is incorporated in the present specification.

In a case where the resin composition contains a photopolymerization initiator, the content thereof is preferably 0.1% to 30% by mass, more preferably 0.1% to 20% by mass, still more preferably 0.5% to 15% by mass, and even still more preferably 1.0% to 10% by mass with respect to the total solid content of the resin composition. Only one kind of photopolymerization initiator may be contained, or two or more kinds thereof may be contained. In a case where two or more kinds of photopolymerization initiators are contained, the total amount thereof is preferably within the above-described range.

It is noted that since the photopolymerization initiator may also function as a thermal polymerization initiator, crosslinking with the photopolymerization initiator may be further allowed to proceed by heating an oven, a hot plate, or the like.

### [Sensitizing Agent]

The resin composition may contain a sensitizing agent. The sensitizing agent absorbs a specific radioactive ray to be in an electronically excited state. The sensitizing agent in the electronically excited state is brought into contact with a thermal radical polymerization initiator, a photoradical polymerization initiator, or the like, to cause actions such as electron migration, energy transfer, and heat generation. As a result, the thermal radical polymerization initiator and the photoradical polymerization initiator undergo a chemical change and decompose to generate a radical, an acid, or a base.

In addition, as a usable sensitizing agent, a benzophenone-based, a Michler's ketone-based, a coumarin-based, a pyrazole azo-based, an anilino azo-based, a triphenylmethane-based, an anthraquinone-based, an anthracene-based, an anthrapylidene-based, a benzylidene-based, an oxonol-based, a pyrazolotriazole azo-based, a pyridone azo-based, a cyanine-based, a phenothiazine-based, a pyrrolopyrazole azomethine-based, a xanthene-based, a phthalocyanine-based, a benzopyran-based, and an indigo-based compound can be used.

Examples of the sensitizing agent include, Michler's ketone, 4,4′-bis(diethylamino)benzophenone, 2,5-bis(4′-diethylaminobenzal)cyclopentane, 2,6-bis(4′-diethylaminobenzal)cyclohexanone, 2,6-bis(4′-diethylaminobenzal)-4-methylcyclohexanone, 4,4′-bis(dimethylamino)chalcone, 4,4′-bis(diethylamino)chalcone, p-dimethylaminocinnamylidene indanone, p-dimethylamino benzylidene indanone, 2-(p-dimethylaminophenylbiphenylene)-benzothiazole, 2-(p-dimethylaminophenylvinylene)benzothiazole, 2-(p-dimethylaminophenylvinylene)isonaphtothiazole, 1,3-bis(4′-dimethylaminobenzal)acetone, 1,3-bis(4′-diethylaminobenzal)acetone, 3,3′-carbonyl-bis(7-diethylaminocoumarin), 3-acetyl-7-dimethylaminocoumarin, 3-ethoxycarbonyl-7-dimethylaminocoumarin, 3-benzyloxycarbonyl-7-dimethylaminocoumarin, 3-methoxycarbonyl-7-diethylaminocoumarin, 3-ethoxycarbonyl-7-diethylaminocoumarin (ethyl 7-(diethylamino)coumarin-3-carboxylate), N-phenyl-N′-ethylethanolamine, N-phenyldiethanolamine, N-p-tolyldiethanolamine, N-phenylethanolamine, 4-morpholinobenzophenone, isoamyl dimethylaminobenzoate, isoamyl diethylaminobenzoate, 2-mercaptobenzimidazole, 1-phenyl-5-mercaptotetrazole, 2-mercaptobenzothiazole, 2-(p-dimethylaminostyryl)benzoxazole, 2-(p-dimethylaminostyryl)benzothiazole, 2-(p-dimethylaminostyryl)naphtho (1,2-d)thiazole, 2-(p-dimethylaminobenzoyl)styrene, diphenylacetamide, benzanilide, N-methylacetanilide, and 3′,4′-dimethylacetanilide.

In addition, other sensitizing dyes may be used.

For details of the sensitizing dye, reference can be made to the description in paragraphs 0161 to 0163 of JP2016-027357A, the content of which is incorporated in the present specification.

In a case where the resin composition contains a sensitizing agent, the content of the sensitizing agent is preferably 0.01% to 20% by mass, more preferably 0.1% to 15% by mass, and still more preferably 0.5% to 10% by mass with respect to the total solid content of the resin composition. One kind of sensitizing agent may be used alone, or two or more kinds thereof may be used in combination.

### [Chain Transfer Agent]

The resin composition according to the embodiment of the present invention may contain a chain transfer agent. The chain transfer agent is defined, for example, in Polymer Dictionary, 3rd Edition, pp. 683 to 684 (edited by The Society of Polymer Science, 2005).

As the chain transfer agent, for example, the following compound is used; a group of compounds having —S—S—, —SO₂—S—, —N—O—, SH, PH, SiH, or GeH in the molecule, or a dithiobenzoate compound, a trithiocarbonate compound, dithiocarbamate, or a xanthate compound, which has a thiocarbonylthio group that is used for the reversible addition fragmentation chain transfer (RAFT) polymerization. These can donate hydrogen to a low active radical to generate a radical or can be oxidized and then deprotonated to generate a radical. In particular, a thiol compound can be preferably used.

In addition, as the chain transfer agent, the compounds described in paragraphs 0152 and 0153 of WO2015/199219A can also be used, the content of which is incorporated in the present specification.

In a case where the resin composition has a chain transfer agent, the content of the chain transfer agent is preferably 0.01 to 20 parts by mass, more preferably 0.1 to 10 parts by mass, and still more preferably 0.5 to 5 parts by mass, with respect to 100 parts by mass of the total solid content of the resin composition. Only one kind of chain transfer agent may be used alone, or two or more kinds thereof may be used. In a case where two or more kinds of chain transfer agents are used, the total thereof is preferably within the above-described range.

In addition, the polymerization initiator may be a photoacid generator. The photoacid generator is preferably a photoacid generator that generates a radical.

Specifically, the compound is preferably a compound that absorbs light, decomposes the absorbed light to generate a radical, and abstracts hydrogen from a solvent, an acid generator itself, or the like to generate an acid.

Examples of the photoacid generator include a quinone diazide compound, an oxime sulfonate compound, an organic halogenated compound, an organic borate compound, a disulfone compound, and an onium salt, and an onium salt is preferable.

Examples of the onium salt include a diazonium salt, a phosphonium salt, a sulfonium salt, and an iodonium salt.

Further, the onium salt is a salt of a cation having an onium structure and an anion, and the cation and the anion may be or may not be bonded through a covalent bond.

That is, the onium salt may be an intramolecular salt having a cation moiety and an anion moiety in the same molecular structure, or may be an intermolecular salt in which a cation molecule and an anion molecule, which are separate molecules, are ionically bonded; however, an intermolecular salt is preferred. Further, in the composition according to the embodiment of the present invention, the cation moiety or the cation molecule and the anion moiety or the anion molecule may be bonded by the ionic bonding or be dissociated from each other.

### [Sulfonium Salt]

In the present invention, the sulfonium salt means a salt of a sulfonium cation and an anion.

### —Sulfonium Cation —

The sulfonium cation is preferably a tertiary sulfonium cation and more preferably a triaryl sulfonium cation.

In addition, the sulfonium cation is preferably a cation represented by Formula (103).

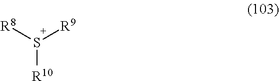

In Formula (103), R¹ to R¹⁰ each independently represent a hydrocarbon group.

R¹ to R¹⁰ are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R¹ to R¹⁰ may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R¹ to R¹⁰ preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R¹ to R¹⁰ may be the same group or different groups; however, from the viewpoint of synthesis compatibility, R⁸ to R¹⁰ are preferably the same group.

### —Anion—

The anion is not particularly limited, and may be selected in consideration of the acid generated. Examples of the anion include boron-based anions such as B(C₆F₅)₄⁻ and BF₄⁻, phosphorus-based anions such as (Rf)~PF₆₋ₙ⁻, PF₃(C₂F₅)₃⁻ and PF₆⁻, antimony-based anions such as SbF₆⁻, and other carboxylate anions, sulfonate anions, and the like.

### [Iodonium Salt]

In the present invention, the iodonium salt means a salt of an iodonium cation and an anion. Examples of the anion include the same anions as those in the sulfonium salt described above, and the same applies to the preferred aspect.

### —Iodonium Cation —

The iodonium cation is preferably a diaryl iodonium cation.

In addition, the iodonium cation is preferably a cation represented by Formula (104).

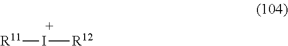

In Formula (104), R¹¹ and R¹² each independently represent a hydrocarbon group.

R¹¹ and R¹² are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R¹¹ and R¹² may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R¹¹ to R¹² preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R¹¹ and R¹² may be the same group or different groups; however, from the viewpoint of synthesis compatibility, R¹¹ and R¹² are preferably the same group.

### [Phosphonium Salt]

In the present invention, the phosphonium salt means a salt of a phosphonium cation and an anion. Examples of the anion include the same anions as those in the sulfonium salt described above, and the same applies to the preferred aspect.

### —Phosphonium Cation —

The phosphonium cation is preferably a quaternary phosphonium cation, and examples thereof include a tetraalkylphosphonium cation and a triarylmonoalkylphosphonium cation.

In addition, the phosphonium cation is preferably a cation represented by Formula (105).

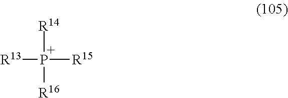

In Formula (105), R¹³ to R¹⁶ each independently represent a hydrogen atom or a hydrocarbon group.

R¹³ to R¹⁶ are each independently preferably an alkyl group or an aryl group, more preferably an alkyl group having 1 to 10 carbon atoms or an aryl group having 6 to 12 carbon atoms, still more preferably an aryl group having 6 to 12 carbon atoms, and even still more preferably a phenyl group.

R¹³ to R¹⁶ may have a substituent, and examples of the substituent include a hydroxy group, an aryl group, an alkoxy group, an aryloxy group, an arylcarbonyl group, an alkylcarbonyl group, an alkoxycarbonyl group, an aryloxycarbonyl group, and an acyloxy group. Among these, R¹³ to R¹⁶ preferably have an alkyl group or an alkoxy group as the substituent, more preferably have a branched alkyl group or an alkoxy group, and still more preferably have a branched alkyl group having 3 to 10 carbon atoms or an alkoxy group having 1 to 10 carbon atoms.

R¹³ to R¹⁶ may be the same group or different groups; however, from the viewpoint of synthesis compatibility, R¹³ to R¹⁶ are preferably the same group.

The content of the photoacid generator is preferably 0.1% to 20% by mass, more preferably 0.5% to 18% by mass, still more preferably 0.5% to 10% by mass, even more preferably 0.5% to 3% by mass, and even still more preferably 0.5% to 1.2% by mass, with respect to the total solid content of the resin composition.

One kind of photoacid generator may be used alone, or a plurality of kinds thereof may be used in combination. In the case of the combination of a plurality of kinds, it is preferable that the total amount thereof is within the above range.

In addition, in order to impart photosensitivity to a desired light source, it is also preferable to use a sensitizing agent in combination.

In addition, one of the preferred aspects of the present invention is that the resin composition according to the embodiment of the present invention contains two or more kinds of polymerization initiators as the polymerization initiator.

Specifically, the resin composition according to the embodiment of the present invention preferably contains a photopolymerization initiator and a thermal polymerization initiator described later, or contains the above-described photoradical polymerization initiator and the above-described photoacid generator.

By containing the photopolymerization initiator and the thermal polymerization initiator described later, pattern formation by exposure is possible, and radical polymerization is also likely to proceed during curing in the heating step described later, and thus the performance such as chemical resistance may be improved.

In the content ratio in a case where the photopolymerization initiator and the thermal polymerization initiator described later are contained, the content of the thermal polymerization initiator is preferably 20% to 70% by mass and more preferably 30% to 60% by mass with respect to the total content of the photopolymerization initiator and the thermal polymerization initiator.

The performance such as resolution may be improved by containing a photoradical polymerization initiator and a photoacid generator.

In the content ratio in a case where the photoradical polymerization initiator and the photoacid generator are contained, the content of the photoacid generator is preferably 20% to 70% by mass, and more preferably 30% to 60% by mass with respect to the total content of the photoradical polymerization initiator and the photoacid generator.

### [Thermal Polymerization Initiator]

Examples of the thermal polymerization initiator include a thermal radical polymerization initiator. The thermal radical polymerization initiator is a compound that generates a radical by heat energy and initiates or accelerates a polymerization reaction of a compound having polymerization properties. In a case where a thermal radical polymerization initiator is added, the polymerization reaction of the resin and the polymerizable compound can be allowed to proceed, and thus the solvent resistance can be further improved.

Specific examples of thermal radical polymerization initiator include compounds described in paragraphs 0074 to 0118 of JP2008-063554A, the content of which is incorporated in the present specification.

In a case where a thermal polymerization initiator is contained, the content thereof is preferably 0.1% to 30% by mass, more preferably 0.1% to 20% by mass, and still more preferably 0.5% to 15% by mass with respect to the total solid content of the resin composition. Only one kind of thermal polymerization initiator may be contained, or two or more kinds thereof may be contained. In a case where two or more kinds of thermal polymerization initiators are contained, the total amount thereof is preferably within the above-described range.

### <Base Generator>

The resin composition according to the embodiment of the present invention may contain a base generator. Here, the base generator is a compound that is capable of generating a base under a physical or chemical action. Examples of the preferred base generator include a thermal-base generator and a photobase generator.

In particular, in a case where the resin composition contains a precursor of a cyclization resin, it is preferable that the resin composition contains a base generator. In a case where the resin composition contains a thermal-base generator, it is possible to, for example, accelerate the cyclization reaction of the precursor by heating, whereby the mechanical properties and chemical resistance of the cured substance are improved and for example, the performance as an interlayer insulating film for a re-distribution layer, included in a semiconductor package, is improved.

The base generator may be an ionic base generator or may be a nonionic base generator. Examples of the base that is generated from the base generator include a secondary amine and a tertiary amine.

The base generator is not particularly limited, and a publicly known base generator can be used. Examples of the publicly known base generator include a carbamoyloxime compound, a carbamoylhydroxylamine compound, a carbamic acid compound, a formamide compound, an acetamide compound, a carbamate compound, a benzylcarbamate compound, a nitrobenzylcarbamate compound, a sulfonamide compound, an imidazole derivative compound, an aminimide compound, a pyridine derivative compound, an α-aminoacetophenone derivative compound, a quaternary ammonium salt derivative compound, an iminium salt, a pyridinium salt, an α-lactone ring derivative compound, a phthalimide derivative compound, and an acyloxyimino compound.

Specific examples of the compound of the nonionic base generator include compounds represented by Formula (B1), Formula (B2), or Formula (B3).

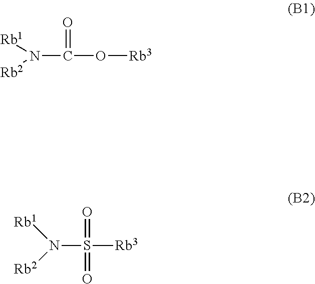

In Formulae (B1) and (B2), Rb¹, Rb², and Rb³ each independently represent an organic group that does not have a tertiary amine structure, a halogen atom, or a hydrogen atom. However, Rb¹ and Rb² are not hydrogen atoms at the same time. Further, none of Rb¹, Rb², and Rb³ have a carboxy group. In the present specification, the tertiary amine structure refers to a structure in which all three bonding sites of a trivalent nitrogen atom are covalently bonded to carbon atoms of the hydrocarbon group. Therefore, in a case where the carbon atom bonded to the trivalent nitrogen atom is a carbon atom constituting a carbonyl group, that is, in a case where an amide group is formed together with the nitrogen atom, it is not a tertiary amine structure.

In Formula (B1) and Formula (B2), a cyclic structure is preferably contained in at least one of Rb¹, Rb², or Rb³ and is more preferably contained in at least two thereof. The cyclic structure may be any one of a monocyclic ring or a fused ring and is preferably a monocyclic ring or a fused ring in which two monocyclic rings are fused. The monocyclic ring is preferably a 5-membered ring or a 6-membered ring, and more preferably a 6-membered ring. The monocyclic ring is preferably a cyclohexane ring or a benzene ring, and more preferably a cyclohexane ring.

More specifically, Rb¹ and Rb² are preferably a hydrogen atom, an alkyl group (preferably having 1 to 24 carbon atoms, more preferably having 2 to 18 carbon atoms, and still more preferably having 3 to 12 carbon atoms), an alkenyl group (preferably having 2 to 24 carbon atoms, more preferably having 2 to 18 carbon atoms, and still more preferably having 3 to 12 carbon atoms), an aryl group (preferably having 6 to 22 carbon atoms, more preferably having 6 to 18 carbon atoms, and still more preferably having 6 to 10 carbon atoms), or an arylalkyl group (preferably having 7 to 25 carbon atoms, more preferably having 7 to 19 carbon atoms, and still more preferably having 7 to 12 carbon atoms). These groups may have a substituent. Rb¹ and Rb² may be bonded to each other to form a ring. The ring to be formed is preferably a 4- to 7-membered nitrogen-containing heterocycle. Rb¹ and Rb² are preferably a linear, branched, or cyclic alkyl groups (preferably having 1 to 24 carbon atoms, more preferably 2 to 18 carbon atoms, and still more preferably having 3 to 12 carbon atoms) which may have a substituent, more preferably a cycloalkyl group (preferably having 3 to 24 carbon atoms, more preferably having 3 to 18 carbon atoms, and still more preferably having 3 to 12 carbon atoms) which may have a substituent, and still more preferably a cyclohexyl group which may have a substituent.

Examples of Rb³ include an alkyl group (preferably having 1 to 24 carbon atoms, more preferably having 2 to 18 carbon atoms, and still more preferably having 3 to 12 carbon atoms), an aryl group (preferably having 6 to 22 carbon atoms, more preferably having 6 to 18 carbon atoms, and still more preferably having 6 to 10 carbon atoms), an alkenyl group (preferably having 2 to 24 carbon atoms, more preferably having 2 to 12 carbon atoms, and still more preferably having 2 to 6 carbon atoms), an arylalkyl group (preferably having 7 to 23 carbon atoms, more preferably having 7 to 19 carbon atoms, and still more preferably having 7 to 12 carbon atoms), an arylalkenyl group (preferably having 8 to 24 carbon atoms, more preferably having 8 to 20 carbon atoms, and still more preferably having 8 to 16 carbon atoms), an alkoxyl group (preferably having 1 to 24 carbon atoms, more preferably having 2 to 18 carbon atoms, and still more preferably having 3 to 12 carbon atoms), an aryloxy group (preferably having 6 to 22 carbon atoms, more preferably having 6 to 18 carbon atoms, and still more preferably having 6 to 12 carbon atoms), and an arylalkyloxy group (preferably having 7 to 23 carbon atoms, more preferably having 7 to 19 carbon atoms, and still more preferably having 7 to 12 carbon atoms). Among the above, a cycloalkyl group (preferably having 3 to 24 carbon atoms, more preferably 3 to 18 carbon atoms, and still more preferably 3 to 12 carbon atoms), an arylalkenyl group, or an arylalkyloxy group is preferable. Rb³ may further have a substituent.

The compound represented by Formula (B1) is preferably a compound represented by Formula (B1-1) or Formula (B1-2).

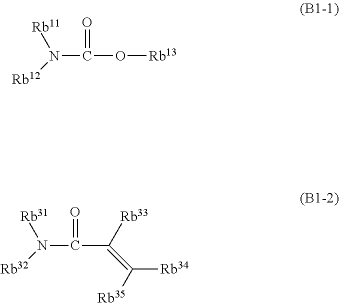

In Formula, Rb¹¹ and Rb¹², and Rb³¹ and Rb³² each have the same meanings as Rb¹ and Rb² in Formula (B1).

Rb¹³ is an alkyl group (preferably having 1 to 24 carbon atoms, more preferably having 2 to 18 carbon atoms, and still more preferably having 3 to 12 carbon atoms), an alkenyl group (preferably having 2 to 24 carbon atoms, more preferably having 2 to 18 carbon atoms, and still more preferably having 3 to 12 carbon atoms), an aryl group (preferably having 6 to 22 carbon atoms, more preferably having 6 to 18 carbon atoms, and still more preferably having 6 to 12 carbon atoms), or an arylalkyl group (preferably having 7 to 23 carbon atoms, more preferably having 7 to 19 carbon atoms, and still more preferably having 7 to 12 carbon atoms), and may have a substituent. Among the above, Rb¹³ is preferably an arylalkyl group.

Rb³³ and Rb³⁴ are each independently a hydrogen atom, an alkyl group (preferably having 1 to 12 carbon atoms, more preferably having 1 to 8 carbon atoms, and still more preferably having 1 to 3 carbon atoms), an alkenyl group (preferably having 2 to 12 carbon atoms, more preferably having 2 to 8 carbon atoms, and still more preferably having 2 or 3 carbon atoms), an aryl group (preferably having 6 to 22 carbon atoms, more preferably having 6 to 18 carbon atoms, and still more preferably having 6 to 10 carbon atoms), or an arylalkyl group (preferably having 7 to 23 carbon atoms, more preferably having 7 to 19 carbon atoms, and still more preferably having 7 to 11 carbon atoms), and preferably a hydrogen atom.

Rb³⁵ is an alkyl group (preferably having 1 to 24 carbon atoms, more preferably having 1 to 12 carbon atoms, and still more preferably having 3 to 8 carbon atoms), an alkenyl group (preferably having 2 to 12 carbon atoms, more preferably having 2 to 10 carbon atoms, and still more preferably having 3 to 8 carbon atoms), an aryl group (preferably having 6 to 22 carbon atoms, more preferably having 6 to 18 carbon atoms, and still more preferably having 6 to 12 carbon atoms), or an arylalkyl group (preferably having 7 to 23 carbon atoms, more preferably having 7 to 19 carbon atoms, and still more preferably having 7 to 12 carbon atoms), and is preferably an aryl group.

The compound represented by Formula (B1-1) is preferably a compound represented by Formula (B1-1a).

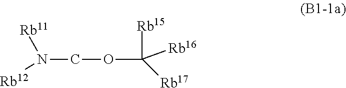

Rb¹¹ and Rb¹² respectively have the same meanings as Rb¹¹ and Rb¹² in Formula (B1-1).

Rb¹⁵ and Rb¹⁶ are a hydrogen atom, an alkyl group (preferably having 1 to 12 carbon atoms, more preferably having 1 to 6 carbon atoms, and still more preferably having 1 to 3 carbon atoms), an alkenyl group (preferably having 2 to 12 carbon atoms, more preferably having 2 to 6 carbon atoms, and still more preferably having 2 or 3 carbon atoms), an aryl group (preferably having 6 to 22 carbon atoms, more preferably having 6 to 18 carbon atoms, and still more preferably having 6 to 10 carbon atoms), or an arylalkyl group (preferably having 7 to 23 carbon atoms, more preferably having 7 to 19 carbon atoms, and still more preferably having 7 to 11 carbon atoms), and preferably a hydrogen atom or a methyl group.

Rb¹⁷ is an alkyl group (preferably having 1 to 24 carbon atoms, more preferably having 1 to 12 carbon atoms, and still more preferably having 3 to 8 carbon atoms), an alkenyl group (preferably having 2 to 12 carbon atoms, more preferably having 2 to 10 carbon atoms, and still more preferably having 3 to 8 carbon atoms), an aryl group (preferably having 6 to 22 carbon atoms, more preferably having 6 to 18 carbon atoms, and still more preferably having 6 to 12 carbon atoms), or an arylalkyl group (preferably having 7 to 23 carbon atoms, more preferably having 7 to 19 carbon atoms, and still more preferably having 7 to 12 carbon atoms), and among the above, is preferably an aryl group.

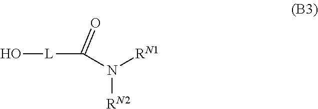

In Formula (B3), L represents a hydrocarbon group which is a divalent hydrocarbon group having a saturated hydrocarbon group on a path of a linking chain that links adjacent oxygen atom and carbon atom and in which the number of the atoms on the path of the linking chain is 3 or more. Further, R^{N1} and R^{N2} each independently represent a monovalent organic group.

In the present specification, the “linking chain” refers to an atomic chain which interconnects linking targets shortest (with the minimum number of atoms) among the atomic chains on the path, which interconnects two atoms or a group of atoms to be linked. For example, in the compound represented by the following Formula, L is composed of a phenylene ethylene group, has an ethylene group as a saturated hydrocarbon group, and the linking chain is composed of four carbon atoms, and the number of atoms (that is, the number of atoms constituting the linking chain, and hereinafter, also referred to as the “linking chain length” or the “length of linking chain”) on the path of the linking chain is 4.

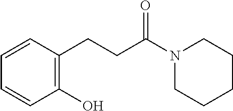 [SMILES: O=C(CCc1ccccc1O)N1CCCCC1]

The number of carbon atoms in L in Formula (B3) (including carbon atoms other than the carbon atoms in the linking chain) is preferably 3 to 24. The upper limit thereof is more preferably 12 or less, still more preferably 10 or less, and particularly preferably 8 or less. The lower limit thereof is more preferably 4 or more. From the viewpoint of rapidly advancing the intramolecular cyclization reaction, the upper limit of the linking chain length of L is preferably 12 or less, more preferably 8 or less, still more preferably 6 or less, and particularly preferably 5 or less. In particular, the linking chain length of L is preferably 4 or 5 and most preferably 4. Specific preferred compounds of the base generator include the compounds described in paragraphs 0102 to 0168 of WO2020/066416A and the compounds described in paragraphs 0143 to 0177 of WO2018/038002A.

In addition, it is also preferable that the base generator includes a compound represented by Formula (N1).

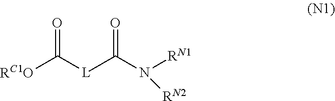

In Formula (N1), R^{N1} and R^{N2} each independently represent a monovalent organic group, R^{C1} represents a hydrogen atom or a protective group, and L represents a divalent linking group.

L is a divalent linking group, and it is preferably a divalent organic group. The linking chain length of the linking group is preferably 1 or more and more preferably 2 or more. The upper limit thereof is preferably 12 or less, more preferably 8 or less, and still more preferably 5 or less. The linking chain length is the number of atoms present in the atom sequence that is the shortest route between the two carbonyl groups in Formula.

R^{N1} and R^{N2} in Formula (N1) each independently represent a monovalent organic group (preferably having 1 to 24 carbon atoms, more preferably 2 to 18 carbon atoms, and still more preferably 3 to 12 carbon atoms), more preferably a hydrocarbon group (preferably having 1 to 24 carbon atoms, more preferably 1 to 12 carbon atoms, and still more preferably 1 to 10 carbon atoms), and specific examples thereof include an aliphatic hydrocarbon group (preferably having 1 to 24 carbon atoms, more preferably 1 to 12 carbon atoms, and still more preferably 1 to 10 carbon atoms) and an aromatic hydrocarbon group (preferably having 6 to 22 carbon atoms, more preferably 6 to 18 carbon atoms, and still more preferably 6 to 10 carbon atoms), and an aliphatic hydrocarbon group is preferable. As R^{N1} and R^{N2}, an aliphatic hydrocarbon group is preferably used since the basicity of the base to be generated is high. It is noted that the aliphatic hydrocarbon group and the aromatic hydrocarbon group may have a substituent, and the aliphatic hydrocarbon group and the aromatic hydrocarbon group may have an oxygen atom in an aliphatic hydrocarbon chain, in an aromatic ring, or in a substituent. In particular, an aspect in which the aliphatic hydrocarbon group has an oxygen atom in a hydrocarbon chain is exemplified.

Examples of the aliphatic hydrocarbon group constituting R^{N1} and R^{N2} include a linear or branched chain-like alkyl group, a cyclic alkyl group, a group including a combination of a chain-like alkyl group and a cyclic alkyl group, and an alkyl group having an oxygen atom in the chain. The linear or branched chain-like alkyl group preferably has 1 to 24 carbon atoms, more preferably 2 to 18, and still more preferably 3 to 12 carbon atoms. Examples of the linear or branched chain-like alkyl group include a methyl group, an ethyl group, a propyl group, a butyl group, a pentyl group, a hexyl group, a heptyl group, an octyl group, a nonyl group, a decyl group, an undecyl group, a dodecyl group, an isopropyl group, an isobutyl group, a secondary butyl group, a tertiary butyl group, an isopentyl group, a neopentyl group, a tertiary pentyl group, and an isohexyl group.

The cyclic alkyl group preferably has 3 to 12 carbon atoms and more preferably 3 to 6 carbon atoms. Examples of the cyclic alkyl group include a cyclopropyl group, a cyclobutyl group, a cyclopentyl group, a cyclohexyl group, and a cyclooctyl group.

The group including a combination of a chain-like alkyl group and a cyclic alkyl group preferably has 4 to 24 carbon atoms, more preferably 4 to 18 carbon atoms, and still more preferably 4 to 12 carbon atoms. Examples of the group including a combination of a chain-like alkyl group and a cyclic alkyl group include a cyclohexylmethyl group, a cyclohexylethyl group, a cyclohexylpropyl group, a methylcyclohexylmethyl group, and an ethylcyclohexylethyl group.

The alkyl group having an oxygen atom in the chain preferably has 2 to 12 carbon atoms, more preferably 2 to 6 carbon atoms, and still more preferably 2 to 4 carbon atoms. The alkyl group having an oxygen atom in the chain may be chain-like or cyclic or may be linear or branched.

Among the above, from the viewpoint of increasing the boiling point of the decomposition-generated base described later, R^{N1} and R^{N2} are preferably an alkyl group having 5 to 12 carbon atoms. However, in a prescription in which adhesiveness in a case of carrying out lamination with a metal (for example, copper) layer is important, a group having a cyclic alkyl group or an alkyl group having 1 to 8 carbon atoms is preferable.

R^{N1} and R^{N2} may be linked to each other to form a cyclic structure. The cyclic structure may have an oxygen atom or the like in the chain. The cyclic structure formed by R^{N1} and R^{N2} can be a monocyclic ring or may be a fused ring, but is preferably a monocyclic ring. The cyclic structure to be formed is preferably a 5-membered ring or a 6-membered ring containing a nitrogen atom in Formula (N1), examples thereof include a pyrrole ring, an imidazole ring, a pyrazole ring, a pyrroline ring, a pyrrolidine ring, an imidazolidine ring, a pyrazolidine ring, a piperidine ring, a piperazine ring, and a morpholine ring, and preferred examples thereof include a pyrroline ring, a pyrrolidine ring, a piperidine ring, a piperazine ring, and a morpholine ring.

R^{C1} represents a hydrogen atom or a protective group, and it is preferably a hydrogen atom.

The protective group is preferably a protective group that decomposes under the action of an acid or a base, and examples thereof preferably include a protective group that decomposes by an acid.

Specific examples of the protective group include a chain-like or cyclic alkyl group and a chain-like or cyclic alkyl group having an oxygen atom in the chain. Examples of the chain-like or cyclic alkyl group include a methyl group, an ethyl group, an isopropyl group, a tert-butyl group, and a cyclohexyl group. Examples of the chain-like alkyl group having an oxygen atom in the chain include an alkyloxyalkyl group, and a methyloxymethyl (MOM) group and an ethyloxyethyl (EE) group are preferable. Examples of the cyclic alkyl group having an oxygen atom in the chain include an epoxy group, a glycidyl group, an oxetanyl group, a tetrahydrofuranyl group, and a tetrahydropyranyl (THP) group.

In Formula (N1), the divalent linking group constituting L is not particularly limited, but is preferably a hydrocarbon group and more preferably an aliphatic hydrocarbon group. The hydrocarbon group may have a substituent or may have an atom other than a carbon atom in the hydrocarbon chain. The divalent linking group is more preferably a divalent hydrocarbon linking group which may have an oxygen atom in the chain, still more preferably a divalent aliphatic hydrocarbon group which may have an oxygen atom in the chain, a divalent aromatic hydrocarbon group, or a group including a combination of a divalent aliphatic hydrocarbon group which may have an oxygen atom in the chain and a divalent aromatic hydrocarbon group, and even still more preferably a divalent aliphatic hydrocarbon group which may have an oxygen atom in the chain. These groups may not have an oxygen atom.

The divalent hydrocarbon linking group preferably has 1 to 24 carbon atoms, more preferably 2 to 12 carbon atoms, and still more preferably 2 to 6 carbon atoms. The divalent aliphatic hydrocarbon group preferably has 1 to 12 carbon atoms, more preferably 2 to 6 carbon atoms, and still more preferably 2 to 4 carbon atoms. The divalent aromatic hydrocarbon group preferably has 6 to 22 carbon atoms, more preferably 6 to 18 carbon atoms, and still more preferably 6 to 10 carbon atoms. The group including a combination of a divalent aliphatic hydrocarbon group and a divalent aromatic hydrocarbon group (for example, an arylene alkyl group) preferably has 7 to 22 carbon atoms, more preferably 7 to 18 carbon atoms, and still more preferably 7 to 10 carbon atoms.

Specifically, the linking group L is preferably a linear or branched chain-like alkylene group, a cyclic alkylene group, a group including a combination of a chain-like alkylene group and a cyclic alkylene group, an alkylene group having an oxygen atom in the chain, a linear or branched chain-like alkenylene group, a cyclic alkenylene group, an arylene group, or an arylene alkylene group.

The linear or branched chain-like alkylene group preferably has 1 to 12 carbon atoms, more preferably 2 to 6 carbon atoms, and still more preferably 2 to 4 carbon atoms.

The cyclic alkylene group preferably has 3 to 12 carbon atoms and more preferably 3 to 6 carbon atoms.

The group including a combination of a chain-like alkylene group and a cyclic alkylene group preferably has 4 to 24 carbon atoms, more preferably 4 to 12 carbon atoms, and still more preferably 4 to 6 carbon atoms.

The alkylene group having an oxygen atom in the chain may be chain-like or cyclic or may be linear or branched. The alkylene group having an oxygen atom in the chain preferably has 1 to 12 carbon atoms, more preferably 1 to 6 carbon atoms, and still more preferably 1 to 3 carbon atoms.

The linear or branched chain-like alkenylene group preferably has 2 to 12 carbon atoms, more preferably 2 to 6 carbon atoms, and still more preferably 2 to 3 carbon atoms. The number of C═C bonds in the linear or branched chain-like alkenylene group is preferably 1 to 10, more preferably 1 to 6, and still more preferably 1 to 3.

The cyclic alkenylene group preferably has 3 to 12 carbon atoms and more preferably 3 to 6 carbon atoms. In the cyclic alkenylene group, the number of C═C bonds is preferably 1 to 6, more preferably 1 to 4, and still more preferably 1 to 2.

The arylene group preferably has 6 to 22 carbon atoms, more preferably 6 to 18 carbon atoms, and still more preferably 6 to 10 carbon atoms.

The arylene alkylene group preferably has 7 to 23 carbon atoms, more preferably 7 to 19, and still more preferably 7 to 11 carbon atoms.

Among the above, a preferred one is a chain-like alkylene group, a cyclic alkylene group, an alkylene group having an oxygen atom in the chain, a chain-like alkenylene group, an arylene group, or an arylene alkylene group, and more preferred one is a 1,2-ethylene group, a propanediyl group (particularly a 1,3-propanediyl group), a cyclohexanediyl group (particularly a 1,2-cyclohexanediyl group), a vinylene group (particularly a cisvinylene group), a phenylene group (a 1,2-phenylene group), a phenylene methylene group (particularly a 1,2-phenylene methylene group) or an ethyleneoxyethylene group (particularly a 1,2-ethyleneoxy-1,2-ethylene group).

Examples of the base generator include the following compounds; however, the base generator is not limited thereto.

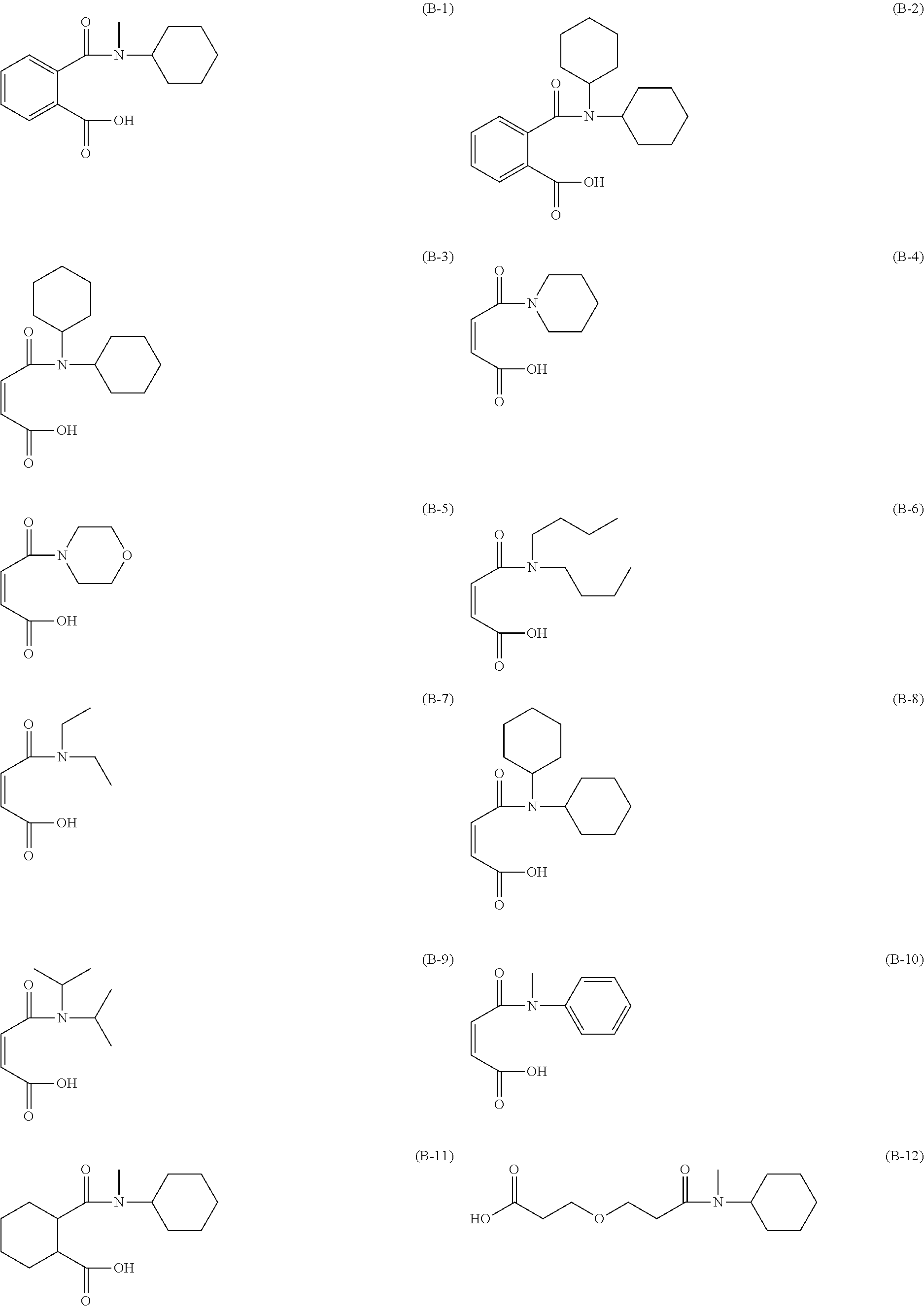

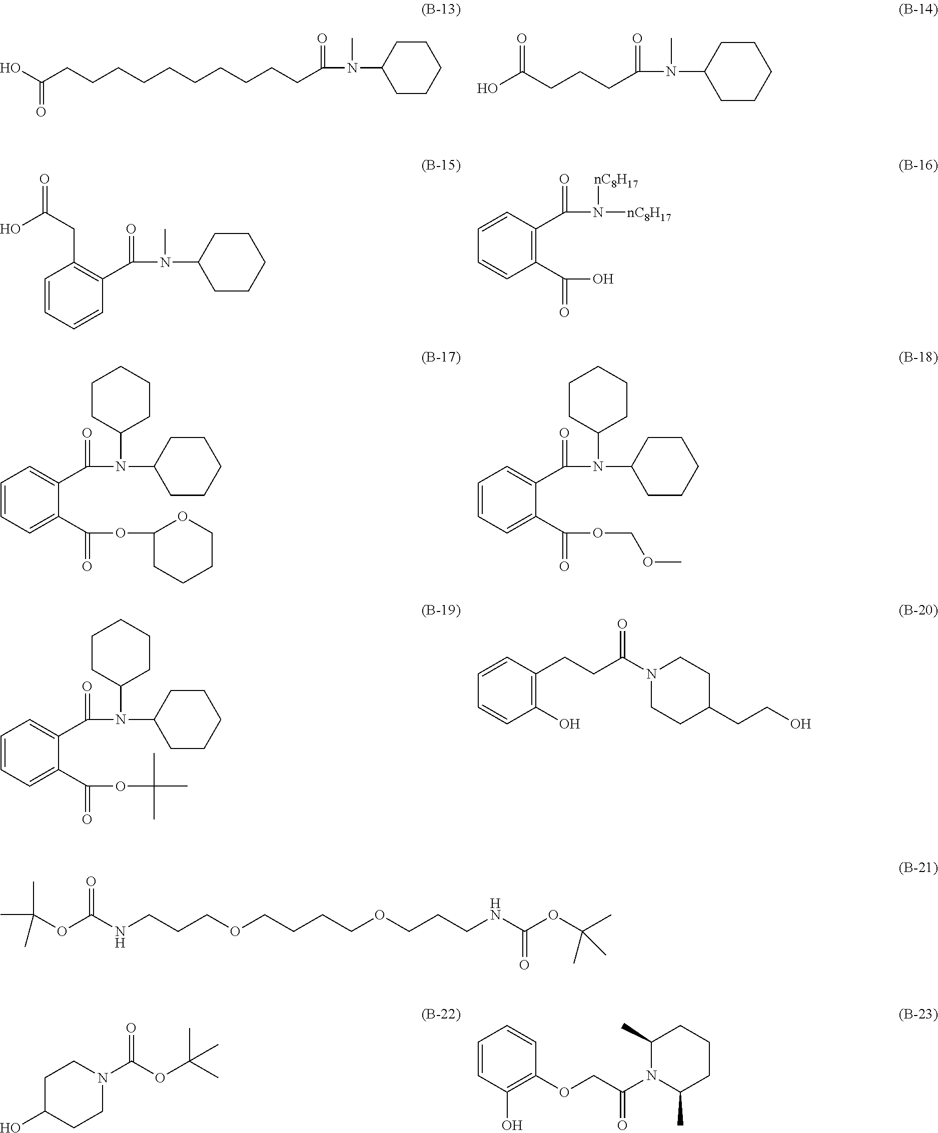

The molecular weight of the nonionic base generator is preferably 800 or less, more preferably 600 or less, and still more preferably 500 or less. The lower limit thereof is preferably 100 or more, more preferably 200 or more, and still more preferably 300 or more.

Examples of the specific preferred compound of the ionic base generator include the compounds described in paragraphs 0148 to 0163 of WO2018/038002A.

Specific examples of the ammonium salt include the following compounds; however, the ammonium salt is not limited thereto.

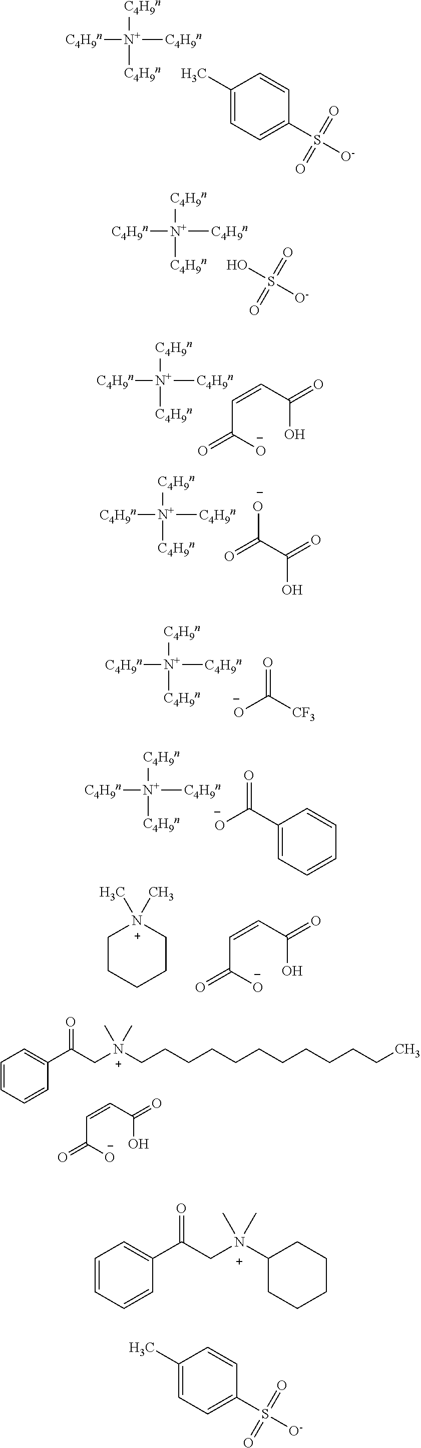

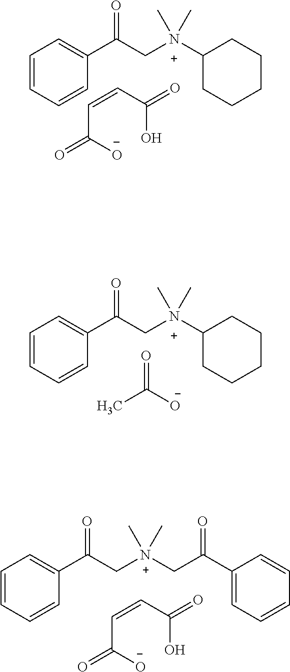

Specific examples of the iminium salt include the following compounds; however, the iminium salt is not limited thereto.

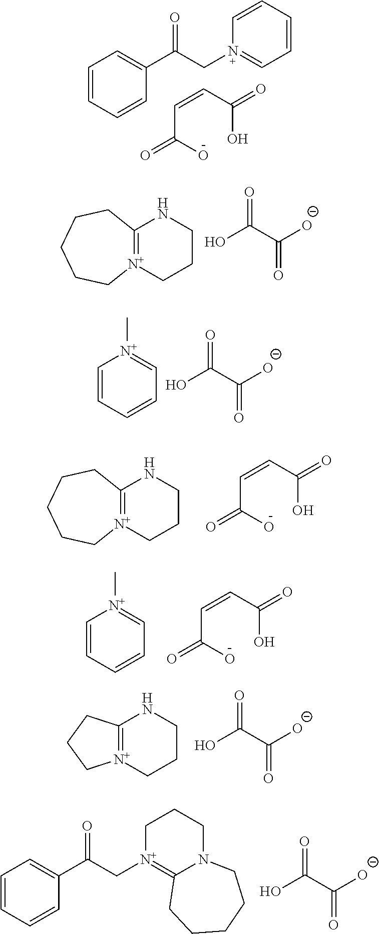

In addition, from the viewpoint of storage stability and generation of a base by deprotection during curing, the base generator is preferably an amine in which an amino group is protected by a t-butoxycarbonyl group.

Examples of the amine compound protected by a t-butoxycarbonyl group include ethanolamine, 3-amino-1-propanol, 1-amino-2-propanol, 2-amino-1-propanol, 4-amino-1-butanol, 2-amino-1-butanol, 1-amino-2-butanol, 3-amino-2,2-dimethyl-1-propanol, 4-amino-2-methyl-1-butanol, valinol, 3-amino-1,2-propanediol, 2-amino-1,3-propanediol, tyramine, norephedrine, 2-amino-1-phenyl-1,3-propanediol, 2-aminocyclohexanol, 4-aminocyclohexanol, 4-aminocyclohexanethanol, 4-(2-aminoethyl)cyclohexanol, N-methylethanolamine, 3-(methylamino)-1-propanol, 3-(isopropylamino)propanol, N-cyclohexylethanolamine, α-[2-(methylamino)ethyl]benzyl alcohol, diethanolamine, diisopropanolamine, 3-pyrrolidinol, 2-pyrrolidinomethanol, 4-hydroxypiperidine, 3-hydroxypiperidine, 4-hydroxy-4-phenylpiperidine, 4-(3-hydroxyphenyl)piperidine, 4-piperidinemethanol, 3-piperidinemethanol, 2-piperidinemethanol, 4-piperidinetanol, 2-piperidinetanol, 2-(4-piperidyl)-2-propanol, 1,4-butanol bis(3-aminopropyl)ether, 1,2-bis(2-aminoethoxy)ethane, 2,2′-oxybis(ethylamine), 1,14-diamino-3,6,9,12-tetraoxatetradecane, 1-aza-15-crown 5-ether, diethylene glycol bis(3-aminopropyl)ether, 1,11-diamino-3,6,9-trioxaundecane, or a compound in which an amino group of an amino acid and a derivative thereof is protected by a t-butoxycarbonyl group, but the present invention is not limited to these examples.

In a case where the resin composition contains a base generator, the content of the base generator is preferably 0.1 to 50 parts by mass with respect to 100 parts by mass of the resin in the resin composition. The lower limit thereof is more preferably 0.3 parts by mass or more and still more preferably 0.5 parts by mass or more. The upper limit thereof is more preferably 30 parts by mass or less, still more preferably 20 parts by mass or less, even still more preferably 10 parts by mass or less, even further still more preferably 5 parts by mass or less, and particularly preferably 4 parts by mass or less.

One kind or two or more kinds of base generators can be used. In a case where two or more kinds thereof are used, the total amount is preferably within the above-described range.

### <Solvent>

The resin composition according to the embodiment of the present invention preferably contains a solvent.

As the solvent, any publicly known solvent can be used. The solvent is preferably an organic solvent. Examples of the organic solvent include compounds such as esters, ethers, ketones, cyclic hydrocarbons, sulfoxides, amides, ureas, and alcohols.

Suitable examples of the esters include ethyl acetate, n-butyl acetate, isobutyl acetate, hexyl acetate, amyl formate, isoamyl acetate, butyl propionate, isopropyl butyrate, ethyl butyrate, butyl butyrate, methyl lactate, ethyl lactate, γ-butyrolactone, ε-caprolactone, δ-valerolactone, γ-valerolactone, alkyl alkyloxyacetate (for example, methyl alkyloxyacetate, ethyl alkyloxyacetate, and butyl alkyloxyacetate (for example, methyl methoxyacetate, ethyl methoxyacetate, butyl methoxyacetate, methyl ethoxyacetate, and ethyl ethoxyacetate)), 3-alkyloxypropionic acid alkyl esters (for example, methyl 3-alkyloxypropionate, and ethyl 3-alkyloxypropionate (for example, methyl 3-methoxypropionate, ethyl 3-methoxypropionate, methyl 3-ethoxypropionate, and ethyl 3-ethoxypropionate)), 2-alkyloxypropionic acid alkyl esters (for example, methyl 2-alkyloxypropionate, ethyl 2-alkyloxypropionate, and propyl 2-alkyloxypropionate (for example, methyl 2-methoxypropionate, ethyl 2-methoxypropionate, propyl 2-methoxypropionate, methyl 2-ethoxypropionate, and ethyl 2-ethoxypropionate)), methyl 2-alkyloxy-2-methylpropionate and ethyl 2-alkyloxy-2-methylpropionate (for example, methyl 2-methoxy-2-methylpropionate and ethyl 2-ethoxy-2-methylpropionate), methyl pyruvate, ethyl pyruvate, propyl pyruvate, methyl acetoacetate, ethyl acetoacetate, methyl 2-oxobutanoate, ethyl 2-oxobutanoate, ethyl hexanoate, ethyl heptanoate, dimethyl malonate, and diethyl malonate.

Suitable examples of ethers include ethylene glycol dimethyl ether, diethylene glycol dimethyl ether, diethylene glycol diethyl ether, diethylene glycol ethyl methyl ether, diethylene glycol butyl methyl ether, triethylene glycol dimethyl ether, tetraethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol dimethyl ether, propylene glycol monomethyl ether acetate, propylene glycol monoethyl ether acetate, ethylene glycol monobutyl ether, ethylene glycol monobutyl ether acetate, propylene glycol monopropyl ether acetate, and dipropylene glycol dimethyl ether.

Suitable examples of the ketones include methyl ethyl ketone, cyclohexanone, cyclopentanone, 2-heptanone, 3-heptanone, 3-methylcyclohexanone, levoglucosenone, and dihydrolevoglucosenone.

Suitable examples of the cyclic hydrocarbon include aromatic hydrocarbons such as toluene, xylene, and anisole, and cyclic terpenes such as limonene.

Suitable examples of the sulfoxides include dimethyl sulfoxide.

Suitable examples of the amide include N-methyl-2-pyrrolidone, N-ethyl-2-pyrrolidone, N-cyclohexyl-2-pyrrolidone, N,N-dimethylacetamide, N,N-dimethylformamide, N,N-dimethylisobutylamide, 3-methoxy-N,N-dimethylpropionamide, 3-butoxy-N,N-dimethylpropionamide, N-formylmorpholine, and N-acetylmorpholine.

Suitable examples of the urea include N,N,N′,N′-tetramethylurea and 1,3-dimethyl-2-imidazolidinone.

Examples of the alcohols include methanol, ethanol, 1-propanol, 2-propanol, 1-butanol, 1-pentanol, 1-hexanol, benzyl alcohol, ethylene glycol monomethyl ether, 1-methoxy-2-propanol, 2-ethoxyethanol, diethylene glycol monoethyl ether, diethylene glycol monohexyl ether, triethylene glycol monomethyl ether, propylene glycol monoethyl ether, propylene glycol monomethyl ether, polyethylene glycol monomethyl ether, polypropylene glycol, tetraethylene glycol, ethylene glycol monobutyl ether, ethylene glycol monobenzyl ether, ethylene glycol monophenyl ether, methylphenyl carbinol, n-amyl alcohol, methylamyl alcohol, and diacetone alcohol.

From the viewpoint of improving the properties of a coated surface or the like, it is also preferable to mix two or more kinds of solvents.

In the present invention, the solvent is preferably one solvent selected from methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclopentanone, γ-butyrolactone, γ-valerolactone, 3-methoxy-N,N-dimethylpropionamide, toluene, dimethyl sulfoxide, ethyl carbitol acetate, butyl carbitol acetate, N-methyl-2-pyrrolidone, propylene glycol methyl ether, and propylene glycol methyl ether acetate, as well as levoglucosenone and dihydrolevoglucosenone, or a mixed solvent composed of two or more of these. A combined use of dimethyl sulfoxide and γ-butyrolactone, a combined use of dimethyl sulfoxide and γ-valerolactone, a combined use of 3-methoxy-N,N-dimethylpropionamide and γ-butyrolactone, a combined use of 3-methoxy-N,N-dimethylpropionamide and dimethyl sulfoxide, or, a combined use of N-methyl-2-pyrrolidone and ethyl lactate is particularly preferable. An aspect in which an amount of about 1% to 10% by mass of toluene with respect to the total mass of the solvent is further added to these combinedly used solvents is also one of the preferred aspects of the present invention.

In particular, from the viewpoint of the storage stability or the like of the resin composition, an aspect in which γ-valerolactone is included as the solvent is also one of the preferred aspects of the present invention. In such an aspect, the content of γ-valerolactone with respect to the total mass of the solvent is preferably 50% by mass or more, more preferably 60% by mass or more, and still more preferably 70% by mass or more. In addition, the upper limit of the above-described content is not particularly limited, and it may be 100% by mass. The above-described content may be determined in consideration of the solubility or the like of the component contained in the resin composition, such as the specific resin.

In addition, in a case where dimethyl sulfoxide and γ-valerolactone are used in combination, with respect to the total mass of the solvent, it is preferable to contain 60% to 90% by mass of γ-valerolactone and 10% to 40% by mass of dimethyl sulfoxide, it is more preferable to contain 70% to 90% by mass of γ-valerolactone and 10% to 30% by mass of dimethyl sulfoxide, and it is still more preferable to contain 75% to 85% by mass of γ-valerolactone and 15% to 25% by mass of dimethyl sulfoxide.

In addition, the resin composition according to the embodiment of the present invention preferably contains a solvent having a boiling point of 50° C. to 300° C. at 1 atm, and more preferably contains a solvent having the above-described boiling point of 100° C. to 260° C. In the present invention, the boiling point of the solvent is a boiling point at 1 atm.

According to such an aspect, it is considered that a cured substance having excellent solvent removability and excellent resolution can be obtained.

The above-described boiling point is preferably 150° C. or higher, more preferably 180° C. or higher, and still more preferably 200° C. or higher. The upper limit of the boiling point is preferably 250° C. or lower, more preferably 240° C. or lower, and still more preferably 230° C. or lower.

In addition, the resin composition according to the embodiment of the present invention preferably contains two or more kinds of solvents having a boiling point of 100° C. to 260° C., more preferably contains two or more kinds of solvents having a boiling point of 150° C. to 250° C., and still more preferably contains two or more kinds of solvents having a boiling point of 180° C. to 230° C.

In addition, a content of the solvent having a boiling point of 100° C. to 260° C. is preferably 40% by mass or more, more preferably 45% by mass or more, and still more preferably 50% by mass or more with respect to the total mass of the composition. In a case of containing two or more kinds of solvents having a boiling point of 100° C. to 260° C., the total amount thereof is preferably within the above-described range.

Regarding the content of the solvent, the amount of the solvent is such that, from the viewpoint of coatability, the concentration of the total solid contents of the resin composition according to the embodiment of the present invention is preferably 5% to 80% by mass, more preferably 5% to 75% by mass, still more preferably 10% to 70% by mass, and even still more preferably 20% to 70% by mass. The content of the solvent may be adjusted depending on the desired thickness of the coating film and the coating method. In a case where two or more kinds of solvents are contained, the total thereof is preferably within the above-described range.

### <Metal Adhesiveness Improving Agent>

From the viewpoint of improving adhesiveness to a metal material used for an electrode, a wire, or the like, the resin composition according to the embodiment of the present invention preferably contains a metal adhesiveness improving agent. Examples of the metal adhesiveness improving agent include a silane coupling agent having an alkoxysilyl group, an aluminum-based auxiliary adhesive agent, a titanium-based auxiliary adhesive agent, a compound having a sulfonamide structure and a compound having a thiourea structure, a phosphoric acid derivative compound, a β-ketoester compound, and an amino compound.

### [Silane Coupling Agent]

Examples of the silane coupling agent include the compounds described in paragraph 0316 of WO2021/112189A and the compounds described in paragraphs 0067 to 0078 of JP2018-173573A, the contents of which are incorporated in the present specification. In addition, it is also preferable to use two or more kinds of different silane coupling agents as described in paragraphs 0050 to 0058 of JP2011-128358A. It is also preferable to use the following compound as the silane coupling agent. In the following Formulae, Me represents a methyl group, and Et represents an ethyl group. In addition, examples of R shown below include a structure derived from a blocking agent in a blocked isocyanate group. The blocking agent may be selected depending on the desorption temperature; however, examples thereof include an alcohol compound, a phenol compound, a pyrazole compound, a triazole compound, a lactam compound, and an active methylene compound. It is preferably caprolactam or the like, for example, from the viewpoint of setting the desorption temperature to 160° C. to 180° C. Examples of the commercially available product of such a compound include X-12-1293 (manufactured by Shin-Etsu Chemical Co., Ltd.).

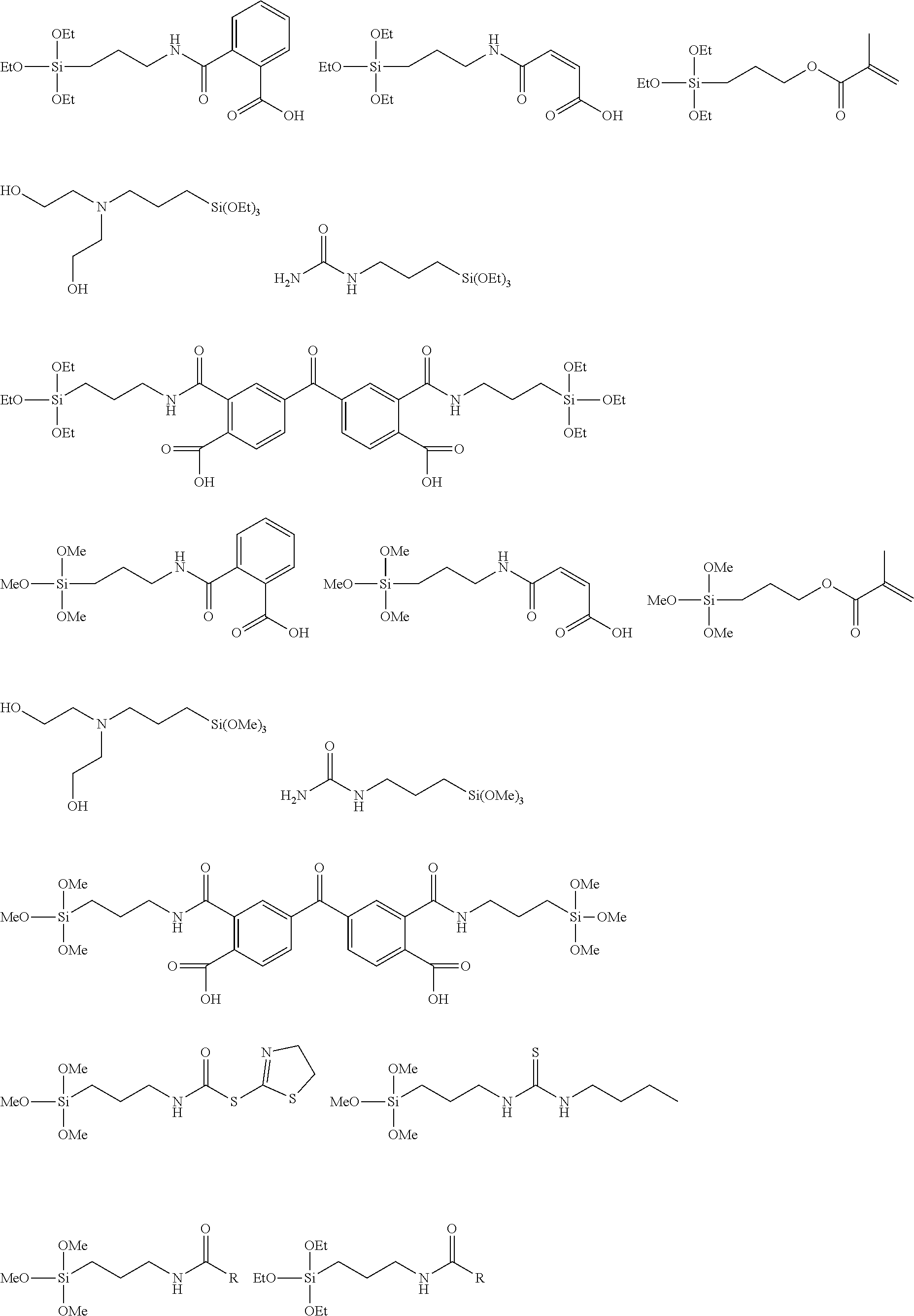

Examples of the other silane coupling agent include vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, p-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene)propylamine, N-phenyl-3-aminopropyltrimethoxysilane, tris-(trimethoxysilylpropyl)isocyanurate, 3-ureidopropyltrialkoxysilane, 3-mercaptopropylmethyldimethoxysilane, 3-mercaptopropyltrimethoxysilane, 3-isocyanatepropyltriethoxysilane, and 3-trimethoxysilylpropylsuccinic acid anhydride. These can be used alone or in a combination of two or more thereof.

In addition, an oligomer type compound having a plurality of alkoxysilyl groups can also be used as the silane coupling agent.

Examples of such an oligomer type compound include a compound containing a repeating unit represented by Formula (S-1).

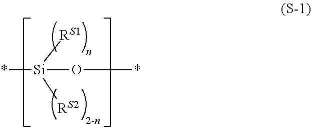

In Formula (S-1), R^{S1} represents a monovalent organic group, R^{S2} represents a hydrogen atom, a hydroxy group, or an alkoxy group, and n represents an integer of 0 to 2.

R^{S1} preferably has a structure including a polymerizable group. Examples of the polymerizable group include a group having an ethylenically unsaturated bond, an epoxy group, an oxetanyl group, a benzoxazolyl group, a blocked isocyanate group, and an amino group. Examples of the group having an ethylenically unsaturated bond include a vinyl group, an allyl group, an isoallyl group, a 2-methylallyl group, a group (for example, a vinylphenyl group) having an aromatic ring that is directly bonded to a vinyl group, and a (meth)acrylamide group, a (meth)acryloyloxy group, where a vinylphenyl group, a (meth)acrylamide group, or a (meth)acryloyloxy group is preferable, a vinylphenyl group or a (meth)acryloyloxy group is more preferable, and a (meth)acryloyloxy group is still more preferable.

R^{S2} is preferably an alkoxy group, and more preferably a methoxy group or an ethoxy group.

n represents an integer of 0 to 2, and it is preferably 1.

Here, structures of a plurality of repeating units represented by Formula (S-1) may be the same, where the plurality of repeating units are contained in the oligomer type compound.

Here, among a plurality of repeating units represented by Formula (S-1), which are contained in the oligomer type compound, it is preferable that at least one repeating unit has n of 1 or 2, it is more preferable that at least two repeating units have n of 1 or 2, and it is still more preferable that at least two repeating units have n of 1.

As such an oligomer type compound, a commercially available product can be used, and examples thereof include KR-513 (manufactured by Shin-Etsu Chemical Co., Ltd.).

### [Aluminum-Based Auxiliary Adhesive Agent]

Examples of the aluminum-based auxiliary adhesive agent include aluminum tris(ethyl acetoacetate), aluminum tris(acetyl acetate), and ethyl acetoacetate aluminum diisopropylate.

As another metal adhesiveness improving agent, the compounds described in paragraphs 0046 to 0049 of JP2014-186186A, and the sulfide-based compounds described in paragraphs 0032 to 0043 of JP2013-072935A can also be used, and the contents of which are incorporated in the present specification.

The content of the metal adhesiveness improving agent is preferably 0.01 to 30 parts by mass, more preferably 0.1 to 10 parts by mass, and still more preferably 0.5 to 5 parts by mass with respect to 100 parts by mass of the specific resin. In a case where the content is set to be equal to or higher than the above lower limit value, good adhesiveness between a pattern and a metal layer is exhibited, and in a case where the content is set to be equal to or lower than the above upper limit value, good heat resistance of the pattern and good mechanical characteristics are exhibited. Only one kind of metal adhesiveness improving agent may be used, or two or more kinds thereof may be used. In a case where two or more kinds thereof are used, the total content thereof is preferably within the above-described range.

### <Migration Suppressing Agent>

The resin composition according to the embodiment of the present invention preferably further contains a migration suppressing agent. For example, in a case where the resin composition is applied to a metal layer (or a metal wiring line) to form a film, it is possible to effectively suppress the migration of metal ions derived from the metal layer (or the metal wiring line) into the film, in a case where a migration suppressing agent is contained.

The migration suppressing agent is not particularly limited; however, examples thereof include a compound having a heterocyclic ring (a pyrrole ring, a furan ring, a thiophene ring, an imidazole ring, an oxazole ring, a thiazole ring, a pyrazole ring, an isoxazole ring, an isothiazole ring, a tetrazole ring, a pyridine ring, a pyridazine ring, a pyrimidine ring, a pyrazine ring, a piperidine ring, a piperazine ring, a morpholine ring, a 2H-pyran ring and a 6H-pyran ring, or a triazine ring), a compound having thioureas and a sulfanyl group, a hindered phenol-based compound, a salicylic acid derivative-based compound, and a hydrazide derivative-based compound. In particular, it is possible to preferably use a triazole-based compound such as 1,2,4-triazole, benzotriazole, 3-amino-1,2,4-triazole, or 3,5-diamino-1,2,4-triazole, or a tetrazole-based compound such as 1H-tetrazole, 5-phenyltetrazole or 5-amino-1H-tetrazole.

Among these, the resin composition according to the embodiment of the present invention preferably contains an azole compound.

The azole compound is a compound containing an azole structure, and the azole structure refers to a 5-membered ring structure including a nitrogen atom as a ring member, and is preferably a 5-membered ring structure including two or more nitrogen atoms as a ring member. Specific examples of the azole structure include an imidazole structure, a triazole structure, and a tetrazole structure. These structures may form a polycycle by fusion with another ring structure, such as benzimidazole or benzotriazole.

In addition, as the compound having an azole structure, a compound in which a group represented by Formula (R-1) or Formula (R-2) is directly bonded to the azole structure is also preferable.

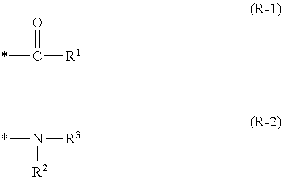

In Formula (R-1), R¹ represents a monovalent organic group, and * represents a bonding site to an azole structure.

In Formula (R-2), R² represents a hydrogen atom or a monovalent organic group, R³ represents a monovalent organic group, and * represents a bonding site to the azole structure.

In Formula (R-1), R¹ is preferably a hydrocarbon group or a group represented by a bond between a hydrocarbon group and at least one group selected from the group consisting of —O—, —C(═O)—, —S—, —S(═O)₂—, and —NR^{N}—. R^{N} is as described above.

The above-described hydrocarbon group is preferably an aliphatic hydrocarbon group, an aromatic hydrocarbon group, or a group represented by a combination thereof.

In addition, the total number of carbon atoms in R¹ is preferably 1 to 30, more preferably 2 to 25, and still more preferably 3 to 20.

In R¹, the bonding site to the carbonyl group in Formula (R-1) is preferably a hydrocarbon group or —NR^{N}—.

In Formula (R-1), * represents a bonding site to an azole structure, and is preferably a bonding site to a carbon atom which is a ring member of the azole structure.

In Formula (R-2), R² is preferably a hydrogen atom.

In a case where R² is a monovalent organic group, R² is preferably a hydrocarbon group or a group represented by a bond between a hydrocarbon group and at least one group selected from the group consisting of —O—, —C(═O)—, —S—, —S(═O)₂—, and —NR^{N}—. R^{N} is as described above.

The above-described hydrocarbon group is preferably an aliphatic hydrocarbon group, an aromatic hydrocarbon group, or a group represented by a combination thereof.

In addition, in a case where R² is a monovalent organic group, the total number of carbon atoms is preferably 1 to 30, more preferably 2 to 25, and still more preferably 3 to 20.

In a case where R² is a monovalent organic group, a bonding site to a nitrogen atom in Formula (R-2) in R² is preferably a hydrocarbon group or —C(═O)—.

In Formula (R-2), R³ is preferably a hydrocarbon group or a group represented by a bond between a hydrocarbon group and at least one group selected from the group consisting of —O—, —C(═O)—, —S—, —S(═O)₂—, and —NR^{N}—. R^{N} represents a hydrogen atom or a hydrocarbon group, and a hydrogen atom is preferable.

The above-described hydrocarbon group is preferably an aliphatic hydrocarbon group, an aromatic hydrocarbon group, or a group represented by a combination thereof.

In addition, in a case where R³ is a monovalent organic group, the total number of carbon atoms is preferably 1 to 30, more preferably 2 to 25, and still more preferably 3 to 20.

In R³, the bonding site to the nitrogen atom in Formula (R-2) is preferably a hydrocarbon group or —C(═O)—.

In Formula (R-2), * represents a bonding site to an azole structure, and is preferably a bonding site to a carbon atom which is a ring member of the azole structure.

As the migration suppressing agent, an ion trap agent that captures an anion such as a halogen ion can also be used.

The rust inhibitors described in paragraph 0094 of JP2013-015701A, the compounds described in paragraphs 0073 to 0076 of JP2009-283711A, the compounds described in paragraph 0052 of JP2011-059656A, the compounds described in paragraphs 0114, 0116, and 0118 of JP2012-194520A, the compounds described in paragraph 0166 of WO2015/199219A, or the like can be used as the other migration suppressing agents, the contents of which are incorporated in the present specification.

Specific examples of the migration suppressing agent include the following compounds.

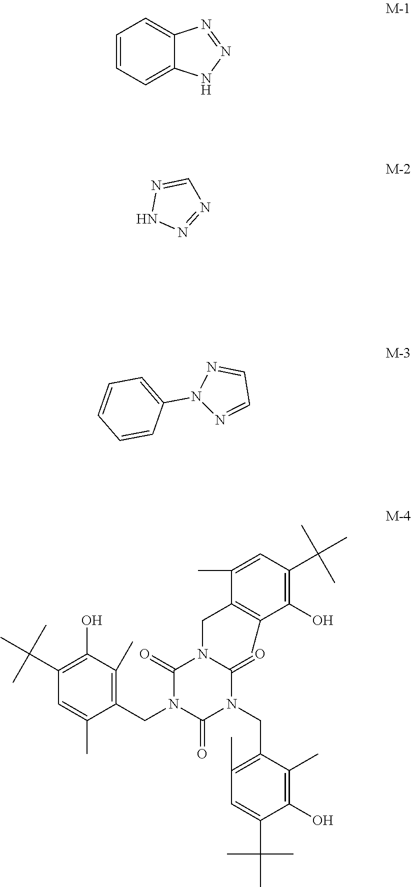 [SMILES: c1ccc2[nH]nnc2c1 ; c1nn[nH]n1 ; c1ccc(-n2nccn2)cc1 ; Cc1cc(C(C)(C)C)c(O)c(C)c1Cn1c(=O)n(Cc2c(C)cc(C(C)(C)C)c(O)c2C)c(=O)n(Cc2c(C)cc(C(C)(C)C)c(O)c2C)c1=O]

In a case where the resin composition according to the embodiment of the present invention contains the migration suppressing agent, the content of the migration suppressing agent is preferably 0.01% to 5.0% by mass, more preferably 0.05% to 2.0% by mass, and still more preferably 0.1% to 1.0% by mass, with respect to the total solid content of the resin composition.

Only one kind of migration suppressing agent may be used alone, or two or more kinds thereof may be used. In a case where two or more kinds of migration suppressing agents are used, the total thereof is preferably within the above-described range.

### <Light Absorbing Agent>

The resin composition according to the embodiment of the present invention also preferably contains a compound (a light absorbing agent) having a small absorbance at an exposure wavelength thereof upon exposure.

Examples of the light absorbing agent include compounds described in paragraphs 0159 to 0183 of WO2022/202647A and compounds described in paragraphs 0088 to 0108 of JP2019-206689A. The content thereof is incorporated in the present specification.

In particular, from the viewpoint of improving the adhesiveness to the base material, the resin composition according to the embodiment of the present invention preferably further contains the above-described azole compound and the above-described silane coupling agent.

By containing these compounds, particularly, the adhesiveness to the base material is likely to be maintained even after the cured substance is exposed to high temperature and high humidity conditions.

### <Polymerization Inhibitor>

The resin composition according to the embodiment of the present invention preferably contains a polymerization inhibitor. Examples of the polymerization inhibitor include a phenol-based compound, a quinone-based compound, an amino-based compound, an N-oxyl-free radical-based compound, a nitro-based compound, a nitroso-based compound, a heteroaromatic ring-based compound, and a metal compound.

Specific examples of the compound of the polymerization inhibitor include the compounds described in paragraph 0310 of WO2021/112189A, p-hydroquinone, o-hydroquinone, a 4-hydroxy-2,2,6,6-tetramethylpiperidine-1-oxyl free radical, phenoxazine, and 1,4,4-trimethyl-2,3-diazabicyclo[3.2.2]nona-2-en-N,N-dioxide. The content thereof is incorporated in the present specification.

In a case where the resin composition according to the embodiment of the present invention has a polymerization inhibitor, the content of the polymerization inhibitor is preferably 0.01% to 20% by mass, more preferably 0.02% to 15% by mass, and still more preferably 0.05% to 10% by mass, with respect to the total solid content of the resin composition.

One kind of polymerization inhibitor may be used, or two or more kinds thereof may be used. In a case where two or more kinds of polymerization inhibitors are used, the total thereof is preferably within the above-described range.

### <Other Additives>

The resin composition according to the embodiment of the present invention may contain various additives as necessary, for example, a surfactant, a higher fatty acid derivative, a thermal polymerization initiator, inorganic particles, an ultraviolet absorbing agent, an organic titanium compounds, an antioxidant, a photoacid generator, an aggregation inhibitor, a phenol-based compound, another polymer compound, a plasticizer, and other auxiliary agents (for example, an anti-foaming agent, and a flame retardant) within the scope in which the effect of the present invention is obtained. By appropriately containing these components, properties such as film properties can be adjusted. The details of the components can be found in, for example, paragraph “0183” and subsequent paragraphs of JP2012-003225A (corresponding to paragraph 0237 of US2013/0034812A) and paragraphs 0101 to 0104 and 0107 to 0109 of JP2008-250074A, the contents of which are incorporated in the present specification. In a case where these additives are blended, the total content thereof is preferably 3% by mass or less of the solid content of the resin composition according to the embodiment of the present invention.

### [Surfactant]

As the surfactant, various surfactants such as a fluorine-based surfactant, a silicone-based surfactant, and a hydrocarbon-based surfactant can be used. The surfactant may be a nonionic surfactant, a cationic surfactant, or an anionic surfactant.

In a case where a surfactant is contained in the photosensitive resin composition according to the embodiment of the present invention, the liquid characteristics (particularly, the fluidity) in a case where a coating liquid composition is prepared are further improved, and thus the uniformity of the coating thickness and the liquid saving property can be further improved. That is, in a case where a film is formed using a coating liquid containing a surfactant, the interfacial tension between a surface to be coated and a coating liquid is reduced, the wettability to the surface to be coated is improved, and thus the coatability to the surface to be coated is improved. Therefore, it is possible to more suitably form a uniform film having a small thickness unevenness.

Examples of the fluorine-based surfactant include the compounds described in paragraph 0328 of WO2021/112189A, the contents of which are incorporated in the present specification.

As the fluorine-based surfactant, a fluorine-containing polymer compound containing a repeating unit derived from a (meth)acrylate compound having a fluorine atom and a repeating unit derived from a (meth)acrylate compound having 2 or more (preferably 5 or more) alkyleneoxy groups (preferably ethyleneoxy groups or propyleneoxy groups) can also be preferably used, and examples thereof include the following compounds.

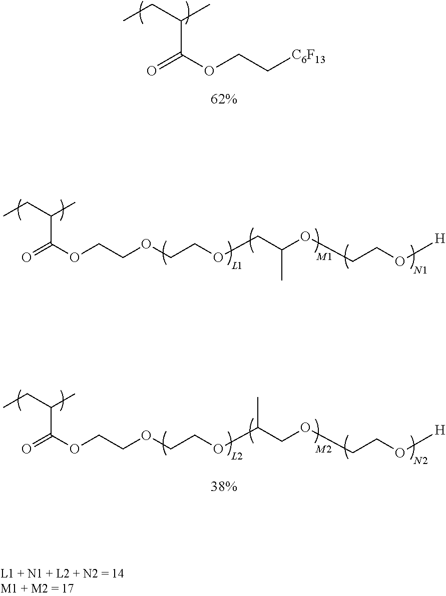

The weight-average molecular weight of the above-described compound is preferably 3,000 to 50,000 and more preferably 5,000 to 30,000.

Regarding the fluorine-based surfactant, a fluorine-containing polymer having an ethylenically unsaturated group in the side chain can also be used as the fluorine-based surfactant. Specific examples thereof include the compounds described in paragraphs “0050” to “0090” and “0289” to “0295” of JP2010-164965A, the content of which is incorporated in the present specification. Further, examples of the commercially available product thereof include MEGAFACE RS-101, RS-102, and RS-718K, all manufactured by DIC Corporation.

The content of fluorine in the fluorine-based surfactant is preferably 3% to 40% by mass, more preferably 5% to 30% by mass, and particularly preferably 7% to 25% by mass. The fluorine-based surfactant in which the fluorine content is in the above-described range is effective from the viewpoints of the uniformity in the thickness of the coating film and liquid saving properties, and the solubility thereof in the composition is also excellent.

Examples of the silicone-based surfactant, the hydrocarbon-based surfactant, the nonionic surfactant, the cationic surfactant, and the anionic surfactant include the compounds described in paragraphs 0329 to 0334 of WO2021/112189A, the contents of which are incorporated in the present specification.

Only one kind of surfactant may be used, or two or more kinds thereof may be used in combination.

The content of the surfactant is preferably 0.001% to 2.0% by mass and more preferably 0.005% to 1.0% by mass with respect to the total solid content of the composition.

### [Inorganic Particle]

Specific examples of the inorganic particle include calcium carbonate, calcium phosphate, silica, kaolin, talc, titanium dioxide, alumina, barium sulfate, calcium fluoride, lithium fluoride, zeolite, molybdenum sulfide, and glass.

The average particle diameter of the inorganic particles is preferably 0.01 to 2.0 m, more preferably 0.02 to 1.5 m, still more preferably 0.03 to 1.0 m, and particularly preferably 0.04 to 0.5 m.

The average particle diameter of the inorganic particles is the primary particle diameter and the volume average particle diameter. The volume average particle diameter can be measured by, for example, a dynamic light scattering method with Nanotrac WAVE II EX-150 (manufactured by Nikkiso Co., Ltd.).

In a case where the above measurement is difficult, the measurement can also be carried out by a centrifugal sedimentation light transmission method, an X-ray transmission method, or a laser diffraction/light scattering method.

### [Organic Titanium Compound]

In a case where the resin composition contains an organic titanium compound, it is possible to form a resin layer having excellent chemical resistance even in a case where curing is carried out at a low temperature.

Examples of the usable organic titanium compound include those in which an organic group is bonded to a titanium atom through a covalent bond or an ionic bond.

Specific examples of the organic titanium compound are described in I) to VII) below.

I) Titanium chelate compounds: a titanium chelate compound having two or more alkoxy groups is more preferable since the resin composition has good storage stability and a good curing pattern is obtained. Specific examples thereof include titanium bis(triethanolamine)diisopropoxide, titanium di(n-butoxide) bis(2,4-pentanedionate), titanium diisopropoxide bis(2,4-pentanedionate), titanium diisopropoxide bis(tetramethylheptandionate), and titanium diisopropoxide bis(ethyl acetoacetate).

II) Tetraalkoxytitanium compounds: examples thereof include titanium tetra(n-butoxide), titanium tetraethoxide, titanium tetra(2-ethylhexoxide), titanium tetraisobutoxide, titanium tetraisopropoxide, titanium tetramethoxide, titanium tetramethoxypropoxide, titanium tetramethylphenoxide, titanium tetra(n-nonyloxide), titanium tetra(n-propoxide), titanium tetrastearyloxide, and titanium tetrakis[bis{2,2-(aryloxymethyl)butoxide}].

III) Titanocene compounds: examples thereof include pentamethylcyclopentadienyl titanium trimethoxide, bis(η5-2,4-cyclopentadien-1-yl)bis(2,6-difluorophenyl)titanium, and bis(η5-2,4-cyclopentadien-1-yl)bis(2,6-difluoro-3-(1H-pyrrol-1-yl)phenyl)titanium.

IV) Monoalkoxytitanium compounds: examples thereof include titanium tris(dioctyl phosphate)isopropoxide, and titanium tris(dodecylbenzene sulfonate)isopropoxide.

V) Titanium oxide compounds: examples thereof include titanium oxide bis(pentanedionate), titanium oxide bis(tetramethylheptandionate), and phthalocyanine titanium oxide.

VI) Titanium tetraacetylacetonate compounds: examples thereof include titanium tetraacetylacetonate.

VII) Titanate coupling agents: examples thereof include isopropyltridodecylbenzenesulfonyl titanate.

Among these, it is preferable that the organic titanium compound is at least one compound selected from the group consisting of the above-described I) titanium chelate compound, II) tetraalkoxytitanium compound, and III) titanocene compound, from the viewpoint of more favorable chemical resistance. In particular, titanium diisopropoxide bis(ethyl acetoacetate), titanium tetra(n-butoxide), or bis(η5-2,4-cyclopentadiene-1-yl) bis(2,6-difluoro-3-(1H-pyrrole-1-yl)phenyl)titanium is preferable.

In addition, it is also preferable that a compound represented by Formula (T-1) is contained as the organic titanium compound or instead of the organic titanium compound.

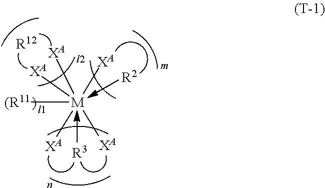

In Formula (T-1), M is titanium, zirconium, or hafnium, l1 is an integer of 0 to 2, 12 is 0 or 1, l1+l2×2 is an integer of 0 to 2, m is an integer of 0 to 4, n is an integer of 0 to 2, l1+l2+m+n×2=4 is satisfied, R¹¹'s are each independently a substituted or unsubstituted cyclopentadienyl group, a substituted or unsubstituted alkoxy group, or a substituted or unsubstituted phenoxy group, R¹² is a substituted or unsubstituted hydrocarbon group, R²'s are each independently a group including a structure represented by Formula (T-2), R³'s are each independently a group including a structure represented by Formula (T-2), and X^{A}'s are each independently an oxygen atom or a sulfur atom.

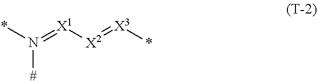

In Formula (T-2), X¹ to X³ each independently represent —C(—*)= or —N═, *'s each represent a bonding site to another structure, and # represents a bonding site to a metal atom.

In Formula (T-1), it is preferable that M represents titanium from the viewpoint of the storage stability of the composition.

An aspect in which 11 and 12 in Formula (T-1) are 0 is also one of the preferred aspects of the present invention.

In Formula (T-1), m is preferably 2 or 4, and more preferably 2.

In Formula (T-1), n is preferably 1 or 2, and more preferably 1.

Here, in Formula (T-1), it is also preferable that 11 and 12 are 0 and m is 0, 2, or 4.

In Formula (T-1), R¹¹ is preferably a substituted or unsubstituted cyclopentadienyl ligand from the viewpoint of the stability of the specific metal complex.

In addition, the cyclopentadienyl group, the alkoxy group, and the phenoxy group as R¹¹ may be substituted; however, an aspect in which these are unsubstituted is also one of the preferred aspects of the present invention.

In Formula (T-1), R¹² is preferably a hydrocarbon group having 1 to 20 carbon atoms and more preferably a hydrocarbon group having 2 to 10 carbon atoms.

The hydrocarbon group as R¹² may be any of an aliphatic hydrocarbon group or an aromatic hydrocarbon group; however, it is preferably an aromatic hydrocarbon group.

The aliphatic hydrocarbon group may be a saturated aliphatic hydrocarbon group or an unsaturated aliphatic hydrocarbon group; however, it is preferably a saturated aliphatic hydrocarbon group.

The aromatic hydrocarbon group is preferably an aromatic hydrocarbon group having 6 to 20 carbon atoms, more preferably an aromatic hydrocarbon group having 6 to 10 carbon atoms, and still more preferably a phenylene group.

The substituent as R¹² is preferably a monovalent substituent, and examples thereof include a halogen atom. In addition, in a case where R¹² is an aromatic hydrocarbon group, it may have an alkyl group as a substituent.

Among these, in Formula (T-1), R¹² is preferably an unsubstituted phenylene group.

In addition, the phenylene group as R¹² is preferably a 1,2-phenylene group.

In Formula (T-1), in a case where m is 2 or more and two or more R²'s contained, the structures of the two or more R²'s may be the same or different from each other.

In Formula (T-1), in a case where n is 2 or more and two or more R³'s contained, the structures of the two or more R³'s may be the same or different from each other.

In Formula (T-2), X¹ to X³ each independently represent —C(—*)= or —N═. It is preferable that at least one thereof represents —C(—*)=, and it is more preferable that at least two thereof represent —C(—*)=.

Specific examples of the compound represented by Formula (T-1) include compounds corresponding to I-3 in Examples, but the present invention is not limited thereto.

In a case of containing an organic titanium compound, the content thereof is preferably 0.05 to 10 parts by mass and more preferably 0.1 to 5 parts by mass with respect to 100 parts by mass of the specific resin. In a case where the content thereof is 0.05 parts by mass or more, the heat resistance and the chemical resistance of the cured pattern to be obtained are further improved, and in a case where the content thereof is 10 parts by mass or less, the storage stability of the composition is more excellent.

In a case of containing an organic titanium compound, the content thereof is preferably 0.05 to 10 parts by mass and more preferably 0.1 to 2 parts by mass with respect to 100 parts by mass of the specific resin. In a case where the content thereof is 0.05 parts by mass or more, the heat resistance and the chemical resistance of the cured pattern to be obtained are further improved, and in a case where the content thereof is 10 parts by mass or less, the storage stability of the composition is more excellent.

Examples of the other additives include the compounds described in paragraphs 0249 to 0282, 0316 to 0358 of WO2022/145355A. The above description is incorporated in the present specification.

### <Characteristics of Resin Composition>

The viscosity of the resin composition according to the embodiment of the present invention can be adjusted by adjusting the concentration of solid contents of the resin composition. From the viewpoint of the coating film thickness, it is preferably 1,000 mm²/s to 12,000 mm²/s, more preferably 2,000 mm²/s to 10,000 mm²/s, and still more preferably 2,500 mm²/s to 8,000 mm²/s. Within the above range, it is easy to obtain a coating film having high uniformity. In a case of being 1,000 mm²/s or more, it is easy to carry out coating at a film thickness to be required as, for example, an insulating film for re-distribution of a wiring line, and in a case of being 12,000 mm²/s or less, a coating film having an excellent coating surface shape is obtained.

In a case where a cured substance having a film thickness of 10 m is formed using the resin composition according to the embodiment of the present invention, the light transmittance of the cured substance at a wavelength of 365 nm is preferably 15% or more, more preferably 20% or more, and more preferably 25% or more.

The upper limit of the transmittance is not particularly limited and may be 100%.

The cured substance can be obtained, for example, by applying the resin composition according to the embodiment of the present invention to a silicon wafer, drying the silicon wafer at 100° C. for 5 minutes, performing full-surface exposure with i-rays at an exposure energy of 500 mJ/cm², raising the temperature at a temperature rising rate of 10° C./min in a nitrogen atmosphere, and heating the silicon wafer at 230° C. for 180 minutes.

### <Restrictions on Substances Contained in Resin Composition>

The moisture content of the resin composition according to the embodiment of the present invention is preferably less than 2.0% by mass, more preferably less than 1.5% by mass, and still more preferably less than 1.0% by mass. In a case of being less than 2.0%, the storage stability of the resin composition is improved.

Examples of the method for maintaining the moisture content include adjusting the humidity under storage conditions and reducing the void ratio of the storage container during storage.

From the viewpoint of insulating properties, the metal content of the resin composition according to the embodiment of the present invention is preferably less than 5 parts per million (ppm) by mass, more preferably less than 1 ppm by mass, and still more preferably less than 0.5 ppm by mass. Examples of the metal include sodium, potassium, magnesium, calcium, iron, copper chromium, and nickel, however, a metal contained as a complex of an organic compound and a metal is excluded. In a case where a plurality of metals are contained, the total of these metals is preferably within the above-described range.

In addition, as a method for reducing metal impurities which are unintentionally contained in the resin composition according to the embodiment of the present invention, a method for selecting a raw material containing a low metal content as the raw material that constitutes the resin composition according to the embodiment of the present invention, a method for filtering a raw material constituting the resin composition according to the embodiment of the present invention, a method for distilling under the conditions in which the inside of the device is lined with polytetrafluoroethylene or the like to suppress the contamination as little as possible, and the like can be mentioned.

In the resin composition according to the embodiment of the present invention, in a case of considering the use application as a semiconductor material, the content of halogen atoms is preferably less than 500 ppm by mass, more preferably less than 300 ppm by mass, and still more preferably less than 200 ppm by mass, from the viewpoint of wire corrosiveness. Among these, in a case of being present in a halogen ion state, the content is preferably less than 5 ppm by mass, more preferably less than 1 ppm by mass, and still more preferably less than 0.5 ppm by mass. Examples of the halogen atom include a chlorine atom and a bromine atom. It is preferable that the total content of the chlorine atom and the bromine atom, or the total content of the chlorine ion and the bromine ion is within the above-described range.

Preferred examples of the method for adjusting the content of halogen atoms include ion exchange treatment.

A storage container publicly known in the related art can be used as a storage container for the resin composition according to the embodiment of the present invention. As the storage container, for the intended purpose of suppressing the incorporation of impurities into the raw materials and the resin composition according to the embodiment of the present invention, a multilayer bottle in which an inner wall of a container is composed of six kinds of six layers of resin, and a bottle with six kinds of resin being made as a seven-layer structure are preferably used. Examples of such a container include the container described in JP2015-123351A.

### <Cured Substance of Resin Composition>

In a case of curing the resin composition according to the embodiment of the present invention, it is possible to obtain a cured substance of the resin composition.

The cured substance according to the embodiment of the present invention is a cured substance formed by curing the resin composition.

The curing of the resin composition is preferably carried out by heating. The heating temperature is more preferably 120° C. to 400° C., still more preferably 140° C. to 380° C., and particularly preferably 170° C. to 350° C. The form of the cured substance of the resin composition is not particularly limited and can be selected depending on the use application, where the form includes a film shape, a rod shape, a spherical shape, a pellet shape, and the like. In the present invention, the cured substance preferably has a film shape. The shape of the cured substance can also be selected depending on the use application by the pattern processing of the resin composition, where the use application includes the formation of a protective film on a wall surface, formation of via holes for conduction, adjustment of impedance, electrostatic capacity, or internal stress, impartment of heat radiation function, and the like. The film thickness of the cured substance (the film consisting of the cured substance) is preferably 0.5 m or more and 150 m or less.

The shrinkage ratio of the resin composition according to the embodiment of the present invention after curing is preferably 50% or less, more preferably 45% or less, and still more preferably 40% or less. Here, the shrinkage ratio refers to a percentage of a change in the volume of the resin composition before and after curing, and it can be calculated according to the following expression.

Shrinkage ratio [%]=100−(volume after curing/volume before curing)×100

### <Characteristics of Cured Substance of Resin Composition>

The imidization reaction rate of the cured substance of the resin composition according to the embodiment of the present invention is preferably 70% or more, more preferably 80% or more, and still more preferably 90% or more. In a case of being 70% or more, a cured substance having excellent mechanical properties may be obtained.

The breaking elongation of the cured substance of the resin composition according to the embodiment of the present invention is preferably 30% or more, more preferably 40% or more, and still more preferably 50% or more.

The glass transition temperature (Tg) of the cured substance of the resin composition according to the embodiment of the present invention is preferably 180° C. or higher, more preferably 210° C. or higher, and still more preferably 230° C. or higher.

### <Preparation of Resin Composition>

The resin composition according to the embodiment of the present invention can be prepared by mixing the above-described components. The mixing method is not particularly limited, and mixing can be carried out by methods publicly known in the related art.

Examples of the mixing method include mixing with a stirring blade, mixing with a ball mill, and mixing by rotating a tank.

The temperature during the mixing is preferably 10° C. to 30° C., and more preferably 15° C. to 25° C.

It is preferable to carry out filtration using a filter for the intended purpose of removing foreign substances such as dust and fine particles in the resin composition according to the embodiment of the present invention. The filter pore diameter is, for example, preferably 5 m or less, more preferably 1 m or less, still more preferably 0.5 m or less, and even still more preferably 0.1 m or less. The material of the filter is preferably polytetrafluoroethylene, polyethylene, or nylon. In a case where the material of the filter is polyethylene, it is more preferable to use high density polyethylene (HDPE). As the filter, a filter which has been washed with an organic solvent in advance may be used. In the filtration step using the filter, a plurality of kinds of filters may be connected in series or in parallel and used. In a case where a plurality of kinds of filters are used, filters having different pore diameters or different materials may be used in combination. Examples of the connection aspect include an aspect in which an HDPE filter having a pore diameter of 1 m is connected in series as the first stage and an HDPE filter having a pore diameter of 0.2 m is connected in series as the second stage. In addition, various materials may be filtered a plurality of times. In a case of being filtered a plurality of times, circulation filtration may be used. In addition, filtration may be carried out under pressure. In a case of carrying out pressurization to carry out filtration, the pressure for pressurization is, for example, preferably 0.01 MPa or more and 1.0 MPa or less, more preferably 0.03 MPa or more and 0.9 MPa or less, still more preferably 0.05 MPa or more and 0.7 MPa or less, and even still more preferably 0.05 MPa or more and 0.5 MPa or less.

In addition to filtration using a filter, impurity removal treatment using an adsorbing material may be carried out. The filtration using a filter and the impurity removal treatment using an adsorbing material may be combined. As the adsorbing material, a publicly known adsorbing material can be used. Examples thereof include an inorganic adsorbing material such as silica gel and zeolite and an organic adsorbing material such as activated carbon.

After filtration using a filter, a step of placing a bottle filled with the resin composition under reduced pressure to carry out degassing may be provided.

### (Manufacturing Method for Cured Substance)

The manufacturing method for a cured substance according to the embodiment of the present invention preferably includes a film forming step of applying a resin composition onto a base material to form a film.

The manufacturing method for a cured substance more preferably includes the above-described film forming step, an exposure step of selectively exposing the film formed by the film forming step, and a development step of developing the film exposed by the exposure step using a developer to form a pattern.

It is particularly preferable that the manufacturing method for a cured substance includes the film forming step, the exposure step, the development step, and at least one of a heating step of heating a pattern obtained by the development step or a post-development exposure step of exposing the pattern obtained by the development step.

In addition, it is also preferable that the manufacturing method for a cured substance includes the above-described film forming step and a step of heating the film.

Hereinafter, details of each step will be described.

### <Film Forming Step>

The resin composition according to the embodiment of the present invention can be applied onto a base material, thereby being used in a film forming step of forming a film.

The manufacturing method for a cured substance according to the embodiment of the present invention preferably includes a film forming step of applying a resin composition onto a base material to form a film.

### [Base Material]

The kind of the base material can be appropriately determined depending on the use application and is not particularly limited. Examples of the base material include a base material for semiconductor production, such as silicon, silicon nitride, polysilicon, silicon oxide, or amorphous silicon, quartz, glass, an optical film, a ceramic material, a vapor-deposited film, a magnetic film, a reflective film, a metal base material (for example, it may be any one of a base material formed from a metal or a base material having a metal layer formed by plating, vapor deposition, or the like) such as Ni, Cu, Cr, or Fe, paper, spin-on-glass (SOG), a thin film transistor (TFT) array base material, a mold base material, and an electrode plate of a plasma display panel (PDP). In particular, the base material is preferably a base material for semiconductor production, and more preferably a silicon base material, a Cu base material, or a mold base material.

A layer such as an intimate attachment layer made of hexamethyl disilazane (HMDS) or the like, or an oxide layer may be provided on the surface of these base materials.

The shape of the base material is not particularly limited, and it may be a circular shape or may be a rectangular shape.

In a case where the base material has a circular shape, the size of the base material is, for example, preferably a diameter of 100 to 450 mm and more preferably 200 to 450 mm.

In a case of a rectangular shape, the length of the short side is, for example, preferably 100 to 1,000 mm and more preferably 200 to 700 mm.

As the base material, for example, a base material having a plate shape and preferably a base material (a substrate) having a panel shape are used.

In a case where the resin composition is applied to form a film on a surface of a resin layer (for example, a layer consisting of a cured substance) or on a surface of a metal layer, the resin layer or the metal layer serves as the base material.

The means for applying the resin composition onto a base material is preferably coating.

Specific examples of the means for application include a dip coating method, an air knife coating method, a curtain coating method, a wire bar coating method, a gravure coating method, an extrusion coating method, a spray coating method, a spin coating method, a slit coating method, and an ink jet method. From the viewpoint of the uniformity of the film thickness, a spin coating method, a slit coating method, a spray coating method, or an ink jet method is more preferable, and from the viewpoint of the uniformity of the film thickness and the viewpoint of productivity, a spin coating method or a slit coating method is more preferable. A film having a desired thickness can be obtained by adjusting the concentration of solid contents of the resin composition and application conditions according to the means to be applied. In addition, the coating method can be appropriately selected depending on the shape of the base material. In a case where a circular base material such as a wafer is used, a spin coating method, a spray coating method, an ink jet method, or the like is preferable, and in a case where a rectangular base material is used, a slit coating method, a spray coating method, an ink jet method, or the like is preferable. For example, the spin coating method can be applied at a rotation speed of 500 to 3,500 rpm for about 10 seconds to 3 minutes.

In addition, it is also possible to apply a method for transferring a coating film formed in advance on a temporary support by the above-described coating method, onto a base material.

Regarding the transfer method, the production methods described in paragraphs 0023 and 0036 to 0051 of JP2006-023696A and paragraphs 0096 to 0108 of JP2006-047592A can also be suitably used.

In addition, a step of removing an unnecessary film at the end part of the base material may be carried out. Examples of such a step include edge bead rinsing (EBR) and back rinsing.

A pre-wetting step of applying various solvents onto the base material before applying the resin composition onto the base material to improve the wettability of the base material and then applying the resin composition may be adopted.

### <Drying Step>

The above film may be subjected to a step (a drying step) of drying the film (or the layer) formed for removing the solvent, after the film forming step (the layer forming step).

That is, the manufacturing method for a cured substance according to the embodiment of the present invention may include a drying step of drying the film formed by the film forming step.

It is preferable that the drying step is carried out after the film forming step and before the exposure step.

The drying temperature of the film in the drying step is preferably 50° C. to 150° C., more preferably 70° C. to 130° C., and still more preferably 90° C. to 110° C. In addition, the drying may be carried out by reducing the pressure. Examples of the drying time include 30 seconds to 20 minutes, and the drying time is preferably 1 minute to 10 minutes and more preferably 2 minutes to 7 minutes.

### <Exposure Step>

The film may be subjected to an exposure step of selectively exposing the film.

The manufacturing method for a cured substance may include an exposure step of selectively exposing the film formed by the film forming step.

The selective exposure means that a part of the film is exposed. In addition, by selectively exposing the film, an exposed region (an exposed portion) and an unexposed region (a non-exposed portion) are formed in the film.

The exposure amount is not particularly limited as long as the resin composition according to the embodiment of the present invention can be cured; however, it is, for example, preferably 50 to 10,000 mJ/cm² and more preferably 200 to 8,000 mJ/cm² in terms of conversion of exposure energy at a wavelength of 365 nm.

The exposure wavelength can be appropriately determined in a range of 190 to 1,000 nm and preferably in a range of 240 to 550 nm.

Examples of the exposure wavelength, which are mentioned in the relationship with the light source, include (1) a semiconductor laser (wavelength: 830 nm, 532 nm, 488 nm, 405 nm, 375 nm, 355 nm, or the like); (2) a metal halide lamp; (3) a high pressure mercury lamp, a g-line (wavelength: 436 nm), an h-line (wavelength: 405 nm), an i-line (wavelength: 365 nm), or Broadband light (three wavelengths of the g, h, and i-line); (4) an excimer laser, a KrF excimer laser (wavelength: 248 nm), an ArF excimer laser (wavelength: 193 nm), or an F₂ excimer laser (wavelength: 157 nm); (5) an extreme ultraviolet ray: EUV (wavelength: 13.6 nm); (6) an electron beam; and (7) a second harmonic wave of 532 nm and a third harmonic wave of 355 nm of a YAG laser. Regarding the resin composition according to the embodiment of the present invention, exposure with a high pressure mercury lamp is particularly preferable, and from the viewpoint of exposure sensitivity, exposure with an i-line is more preferable.

The exposure method is not particularly limited as long as at least a part of the film consisting of the resin composition according to the embodiment of the present invention is exposed; however, examples thereof include exposure using a photo mask and exposure by a laser direct imaging method.

### <Post-Exposure Heating Step>

The film may be subjected to a step of carrying out heating after the exposure (a post-exposure heating step).

That is, the manufacturing method for a cured substance according to the embodiment of the present invention may include a post-exposure heating step of heating the film exposed in the exposure step.

The post-exposure heating step can be carried out after the exposure step and before the development step.

The heating temperature in the post-exposure heating step is preferably 50° C. to 140° C. and more preferably 60° C. to 120° C.

The heating time in the post-exposure heating step is preferably 30 seconds to 300 minutes and more preferably 1 minute to 10 minutes.

In the post-exposure heating step, the temperature rising rate from the temperature at the start of heating to the maximum heating temperature is preferably 1 to 12° C./min, more preferably 2 to 10° C./min, and still more preferably 3 to 10° C./min.

In addition, the temperature rising rate may be appropriately changed during heating.

The heating means in the post-exposure heating step is not particularly limited, and a publicly known hot plate, oven, infrared heater, or the like can be used.

In addition, it is also preferable to carry out the heating in an atmosphere having a low oxygen concentration by allowing an inert gas such as nitrogen, helium, argon, or the like to flow.

### <Development Step>

The exposed film may be subjected to a development step of carrying out development using a developer to form a pattern.

That is, the manufacturing method for a cured substance according to the embodiment of the present invention may include a development step of developing the film exposed in the exposure step using a developer to form a pattern.

By carrying out the development, one of the exposed portion and the non-exposed portion of the film is removed, and a pattern is formed.

Here, the development in which the non-exposed portion of the film is removed by the development step is referred to as negative-tone development, and the development in which the exposed portion of the film is removed by the development step is referred to as positive-tone development.

### [Developer]

Examples of the developer that is used in the development step include a developer containing an alkaline aqueous solution or an organic solvent.

In a case where the developer is an alkaline aqueous solution, examples of the basic compound that can be contained in the alkaline aqueous solution include inorganic alkalis, primary amines, secondary amines, tertiary amines, and a quaternary ammonium salt. The basic compound is preferably tetramethylammonium hydroxide (TMAH), potassium hydroxide, sodium carbonate, sodium hydroxide, sodium silicate, sodium metasilicate, ammonia, ethylamine, n-propylamine, diethylamine, di-n-butylamine, triethylamine, methyldiethylamine, dimethylethanolamine, triethanolamine, tetraethylammonium hydroxide, tetrapropylammonium hydroxide, tetrabutylammonium hydroxide, tetrapentylammonium hydroxide, tetrahexylammonium hydroxide, tetraoctylammonium hydroxide, ethyltrimethylammonium hydroxide, butyltrimethylammonium hydroxide, methyltriamylammonium hydroxide, dibutyldipentylammonium hydroxide, dimethylbis(2-hydroxyethyl)ammonium hydroxide, trimethylphenylammonium hydroxide, trimethylbenzylammonium hydroxide, triethylbenzylammonium hydroxide, pyrrole, or piperidine, and it is more preferably TMAH. The content of the basic compound in the developer is preferably 0.01% to 10% by mass, more preferably 0.1% to 5% by mass, and still more preferably 0.3% to 3% by mass in the total mass of the developer.

In a case where the developer contains an organic solvent, the compounds described in paragraph 0387 of WO2021/112189A can be used as the organic solvent. The content thereof is incorporated in the present specification. In addition, suitable examples of the alcohols include methanol, ethanol, propanol, isopropanol, butanol, pentanol, octanol, diethylene glycol, propylene glycol, methyl isobutyl carbinol, and triethylene glycol, and suitable examples of the amides include N-methylpyrrolidone, N-ethylpyrrolidone, and dimethylformamide.

In a case where the developer contains an organic solvent, one kind of organic solvent can be used, or two or more kinds thereof can be mixedly used. In the present invention, in particular, a developer containing at least one selected from the group consisting of cyclopentanone, γ-butyrolactone, dimethyl sulfoxide, N-methyl-2-pyrrolidone, and cyclohexanone is preferable, a developer containing at least one selected from the group consisting of cyclopentanone, γ-butyrolactone, and dimethyl sulfoxide is more preferable, and a developer containing cyclopentanone is particularly preferable.

In a case where the developer contains an organic solvent, the content of the organic solvent with respect to the total mass of the developer is preferably 50% by mass or more, more preferably 70% by mass or more, still more preferably 80% by mass or more, and particularly preferably 90% by mass or more. In addition, the above content may be 100% by mass.

The developer may further contain another component.

Examples of the other component include a publicly known surfactant and a publicly known anti-foaming agent.

### [Method for Supplying Developer]

The method for supplying a developer is not particularly limited as long as a desired pattern can be formed, and it includes a method for immersing a base material on which a film has been formed in a developer, puddle development of supplying a developer to a film formed on a base material using a nozzle, and a method for continuously supplying a developer. The kind of nozzle is not particularly limited, and examples thereof include a straight nozzle, a shower nozzle, and a spray nozzle.

From the viewpoint of the permeability of the developer, the removability of the non-image area, and the manufacturing efficiency, a method for supplying a developer with a straight nozzle or a method for continuously supplying a developer with a spray nozzle is preferable, and from the viewpoint of the permeability of the developer into the image area, a method for supplying a developer with a spray nozzle is more preferable.

In addition, after the continuous supply by a straight nozzle, the base material is spun to remove the developer from the base material, and then the developer is continuously supplied by the straight nozzle again after the spin drying, a step of spinning the base material to remove the developer from the base material may be adopted, and this step may be repeated a plurality of times.

Examples of the method for supplying a developer in the development step include a step of continuously supplying a developer to a base material, a step of keeping a developer in a substantially stationary state on a base material, a step of vibrating a developer on a base material by ultrasonic waves or the like, and a step obtained by combining these steps.

The development time is preferably 10 seconds to 10 minutes and more preferably 20 seconds to 5 minutes. The temperature of the developer during development is not particularly determined; however, it is preferably 10° C. to 45° C. and more preferably 18° C. to 30° C.

In the development step, washing (rinsing) of the pattern with a rinsing liquid may be further carried out after the treatment with the developer. In addition, a method such as supplying a rinsing liquid before the developer which is in contact with the pattern is completely dried may be adopted.

### [Rinsing Liquid]

In a case where the developer is an alkaline aqueous solution, it is possible to use, for example, water as the rinsing liquid. In a case where the developer is a developer containing an organic solvent, it is possible to use as the rinsing liquid, for example, a solvent (for example, water, an organic solvent different from the organic solvent contained in the developer) different from the solvent contained in the developer.

Examples of the organic solvent in a case where the rinsing liquid contains an organic solvent include the same organic solvents as the organic solvents exemplified in the above-described case where the developer contains an organic solvent.

The organic solvent contained in the rinsing liquid is preferably an organic solvent different from the organic solvent contained in the developer, and it is more preferably an organic solvent having a solubility of the pattern, which is lower than that of the organic solvent contained in the developer.

In a case where the rinsing liquid contains an organic solvent, one kind of organic solvent can be used, or two or more kinds thereof can be mixedly used. The organic solvent is preferably cyclopentanone, 7-butyrolactone, dimethyl sulfoxide, N-methylpyrrolidone, cyclohexanone, PGMEA, or PGME, more preferably cyclopentanone, 7-butyrolactone, dimethyl sulfoxide, PGMEA, or PGME, and still more preferably cyclohexanone or PGMEA.

In a case where the rinsing liquid contains an organic solvent, the organic solvent is preferably 50% by mass or more, more preferably 70% by mass or more, and still more preferably 90% by mass or more with respect to the total mass of the rinsing liquid. In addition, the organic solvent may be 100% by mass with respect to the total mass of the rinsing liquid.

The rinsing liquid may further contain another component.

Examples of the other component include a publicly known surfactant and a publicly known anti-foaming agent.

### [Method for Supplying Rinsing Liquid]

The method for supplying a rinsing liquid is not particularly limited as long as a desired pattern can be formed and includes a method for immersing a base material in a rinsing liquid, a method for supplying a rinsing liquid to a base material by liquid filling, a method for supplying a rinsing liquid to a base material with a shower, and a method for continuously supplying a rinsing liquid to a base material by means such as a straight nozzle.

From the viewpoint of the permeability of the rinsing liquid, the removability of non-image area, and the manufacturing efficiency, there is a method for supplying a rinsing liquid with a shower nozzle, a straight nozzle, a spray nozzle, or the like, and a method for continuously supplying a rinsing liquid with a spray nozzle is preferable. From the viewpoint of the permeability of the rinsing liquid into the image area, a method for supplying a rinsing liquid with a spray nozzle is more preferable. The kind of nozzle is not particularly limited, and examples thereof include a straight nozzle, a shower nozzle, and a spray nozzle.

That is, the rinsing step is preferably a step of supplying, with a straight nozzle, or continuously supplying a rinsing liquid to the exposed film, and it is more preferably a step of supplying a rinsing liquid with a spray nozzle.

In the method for supplying a rinsing liquid in the rinsing step, a step of continuously supplying a rinsing liquid to a base material, a step of keeping a rinsing liquid in a substantially stationary state on a base material, a step of vibrating a rinsing liquid on the base material by ultrasonic waves or the like, and a step obtained by combining these steps can be adopted.

The rinsing time is preferably 10 seconds to 10 minutes and more preferably 20 seconds to 5 minutes. The temperature of the rinsing liquid during rinsing is not particularly determined; however, it is preferably 10° C. to 45° C. and more preferably 18° C. to 30° C.

### <Heating Step>

The pattern obtained by the development step (a pattern after the rinsing in a case where the rinsing step is carried out) may be subjected to a heating step of heating the pattern obtained by the development.

That is, the manufacturing method for a cured substance according to the embodiment of the present invention may include a heating step of heating the pattern obtained by the development step.

In addition, the manufacturing method for a cured substance according to the embodiment of the present invention may include a pattern obtained by another method without carrying out the development step, or a heating step of heating a film obtained by the film forming step.

In the heating step, the resin such as the polyimide precursor is cyclized to be a resin such as polyimide.

In addition, the crosslinking of unreacted crosslinkable groups in the specific resin or a crosslinking agent other than the specific resin also proceeds.

The heating temperature (the maximum heating temperature) in the heating step is preferably 50° C. to 450° C., more preferably 150° C. to 350° C., still more preferably 150° C. to 250° C., even still more preferably 160° C. to 250° C., and particularly preferably 160° C. to 230° C.

In a case where the resin composition contains a resin corresponding to the polyimide precursor, the heating step is preferably a step of promoting the cyclization reaction of the polyimide precursor in the pattern by the action of the base generated from the base generator or the like by heating.

The heating in the heating step is preferably carried out at a temperature rising rate of 1 to 12° C./min from the temperature at the start of heating to the maximum heating temperature.

The temperature rising rate is more preferably 2 to 10° C./min and still more preferably 3 to 10° C./min. In a case where the above temperature rising rate is set to 1° C./min or higher, the excessive volatilization of the acid or solvent can be prevented while securing productivity, and in a case where the above temperature rising rate is set to 12° C./min or lower, the residual stress of the cured substance can be relaxed.

In addition, in a case of an oven that enables rapid heating, the heating is preferably carried out at a temperature rising rate of 1 to 8° C./see from the temperature at the start of heating to the maximum heating temperature, more preferably 2 to 7° C./see, and still more preferably 3 to 6° C./sec.

The temperature at the start of heating is preferably 20° C. to 150° C., more preferably 20° C. to 130° C., and still more preferably 25° C. to 120° C. The temperature at the start of heating refers to a temperature at which the step of heating to the maximum heating temperature is started. For example, in a case where the resin composition according to the embodiment of the present invention is applied on a base material and then dried, the temperature at the start of heating is the temperature of the film (the layer) after drying, and for example, it is preferable to raise the temperature from a temperature lower by 30° C. to 200° C. than the boiling point of the solvent contained in the resin composition.

The heating time (the heating time at the maximum heating temperature) is preferably 5 to 360 minutes, more preferably 10 to 300 minutes, and still more preferably 15 to 240 minutes.

In particular, in a case of forming a multilayered laminate, the heating temperature is preferably 30° C. or higher, more preferably 80° C. or higher, still more preferably 100° C. or higher, and particularly preferably 120° C. or higher, from the viewpoint of adhesiveness between layers.

The upper limit of the heating temperature is preferably 350° C. or lower, more preferably 250° C. or lower, and still more preferably 240° C. or lower.

The heating may be carried out stepwise. For example, a step in which the temperature is raised from 25° C. to 120° C. at 3° C./min, held at 120° C. for 60 minutes, raised from 120° C. to 180° C. at 2° C./min, and held at 180° C. for 120 minutes, may be carried out. In addition, it is also preferable to carry out the treatment while carrying out irradiation with ultraviolet rays as described in U.S. Pat. No. 9,159,547B. By such a pretreatment step, it is possible to improve the properties of the film. The pretreatment step may be carried out for a short time of about 10 seconds to 2 hours and more preferably 15 seconds to 30 minutes. The pretreatment step may be carried out as a step of two or more stages, for example, a first stage pretreatment step may be carried out in a range of 100° C. to 150° C., and then a second stage pretreatment step may be carried out in a range of 150° C. to 200° C.

Further, cooling may be carried out after heating, and the cooling rate, in this case, is preferably 1 to 5° C./min.

From the viewpoint of preventing the decomposition of the specific resin, it is preferable that the heating step is carried out in an atmosphere of a low oxygen concentration, for example, by allowing an inert gas such as nitrogen, helium, argon, or the like to flow, or carrying out heating under reduced pressure. The oxygen concentration is preferably 50 ppm (volume ratio) or lower, and more preferably 20 ppm (volume ratio) or lower.

The heating means in the heating step is not particularly limited; however, examples thereof include a hot plate, an infrared furnace, an electric heating oven, a hot air oven, and an infrared oven.

### <Post-Development Exposure Step>

The pattern obtained by the development step (a pattern after the rinsing in a case where the rinsing step is carried out) may be subjected to a post-development exposure step of exposing the pattern after the development step, instead of the heating step or in addition to the heating step.

That is, the manufacturing method for a cured substance according to the embodiment of the present invention may include a post-development exposure step of exposing the pattern obtained by the development step. The manufacturing method for a cured substance according to the embodiment of the present invention may include the heating step and the post-development exposure step or may include only one of the heating step and the post-development exposure step.

In the post-development exposure step, it is possible to accelerate, for example, a reaction in which the cyclization of a polyimide precursor or the like proceeds by photosensitization of a photobase generator, a reaction in which the elimination of an acid-decomposable group proceeds by photosensitization of a photoacid generator.

In the post-development exposure step, it is sufficient that at least a part of the pattern obtained in the development step is exposed; however, it is preferable that the whole of the above pattern is exposed.

The exposure amount in the post-development exposure step is preferably 50 to 20,000 mJ/cm² and more preferably 100 to 15,000 mJ/cm² in terms of conversion of exposure energy at the wavelength at which the photosensitive compound has a sensitivity.

The post-development exposure step can be carried out using, for example, the light source in the above-described exposure step, and it is preferable to use broadband light.

### <Metal Layer Forming Step>

The pattern (preferably a pattern that has been subjected to at least one of the heating step or the post-development exposure step) obtained by the development step may be subjected to a metal layer forming step of forming a metal layer on the pattern.

That is, it is preferable that the manufacturing method for a cured substance according to the embodiment of the present invention includes a metal layer forming step of forming a metal layer on the pattern (preferably a pattern that has been subjected to at least one of the heating step or the post-development exposure step) obtained by the development step.

For the metal layer, existing metal kinds can be used without particular limitations. Examples thereof include copper, aluminum, nickel, vanadium, titanium, chromium, cobalt, gold, tungsten, tin, silver, and an alloy including these metals, where copper or aluminum is more preferable, and copper is still more preferable.

The forming method for the metal layer is not particularly limited, and the existing method can be applied. For example, the methods described in JP2007-157879A, JP2001-521288A, JP2004-214501A, JP2004-101850A, U.S. Pat. No. 7,888,181B2, and U.S. Pat. No. 9,177,926B2 can be used. For example, photolithography, physical vapor deposition method (PVD), chemical vapor phase growth method (CVD), lift-off, electrolytic plating, electroless plating, etching, printing, and a method obtained by combining these may be conceivable. More specific examples of the forming method for the metal layer include a patterning method obtained by combining sputtering, photolithography, and etching, and a patterning method combining photolithography and electrolytic plating. Examples of the preferred aspect of the plating include electrolytic plating using a copper sulfate plating liquid or a copper cyanide plating liquid.

The thickness of the metal layer at the thickest portion is preferably 0.01 to 50 m and more preferably 1 to 10 m.

### <Use Application>

Examples of the field to which the manufacturing method for a cured substance according to the embodiment of the present invention or the cured substance according to the embodiment of the present invention can be applied include an insulating film of an electronic device, an interlayer insulating film for a re-distribution layer, and a stress buffer film. In addition, a sealing film, a substrate material (a base film or cover lay of a flexible print substrate, an interlayer insulating film), or such an insulating film in a use application for mounting as described above, which is patterned by etching, is mentioned. For these use applications, for example, Science & Technology Co., Ltd., “High functionality and applied technology of polyimide” April 2008, Technical library CMC TL “Basics and development of polyimide materials” supervised by Masaaki Kakimoto, published in November 2011, “Latest Polyimide Basics and Applications” edited by Japan Polyimide & Aromatic Polymers Study Group, NTS Inc., August 2010, or the like can be referred to.

The manufacturing method for a cured substance according to the embodiment of the present invention and the cured substance according to the embodiment of the present invention can also be used for the production of board surfaces such as an offset board surface or a screen board surface, for etching of molded parts, for the production of protective lacquers and dielectric layers in electronics, in particular, microelectronics.

### (Laminate and Manufacturing Method for Laminate)

A laminate according to the embodiment of the present invention refers to a structure body having a plurality of layers consisting of the cured substance according to the embodiment of the present invention.

The laminate is a laminate including two or more layers consisting of a cured substance, and it may be a laminate in which three or more layers are laminated.

At least one of the two or more layers consisting of a cured substance, which are included in the laminate, is a layer consisting of the cured substance according to the embodiment of the present invention, and from the viewpoint of suppressing the shrinkage of the cured substance, the deformation of the cured substance due to the shrinkage, or the like, it is also preferable that all the layers consisting of a cured substance which are included in the laminate are layers consisting of the cured substance according to the embodiment of the present invention.

That is, it is preferable that the manufacturing method for a laminate according to the embodiment of the present invention includes the manufacturing method for a cured substance according to the embodiment of the present invention, and it is more preferable to include repeating, a plurality of times, the manufacturing method for a cured substance according to the embodiment of the present invention.

In the laminate according to the embodiment of the present invention, an aspect in which two or more layers of layers consisting of a cured substance are included and a metal layer is provided between any of the layers consisting of the cured substance is preferable. The metal layer is preferably formed in the metal layer forming step.

That is, it is preferable that the manufacturing method for a laminate according to the embodiment of the present invention further includes a metal layer forming step of forming a metal layer on a layer consisting of the cured substance, between the manufacturing methods for a cured substance which are carried out a plurality of times. The preferred aspect of the metal layer forming step is as described above.

Examples of the preferred laminate include a laminate including at least a layer structure in which three layers of a layer consisting of a first cured substance, a metal layer, and a layer consisting of a second cured substance are laminated in order.

It is preferable that both the layer consisting of the first cured substance and the layer consisting of the second cured substance are layers consisting of the cured substance according to the embodiment of the present invention. The resin composition according to the embodiment of the present invention which is used for forming a layer consisting of the first cured substance and the resin composition according to the embodiment of the present invention which is used for forming a layer consisting of the second cured substance may have the same composition or may have compositions different from each other. The metal layer in the laminate according to the embodiment of the present invention is preferably used as the metal wiring line of the re-distribution layer or the like.

### <Laminating Step>

The manufacturing method for a laminate according to the embodiment of the present invention preferably further includes a laminating step.

The laminating step is a series of steps including carrying out again, in the following order on the surface of the pattern (the resin layer) or the metal layer, at least one of (a) the film forming step (the layer forming step), (b) the exposure step, (c) the development step, or (d) the heating step and the post-development exposure step. However, the aspect thereof may be such that at least one of (a) the film forming step or (d) the heating step and the post-development exposure step is repeated. In addition, (e) the metal layer forming step may be included after at least one of the heating step or the post-development exposure step of (d). It is needless to say that the laminating step may further include appropriately the above-described drying step or the like.

In a case where another laminating step is further carried out after the laminating step, a surface activation treatment step may be further carried out after the exposure step, the heating step, or the metal layer forming step. Examples of the surface activation treatment include plasma treatment. Details of the surface activation treatment will be described later.

The laminating step is preferably carried out 2 to 20 times and more preferably 2 to 9 times.

For example, a configuration having resin layers of 2 or more layers and 20 or fewer layers, such as a resin layer/a metal layer/a resin layer/a metal layer/a resin layer/a metal layer, is preferable, and a configuration having resin layers of 2 or more layers and 9 or fewer layers is still more preferable.

In the above layers, the compositions, shapes, film thicknesses, and the like may be the same or may be different from each other.

In the present invention, an aspect in which a metal layer is provided, and then furthermore, a cured substance (a resin layer) of the resin composition according to the embodiment of the present invention is formed to cover the metal layer is particularly preferable. Specific examples thereof include an aspect in which (a) the film forming step, (b) the exposure step, (c) the development step, (d) at least one of the heating step or the post-development exposure step, (e) the metal layer forming step are repeated in this order, and an aspect in which (a) the film forming step, (d) at least one of the heating step or the post-development exposure step, and (e) the metal layer forming step are repeated in order.

By alternately carrying out the laminating step of laminating the resin composition layer (the resin layer) according to the embodiment of the present invention and the metal layer forming step, the resin composition layer (the resin layer) according to the embodiment of the present invention and the metal layer can be alternately laminated.

### (Surface Activation Treatment Step)

The manufacturing method for a laminate according to the embodiment of the present invention preferably includes a surface activation treatment step of subjecting at least a part of the metal layer or a part of the resin composition layer to a surface activation treatment.

The surface activation treatment step is usually carried out after the metal layer forming step (preferably, after at least one of the heating step or the post-development exposure step). However, after the development step, the metal layer forming step may be carried out after the resin composition layer is subjected to the surface activation treatment step.

Only at least a part of the metal layer may be subjected to the surface activation treatment, only at least a part of the resin composition layer after the exposure may be subjected to the surface activation treatment, or both at least a part of the metal layer and at least a part of the resin composition layer after the exposure may be subjected to the surface activation treatment. The surface activation treatment is preferably carried out on at least a part of the metal layer, and is more preferably carried out on a part or whole of the region of the metal layer having a surface on which the resin composition layer is formed. In a case where a surface of the metal layer is subjected to the surface activation treatment in this manner, it is possible to improve the adhesiveness to the resin composition layer (film) to be provided on the surface thereof.

It is preferable that the surface activation treatment is carried out on a part or whole of the resin composition layer (the resin layer) after the exposure. In a case where a surface of the resin composition layer is subjected to the surface activation treatment in this manner, it is possible to improve the adhesiveness to a metal layer or a resin layer to be provided on the surface that has been subjected to the surface activation treatment. In particular, in a case where the resin composition layer is cured, such as in a case where negative-tone development is carried out, it is less likely to be damaged by the surface treatment, and thus the adhesiveness is likely to be improved.

The surface activation treatment can be carried out, for example, according to the method described in paragraph 0415 of WO2021/112189A. The content thereof is incorporated in the present specification.

### (Semiconductor Device and Manufacturing Method Therefor)

The present invention also discloses a semiconductor device, which includes the cured substance according to the embodiment of the present invention or the laminate.

In addition, the present invention also discloses a manufacturing method for a semiconductor device, which includes the manufacturing method for a cured substance according to the embodiment of the present invention or the manufacturing method for a laminate according to the embodiment of the present invention.

As the specific examples of the semiconductor device using the resin composition according to the embodiment of the present invention for forming an interlayer insulating film for a re-distribution layer, the description in paragraphs 0213 to 0218 and the description of FIG. 1 of JP2016-027357A can be referred to, the content of which is incorporated in the present specification.

### (Polyimide and Manufacturing Method for Polyimide)

The polyimide according to the embodiment of the present invention is a polyimide including a repeating unit represented by Formula (1-1).

A preferred aspect of the polyimide according to the embodiment of the present invention is the same as the preferred aspect of the first specific resin described above, except that it is essential to include the repeating unit represented by Formula (1-1).

The manufacturing method for a polyimide according to the embodiment of the present invention includes a step of reacting a polyimide having an acid group or a precursor thereof with the compound represented by Formula (B-1).

A preferred aspect of the manufacturing method for a polyimide according to the embodiment of the present invention is the same as the preferred aspect of the manufacturing method for a specific resin described above.

### (Compound)

The compound according to the first aspect of the present invention is a compound represented by Formula (A-1).

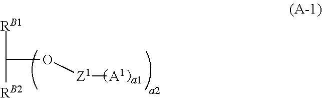

In Formula (A-1), Z¹'s each independently represent a (1+a1)-valent linking group, A¹'s each independently represent a polymerizable group, a1's each independently represent an integer of 1 or more and a maximum substituent number of Z¹ or less, R^{B1} and R^{B2} each independently represent a hydrogen atom or a monovalent organic group, and a2 represents 2.

In Formula (A-1), preferred aspects of Z¹, A¹, a1, R^{B1}, and R^{B2} are the same as the preferred aspects of Z¹, A¹, a1, R^{B1}, and R^{B2} in Formula (B-1) described above.

The compound according to the second aspect of the present invention is the compound represented by Formula (B-1).

Preferred aspects of Formula (B-1) are as described above.

### EXAMPLES

Hereinafter, the present invention will be described in detail using examples. Materials, using amounts, proportions, treatment details, treatment content, and the like shown in the following examples can be appropriately changed without departing from the gist of the present invention. Accordingly, the scope of the present invention is not limited to the following specific examples. Unless otherwise specified, “parts” and “%” are based on mass.

### Synthesis of Crosslinking Agent Raw Material (S-1)

In a flask equipped with a condenser, 60.0 g (439.2 millimoles) of 6-chloro-1-hexanol, 41.7 g (52.8 millimoles) of pyridine, and 0.5 g of N,N-dimethylaminopyridine were dissolved in 180 mL of ethyl acetate, and the solution was cooled to −5° C. to 0° C. while stirring. Subsequently, 50.5 g (483.6 millimoles) of methacrylic acid chloride was added dropwise thereto over 1 hour, and the mixture was stirred at −5° C. to 0° C. for 1 hour, and then heated to 20° C. to 25° C. and stirred for 3 hours. Subsequently, the above-described reaction solution was transferred to a separatory funnel, 500 mL of acetic acid was added thereto to dilute the solution, and the solution was washed twice in the order of 500 mL of water, 200 mL of dilute hydrochloric acid, 300 mL of saturated sodium hydrogen carbonate solution, and 500 mL of saturated saline solution, dried with magnesium sulfate, and filtered with filter paper. The solvent was removed from the above-described filtrate with an evaporator to synthesize S-1 as a crosslinking agent raw material. The structure of S-1 was confirmed by ¹H-NMR.

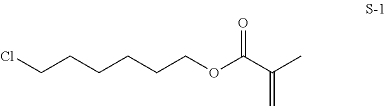

### Synthesis of Crosslinking Agent (BS-1)

In a flask equipped with a condenser, 10.0 g (32.42 millimoles) of 2,2-diallyl bisphenol A and 14.60 g (71.3 millimoles) of S-1 synthesized above were dissolved in 90.0 g of N,N′-dimethylformamide, 13.44 g of potassium carbonate, 1.62 g of potassium iodide, and 0.03 g of 2,2,6,6-tetramethylpiperidine 1-oxyl free radical were added thereto, and the mixture was stirred at 95° C. for 20 hours and cooled to 25° C. Subsequently, the above-described reaction solution was transferred to a separatory funnel, 500 mL of acetic acid was added thereto to dilute the solution, and the solution was washed twice in the order of 500 mL of water, 200 mL of dilute hydrochloric acid, 300 mL of saturated sodium hydrogen carbonate solution, and 500 mL of saturated saline solution, dried with magnesium sulfate, and filtered with filter paper. The solvent was removed from the above-described filtrate with an evaporator, and the residue was purified by column chromatography (ethyl acetate/hexane=1/10 (volume %)) using silica gel, and the solvent was removed with an evaporator to obtain 8.5 g of BS-1. The structure of BS-1 was confirmed by ¹H-NMR.

¹H-NMR data: (heavy DMSO, 400 MHz, internal standard: tetramethylsilane)

δ (ppm)=1.35 to 1.48 (m, 8H), 1.54 (s, 6H), 1.60 to 1.73 (m, 8H), 1.86 (s, 6H), 3.23 to 3.25 (d, 4H), 3.88 to 3.91 (t, 4H), 4.07 to 4.10 (t, 4H), 4.92 (s, 2H), 4.94 to 4.97 (d, 2H), 5.63 (s, 2H), 5.82 to 5.92 (m, 2H), 6.01 (s, 2H), 6.77 to 6.80 (d, 2H), 6.93 (s, 2H), 6.95 to 6.98 (d, 2H)

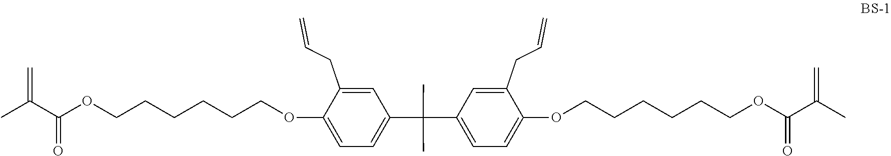 [SMILES: C=CCc1cc(C(C)(C)c2ccc(OCCCCCCOC(=O)C(=C)C)c(CC=C)c2)ccc1OCCCCCCOC(=O)C(=C)C]

### Synthesis of 6-Maleimidehexane Acid Chloride

In an eggplant flask equipped with a thermometer and a calcium chloride tube, 7.92 g (37.5 millimoles) of 6-maleimidehexanoic acid was dissolved in 30 g of tetrahydrofuran, 0.1 g of N,N′-dimethylformamide was added thereto, and the mixture was cooled to 0° C. while stirring with a magnetic stirrer. Subsequently, 4.85 g (38.25 millimoles) of oxalyl chloride was added dropwise thereto, and the mixture was stirred at 0° C. to 10° C. for 1 hour. Subsequently, the temperature was raised to 25° C., and the mixture was stirred for 2 hours to synthesize 6-maleimidehexane acid chloride.

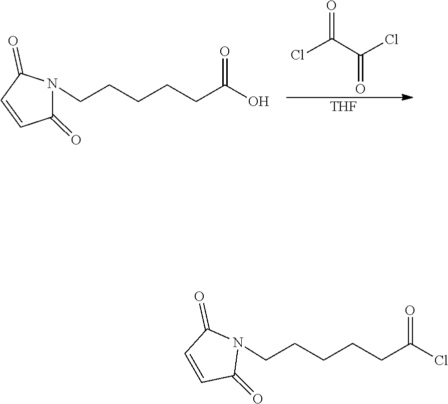 [SMILES: O=C(O)CCCCCN1C(=O)C=CC1=O ; O=C(Cl)C(=O)Cl ; O=C(Cl)CCCCCN1C(=O)C=CC1=O ; "THF"]

### Synthesis of Crosslinking Agent (BS-2)

In a flask equipped with a condenser, 7.72 g (25 millimoles) of 2,2-diallyl bisphenol A and 0.01 g of 2,2,6,6-tetramethylpiperidine 1-oxyl free radical were dissolved in 50.0 g of dehydrated tetrahydrofuran, and the mixture was cooled to 0° C. Subsequently, 12.60 g (55 millimoles) of 6-maleimidehexane acid chloride (M-1) synthesized above was added dropwise thereto over 1 hour, and the mixture was stirred at 25° C. for 5 hours. Subsequently, the above-described reaction solution was transferred to a separatory funnel, 500 mL of acetic acid was added thereto to dilute the solution, and the solution was washed twice in the order of 500 mL of water, 200 mL of dilute hydrochloric acid, 300 mL of saturated sodium hydrogen carbonate solution, and 500 mL of saturated saline solution, dried with sodium sulfate, and filtered with filter paper. The solvent was removed from the above-described filtrate with an evaporator, and the residue was purified by column chromatography (ethyl acetate/hexane=1/2 (volume %)) using silica gel, and the solvent was removed with an evaporator to obtain 5.52 g of BS-2. The structure of BS-2 was confirmed by ¹H-NMR.

¹H-NMR data: (heavy DMSO, 400 MHz, internal standard: tetramethylsilane)

δ (ppm)=1.26 to 1.34 (m, 4H), 1.50 to 1.57 (m, 4H), 1.60 to 1.60 (m, 10H), 2.54 to 2.57 (t, 4H), 3.18 to 3.20 (d, 4H), 3.39 to 3.43 (t, 4H), 4.95 to 4.97 (d, 2H), 5.00 (s, 2H), 5.75 to 5.85 (m, 2H), 6.94 to 6.96 (d, 2H), 7.01 (s, 4H), 7.07 to 7.09 (d, 2H), 7.13 (s, 2H)

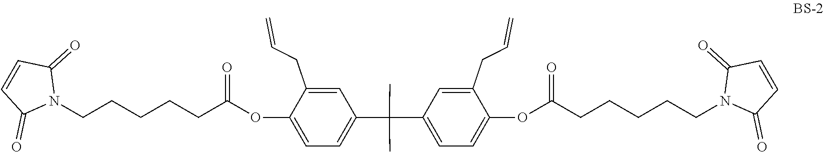 [SMILES: C=CCc1cc(C(C)(C)c2ccc(OC(=O)CCCCCN3C(=O)C=CC3=O)c(CC=C)c2)ccc1OC(=O)CCCCCN1C(=O)C=CC1=O]

### Synthesis of Dinitro Compound A-1

In a flask equipped with a stirrer, a condenser, and a thermometer, 23.06 g (100 millimoles) of 3,5-dinitrobenzyl chloride, 0.005 g of 2,2,6,6-tetramethylpiperidine 1-oxyl free radical, and 9.50 g (120 millimoles) of dehydrated pyridine were dissolved in 80 g of dehydrated tetrahydrofuran, and the solution was cooled to a range of 0° C. to 10° C. Subsequently, 11.92 g (100 millimoles) of 4-aminostyrene was dissolved in 40 g of dehydrated tetrahydrofuran, and the solution was added dropwise thereto at a temperature range of 0° C. to 10° C. over 1 hour, and the mixture was stirred for 1 hour, and then heated to 20° C. to 25° C. and stirred for 2 hours. The above-described reaction solution was crystallized in 1 L of water, filtered, reslurried twice with 500 mL of 0.1N hydrochloric acid aqueous solution, washed with 500 mL of water, and dried at 40° C. for 24 hours to obtain 30.0 g of a dinitro compound A-1 having a vinylphenyl group. The structure of A-1 was confirmed by ¹H-NMR spectrum. ¹H-NMR (BRUKER, AVANCE NEO 400): δ (ppm, DMSO-d6) 5.31 to 5.33 (d, 1H), 5.87 to 5.91 (d, 1H), 6.77 to 6.83 (q, 1H), 7.38 to 7.40 (d, 2H), 7.61 to 7.64 (d, 1H), 9.08 (s, 2H), 9.12 (s, 1H).

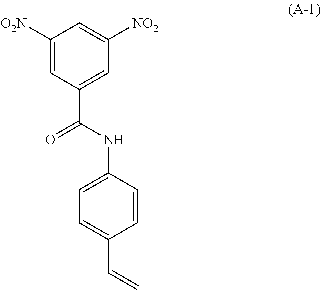 [SMILES: C=Cc1ccc(NC(=O)c2cc([N+](=O)[O-])cc([N+](=O)[O-])c2)cc1]

### Synthesis of Dinitro Compound A-2

A dinitro compound A-2 having a vinylphenyl group was synthesized by the same method as in the synthesis of the dinitro compound A-1, except that the raw materials were appropriately changed. The structure of A-2 was confirmed by ¹H-NMR spectrum.

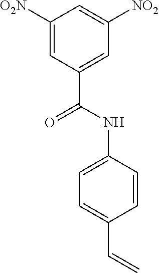 [SMILES: C=Cc1ccc(NC(=O)c2cc([N+](=O)[O-])cc([N+](=O)[O-])c2)cc1]

### Synthesis of Diamine Compound AA-1

In a flask equipped with a stirrer, a condenser, and a thermometer, 5.75 g (95.8 millimoles) of acetic acid, 11.3 g (210 millimoles) of ammonium chloride, 0.01 g of 2,2,6,6-tetramethylpiperidine 1-oxyl free radical, 240 mL of isopropyl alcohol, and 60 g of water were added thereto. Subsequently, 55.7 g (958 millimoles) of reduced iron was added thereto, 30.0 g (95.8 millimoles) of A-1 synthesized above was added thereto in small amounts over 1 hour, and the mixture was stirred for 30 minutes, and then heated to 80° C. and stirred for 4 hours, and the mixture was filtered using a Nutsche equipped with a bed of Celite to recover the filtrate. Subsequently, the above-described filtrate was transferred to a separatory funnel, diluted with 500 mL of ethyl acetate, washed three times with 300 mL of saturated sodium hydrogen carbonate solution and once with 300 mL of saturated sodium hydrogen carbonate solution, dried with sodium sulfate, and the solvent was removed with an evaporator, and the precipitated solid was dried at 40° C. for 24 hours to obtain 22 g of a diamine compound AA-1 having a styryl group.

It was confirmed by ¹H-NMR spectrum that the compound was AA-1.

¹H-NMR (BRUKER, AVANCE NEO 400): δ (ppm, DMSO-d6) 5.10 (s, 4H), 5.26 to 5.29 (d, 1H), 5.81 to 5.85 (d, 1H), 6.10 (s, 1H), 6.58 (s, 2H), 6.72 to 6.80 (q, 1H), 7.16 to 7.19 (d, 2H), 7.53 to 7.55 (d, 2H)

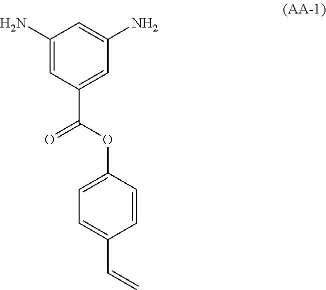 [SMILES: C=Cc1ccc(OC(=O)c2cc(N)cc(N)c2)cc1]

### Synthesis of Diamine Compound AA-2

A diamine compound AA-2 having a vinylphenyl group was synthesized by the same method as in the synthesis of the diamine compound AA-1, except that the raw materials were appropriately changed. The structure of AA-2 was confirmed by ¹H-NMR spectrum.

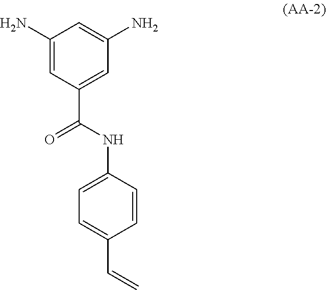 [SMILES: C=Cc1ccc(NC(=O)c2cc(N)cc(N)c2)cc1]

### Synthesis of Di-Anhydride AAA-1 Having Vinylphenyl Group

In a flask equipped with a stirrer, a condenser, and a thermometer, 17.3 g (82 millimoles) of trimellitic acid chloride, 0.005 g of 2,2,6,6-tetramethylpiperidine 1-oxyl free radical, and 6.96 g (88 millimoles) of dehydrated pyridine were dissolved in 80 g of dehydrated tetrahydrofuran, and the solution was cooled to a range of −10° C. to 0° C. Subsequently, 10.17 g (40 millimoles) of AA-1 synthesized above was dissolved in 50 g of dehydrated tetrahydrofuran, and the solution was added dropwise thereto at a temperature range of −10° C. to 0° C. over 2 hours, and the mixture was stirred for 1 hour, and then heated to 20° C. to 25° C. and stirred for 2 hours. The above-described reaction solution was transferred to a separatory funnel, dissolved in 1 L of ethyl acetate, washed twice in the order of 300 mL of water, 200 mL of 0.5N hydrochloric acid, 300 mL of saturated sodium hydrogen carbonate solution, and 300 mL of saturated saline solution, dried with sodium sulfate, and the solvent was removed with an evaporator to obtain crude crystals. Subsequently, the above-described crude crystals were mixed with 100 mL of acetic anhydride, stirred at 80° C. for 3 hours, filtered, washed with 300 mL of hexane, and dried at 40° C. for 24 hours to obtain 18.5 g of a di-anhydride AAA-1 having a vinylphenyl group.

The structure of AAA-1 was confirmed by ¹H-NMR spectrum.

¹H-NMR (BRUKER, AVANCE NEO 400): δ (ppm, DMSO-d6)

5.31 to 5.33 (d, 1H), 5.85 to 5.90 (d, 1H), 6.76 to 6.83 (q, 1H), 7.30 to 7.33 (d, 2H), 7.59 to 7.62 (d, 2H), 8.25 to 8.27 (d, 2H), 8.40 (s, 2H), 8.54 to 8.56 (d, 2H), 8.68 (s, 2H), 8.94 (s, 1H), 11.03 (s, 2H)

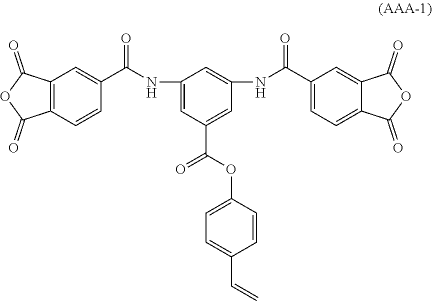 [SMILES: C=Cc1ccc(OC(=O)c2cc(NC(=O)c3ccc4c(c3)C(=O)OC4=O)cc(NC(=O)c3ccc4c(c3)C(=O)OC4=O)c2)cc1]

### Synthesis of Di-Anhydride AAA-2 Having Vinylphenyl Group

A di-anhydride AAA-2 having a vinylphenyl group was synthesized by the same method as in the synthesis of the di-anhydride AAA-1, except that the raw materials were appropriately changed. The structure of AAA-2 was confirmed by ¹H-NMR spectrum.

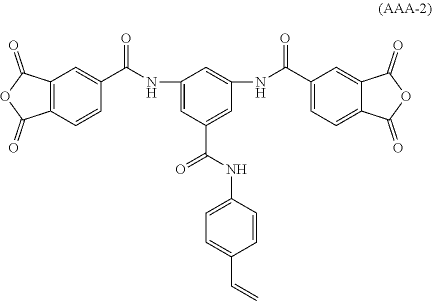 [SMILES: C=Cc1ccc(NC(=O)c2cc(NC(=O)c3ccc4c(c3)C(=O)OC4=O)cc(NC(=O)c3ccc4c(c3)C(=O)OC4=O)c2)cc1]

### Production Example AC-1: Synthesis of Acetal Raw Material (AC-1)

In a flask equipped with a stirrer, a condenser, and a thermometer, 38.0 g (527 millimoles) of isobutyraldehyde, 150.9 g (1159 millimoles) of 2-hydroxyethyl methacrylate, and 0.3 g of p-methoxyphenol were dissolved, 60 g of magnesium sulfate was added thereto, and the mixture was cooled to 0° C. to 10° C. Subsequently, 1.00 g of methanesulfonic acid was added thereto, and the mixture was stirred for 1 hour, and then heated to 50° C. and stirred for 4 hours, and cooled to 25° C. Subsequently, 2.67 g of triethylamine was added thereto, and the mixture was dissolved in 600 mL of ethyl acetate, filtered while adding magnesium sulfate, transferred to a separatory funnel, washed three times with 300 mL of sodium hydrogen carbonate solution and twice with 300 mL of saturated saline solution, dried with sodium sulfate, and the solvent was removed with an evaporator to obtain 153 g of AC-1 as a target substance. It was confirmed by ¹H-NMR spectrum that the compound was AC-1.

¹H-NMR (BRUKER, AVANCE NEO 400): δ (ppm, CDCl₃) 0.91 to 0.93 (d, 6H), 1.95 (s, 6H), 1.97 to 2.00 (m, 1H), 3.71 to 3.75 (t, 2H), 3.80 to 3.83 (t, 2H), 4.19 to 4.21 (d, 1H), 4.29 to 4.32 (t, 4H), 5.58 (s, 2H), 6.12 (s, 2H)

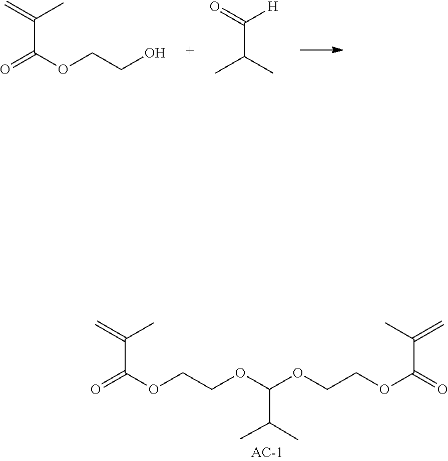

### Production Examples AC-2 to AC-19: Synthesis of Acetal Raw Materials (AC-2 to AC-19)

Acetal raw materials AC-2 to AC-19 were synthesized by the same method as in the synthesis of the acetal raw material AC-1, except that the raw materials were appropriately changed. The structures of AC-2 to AC-19 were confirmed by ¹H-NMR spectrum.

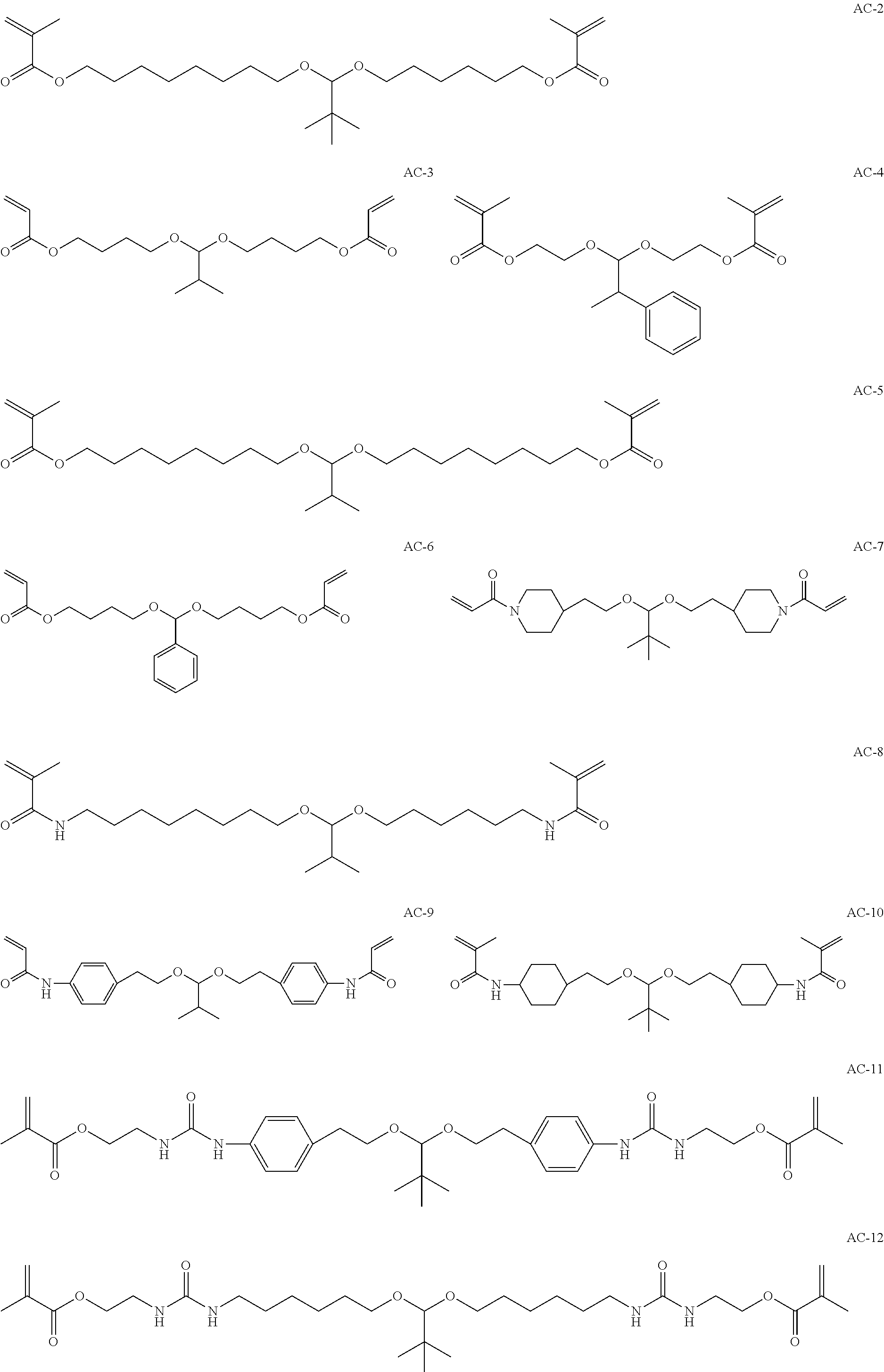

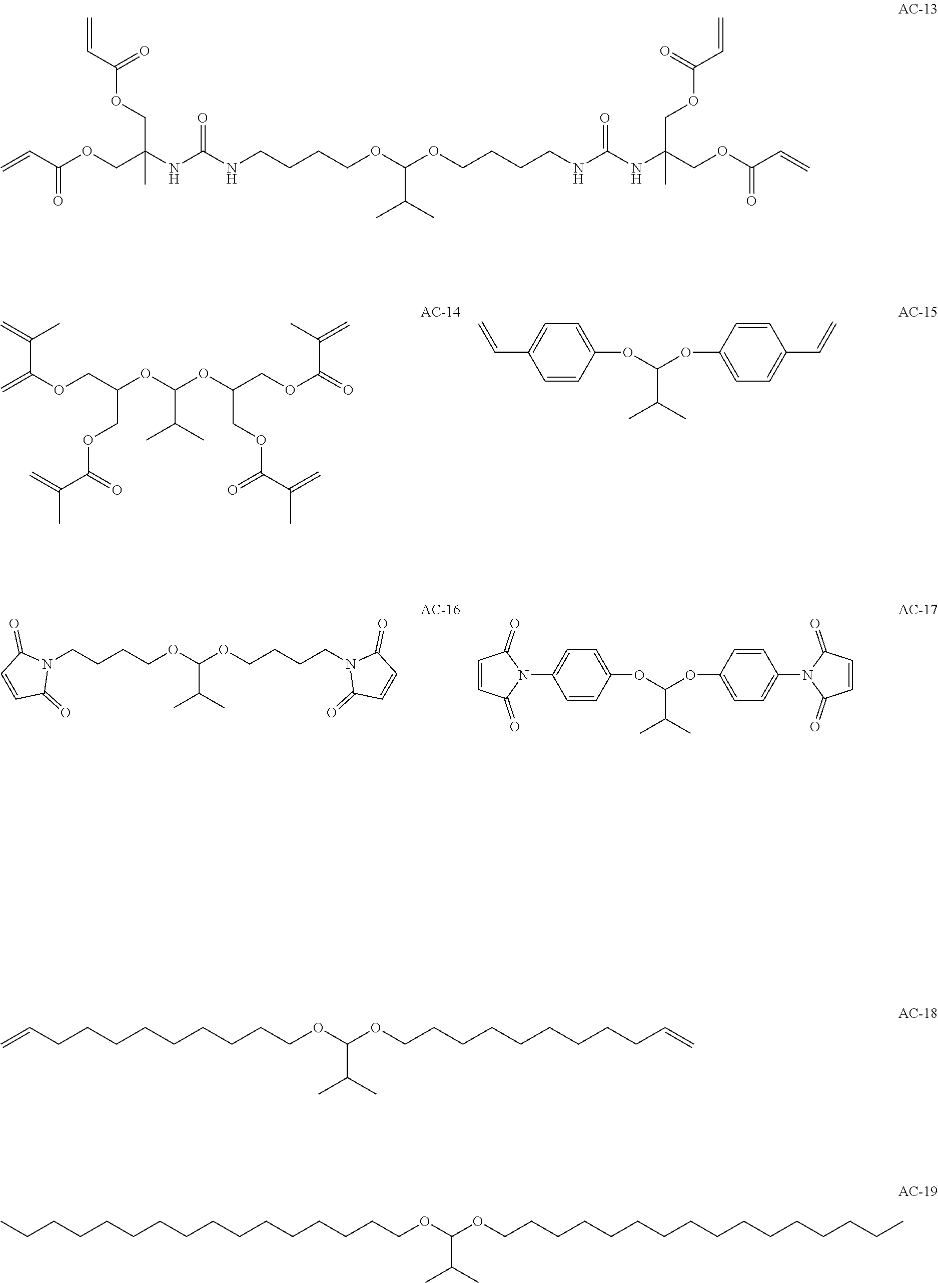

### Production Example BC-1: Synthesis of Acetal Raw Material (BC-1)

In a flask equipped with a stirrer, a condenser, and a thermometer, 153 g (487 millimoles) of AC-1 synthesized above, 49.7 g (633 millimoles) of acetyl chloride, and 0.3 g of 2,2,6,6-tetramethylpiperidine 1-oxyl free radical were dissolved, and the mixture was stirred at 50° C. for 4 hours and cooled to 25° C. Subsequently, acetyl chloride was removed with an evaporator to obtain 198 g of a methacrylic acid 2-hydroxyethyl mixed solution of BC-1 as a target substance. The fact that the compound was a methacrylic acid 2-hydroxyethyl mixed solution of BC-1 was confirmed by ¹H-NMR spectrum.

BC-1: ¹H-NMR (BRUKER, AVANCE NEO 400): δ (ppm, CDCl₃) 1.03 to 1.05 (d, 6H), 1.96 (s, 3H), 2.13 to 2.17 (m, 1H), 4.32 to 4.37 (m, 4H), 5.44 to 5.45 (d, 1H), 5.61 (s, 1H), 6.13 (s, 1H)

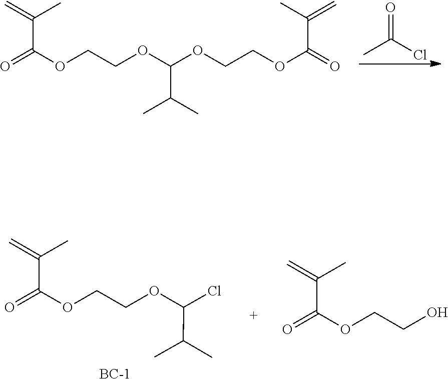

### Production Examples BC-2 to BC-19: Synthesis of Acetal Raw Materials (BC-2 to BC-19)

Acetal raw materials BC-2 to BC-19 were synthesized by the same method as in the synthesis of the acetal raw material BC-1, except that the raw materials were appropriately changed. The structures of BC-2 to BC-19 were confirmed by ¹H-NMR spectrum.

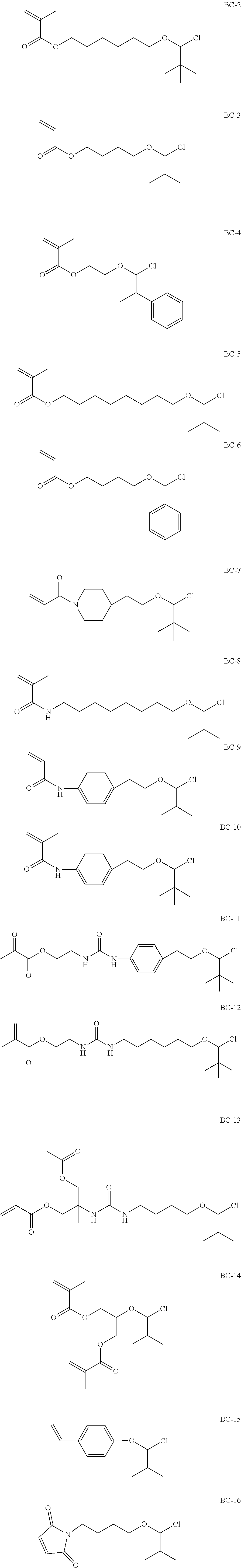

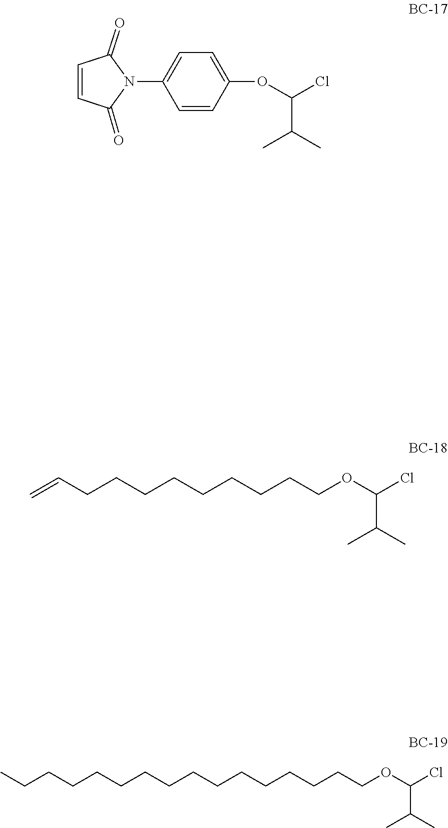 [SMILES: CC(C)C(Cl)Oc1ccc(N2C(=O)C=CC2=O)cc1 ; C=CCCCCCCCCCOC(Cl)C(C)C ; CCCCCCCCCCCCCCCCOC(Cl)C(C)C]

### Production Example SE-1: Synthesis of Polyimide (SE-1) Having Acetal Structure

30.00 g (57.64 mmol) of 4,4′-(4,4′-isopropylidenediphenoxy)diphtalic acid anhydride was dissolved in 150 g of N-methylpyrrolidone (NMP). Subsequently, 3.95 g (25.94 millimoles) of 3,5-diaminobenzoic acid (manufactured by Tokyo Chemical Industry Co., Ltd.), 9.56 g (25.94 millimoles) of 4,4′-bis(3-aminophenoxy)biphenyl, 3.62 g (10.38 millimoles) of hexadecylamine, and 10 g of toluene were added thereto while washing with 30 g of NMP, and the mixture was stirred for 15 minutes, and then reacted at 200° C. for 4 hours while flowing nitrogen, and cooled to 10° C. Subsequently, 13.4 g (103 millimoles) of diisopropylethylamine and 0.1 g of 2,2,6,6-tetramethylpiperidine 1-oxyl free radical were added thereto, 17.2 g (38.9 millimoles) of BC-1 synthesized above was added dropwise thereto over 30 minutes, and the mixture was reacted at 25° C. for 2 hours, and then diluted with 150 g of tetrahydrofuran, and the salt in the reaction solution was filtered with filter paper. Subsequently, the reaction solution was added dropwise to 2 L of methanol, and the mixture was stirred for 15 minutes, and the polyimide resin was filtered. Next, the above-described resin was reslurried in 1 L of methanol, filtered, and dried at 40° C. for 8 hours. Subsequently, the resin dried above was dissolved in 250 g of tetrahydrofuran, 30 g of an ion exchange resin (MB-1, manufactured by Organo Corporation) and 3.0 g of triethylamine were added thereto, and the mixture was stirred for 4 hours, and the ion exchange resin was filtered and removed, and the polyimide resin was precipitated in 2 L of methanol and stirred for 15 minutes. The polyimide resin was filtered and acquired, and dried at 40° C. for 1 day under reduced pressure to obtain a polyimide resin (SE-1). The obtained polyimide resin (SE-1) had a weight-average molecular weight of 17,300 and a number-average molecular weight of 7,900. As a result of the confirmation by ¹H-NMR, the imidization rate was 99% or more, the acetal introduction rate (carboxylic acid protection rate) was 99%, the acetal equivalent was 0.60 (millimoles/g), and the polymerizable group equivalent was 0.60 (millimoles/g). The structure of the repeating unit was determined from a ¹H-NMR spectrum. In the following structures, the subscripts of the repeating units represent the content molar ratio of the respective repeating units.

In the above and the following production examples, unless otherwise specified, the weight-average molecular weight and the number-average molecular weight were measured by the following method.

Using a high-speed GPC device HLC-8420GPC (manufactured by Tosoh Corporation), a guard column of TSK guardcolumn Super AW-H (4.6 mm×35 mm) and two columns of TSKgel Super AWM-H (4.6 mm×150 mm) were connected in series as columns to perform GPC measurement. As an eluent, an NMP (N-methyl-2-pyrrolidone) solution of 0.01 mol/L of lithium bromide was used.

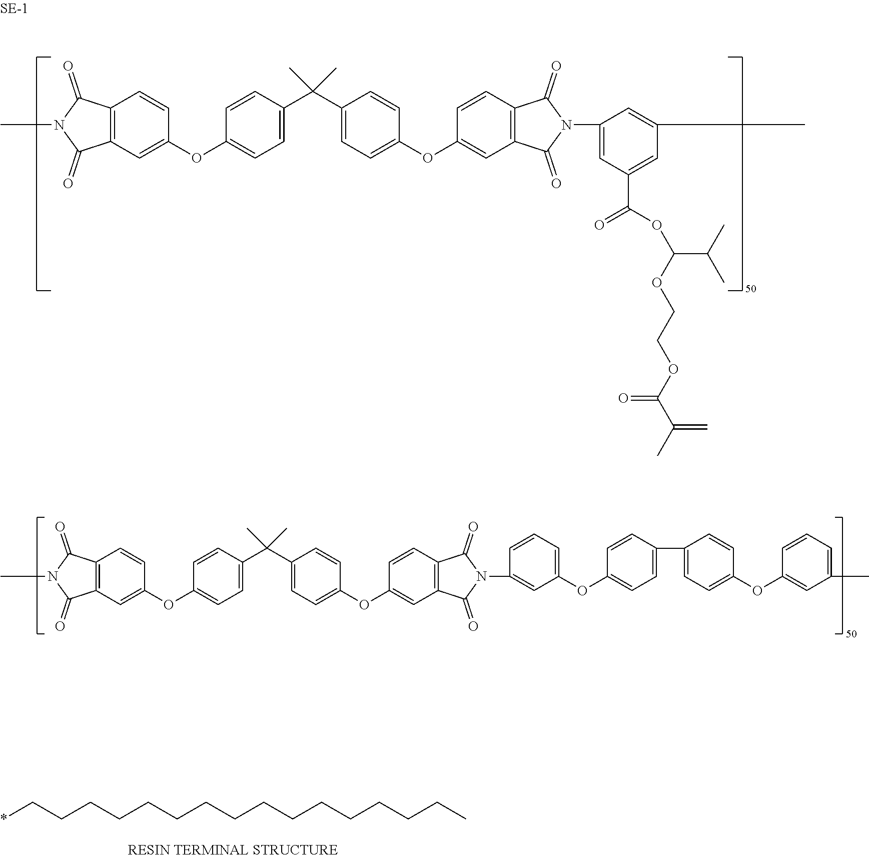

### Production Examples SE-2 to 8, 14, 16 to 18, and 20 to 23: Synthesis of Polyimide (SE-2 to 8, 14, 16 to 18, and 20 to 23) Having Acetal Structure

Polyimides SE-2 to 8, 14, 16 to 18, and 20 to 23 were synthesized by the same method as in the synthesis of the polyimide SE-1, except that the raw materials were appropriately changed. The weight-average molecular weight, the number-average molecular weight, the imidization rate, the acetal introduction rate, the acetal equivalent, and the polymerizable group equivalent of these resins are described in the table shown later. The structure of the repeating unit was determined from a ¹H-NMR spectrum. In the following structures, the subscripts of the repeating units represent the content molar ratio of the respective repeating units.

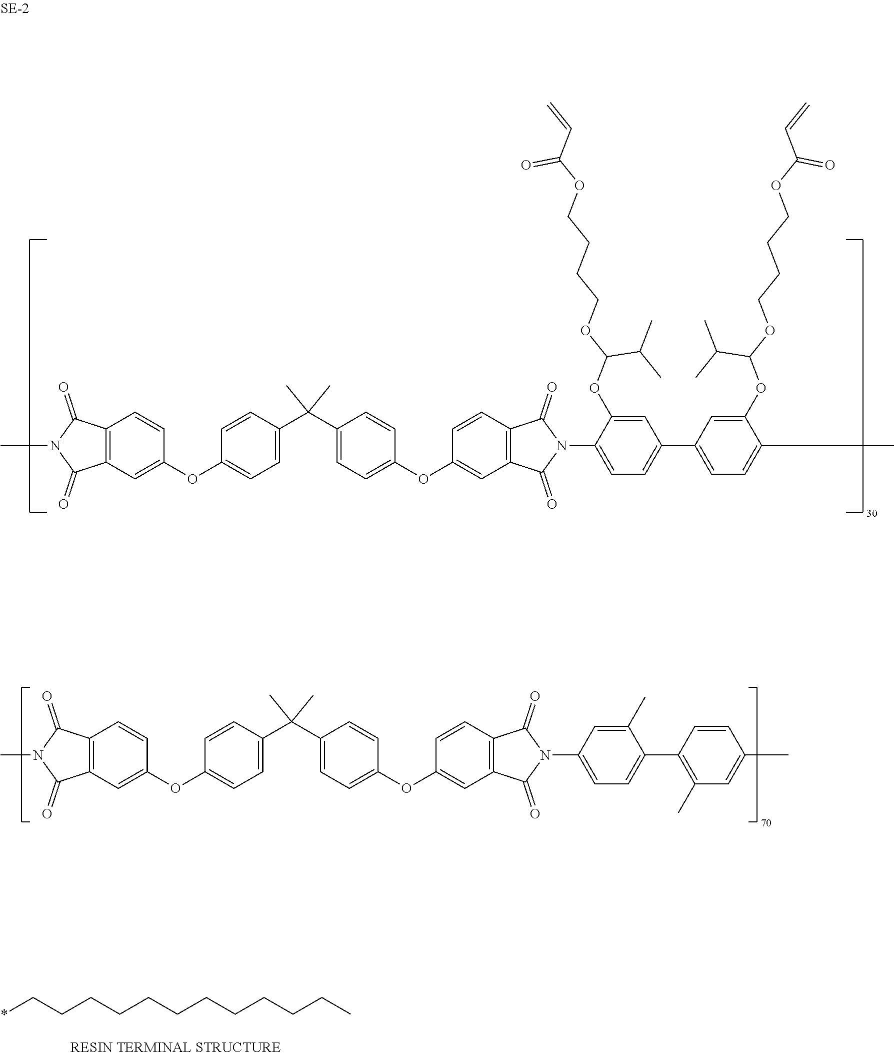

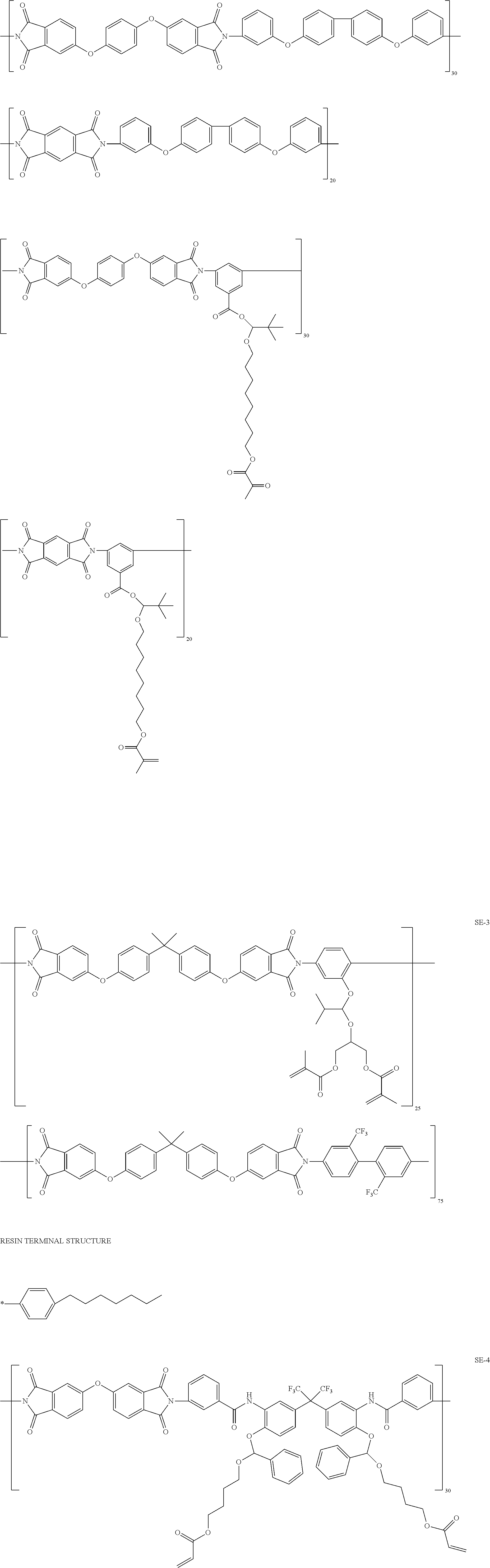

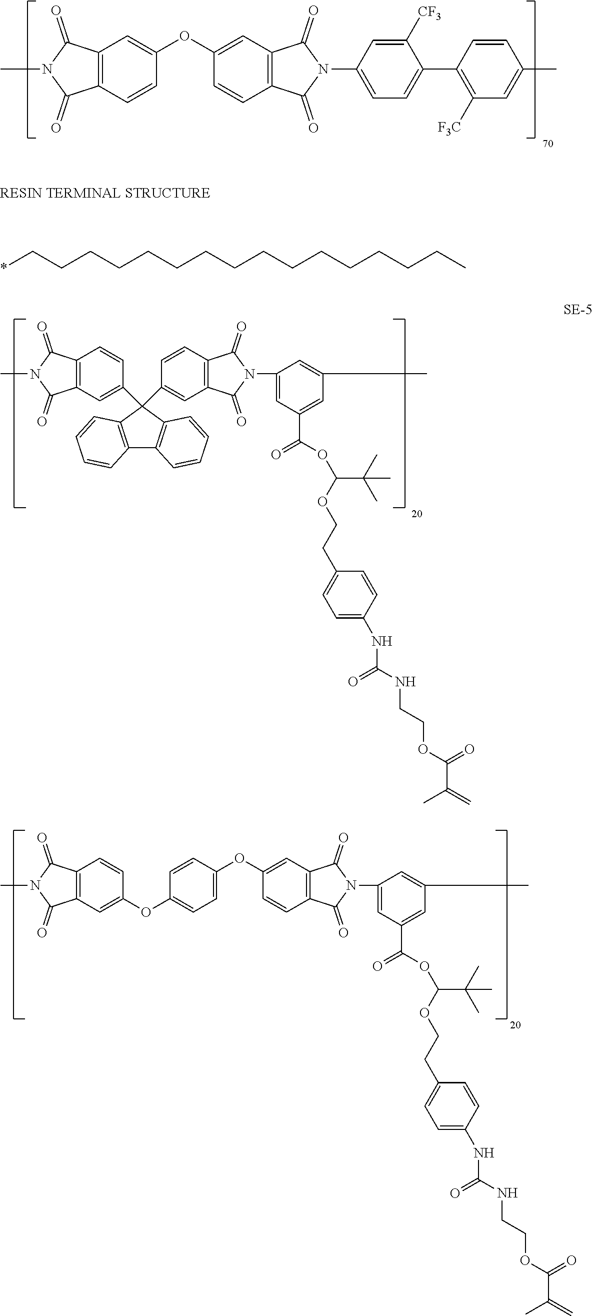

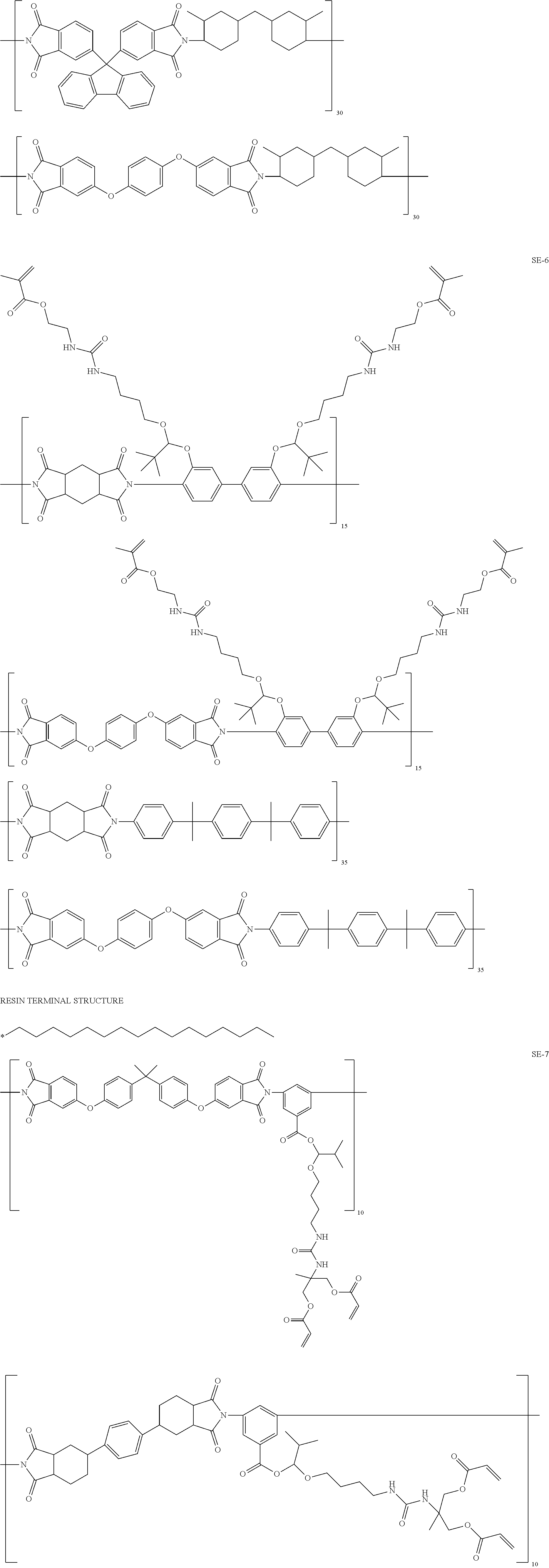

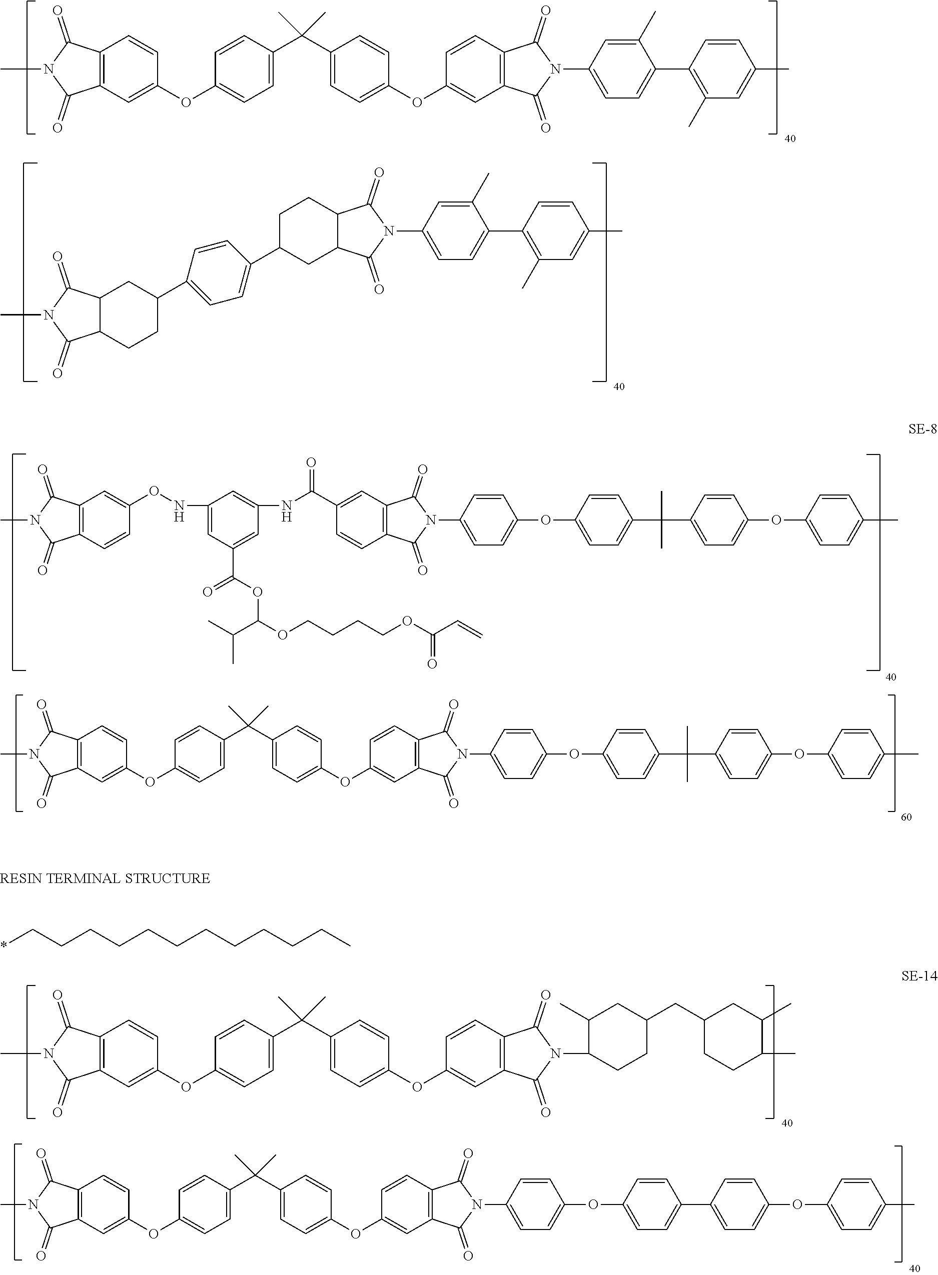

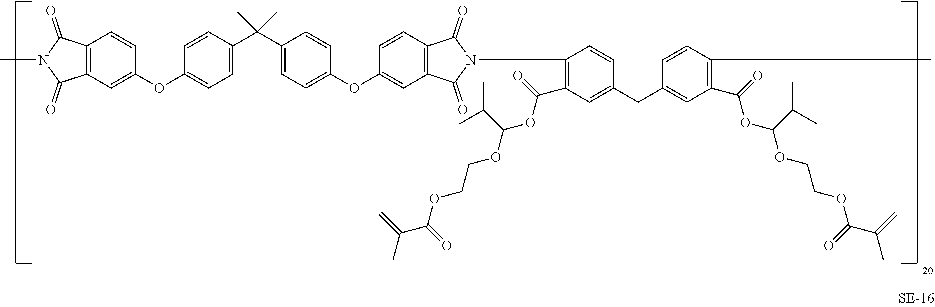

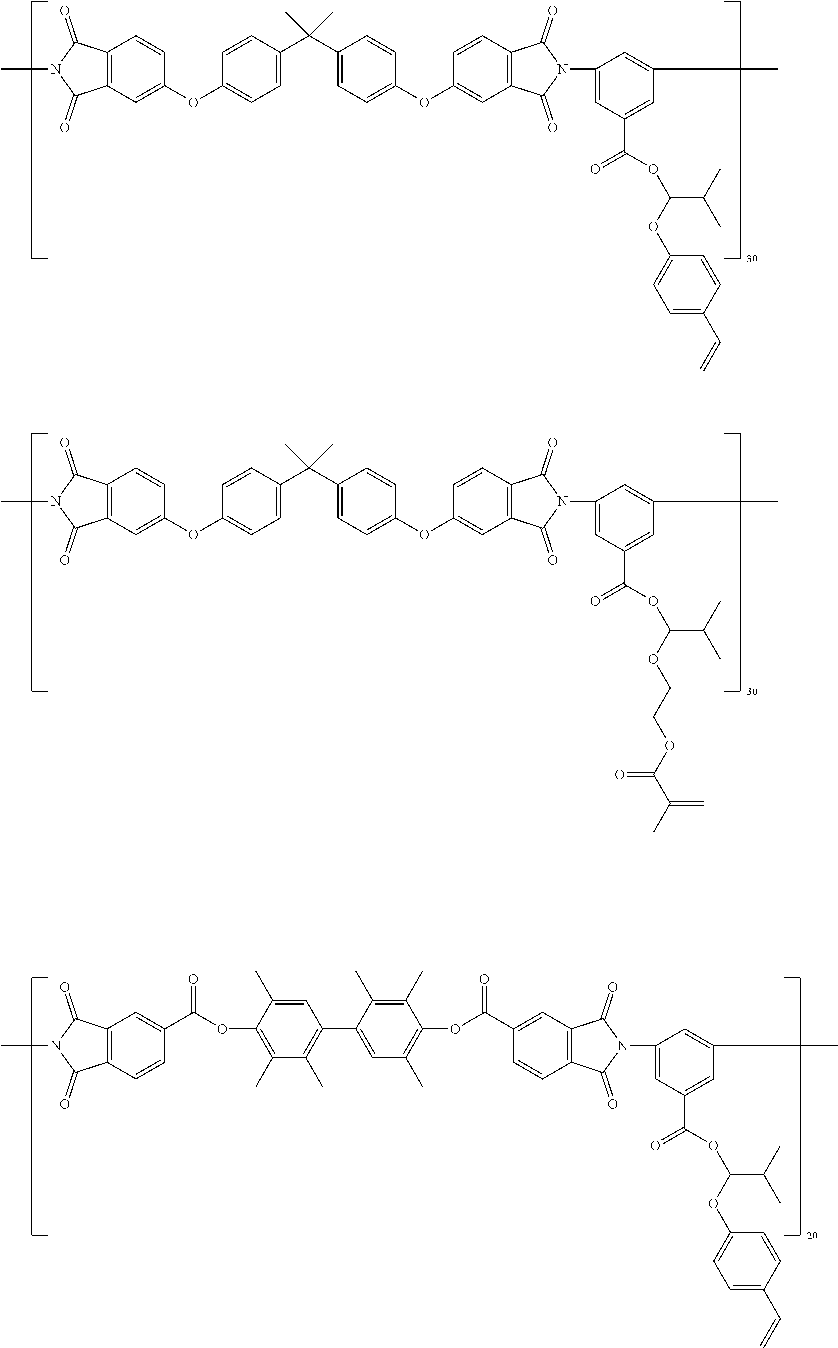

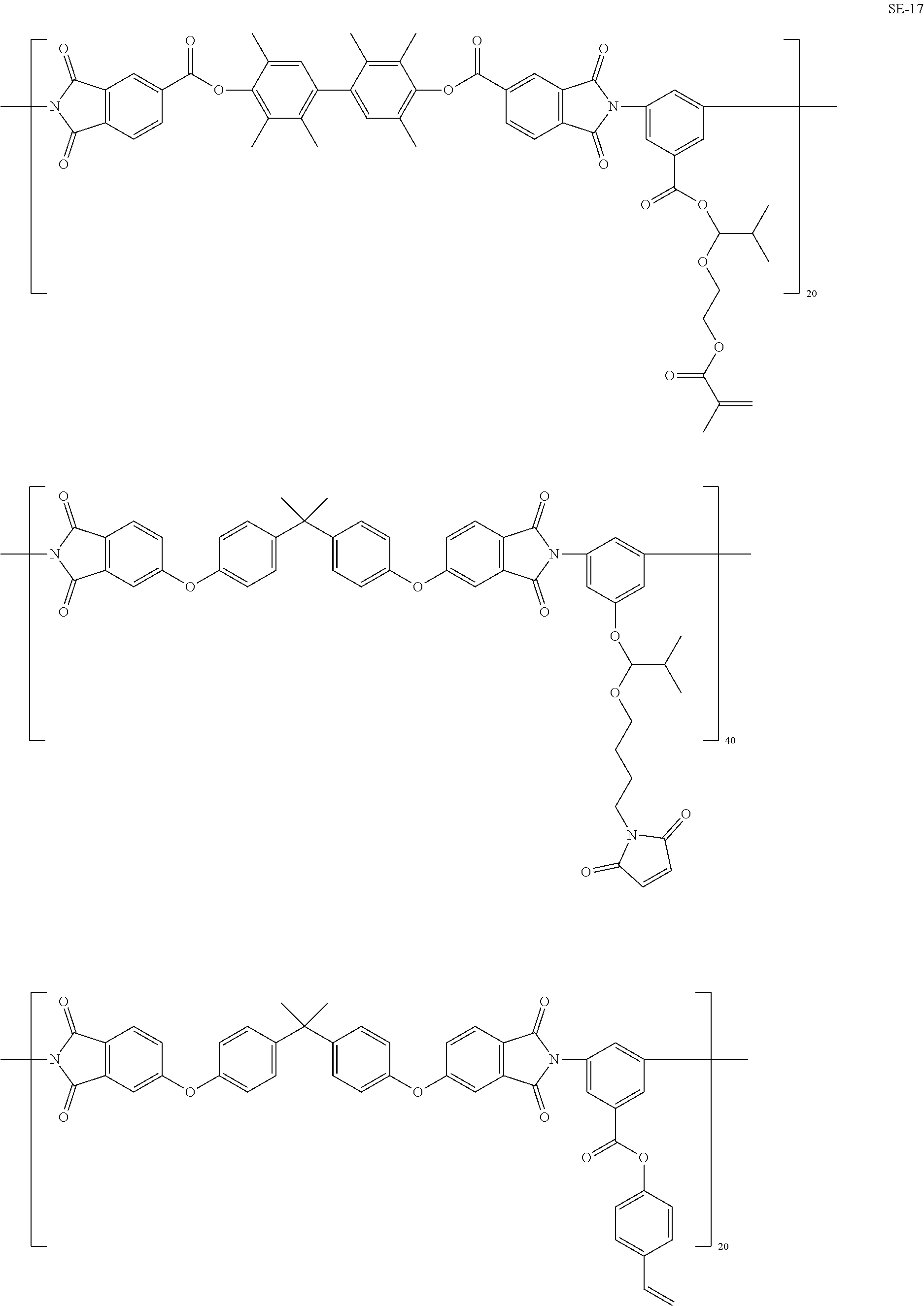

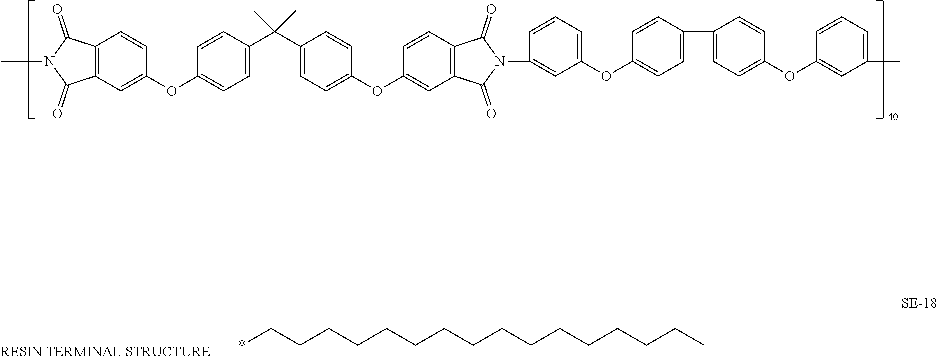

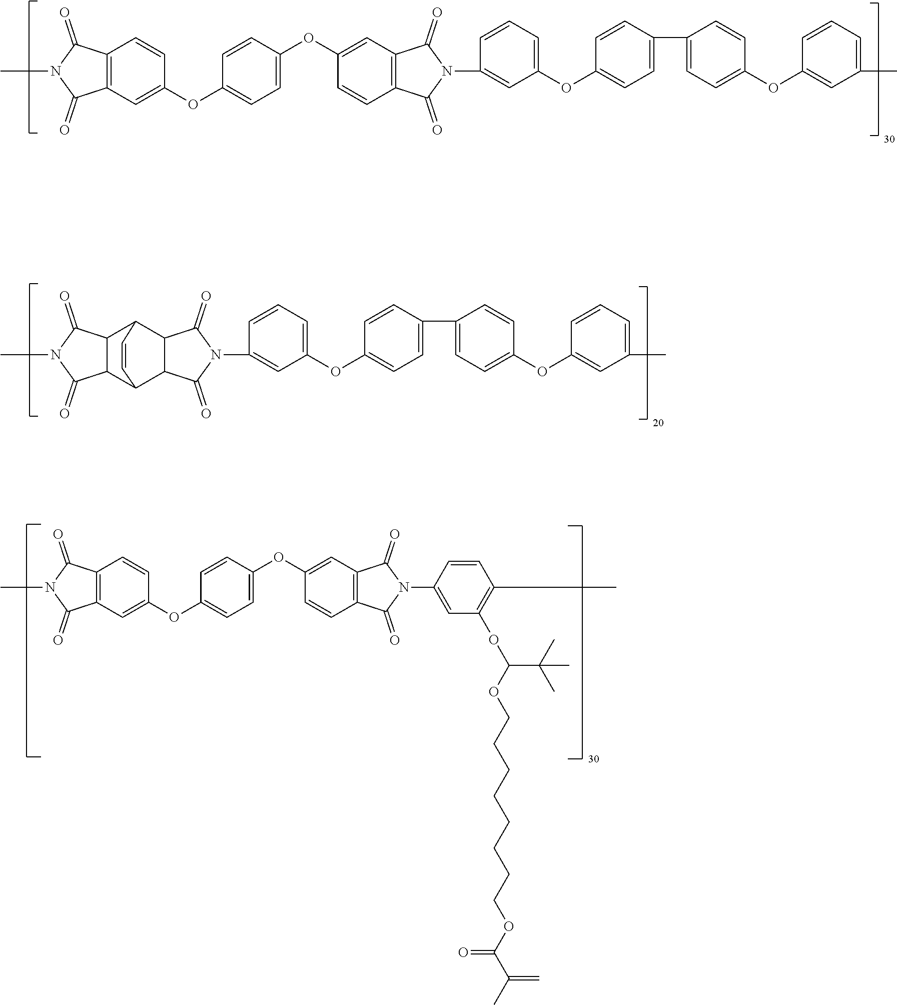

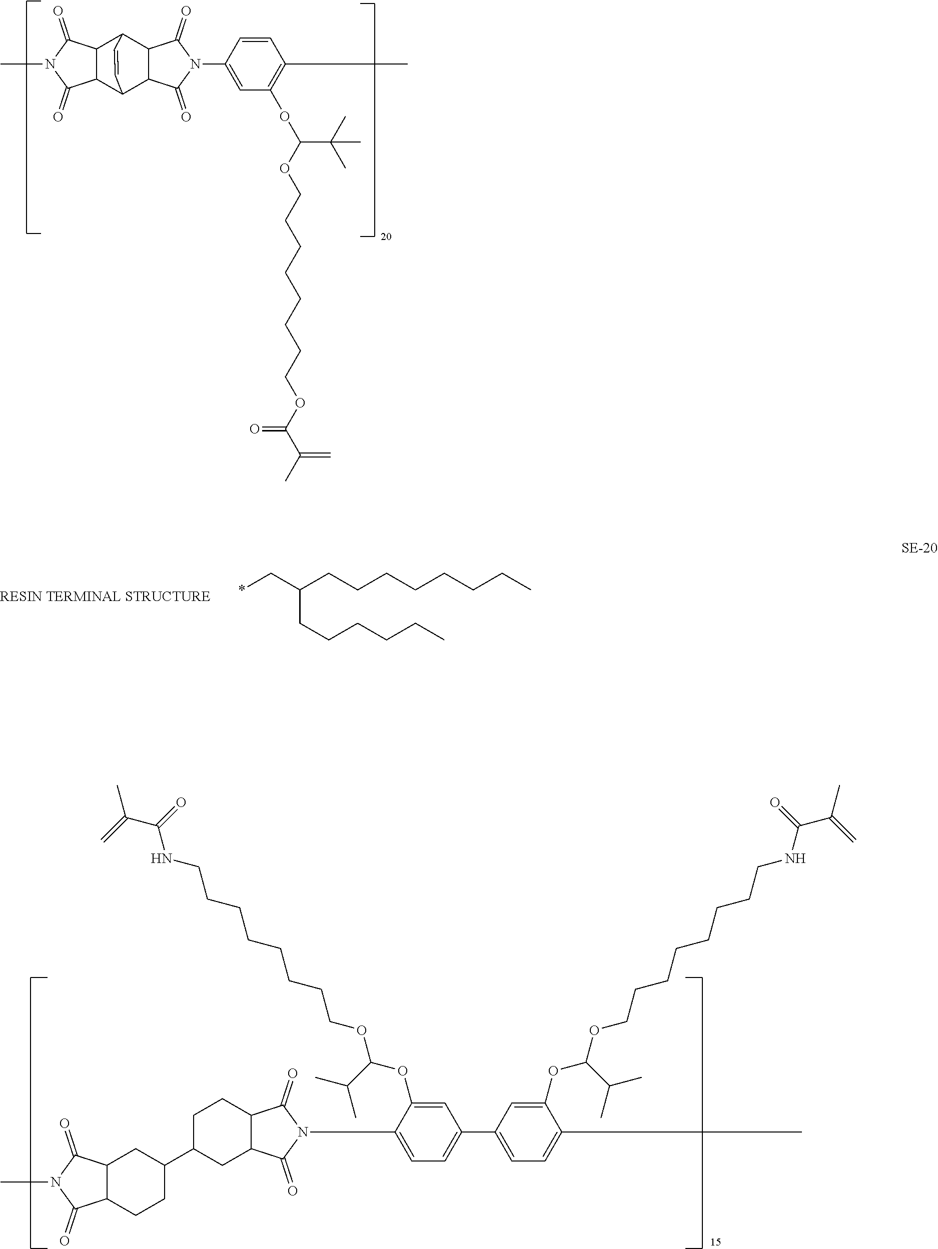

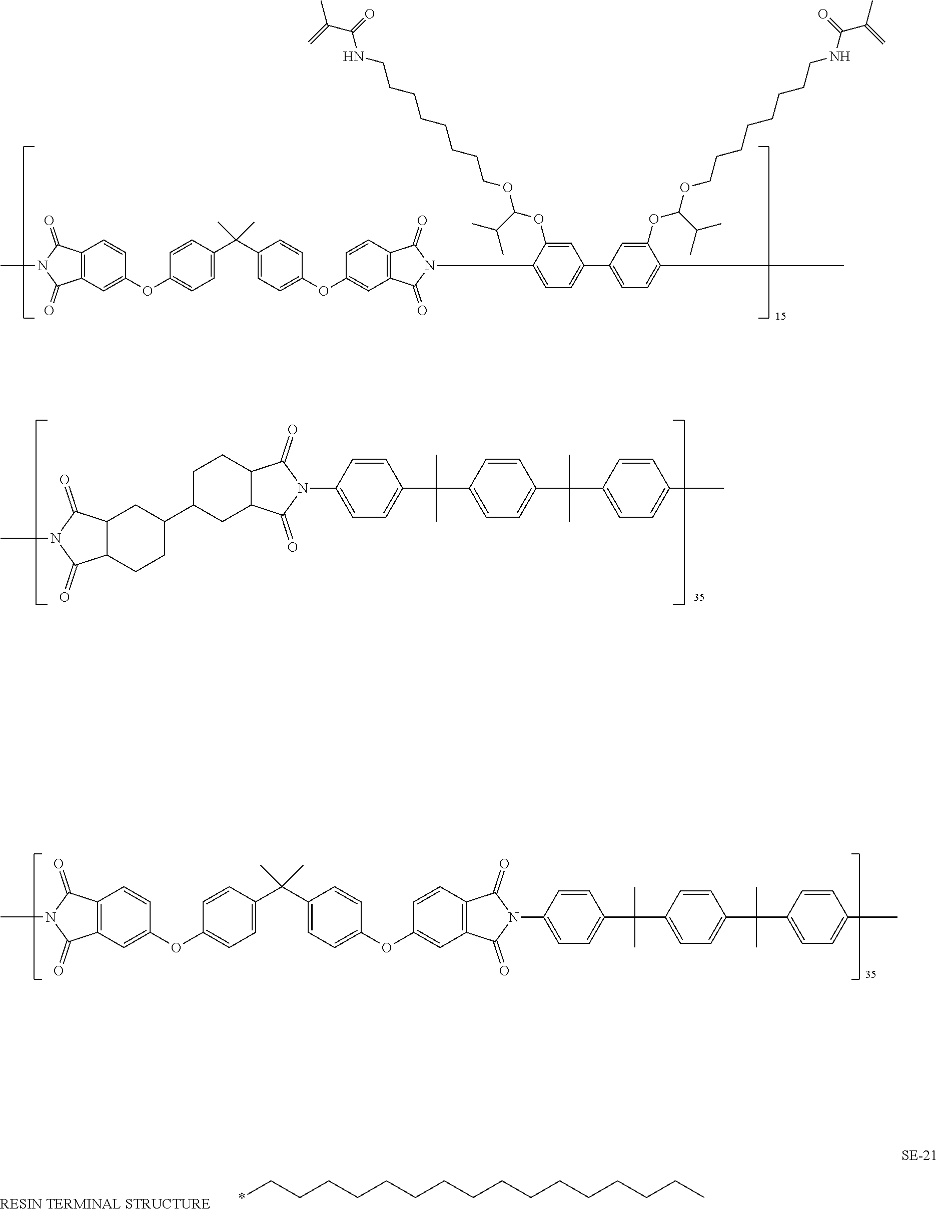

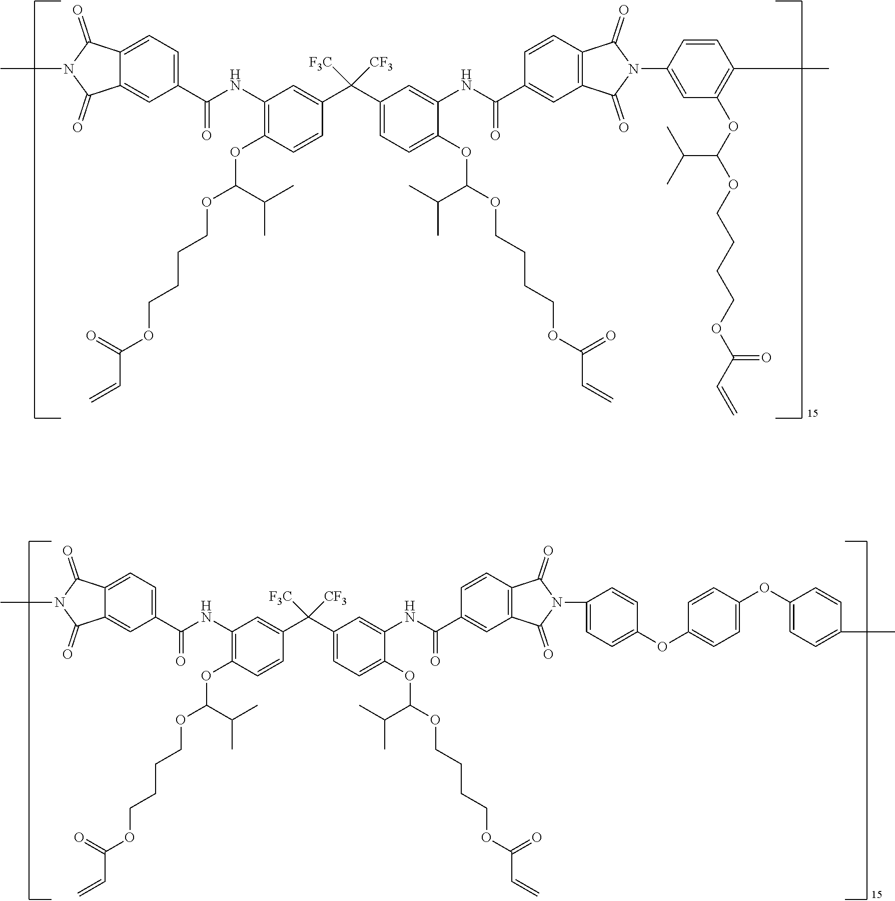

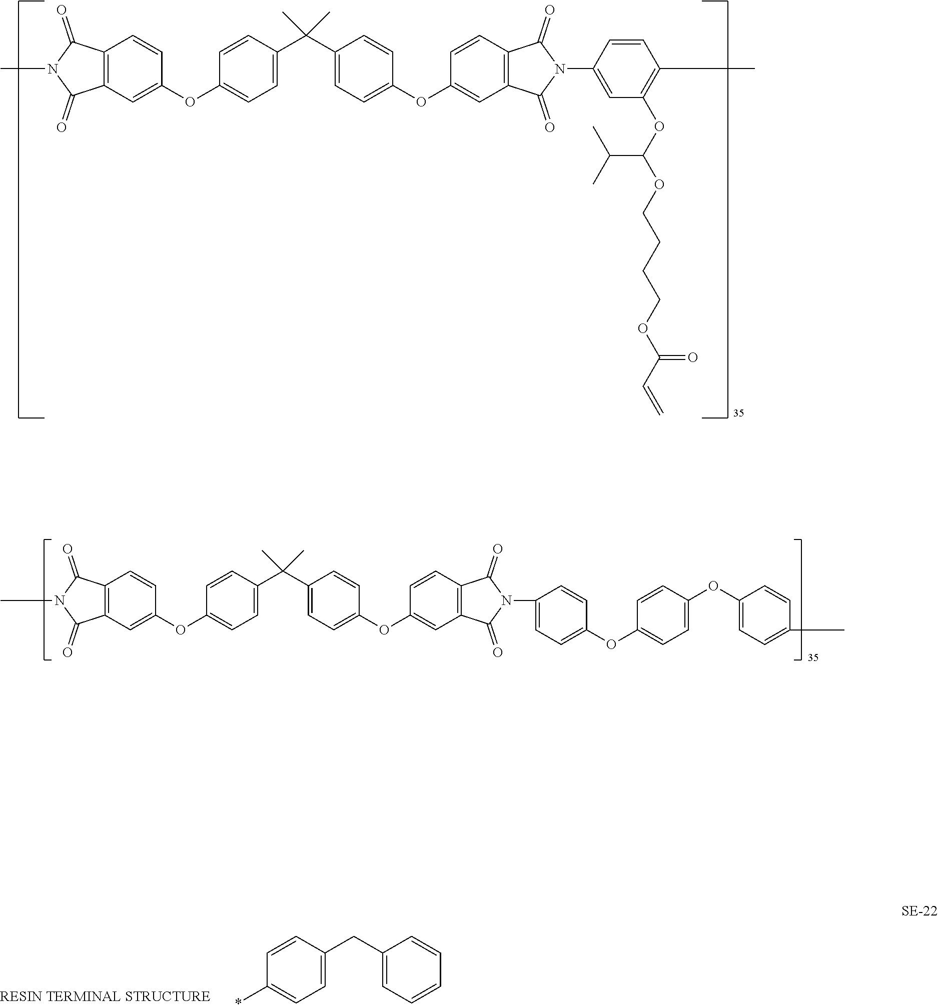

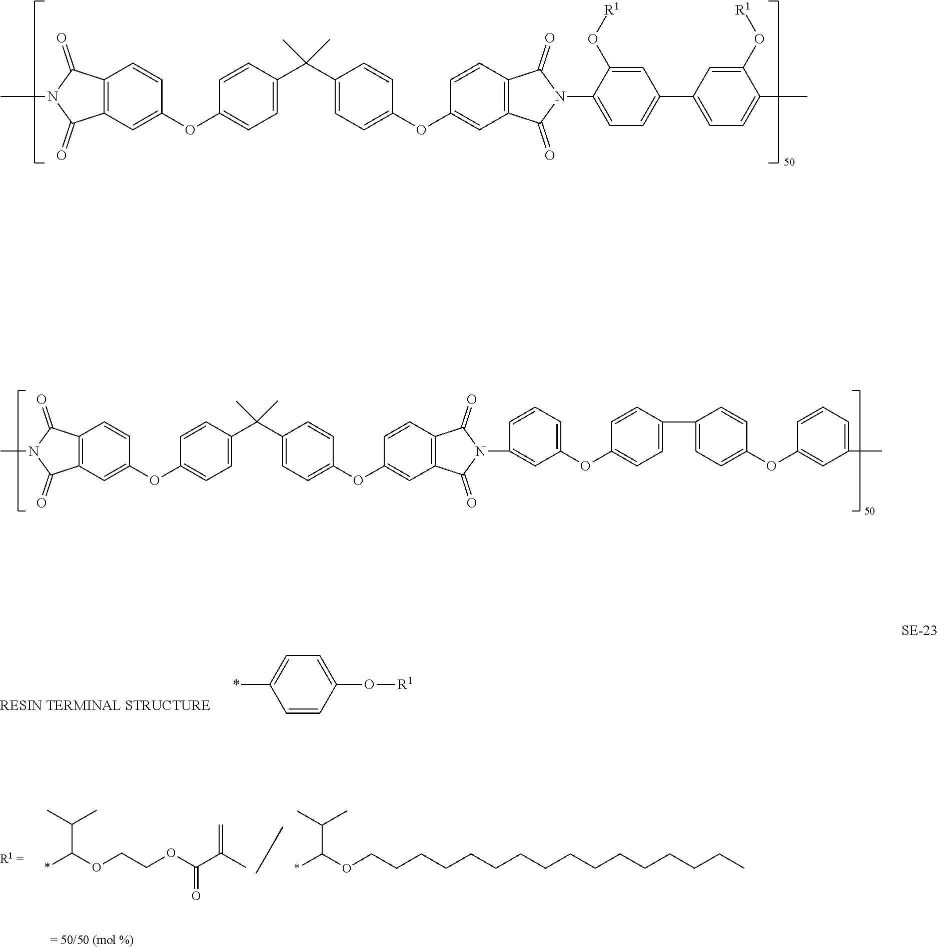

### Production Example SE-9: Synthesis of Polyimide (SE-9) Having Acetal Structure

30.0 g (57.64 mmol) of 4,4′-(4,4′-isopropylidenediphenoxy)diphtalic acid anhydride and 0.1 g of 2,2,6,6-tetramethylpiperidine 1-oxyl free radical were dissolved in 120 g of N-methylpyrrolidone (NMP) to obtain a solution. Subsequently, 4.74 g (31.1 millimoles) of 3,5-diaminobenzoic acid and 8.49 g (23.1 millimoles) of 4,4′-bis(3-aminophenoxy)biphenyl were dissolved in 80 g of NMP, and the solution was added dropwise to the above-described dissolved solution at a temperature of 10° C. to 25° C. over 1 hour, and the mixture was stirred at 25° C. for 120 minutes, and then 1.21 g (6.34 millimoles) of 4-heptylaniline was added thereto, and the mixture was further stirred at 25° C. for 60 minutes. Subsequently, 18.2 g of pyridine and 14.7 g of acetic anhydride were added thereto, and the mixture was reacted at 80° C. for 4 hours, cooled to 10° C., 8.03 g (62.2 millimoles) of diisopropylethylamine was added thereto, 26.4 g (46.7 millimoles) of BC-4 synthesized above was added dropwise thereto over 30 minutes, and the mixture was reacted at 25° C. for 2 hours, and then diluted with 200 g of tetrahydrofuran. Subsequently, the reaction solution was added dropwise to 2.0 L of methanol, and the mixture was stirred for 15 minutes, and the polyimide resin was filtered. Next, the above-described resin was reslurried in 1 L of methanol for 30 minutes, filtered, and dried at 40° C. for 12 hours under reduced pressure. Subsequently, the resin dried above was dissolved in 250 g of tetrahydrofuran, 40 g of an ion exchange resin (MB-1, manufactured by Organo Corporation) and 4 g of triethylamine were added thereto, and the mixture was stirred for 4 hours, and the ion exchange resin was filtered and removed, and the polyimide resin was precipitated in 2 L of methanol and stirred for 15 minutes. The polyimide resin was filtered and acquired, and dried at 40° C. for 1 day under reduced pressure to obtain a polyimide resin (SE-9).

The obtained polyimide (SE-9) had a weight-average molecular weight of 21,200 and a number-average molecular weight of 8,500.

As a result of the confirmation by ¹H-NMR, the imidization rate was 99% or more, the acetal introduction rate (carboxylic acid protection rate) was 97%, the acetal equivalent was 0.34 (millimoles/g), and the polymerizable group equivalent was 0.59 (millimoles/g). The structure of the repeating unit was determined from a ¹H-NMR spectrum. In the following structures, the subscripts of the repeating units represent the content molar ratio of the respective repeating units.

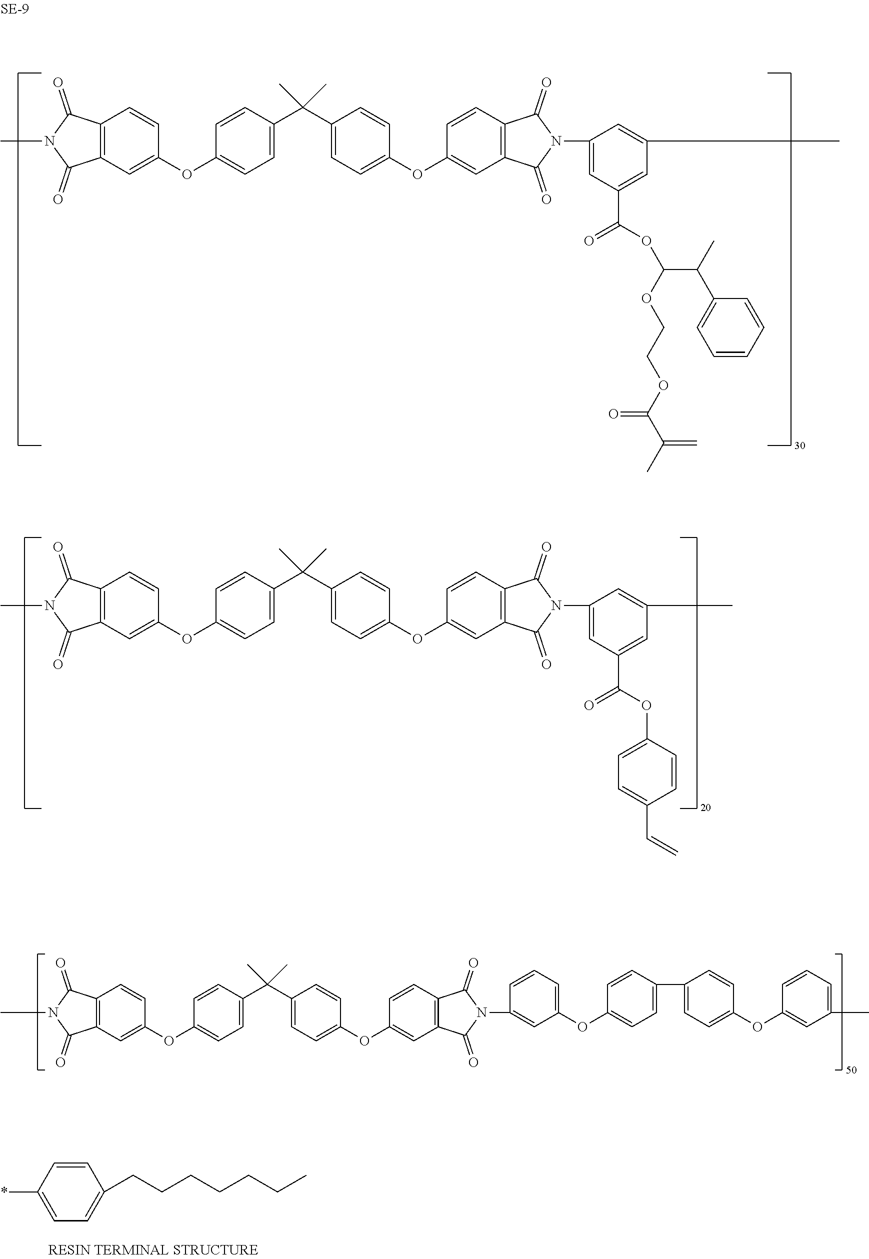

### Production Examples SE-10 to 13, 15, and 19: Synthesis of Polyimide (SE-10 to 13, 15, 19, and 24) Having Acetal Structure

Polyimides SE-10 to 13, 15, 19, and 24 were synthesized by the same method as in the synthesis of the polyimide SE-9, except that the raw materials were appropriately changed. The weight-average molecular weight, the number-average molecular weight, the imidization rate, the acetal introduction rate, the acetal equivalent, and the polymerizable group equivalent of these resins are described in the table shown later. The structure of the repeating unit was determined from a ¹H-NMR spectrum. In the following structures, the subscripts of the repeating units represent the content molar ratio of the respective repeating units.

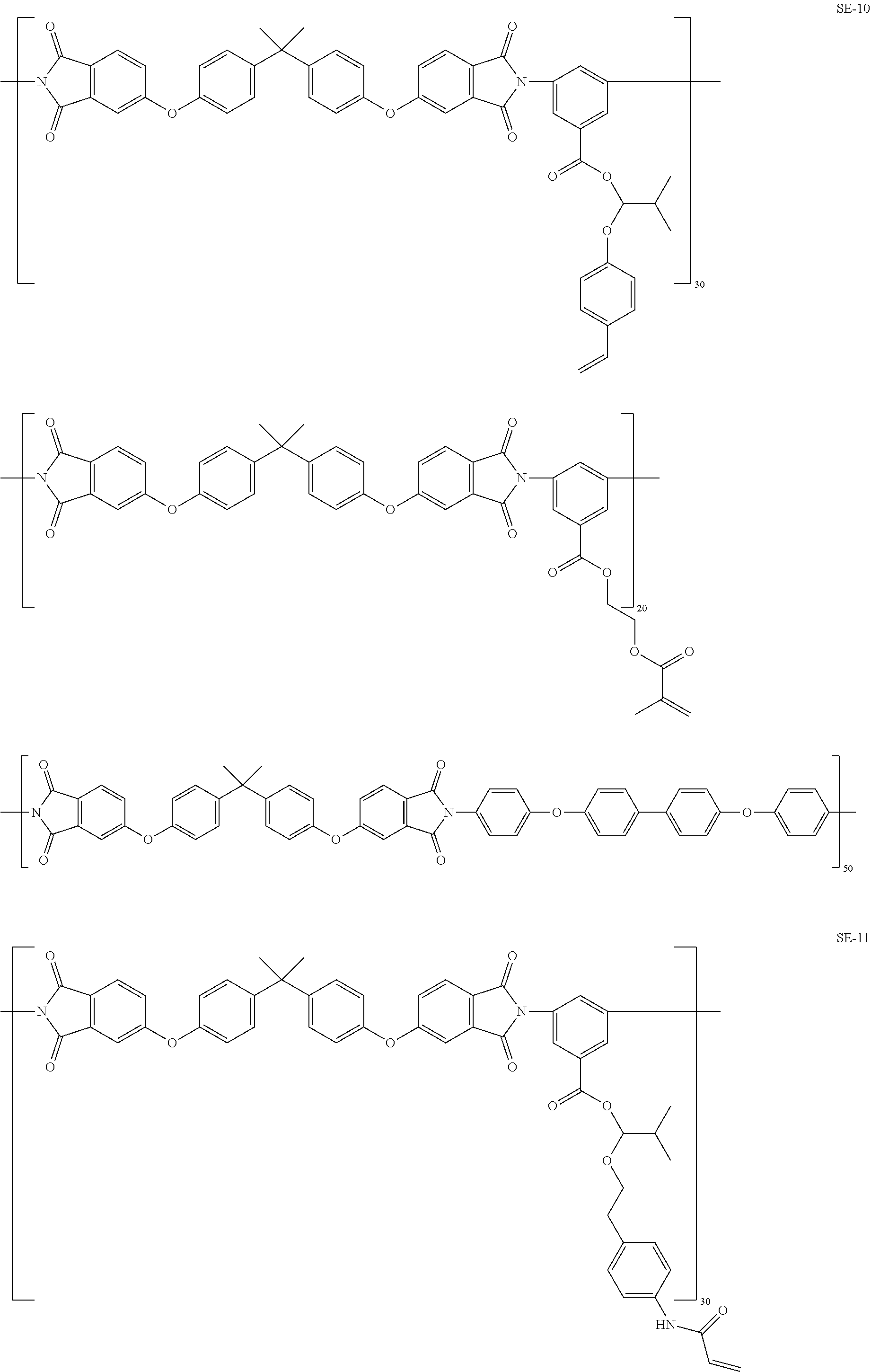

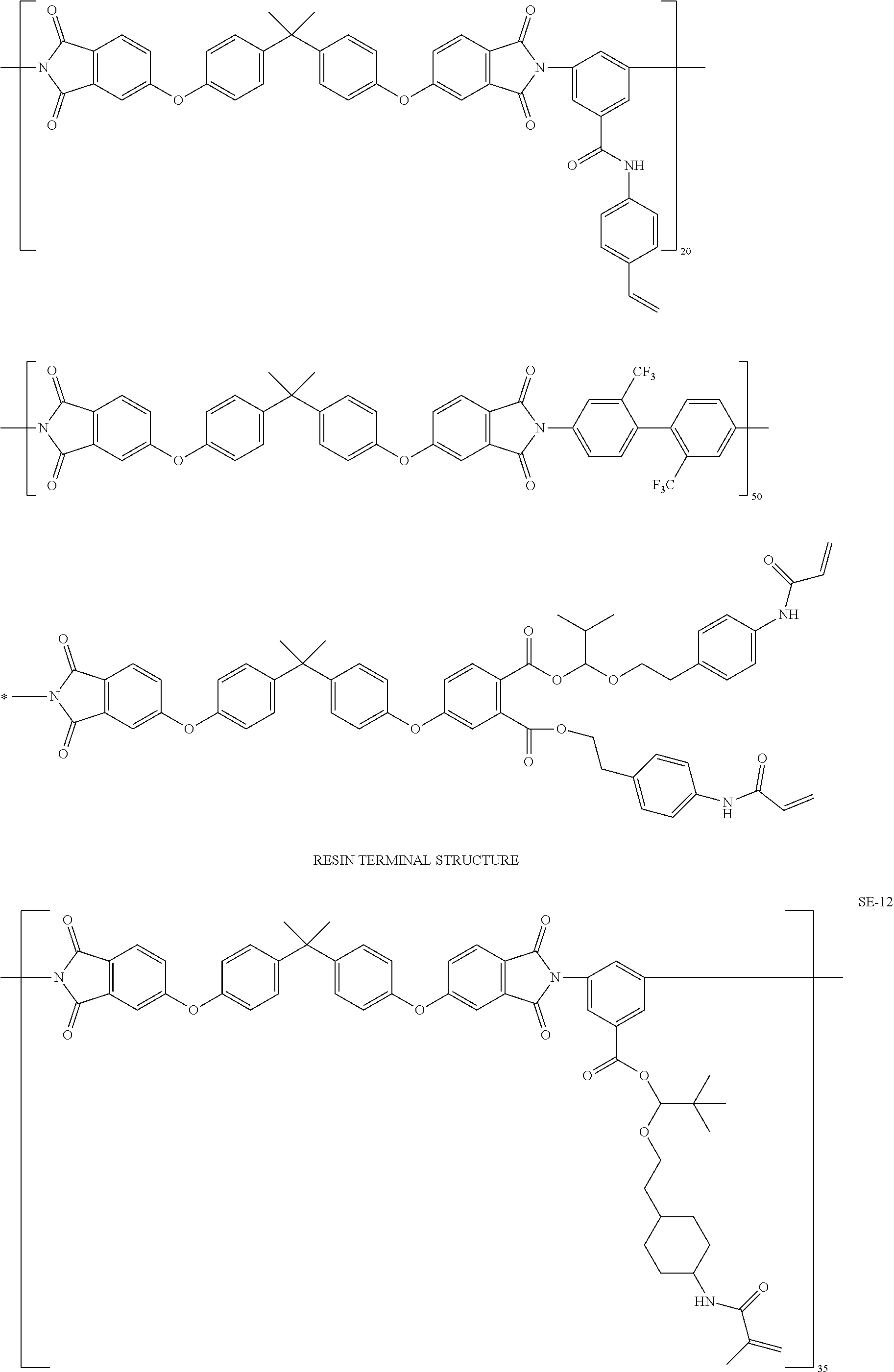

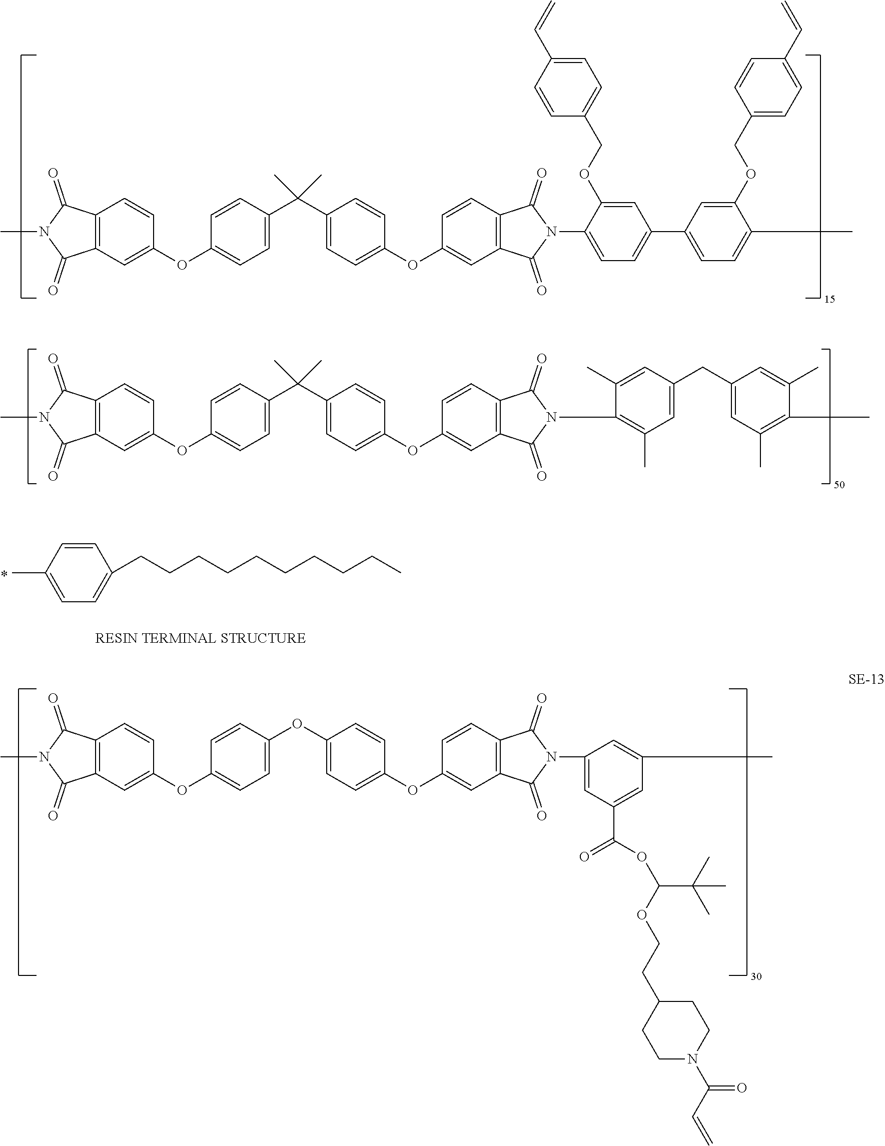

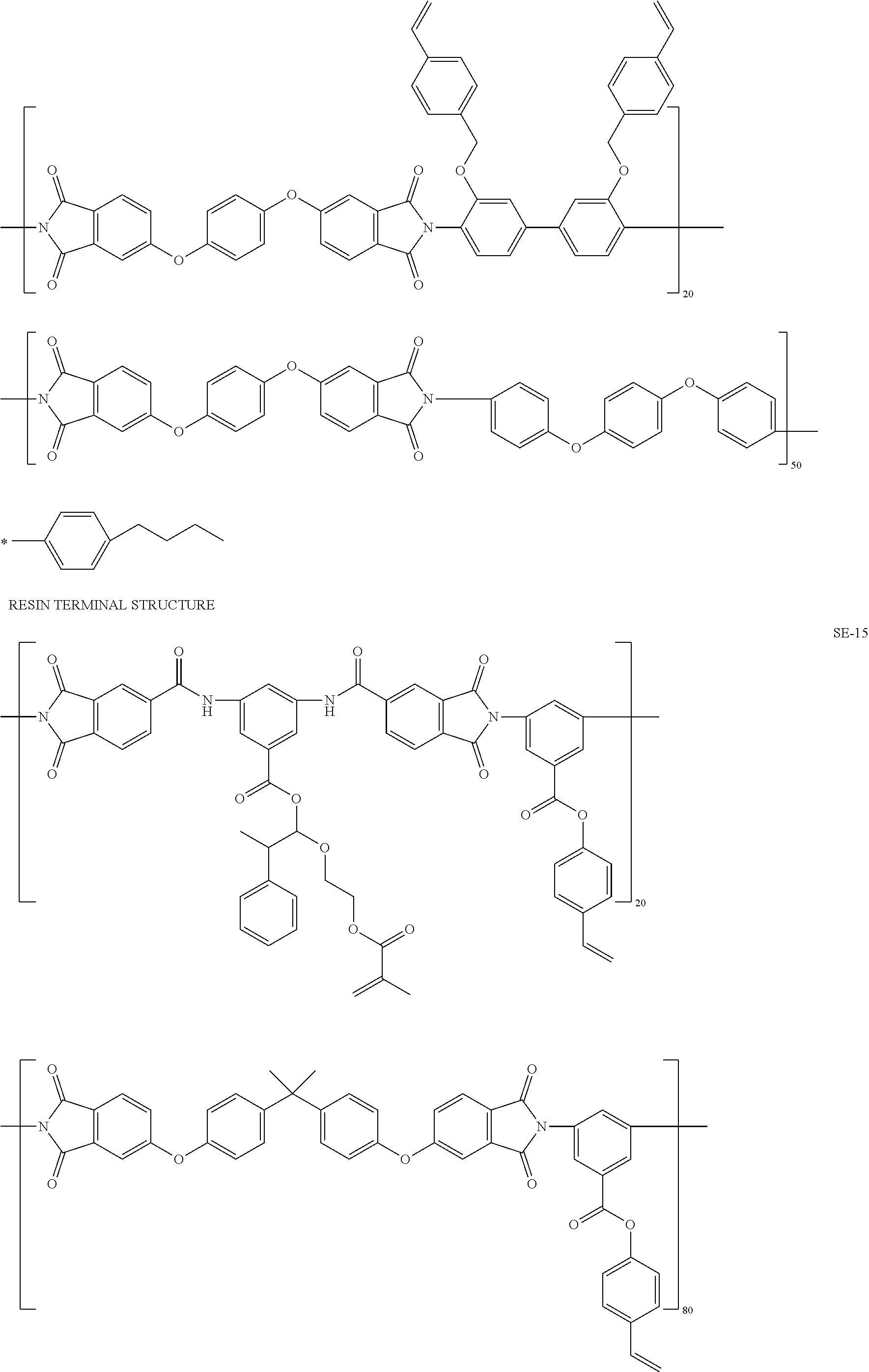

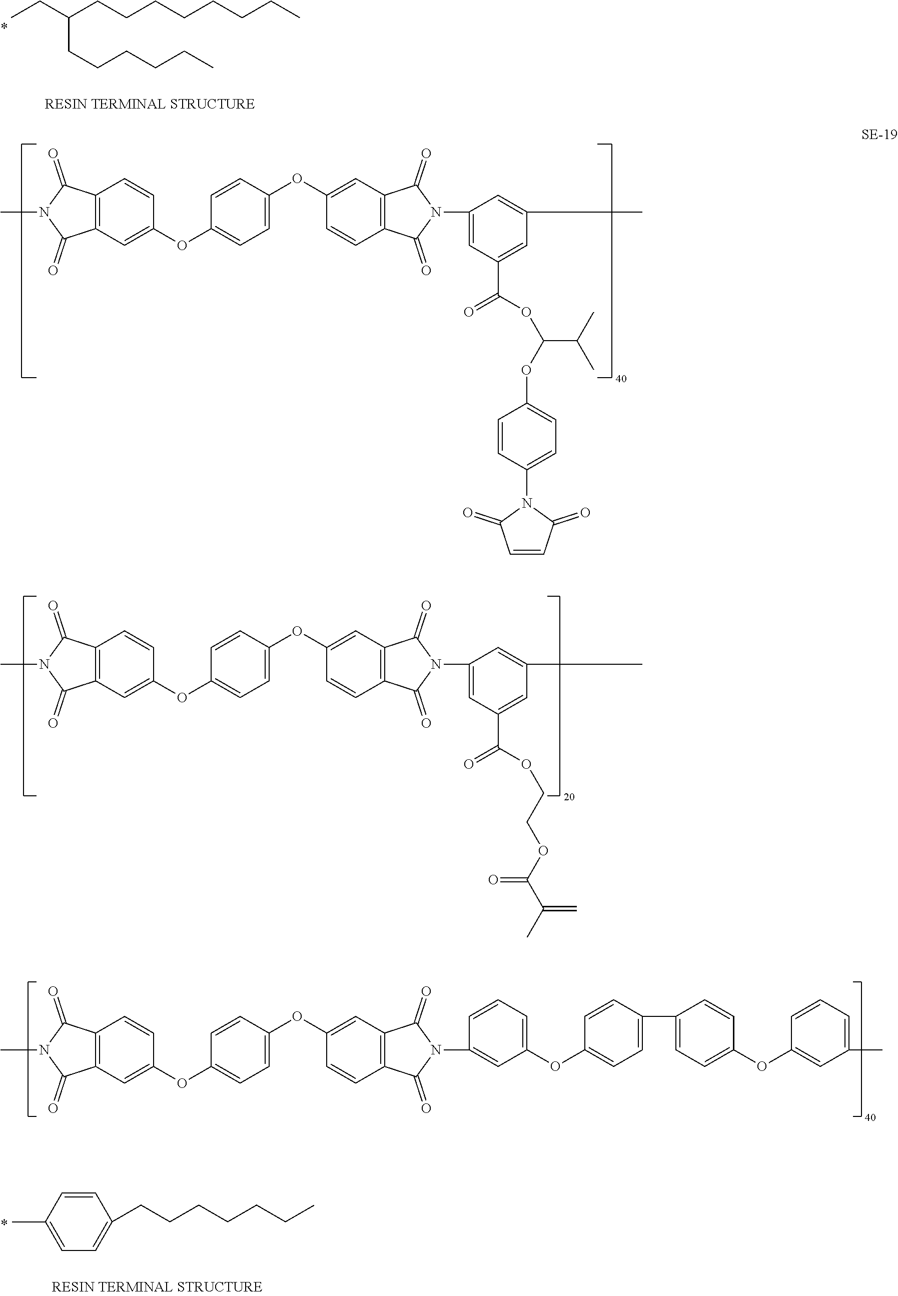

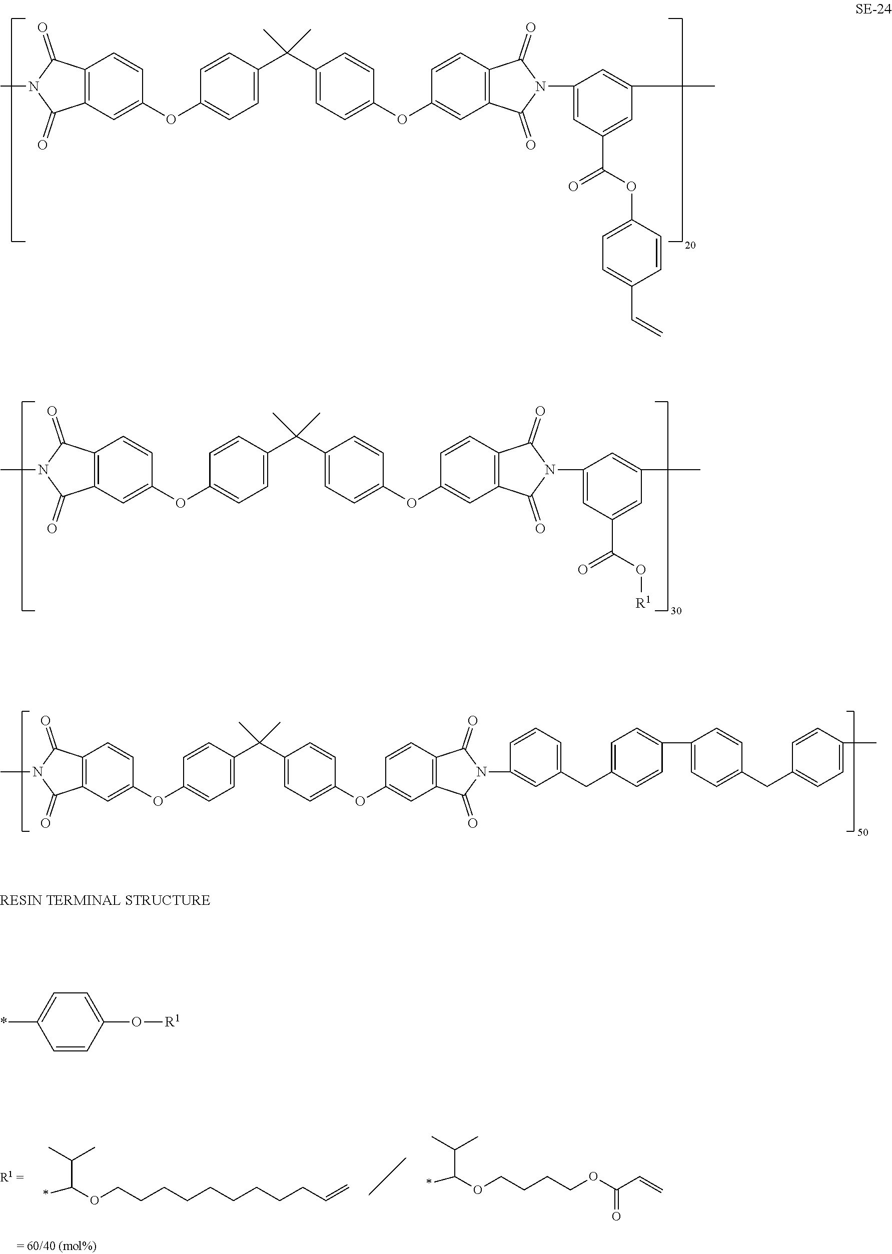

TABLE 1

| | Weight-average | Number-average | Imidization | Acetal | Acetal | Polymerizable |
|---|---|---|---|---|---|---|
| | molecular | molecular | rate | introduction | equivalent | group equivalent |
| | weight | weight | (%) | rate | (mmol/g) | (mmol/g) |
| SE-2 | 22,100 | 9,100 | 99% or more | 85% | 0.62 | 0.73 |
| SE-3 | 32,000 | 12,600 | 99% or more | 99% or more | 0.72 | 0.72 |
| SE-4 | 19,500 | 8,700 | 99% or more | 99% or more | 0.30 | 0.60 |
| SE-5 | 14.600 | 6.600 | 99% or more | 92% | 0.67 | 0.67 |
| SE-6 | 17,100 | 7,400 | 89% | 99% or more | 0.54 | 0.54 |
| SE-7 | 35,800 | 14,300 | 99% or more | 98% | 0.76 | 0.76 |
| SE-8 | 27,400 | 11,800 | 99% or more | 99% or more | 0.58 | 1.16 |
| SE-10 | 16,600 | 6,800 | 92% | 96% | 0.35 | 0.58 |
| SE-11 | 12,800 | 5,500 | 88% | 99% or more | 0.36 | 0.60 |
| SE-12 | 25,300 | 10,600 | 99% or more | 99% or more | 0.43 | 0.79 |
| SE-13 | 19,200 | 8,300 | 89% or more | 99% or more | 0.41 | 0.95 |
| SE-14 | 45,600 | 16,400 | 99% or more | 99% or more | 0.41 | 0.41 |
| SE-15 | 25,200 | 10,100 | 99% or more | 99% or more | 0.26 | 1.53 |
| SE-16 | 33,700 | 12,800 | 95% or more | 99% or more | 0.47 | 0.47 |
| SE-17 | 28,900 | 10,900 | 90% | 99% or more | 1.15 | 1.15 |
| SE-18 | 31,000 | 12,200 | 99% or more | 96% | 0.47 | 0.73 |
| SE-19 | 18,900 | 8,000 | 98% | 99% or more | 0.55 | 0.83 |
| SE-20 | 29,200 | 11,600 | 99% or more | 89% | 0.79 | 0.79 |
| SE-21 | 24,100 | 10,100 | 99% or more | 94% | 0.67 | 0.67 |
| SE-22 | 30,500 | 11,900 | 99% or more | 99% or more | 0.97 | 0.97 |
| SE-23 | 35,500 | 13,200 | 99% or more | 90% | 0.49 | 0.98 |
| SE-24 | 23,900 | 9,100 | 99% or more | 95% | 0.60 | 0.40 |

In all of SE-1 to SE-24, a structure having 2 or more carbon atoms was eliminated by heating at 1 atm, 230° C., and 3 hours.

### Production Example SP-1: Synthesis of Polyimide (SP-1)

30.00 g (57.64 mmol) of 4,4′-(4,4′-isopropylidenediphenoxy)diphtalic acid anhydride was dissolved in 150 g of N-methylpyrrolidone (NM4P). Subsequently, 5.36 g (24.79 millimoles) of HAB3 (manufactured by Wakayama Seika Co., Ltd.), 9.13 g (24.79 millimoles) of 4,4′-bis(3-aminophenoxy)biphenyl, 2.51 g (10.38 millimoles) of hexadecylamine, and 10 g of toluene were added thereto while washing with 30 g of NMP, and the mixture was stirred for 15 minutes, and then reacted at 200° C. for 4 hours while flowing nitrogen, and cooled to 25° C. Subsequently, 15.3 g (100 mmol) of 4-(chloromethyl)styrene, 19.92 g (144 mmol) of potassium carbonate, 2.39 g (14 mmol) of potassium iodide, and 0.1 g of 2,2,6,6-tetramethylpiperidine 1-oxide free radical were added thereto, the mixture was reacted at 95° C. for 15 hours, cooled to 25° C., diluted with 150 g of tetrahydrofuran, and the salts in the reaction solution were filtered through filter paper. Subsequently, the reaction solution was added dropwise to a mixed solution of 1.8 L of methanol and 0.6 L of water, and the mixture was stirred for 15 minutes and a polyimide resin was filtered. Next, the resin was subjected to reslurrying with 1 L of water, filtered, reslurried again with 1 L of methanol, filtered, and dried at 40° C. for 8 hours under reduced pressure. Subsequently, the resin dried as described above was dissolved in 300 g of tetrahydrofuran, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain a polyimide resin (SP-1). The obtained polyimide resin (SP-1) had a weight-average molecular weight of 19,700 and a number-average molecular weight of 8,000. As a result of the confirmation by ¹H-NMR, the imidization rate was 99% or more. The structure of the repeating unit was determined from a ¹H-NMR spectrum. In the following structures, the subscripts of the repeating units represent the content molar ratio of the respective repeating units

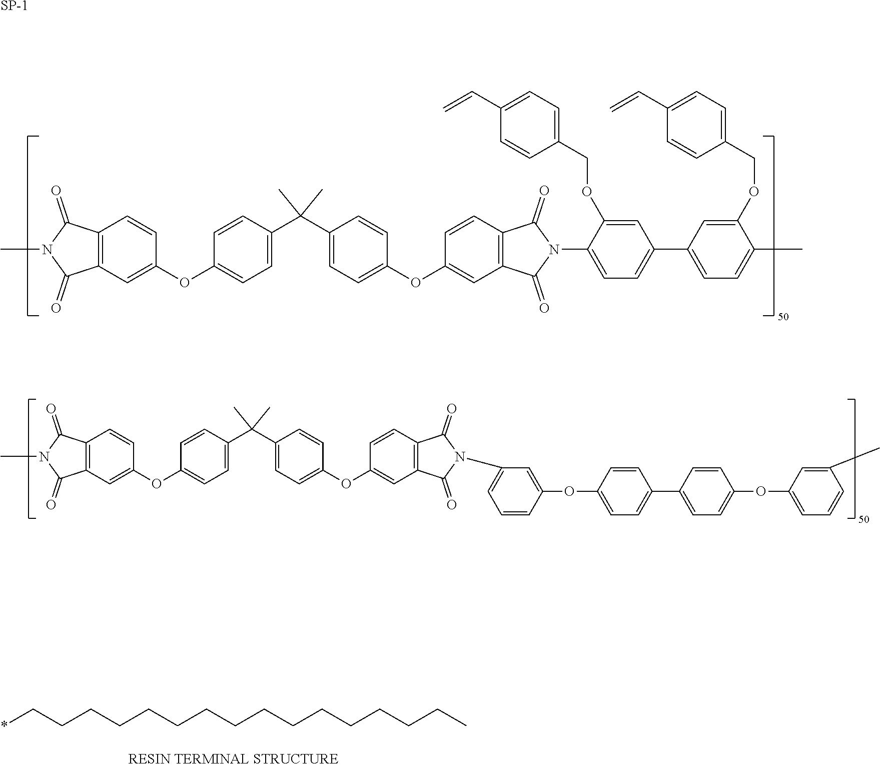

### Production Examples SP-2 to 5. Synthesis of Polyimide (SP-2 to 5)

Polyimides SP-2 to 5 were synthesized by the same method as in the synthesis of the polyimide SP-1, except that the raw materials were appropriately changed. The weight-average molecular weight, the number-average molecular weight, and the imidization rate of these resins are described in the table below. The structure of the repeating unit was determined from a ¹H-NMR spectrum. In the following structures, the subscripts of the repeating units represent the content molar ratio of the respective repeating units.

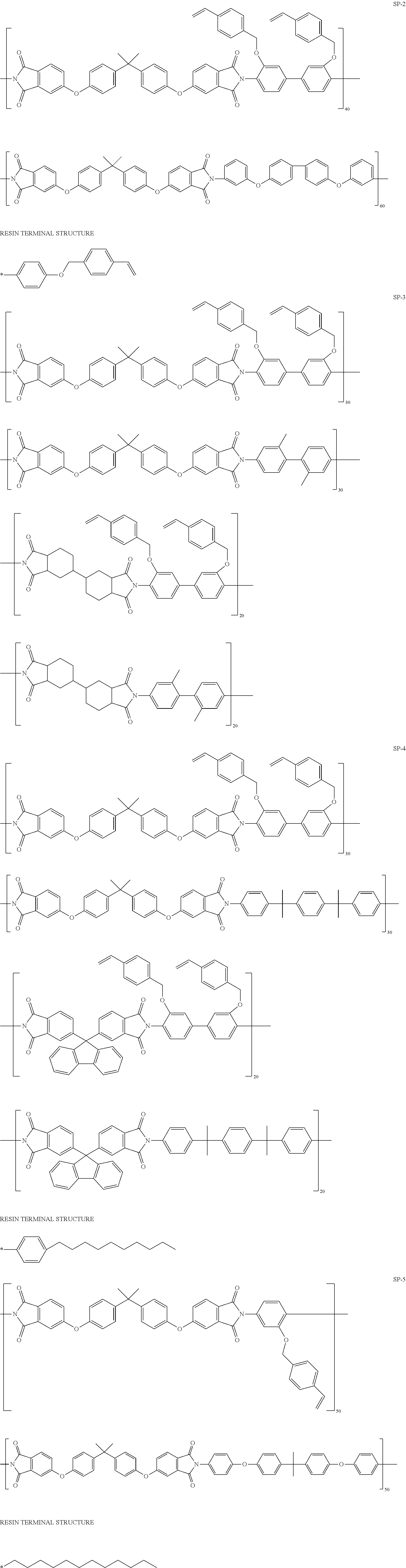

TABLE 2

| | Weight-average | Number-average | Imidization |
|---|---|---|---|
| | molecular weight | molecular weight | rate (%) |
| SP-2 | 25,500 | 10,100 | 99 |
| SP-3 | 14,200 | 6,600 | 99 |
| SP-4 | 19,700 | 8,900 | 98 |
| SP-5 | 30,300 | 13.600 | 99 |

### Synthesis Example AA-1

375 mL of dimethylformamide was mixed with 48.65 g (225 mmol) of 3,3′-dihydroxybenzidine in a flask. 98.21 g (450 millimoles) of di-t-butyl carbonate was added dropwise under ice cooling. Stirring was carried out at a temperature of 60° C. for 5 hours after completion of the dropwise addition. After the completion of the reaction, the mixture was cooled to room temperature, 35 mg of 2,2,6,6-tetramethylpiperidine 1-oxyl free radical, 68.68 g (450 mmol) of p-chloromethylstyrene, 74.63 g (540 mmol) of potassium carbonate, and 8.96 g (54.0 mmol) of potassium iodide were added thereto, and the mixture was stirred at a temperature of 60° C. for 3 hours. After completion of the reaction, filtration was carried out by a suction filtration operation, and the filtrate was added dropwise to 500 mL of water. Since white crystals were precipitated, the precipitated solid was collected by suction filtration. The obtained white solid was recrystallized and purified at 60° C. using 1000 mL of acetone. 125 g of the following intermediate AA-1a (yield: 85.6%) was obtained.

The structure of AA-1a is shown below. The following structure was confirmed from the ¹H-NMR spectrum.

¹H-NMR (BRUKER, AVANCE NEO 400): δ (ppm, DMSO-d6) 8.04-7.94 (s, 2H), 7.75-7.64 (d, 2H), 7.56-7.42 (m, 8H), 7.27-7.20 (d, 2H), 7.19-7.12 (d, 2H), 6.79-6.64 (2H), 5.89-5.77 (2H), 5.30-5.15 (6H), 1.49-1.43 (s, 18H)

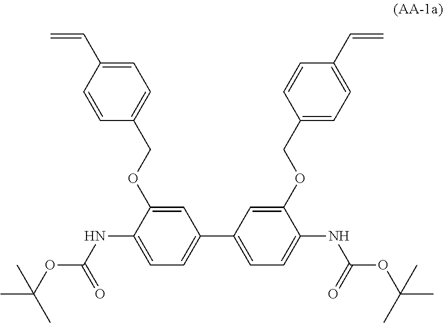 [SMILES: C=Cc1ccc(COc2cc(-c3ccc(NC(=O)OC(C)(C)C)c(OCc4ccc(C=C)cc4)c3)ccc2NC(=O)OC(C)(C)C)cc1]

75.0 g (115.6 millimoles) of (AA-1a) and 500 mL of methylene chloride were mixed in a flask. 131.8 g (1156 mmol) of trifluoroacetic acid was added thereto at room temperature, and the mixture was stirred at a temperature of 40° C. for 5 hours. After completion of the reaction, 250 mL of methanol and then 117.0 g (1156 mmol) of triethylamine were added dropwise under ice cooling. Since pale yellow crystals were precipitated, the precipitated solid was collected by suction filtration. Suspension washing was carried out with 750 mL of methanol to obtain 40.5 g (yield: 73%) of (AA-1). The structure of AA-1 is shown below. The following structure was confirmed from the ¹H-NMR spectrum.

¹H-NMR (BRUKER, AVANCE NEO 400): δ (ppm, DMSO-d6) 7.53-7.45 (s, 8H), 7.05-6.98 (d, 2H), 6.92-6.85 (d, 2H), 6.79-6.63 (4H), 5.89-5.78 (d, 2H), 5.29-5.22 (d, 2H), 5.20-5.13 (s, 4H), 4.92-4.64 (4H)

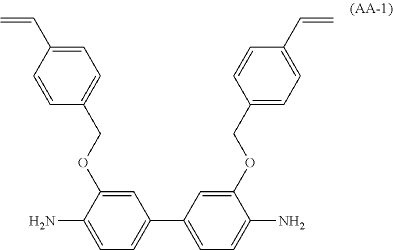 [SMILES: C=Cc1ccc(COc2cc(-c3ccc(N)c(OCc4ccc(C=C)cc4)c3)ccc2N)cc1]

### Production Example SP-6: Synthesis of Polyimide (SP-6)

6.05 g (28.5 millimoles) of m-tolidine, 12.78 g (28.5 millimoles) of AA-1, and 0.08 g of 2,2,6,6-tetramethylpiperidine 1-oxyl free radical were dissolved in 100 g of N-methylpyrrolidone (NMP) to obtain a dissolved solution. Subsequently, 26.68 g (51.3 millimoles) of 4,4′-(4,4′-isopropylidenediphenoxy)diphthalic acid anhydride was dissolved in 100 g of NMP, and the solution was added dropwise to the above-described dissolved solution at a temperature of 10° C. to 25° C. over 1 hour, and the mixture was stirred at 25° C. for 60 minutes, and then 1.87 g (8.55 millimoles) of di-tert-butyl dicarbonate was added dropwise thereto, and the mixture was stirred at 45° C. for 3 hours. Subsequently, 18.2 g of pyridine and 14.7 g of acetic anhydride were added thereto, and the mixture was reacted at 80° C. for 4 hours. After completion of the reaction, the mixture was cooled to 25° C. and diluted with 200 g of tetrahydrofuran. Subsequently, the reaction solution was added dropwise to a mixed solution of 1.5 L of methanol and 0.5 L of water, and the mixture was stirred for 15 minutes and a polyimide resin was filtered. Next, the above-described resin was reslurried in 1 L of water for 30 minutes, filtered, reslurried in 1 L of methanol, filtered, and dried at 40° C. for 10 hours under reduced pressure. Subsequently, the resin dried as described above was dissolved in 250 g of tetrahydrofuran, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain a polyimide resin (SP-6).

The obtained polyimide (SP-6) had a weight-average molecular weight of 22,500 and a number-average molecular weight of 7,200.

As a result of the confirmation by ¹H-NMR, the imidization rate was 96%.

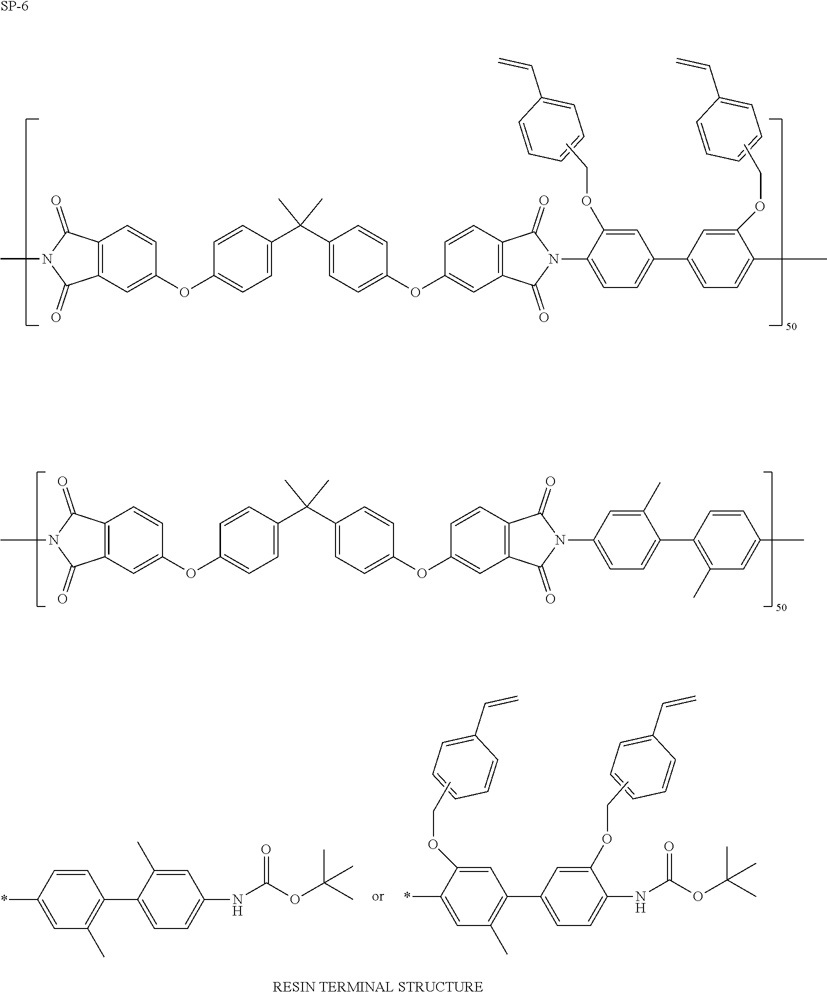

### Production Example SP-7: Synthesis of Polyimide (SP-7)

30.0 g (57.64 mmol) of 4,4′-(4,4′-isopropylidenediphenoxy)diphtalic acid anhydride and 0.08 g of 2,2,6,6-tetramethylpiperidine 1-oxyl free radical were dissolved in 120 g of N-methylpyrrolidone (NMP) to obtain a solution. Subsequently, 5.14 g (24.2 millimoles) of m-tolidine and 10.86 g (24.2 millimoles) of AA-1 were dissolved in 80 g of NMP, and the solution was added dropwise to the above-described dissolved solution at a temperature of 10° C. to 25° C. over 1 hour, and the mixture was stirred at 25° C. for 60 minutes, and then 18.2 g of pyridine and 14.7 g of acetic anhydride were added thereto, and the mixture was reacted at 80° C. for 4 hours. After completion of the reaction, the mixture was cooled to 25° C. and diluted with 200 g of tetrahydrofuran. Subsequently, the reaction solution was added dropwise to 2.0 L of water, and the mixture was stirred for 15 minutes, and the polyimide resin was filtered. Next, the above-described resin was reslurried in 1 L of water for 30 minutes, filtered, reslurried in 1 L of methanol, filtered, and dried at 40° C. for 10 hours under reduced pressure. Subsequently, the resin dried as described above was dissolved in 250 g of tetrahydrofuran and 20 g of water, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain a polyimide resin (SP-7).

The obtained polyimide (SP-7) had a weight-average molecular weight of 20,500 and a number-average molecular weight of 7,500.

As a result of the confirmation by ¹H-NMR, the imidization rate was 99% or more.

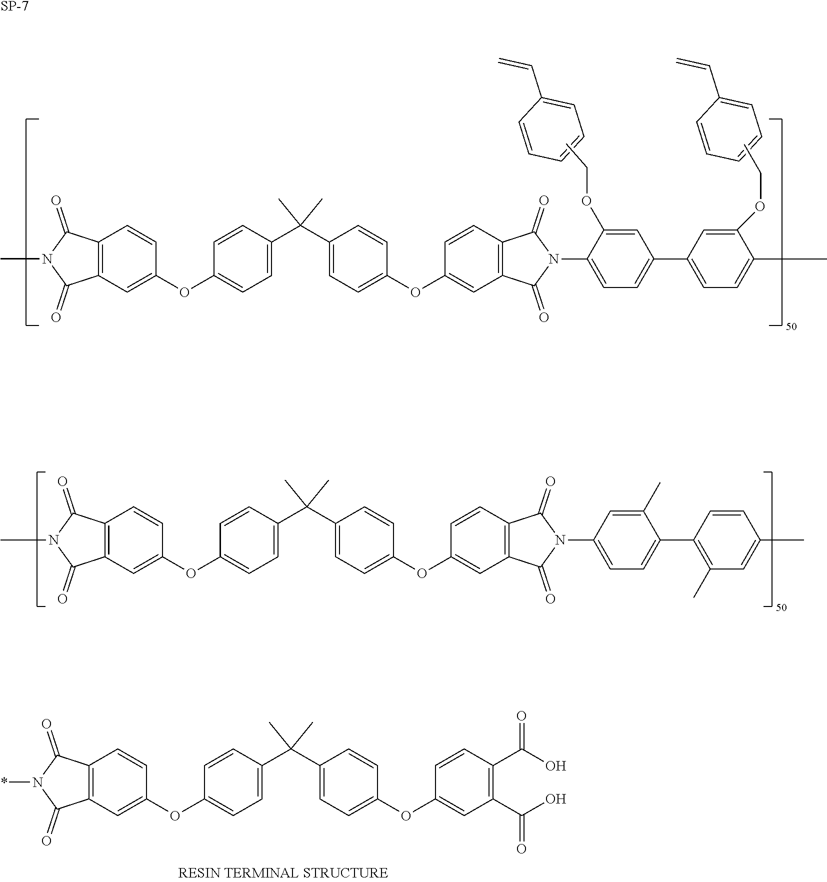

### Production Example SP-8: Synthesis of Polyimide (SP-8)

30.0 g (57.64 mmol) of 4,4′-(4,4′-isopropylidenediphenoxy)diphtalic acid anhydride was dissolved in 130 g of N-methylpyrrolidone (NMP). Subsequently, 3.20 g (12.39 millimoles) of 2,2-bis(3-amino-4-hydroxyphenyl)propane, 7.89 g (37.18 millimoles) of m-tolidine, and 2.78 g (11.53 millimoles) of hexadecylamine were added thereto while washing with 50 g of NMP, and the mixture was stirred at a temperature of 20° C. to 50° C. for 30 minutes, and then 10 g of toluene was added thereto, and the mixture was reacted at 200° C. for 4 hours while flowing nitrogen, and cooled to 25° C. Subsequently, 6-maleimidehexane acid chloride THF solution (37.5 millimoles) synthesized above, 6.13 g (77.5 millimoles) of pyridine, and 0.10 g of 2,2,6,6-tetramethylpiperidine 1-oxyl free radical were added thereto, and the mixture was reacted at 25° C. for 2 hours, and then heated to 45° C. and stirred for 10 hours. Subsequently, the reaction solution was cooled to 25° C., diluted with 200 g of tetrahydrofuran, and the reaction solution was added dropwise to a mixed solution of 2.0 L of methanol and 0.5 L of water, and the mixture was stirred for 15 minutes, and the polyimide resin was filtered. Next, the resin was subjected to reslurrying with 1 L of water, filtered, reslurried again with 1 L of methanol, filtered, and dried at 40° C. for 10 hours under reduced pressure. Subsequently, the resin dried as described above was dissolved in 250 g of tetrahydrofuran, 40 g of an ion exchange resin (MB-1: manufactured by ORGANO CORPORATION) was added thereto, the mixture was stirred for 4 hours, the ion exchange resin was filtered and removed, and then the polyimide resin was precipitated in 2 L of methanol and stirred for 15 minutes. The polyimide resin was filtered and acquired, and dried at 45° C. for 1 day under reduced pressure to obtain a polyimide resin (SP-8). The obtained polyimide resin (SP-8) had a weight-average molecular weight of 23,700 and a number-average molecular weight of 8,900.

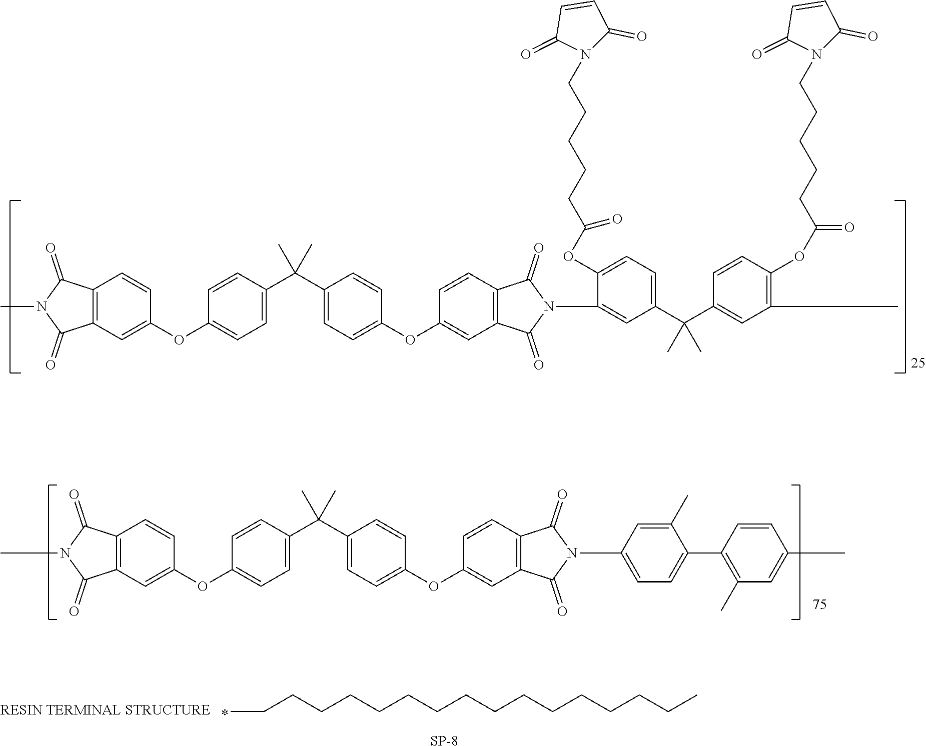

### Synthesis of Polyimide Precursor

### Production Example SA-1: Synthesis of Polyimide Precursor (SA-1)

19.83 g (38.1 millimoles) of 4,4′-(4,4′-isopropylidenediphenoxy)diphthalic acid anhydride, 10.12 g (77.8 millimoles) of 2-hydroxyethyl methacrylate, 0.05 g of hydroquinone, 13.40 g (169 millimoles) of pyridine, and 70 g of diethylene glycol monomethyl ether were mixed and stirred at a temperature of 60° C. for 5 hours to produce a diester of 4,4′-(4,4′-isopropylidenediphenoxy)bis(phthalic acid anhydride) and glycerol dimethacrylate. Next, the mixture was cooled to −10° C., 9.53 g (79.2 millimoles) of thionyl chloride was subsequently added dropwise over 90 minutes, and stirring was carried out for 2 hours to obtain a white precipitate of pyridinium hydrochloride. Next, a solution obtained by dissolving 7.12 g (33.5 millimoles) of m-tolidine in 100 mL of NMP (N-methyl-2-pyrrolidone) was added dropwise thereto over 2 hours. Next, 10.0 g (217 millimoles) of ethanol was added, and the mixture was stirred for 2 hours. Next, the polyimide precursor resin was precipitated in 4 L of water and the water-polyimide precursor resin mixture was stirred for 15 minutes at a speed of 500 rpm. The polyimide precursor resin was filtered and acquired, stirred again in 4 L of water for 30 minutes, filtered again, and dried at 40° C. for 2 days. Subsequently, the resin dried as described above was dissolved in 200 g of tetrahydrofuran, 50 g of an ion exchange resin (MB-1: manufactured by ORGANO CORPORATION) was added thereto, and the mixture was stirred for 6 hours. Next, the polyimide precursor resin was precipitated in 4 L of water and the water-polyimide precursor resin mixture was stirred for 15 minutes at a speed of 500 rpm. The polyimide precursor resin was filtered and acquired, and dried at 45° C. for 2 days under reduced pressure to obtain a polyimide precursor (SA-1). The obtained polyimide precursor (SA-1) had a weight-average molecular weight of 24,600 and a number-average molecular weight of 9,400.

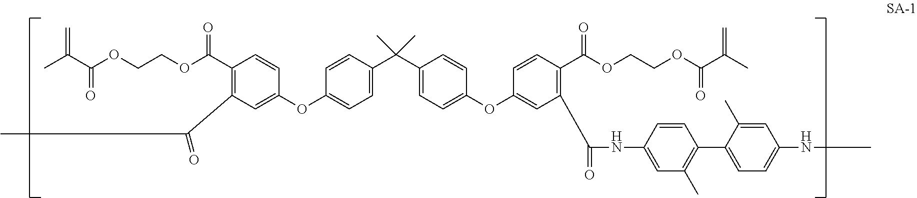

### Synthesis of Comparative Compound A-1

41.02 g (112 mmol) of 2,2-bis(3-amino-4-hydroxyphenyl)hexafluoropropane, 57.29 g (125.0 mmol) of bis(1,3-dioxo-1,3-dihydroisobenzofuran-5-carboxylic acid) 1,4-phenylene, and 492.43 g of γ-butyrolactone were added to a drying reactor equipped with a flat bottom joint, a stirrer, a condenser, and an internal thermometer, and the mixture was stirred at a temperature of 60° C. for 1.5 hours while removing moisture. Next, 50 mL of toluene was added thereto, the temperature was raised to 180° C. while flowing nitrogen at a flow rate of 200 mL/min, stirring was carried out for 3 hours, and cooling was carried out to room temperature. The obtained polymerization solution was diluted with acetone to prepare a diluted solution, and the diluted solution was added dropwise to a mixed solution of water/methanol=3/1 to precipitate a white solid. The obtained white solid was recovered and vacuum-dried at a temperature of 120° C. to obtain 90 g of a polymer.

Subsequently, 73.86 g (150.0 mmol in terms of hydroxyl group) of the polymer obtained above, 21.17 g (150.0 mmol) of 2-isocyanatoethyl acrylate (hereinafter, also referred to as AOI, manufactured by Showa Denko K.K.), and 828.26 g of γ-butyrolactone (GBL) were placed in a reaction container comprising a stirrer and a cooling pipe. Thereafter, the temperature was raised to 120° C. with stirring, and the mixture was reacted for 6 hours. Subsequently, the obtained reaction solution was diluted with acetone to prepare a diluted solution, and the diluted solution was added dropwise to a mixed solution of water/methanol=2/1 to precipitate a white solid. The obtained white solid was recovered and vacuum-dried at a temperature of 40° C. to obtain 85.8 g of A-1. The weight-average molecular weight (Mw) of A-1 was 25,100, and the number-average molecular weight (Mn) thereof was 11,200. It was confirmed by ¹H-NMR spectrum that the structure of A-1 was a structure represented by Formula (A-1). From the measurement results of ¹H-NMR, the introduction rate of the crosslinking group was 50%. As a result of the confirmation by ¹H-NMR, the imidization rate was 99% or more.

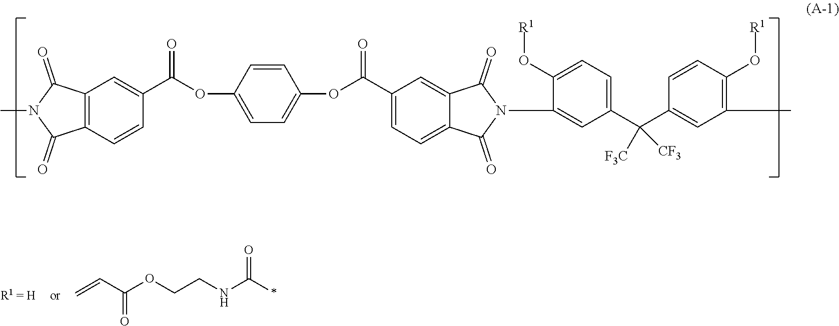

### Examples and Comparative Examples

In each of Examples, the components shown in the table below were mixed to obtain each of the resin compositions. Further, in each of Comparative Examples, components shown in the following table were mixed to obtain a comparative composition.

Specifically, the content of each component described in the table was set to the amount (in terms of parts by mass) described in the column of “Adding amount” of each column of the table.

The obtained resin composition and the comparative composition were filtered under pressure using a filter made of polytetrafluoroethylene, having a micropore width of 0.5 m.

In addition, in the table, the description of “-” indicates that the corresponding component is not contained in the composition.

TABLE 3

| | | | | | | | | | | | | | Metal | |
|---|---|---|---|---|---|---|---|---|---|---|---|---|---|---|
| | | | | | | | | | | | Migration | | adhesiveness | |
| | | | | | Polymenization | | | | Polymerizable | | suppressing | | improving | |
| | Resin | | | | initiator | | Solvent | | compound | | agent | | agent | |
| | | Adding | | Adding | | Adding | | Adding | | Adding | | Adding | | Adding |
| | Type | amount | Type | amount | Type | amount | Type | amount | Type | amount | Type | amount | Type | amount |
| Example 1 | SE-1 | 30.4 | — | — | OXE-02 | 1.66 | GBL | 60 | D-1 | 65 | E-1 | 0.10 | F-4 | 0.50 |
| Example 2 | SE-2 | 30.4 | — | — | OXE-02 | 1.66 | GBL | 60 | D-1 | 65 | E-1 | 0.10 | F-4 | 0.50 |
| Example 3 | SE-3 | 30.4 | — | — | OXE-02 | 1.66 | GBL | 60 | D-1 | 65 | E-1 | 0.10 | F-4 | 0.50 |
| Example 4 | SE-4 | 30.4 | — | — | OXE-02 | 1.66 | GBL | 60 | D-1 | 65 | E-1 | 0.10 | F-4 | 0.50 |
| Example 5 | SE-5 | 30.4 | — | — | OXE-02 | 1.66 | GBL | 60 | D-1 | 65 | E-1 | 0.10 | F-4 | 0.50 |
| Example 6 | SE-6 | 30.4 | — | — | OXE-02 | 1.66 | GBL | 60 | D-1 | 65 | E-1 | 0.10 | F-4 | 0.50 |
| Example 7 | SE-7 | 30.4 | — | — | OXE-02 | 1.66 | GBL | 60 | D-1 | 65 | E-1 | 0.10 | F-4 | 0.50 |
| Example 8 | SE-8 | 30.4 | — | — | OXE-02 | 1.66 | GBL | 60 | D-1 | 65 | E-1 | 0.10 | F-4 | 0.50 |
| Example 9 | SE-9 | 30.4 | — | — | OXE-02 | 1.66 | GBL | 60 | D-1 | 65 | E-1 | 0.10 | F-4 | 0.50 |
| Example 10 | SE-10 | 30.4 | — | — | OXE-02 | 1.66 | GBL | 60 | D-1 | 65 | E-1 | 0.10 | F-4 | 0.50 |
| Example 11 | SE-11 | 30.4 | — | — | OXE-02 | 1.66 | GBL | 60 | D-1 | 65 | E-1 | 0.10 | F-4 | 0.50 |
| Example 12 | SE-12 | 30.4 | — | — | OXE-02 | 1.66 | GBL | 60 | D-1 | 65 | E-1 | 0.10 | F-4 | 0.50 |
| Example 13 | SE-13 | 30.4 | — | — | OXE-02 | 1.66 | GBL | 60 | D-1 | 65 | E-1 | 0.10 | F-4 | 0.50 |
| Example 14 | SE-14 | 30.4 | — | — | OXE-02 | 1.66 | GBL | 60 | D-1 | 65 | E-1 | 0.10 | F-4 | 0.50 |
| Example 15 | SE-15 | 30.4 | — | — | OXE-02 | 1.66 | GBL | 60 | D-1 | 65 | E-1 | 0.10 | F-4 | 0.50 |
| | Polymerization | | | | | | Curing | | | | Dielectric | Development | | |
| | inhibitor | | Addtive | | Surfactant | | condition | | | | loss | residual | | |
| | | Adding | | Adding | | Adding | Tem- | Curing | | Breaking | tangent | film | Insulatior | |
| | Type | amount | Type | amount | Type | amount | perature | time | Developer | elongatior | (Df) | property | reliability | |
| Example 1 | G-2 | 0.04 | I-3 | 0.8 | — | — | 230° C. | 3 hours | Cyclo- | A | A | A | A | |
| | | | | | | | | | pentanone | | | | | |
| Example 2 | G-2 | 0.04 | I-3 | 0.8 | — | — | 230° C. | 3 hours | Cyclo- | A | A | A | A | |
| | | | | | | | | | pentanone | | | | | |
| Example 3 | G-2 | 0.04 | I-3 | 0.8 | — | — | 230° C. | 3 hours | Cyclo- | A | A | A | A | |
| | | | | | | | | | pentanone | | | | | |
| Example 4 | G-2 | 0.04 | I-3 | 0.8 | — | — | 230° C. | 3 hours | Cyclo- | B | B | A | B | |
| | | | | | | | | | pentanone | | | | | |
| Example 5 | G-2 | 0.04 | I-3 | 0.8 | — | — | 230° C. | 3 hours | Cyclo- | B | A | A | A | |
| | | | | | | | | | pentanone | | | | | |
| Example 6 | G-2 | 0.04 | I-3 | 0.8 | — | — | 230° C. | 3 hours | Cyclo- | B | A | A | A | |
| | | | | | | | | | pentanone | | | | | |
| Example 7 | G-2 | 0.04 | I-3 | 0.8 | — | — | 230° C. | 3 hours | Cyclo- | B | B | A | B | |
| | | | | | | | | | pentanone | | | | | |
| Example 8 | G-2 | 0.04 | I-3 | 0.8 | — | — | 230° C. | 3 hours | Cyclo- | A | B | B | A | |
| | | | | | | | | | pentanone | | | | | |
| Example 9 | G-2 | 0.04 | I-3 | 0.8 | — | — | 230° C. | 3 hours | Cyclo- | A | A | A | A | |
| | | | | | | | | | pentanone | | | | | |
| Example 10 | G-2 | 0.04 | I-3 | 0.8 | — | — | 230° C. | 3 hours | Cyclo- | A | A | B | A | |
| | | | | | | | | | pentanone | | | | | |
| Example 11 | G-2 | 0.04 | I-3 | 0.8 | — | — | 230° C. | 3 hours | Cyclo- | B | B | A | A | |
| | | | | | | | | | pentanone | | | | | |
| Example 12 | G-2 | 0.04 | I-3 | 0.8 | — | — | 230° C. | 3 hours | Cyclo- | A | A | A | A | |
| | | | | | | | | | pentanone | | | | | |
| Example 13 | G-2 | 0.04 | I-3 | 0.8 | — | — | 230° C. | 3 hours | Cyclo- | A | A | A | A | |
| | | | | | | | | | pentanone | | | | | |
| Example 14 | G-2 | 0.04 | I-3 | 0.8 | — | — | 230° C. | 3 hours | Cyclo- | B | B | A | B | |
| | | | | | | | | | pentanone | | | | | |
| Example 15 | G-2 | 0.04 | I-3 | 0.8 | — | — | 230° C. | 3 hours | Cyclo- | B | A | A | A | |
| | | | | | | | | | pentanone | | | | | |

TABLE 4

| | | | | | | | | | | | | | Metal | |
|---|---|---|---|---|---|---|---|---|---|---|---|---|---|---|
| | | | | | | | | | | | Migration | | adhesiveness | |
| | | | | | Polymenization | | | | Polymerizable | | suppressing | | improving | |
| | Resin | | | | initiator | | Solvent | | compound | | agent | | agent | |
| | | Adding | | Adding | | Adding | | Adding | | Adding | | Adding | | Adding |
| | Type | amount | Type | amount | Type | amount | Type | amount | Type | amount | Type | amount | Type | amount |
| Example | SE-16 | 30.4 | — | — | OXE-02 | 1.66 | GBL | 60 | D-1 | 65 | E-1 | 0.10 | F-4 | 0.50 |
| 16 | | | | | | | | | | | | | | |
| Example | SE-17 | 30.4 | — | — | OXE-02 | 1.66 | GBL | 60 | D-1 | 65 | E-1 | 0.10 | F-4 | 0.50 |
| 17 | | | | | | | | | | | | | | |
| Example | SE-18 | 30.4 | — | — | OXE-02 | 1.66 | GBL | 60 | D-1 | 65 | E-1 | 0.10 | F-4 | 0.50 |
| 18 | | | | | | | | | | | | | | |
| Example | SE-19 | 30.4 | — | — | OXE-02 | 1.66 | GBL | 60 | D-1 | 65 | E-1 | 0.10 | F-4 | 0.50 |
| 19 | | | | | | | | | | | | | | |
| Example | SE-20 | 30.4 | — | — | OXE-02 | 1.66 | GBL | 60 | D-1 | 65 | E-1 | 0.10 | F-4 | 0.50 |
| 20 | | | | | | | | | | | | | | |
| Example | SE-21 | 30.4 | — | — | OXE-02 | 1.66 | GBL | 60 | D-1 | 65 | E-1 | 0.10 | F-4 | 0.50 |
| 21 | | | | | | | | | | | | | | |
| Example | SE-22 | 30.4 | — | — | OXE-02 | 1.66 | GBL | 60 | D-1 | 65 | E-1 | 0.10 | F-4 | 0.50 |
| 22 | | | | | | | | | | | | | | |
| Example | SE-1 | 24.8 | SP-1 | 6.6 | OXE-01/ | 1.00/ | GBL | 60 | D-1/ | 3.0/ | E-3 | 0.10 | F-6 | 0.70 |
| 23 | | | | | B4 | 0.66 | | | BS-2 | 3.0 | | | | |
| Example | SE-9 | 30.4 | — | — | OXE-01/ | 1.00/ | GBL | 60 | D-1/ | 3.0 | E-3 | 0.10 | F-6 | 0.70 |
| 24 | | | | | B-3 | 0.66 | | | BS-2 | 3.0 | | | | |
| Example | SE-9 | 15.0 | SP-2 | 15.0 | OXE-02 | 1.64 | γ-valerolactone/ | 30/ | D3 | 5.7 | E-4 | 0.10 | F-1 | 0.35 |
| 25 | | | | | | | NMP | 30 | | | | | | |
| Example | SE-10 | 20.0 | SP-3 | 12.0 | B-1 | 1.65 | GBL | 60 | D-1 | 5.5 | E-5 | 0.10 | F-2 | 0.70 |
| 26 | | | | | | | | | | | | | | |
| Example | SE-1 | 20.0 | SP-4 | 11.3 | B-2 | 1.65 | γ-valerolactone/ | 45/15 | D-1 | 65 | E-6 | 0.10 | F-3 | 0.40 |
| 27 | | | | | | | DMSO | | | | | | | |
| Example | SE-1 | 26.9 | SP-5 | 3.6 | OXE-02 | 1.55 | GBL | 60 | D-1 | 63 | E-7 | 0.10 | F-1 | 0.70 |
| 28 | | | | | | | | | | | | | | |
| Example | SE-1 | 15.4 | SP-6 | 15.4 | OXE-02 | 1.85 | MDMPA | 60 | D-2 | 65 | E-2 | 0.10 | F-2 | 0.71 |
| 29 | | | | | | | | | | | | | | |
| Example | SE-9 | 16.8 | SP-6/ | 7.0/ | OXE-01 | 1.65 | γ-valerolactone | 60 | D-3 | 67 | E-2 | 0.11 | F-3 | 0.70 |
| 30 | | | SP-7 | 7.0 | | | | | | | | | | |
| | Polymerization | | | | | | Curing | | | | Dielectric | Development | | |
| | inhibitor | | Addtive | | Surfactant | | condition | | | | loss | residual | | |
| | | Adding | | Adding | | Adding | Tem- | Curing | | Breaking | tangent | film | Insulatior | |
| | Type | amount | Type | amount | Type | amount | perature | time | Developer | elongatior | (Df) | property | reliability | |
| Example | G-2 | 0.04 | I-3 | 0.8 | — | — | 230° C. | 3 hours | Cyclo- | A | A | A | A | |
| 16 | | | | | | | | | pentanone | | | | | |
| Example | G-2 | 0.04 | I-3 | 0.8 | — | — | 230° C. | 3 hours | Cyclo- | A | B | A | B | |
| 17 | | | | | | | | | pentanone | | | | | |
| Example | G-2 | 0.04 | I-3 | 0.8 | — | — | 230° C. | 3 hours | Cyclo- | A | A | A | A | |
| 18 | | | | | | | | | pentanone | | | | | |
| Example | G-2 | 0.04 | I-3 | 0.8 | — | — | 230° C. | 3 hours | Cyclo- | B | B | A | A | |
| 19 | | | | | | | | | pentanone | | | | | |
| Example | G-2 | 0.04 | I-3 | 0.8 | — | — | 230° C. | 3 hours | Cyclo- | A | A | A | A | |
| 20 | | | | | | | | | pentanone | | | | | |
| Example | G-2 | 0.04 | I-3 | 0.8 | — | — | 230° C. | 3 hours | Cyclo- | A | A | A | A | |
| 21 | | | | | | | | | pentanone | | | | | |
| Example | G-2 | 0.04 | I-3 | 0.8 | — | — | 230° C. | 3 hours | Cyclo- | A | A | A | A | |
| 22 | | | | | | | | | pentanone | | | | | |
| Example | G-1 | 0.04 | I-1 | 0.2 | — | — | 230° C. | 3 hours | Cyclo- | A | A | A | A | |
| 23 | | | | | | | | | pentanone | | | | | |
| Example | G-1 | 0.04 | I-1 | 1.2 | — | — | 170° C. | 2 hours | Cyclo- | A | A | A | A | |
| 24 | | | | | | | | | pentanone | | | | | |
| Example | G-2 | 0.05 | I-2 | 0.5 | — | — | 230° C. | 4 hours | Cyclo- | B | A | B | A | |
| 25 | | | | | | | | | pentanone | | | | | |
| Example | G-3 | 0.05 | — | — | — | — | 200° C. | 3 hours | Cyclo- | B | B | B | B | |
| 26 | | | | | | | | | pentanone | | | | | |
| Example | G-4 | 0.05 | — | — | — | — | 230° C. | 3 hours | Cyclo- | B | A | B | A | |
| 27 | | | | | | | | | pentanone | | | | | |
| Example | G-2 | 0.05 | I-3 | 0.8 | — | — | 230° C. | 3 hours | Cyclo- | B | A | A | A | |
| 28 | | | | | | | | | pentanone | | | | | |
| Example | G-2 | 0.04 | — | — | — | — | 230° C. | 1 hour | Cyclo- | B | A | A | A | |
| 29 | | | | | | | | | pentanone | | | | | |
| Example | G-2 | 0.04 | — | — | — | — | 230° C. | 3 hours | Cyclo- | A | A | A | A | |
| 30 | | | | | | | | | pentanone | | | | | |

TABLE 5

| | | | | | | | | | | | | | Metal | |
|---|---|---|---|---|---|---|---|---|---|---|---|---|---|---|
| | | | | | | | | | | | Migration | | adhesiveness | |
| | | | | | Polymenization | | | | Polymerizable | | suppressing | | improving | |
| | Resin | | | | initiator | | Solvent | | compound | | agent | | agent | |
| | | Adding | | Adding | | Adding | | Adding | | Ading | | Adding | | Adding |
| | Type | amount | Type | amount | Type | amount | Type | amount | Type | amount | Type | amount | Type | amount |
| Example 31 | SE-9 | 20.2 | SP-8 | 10.0 | OXE-01 | 1.66 | NMP | 60 | D-2/ | 3.0/3.3 | E.2 | 0.10 | F-4 | 0.70 |
| | | | | | | | | | BS-1 | | | | | |
| Example 32 | SE-4 | 20.0 | SA-1 | 10.1 | OXE-02 | 1.65 | γ- | 30/30 | D-4 | 6.6 | E-2 | 0.10 | F-5 | 0.70 |
| | | | | | | | valerolactone/ | | | | | | | |
| | | | | | | | GBL | | | | | | | |
| Example 33 | SE-3 | 31.9 | — | — | B-3 | 1.66 | NMP | 60 | D-5 | 5.0 | E-1 | 0.10 | F-4 | 0.50 |
| Example 34 | SE-1 | 30.4 | — | — | OXE-02 | 1.66 | GBL | 60 | D-1 | 6.5 | E-1 | 0.10 | F-4 | 0.50 |
| Example 35 | SE-9 | 15.0 | SP-2 | 15.0 | OXE-02 | 1.64 | γ- | 30/30 | D-3 | 5.7 | E-4 | 0.10 | F-1 | 0.35 |
| | | | | | | | valerolactone/ | | | | | | | |
| | | | | | | | NMP | | | | | | | |
| Example 36 | SE-3 | 31.9 | — | — | B-3 | 1.66 | NMP | 60 | D-5 | 5.0 | E-1 | 0.10 | F-4 | 0.50 |
| Example 37 | SE-23 | 30.4 | — | — | OXE-02 | 1.66 | GBL | 60 | D-1 | 6.5 | E-1 | 0.10 | F-4 | 0.50 |
| Example 38 | SE-24 | 30.4 | | | OXE-02 | 1.66 | GBL | 60 | D-1 | 6.5 | E-1 | 0.10 | F-4 | 0.50 |
| Comparative | A-1 | 30.0 | — | — | OXE-01 | 1.30 | DMSO/ | 60 | D-4 | 7.7 | E-2 | 0.20 | F-2 | 0.70 |
| Example 1 | | | | | | | GBL | | | | | | | |
| | Polymerization | | | | | | Curing | | | | Dieletric | Development | | |
| | inhibitor | | Addtive | | Surfactant | | condition | | | | loss | residual | | |
| | | Adding | | Adding | | Adding | Tem- | Curing | | Breaking | tangent | film | Insulation | |
| | Type | amount | Type | amount | Type | amount | perature | time | Developer | elongatior | (Df) | property | reliability | |
| Example 31 | G-3 | 0.04 | I-1 | 1.0 | — | — | 170° C. | 2 hours | Cyclo- | B | A | A | A | |
| | | | | | | | | | pentanone | | | | | |
| Example 32 | G-4 | 0.05 | I-3 | 0.8 | — | — | 230° C. | 3 hours | Cyclo- | B | B | B | B | |
| | | | | | | | | | pentanone | | | | | |
| Example 33 | G-2 | 0.04 | I-3 | 0.8 | — | — | 230° C. | 3 hours | Alkali | B | B | B | B | |
| Example 34 | G-2 | 0.04 | I-3 | 0.7 | J-1 | 0.1 | 230° C. | 3 hours | Cyclo- | A | A | A | A | |
| | | | | | | | | | pentanone | | | | | |
| Example 35 | G-2 | 0.05 | I-2 | 0.4 | J-2 | 0.1 | 230° C. | 4 hours | Cyclo- | B | A | B | A | |
| | | | | | | | | | pentanone | | | | | |
| Example 36 | G-2 | 0.04 | I-3 | 0.7 | J-3 | 0.1 | 230° C. | 3 hours | Alkali | B | B | B | B | |
| Example 37 | G-2 | 0.04 | I-3 | 0.8 | — | — | 230° C. | 3 hours | Cyclo- | A | A | A | A | |
| | | | | | | | | | pentanone | | | | | |
| Example 38 | G-2 | 0.04 | I-3 | 0.8 | — | — | 230° C. | 3 hours | Cyclo- | B | A | A | A | |
| | | | | | | | | | pentanone | | | | | |
| Comparative | G-3 | 0.10 | — | — | — | — | 230° C. | 3 hours | Cyclo- | D | C | C | C | |
| Example 1 | | | | | | | | | pentanone | | | | | |

### [Resin]

SE-1 to SE-22: SE-1 to SE-24 synthesized above
SP-1 to SP-8: SP-1 to SP-8 synthesized above
SA-1: SA-1 synthesized above
A-1: A-1 synthesized above

### [Polymerization Initiator]

OXE-01: IRGACURE OXE 01 (manufactured by BASF SE)
OXE-02: IRGACURE OXE 02 (manufactured by BASF SE)
B-1: Omnirad 1312 (manufactured by IGM Resins)
B-2: Omnirad TPO H (manufactured by IGM Resins)
B-3: CPI-310B (manufactured by San-Apro Ltd.)
B-4: benzoyl peroxide (manufactured by Tokyo Chemical Industry Co., Ltd.)

### [Solvent]

DMSO: dimethyl sulfoxide
GBL: γ-butyrolactone
NMP: N-methylpyrrolidone
γ-valerolactone: γ-valerolactone

In the table, the description of “DMSO/GBL” indicates that a mixture of DMSO and GBL at a mixing ratio (mass ratio) of DMSO:GBL=20:80 was used.
MDMPA: KJCMPA-100 (manufactured by KJ Chemicals Co., Ltd.)

### [Polymerizable Compound]

D-1: 1,12-dodecanediol dimethacrylate (melting point: 25° C. or lower)
D-2: 1,9-nonanediol dimethacrylate (melting point: 25° C. or lower)
D-3: 1,10-decanediol dimethacrylate (melting point: 25° C. or lower)
D-4: SR-209 (manufactured by Sartomer Japan Inc., melting point: 25° C. or lower)
D-5: 2,2-bis(4-glycidyloxyphenyl)propane (manufactured by Tokyo Chemical Industry Co., Ltd., melting point: 25° C. or lower)
BS-1: BS-1 synthesized above (melting point: 25° C. or lower)
BS-2: BS-2 synthesized above (melting point: 25° C. or lower)

### [Migration Suppressing Agent]

E-1 to E-7: Compounds having the following structures

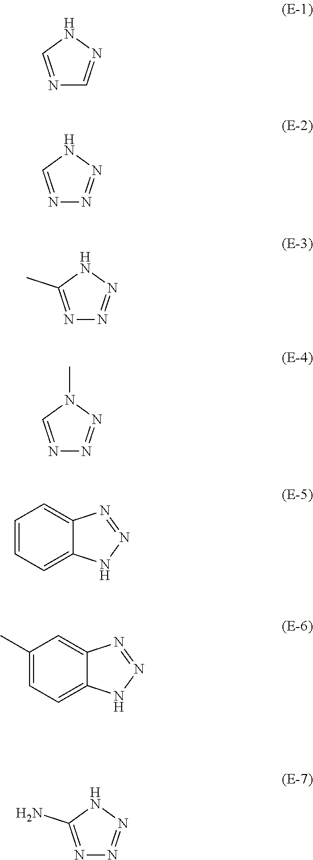

### [Metal Adhesiveness Improving Agent]

F-1 to F-3: Compounds having the following structures

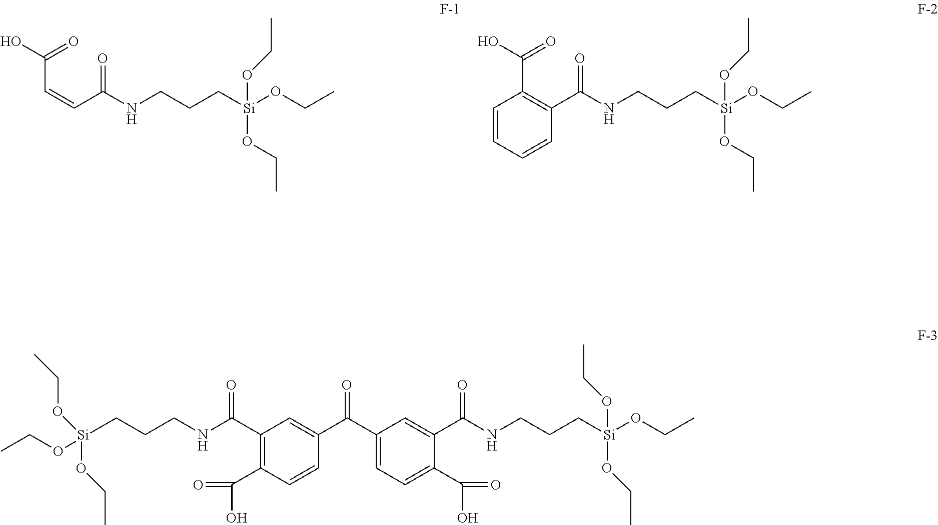 [SMILES: CCO[Si](CCCNC(=O)/C=C\C(=O)O)(OCC)OCC ; CCO[Si](CCCNC(=O)c1ccccc1C(=O)O)(OCC)OCC ; CCO[Si](CCCNC(=O)c1cc(C(=O)c2ccc(C(=O)O)c(C(=O)NCCC[Si](OCC)(OCC)OCC)c2)ccc1C(=O)O)(OCC)OCC]

F-4: X-12-1293 (manufactured by Shin-Etsu Chemical Co., Ltd.)
F-5: KBM-513 (manufactured by Shin-Etsu Chemical Co., Ltd.)
F-6: X-12-1214A (manufactured by Shin-Etsu Chemical Co., Ltd.)

### [Polymerization Inhibitor]

G-1: 1,4-benzoquinone
G-2: 4-methoxyphenol
G-3: 1,4-dihydroxybenzene
G-4: A compound having the following structure

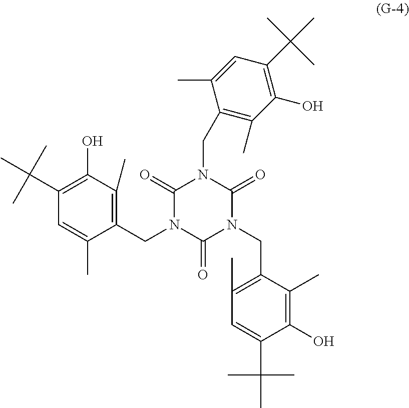 [SMILES: Cc1cc(C(C)(C)C)c(O)c(C)c1Cn1c(=O)n(Cc2c(C)cc(C(C)(C)C)c(O)c2C)c(=O)n(Cc2c(C)cc(C(C)(C)C)c(O)c2C)c1=O]

### [Additive]

I-1: N-phenyldiethanolamine (manufactured by Tokyo Chemical Industry Co., Ltd.)
I-2: TC-401 (manufactured by Matsumoto Fine Chemical Co., Ltd.)
I-3: compound having the following structure

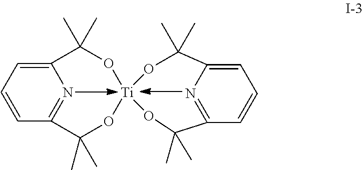

### [Surfactant]

J-1: MEGAFACE EFS-801 (manufactured by Dainippon Ink and Chemicals, Inc.)
J-2: Shin-Etsu Silicone KF6028 (manufactured by Shin-Etsu Chemical Co., Ltd.)
J-3: Nonion E-212 (manufactured by NOF Corporation)

### <Evaluation>

### [Evaluation of Dielectric Loss Tangent (Df)]

The resin compositions or comparative compositions prepared in each of Examples and Comparative Examples were applied onto a 12-inch silicon wafer by a spin coating method to form a resin composition layer. The silicon wafer to which the obtained resin composition layer was applied was dried on a hot plate at 100° C. for 5 minutes, and a resin composition layer having a uniform thickness of 15 m was formed on the silicon wafer. The resin composition layer on the silicon wafer was exposed over the entire surface using a stepper (Nikon NSR 2005 i9C) at an exposure energy of 500 mJ/cm², the exposed resin composition layer (resin layer) was heated at a temperature rising rate of 10° C./min in a nitrogen atmosphere, and heated at the temperature described in the column of “Temperature” in “Curing condition” in the table for 180 minutes, thereby obtaining a cured layer (resin layer) of the resin composition layer.

The cured layer (resin film) after curing was immersed in a 4.9% by mass hydrofluoric acid aqueous solution, and the cured film was peeled off from the silicon wafer.

The film sample was measured for relative permittivity (Dk) and dielectric loss tangent (Df) at 28 GHz by a resonator perturbation method.

### <Measurement Method>

Split cylinder resonator (CR-728)

### (Device Configuration)

Network analyzer: N5230A (manufactured by Keysight Technologies, Inc.)

### (Evaluation Standards)

Dielectric loss tangent (Df)
A: The dielectric loss tangent (Df) was less than 0.07.
B: The dielectric loss tangent (Df) was 0.07 to less than 0.09.
C: The dielectric loss tangent (Df) was 0.09 to less than 0.12.
D: The dielectric loss tangent (Df) was 0.12 or more.

### [Evaluation of Insulation Reliability]

The resin composition and the comparative composition prepared in each of Examples and Comparative Examples were each applied in a layer shape onto a copper substrate by a spin coating method to form a resin composition layer or a comparative composition layer. The copper substrate on which the obtained resin composition layer or comparative composition layer was formed was dried on a hot plate at 100° C. for 5 minutes to form a resin composition layer or a comparative composition layer having a thickness of 5 m and a uniform thickness on the copper substrate. The resin composition layer or the comparative composition layer on the copper substrate was exposed to i-rays using a photo mask in which a square non-masked portion having a square shape of 100 μm square was formed at an exposure energy of 500 mJ/cm² using a stepper (Nikon NSR 2005 i9C), developed for 60 seconds with a developer described in the column of “Developer” in the table, and rinsed with propylene glycol monomethyl ether acetate (PGMEA) to obtain a resin layer having a square shape of 100 m square. Furthermore, the resin layer (pattern) was formed by heating the resin layer at the temperature described in the column of “Temperature (° C.)” in the table for the time described in the column of “Curing time” in the table using a heating oven in a nitrogen atmosphere.

After the resin layer and the copper substrate had been allowed to stand in a constant temperature tank at 175° C. for 1,000 hours, a cross-sectional scanning electron microscope (SEM) measurement was performed to evaluate a void area ratio between the copper substrate and the resin layer. The void area ratio was calculated by the following expression.

$$Void area ratio ( \% ) = ( area of void portion observed by SEM measurement ) / ( total area of resin layer ) \times 100$$

The obtained void area ratio value was evaluated according to the following evaluation standard. The evaluation results are described in the column of “Insulation reliability” in the table. It can be said that the reliability of the cured film after a high temperature storage test (HTS) is excellent as the void area ratio is smaller, and it can be said that the void is unlikely to occur between the metal layer and the cured substance even after a long period of time, and the insulation reliability is favorable.

### —Evaluation Standards —

A: The void area ratio was 0.1% or less.
B: The void area ratio was more than 0.1% and 0.3% or less.
C: The void area ratio was more than 0.3% and 0.5% or less.
D: The void area ratio was more than 0.5%.
   [Evaluation of Residual Film Rate after Development]

Each of the resin compositions used in each of Examples and Comparative Examples was applied onto the surface of a copper thin layer formed on the surface of a resin substrate in a layer shape by a spin coating method, dried at 100° C. for 5 minutes, and a resin composition layer having a film thickness of 10 m after film formation was formed, and a film thickness (film thickness A) before development was measured by a scanning electron microscope (SEM). Subsequently, the above-described coating film was exposed using a stepper (FPA-3000 i5 (manufactured by Canon Inc.)) at NA=0.50 and 300 mJ/cm². The exposure was performed at a wavelength of 365 nm through a hole pattern mask formed with a hole pattern having a diameter of 15 km. Subsequently, the film was developed with cyclopentanone for 15 seconds, rinsed with PGMEA for 30 seconds, and the film thickness (film thickness B) after development was measured by a scanning electron microscope (SEM). The residual film rate after development was obtained by the following expression, film thickness B/film thickness A×100(%). It can be said that the residual film property after development is excellent as the residual film rate is higher.

### —Evaluation Standards —

A: The residual film ratio was 90% or more.
B: The residual film ratio was 85% or more and less than 90%.
C: The residual film ratio was 80% or more and less than 85%.
D: The residual film ratio was less than 80%.

### [Evaluation of Breaking Elongation]

Each of the resin compositions or comparative compositions in Examples and Comparative Examples was applied onto a silicon wafer by a spin coating method to form a resin composition layer. The silicon wafer to which the obtained resin composition layer was applied was dried on a hot plate at 100° C. for 5 minutes, thereby obtaining a uniform resin composition layer having a thickness of about 15 m on the silicon wafer.

The entire surface of the obtained resin composition layer was exposed to i-rays at an exposure energy of 500 mJ/cm² using a stepper (Nikon NSR 2005 i9C).

The temperature of the above-described resin composition layer (resin layer) after exposure was performed was raised at a temperature rising rate of 10° C./min in a nitrogen atmosphere, and was heated for 3 hours after reaching the temperature described in the column of “Temperature” in “Curing conditions” in the table. The resin layer (cured film) after curing was immersed in a 4.9% by mass hydrofluoric acid aqueous solution, and the cured film was peeled off from the silicon wafer. The peeled cured film was punched out using a punching machine to prepare a test piece having a sample width of 3 mm and a sample length of 30 mm. For the obtained test piece, a tensile tester (Tensilon) was used to measure a breaking elongation rate of the test piece in the longitudinal direction in an environment of 25° C. and 65% RH (relative humidity) at a crosshead speed of 300 mm/min in accordance with JIS-K6251. The evaluation was performed five times each, and the arithmetic average value was used as an index value for the elongation rate (breaking elongation rate) at the time of breaking of the test piece.

The above-described index value was evaluated according to the following evaluation standard, and the evaluation results are described in the column of “Breaking elongation” in the table. It can be said that the film strength (breaking elongation) of the obtained cured film is excellent as the index value is larger.

### (Evaluation Standards)

A: The index value was 60% or more.
B: The index value was 50% or more and less than 60%.
C: The index value was 40% or more and less than 50%.
D: The index value was less than 40%.

### Example 101

The resin composition used in Example 1 was applied in a layer shape onto a surface of a thin copper layer of the resin base material on the surface of which the thin copper layer was formed, by a spin coating method, dried at 100° C. for 4 minutes, and after forming a resin composition layer having a film thickness of 20 m, exposure was carried out using a stepper (NSR1505 i6, manufactured by Nikon Corporation). Exposure was carried out through a mask (a binary mask in which the pattern is a pattern of 1:1 line and space and the line width is 10 m) at a wavelength of 365 nm. After the exposure, heating was carried out at 100° C. for 4 minutes. After the heating, development was carried out with cyclohexanone for 2 minutes and rinsing was carried out with PGMEA for 30 seconds to obtain a layer pattern.

Next, the temperature was raised at a temperature rising rate of 10° C./min in a nitrogen atmosphere, and after the temperature reached 230° C., the temperature was maintained at 230° C. for 3 hours, thereby forming an interlayer insulating film for a re-distribution layer. This interlayer insulating film for a re-distribution layer was excellent in insulating properties.

In addition, in a case where a semiconductor device was manufactured using this interlayer insulating film for a re-distribution layer, it has been confirmed that the semiconductor device operates without any problem.

What is claimed is:

1. A photosensitive resin composition comprising:
a polyimide including a repeating unit represented by Formula (1-1); and
a photosensitizer, 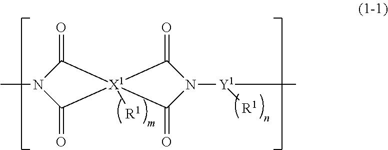
in Formula (1-1), X¹ represents an organic group having 4 or more carbon atoms, Y¹ represents an organic group having 4 or more carbon atoms, R¹'s each independently represent a structure represented by Formula (R-1), n and m each represent an integer of 0 to 4, and n+m represents an integer of 1 or more, 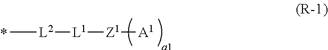
in Formula (R-1), L¹ represents an acetal group, L² represents a single bond or a divalent linking group, Z¹ represents a (1+a1)-valent linking group, A¹ represents a polymerizable group, a1 represents an integer of 0 or more and a maximum substituent number of Z¹ or less, and * represents a bonding site to X¹ or Y¹ in Formula (1-1).

2. The photosensitive resin composition according to claim 1,
wherein L¹ in Formula (R-1) is thermally decomposable.

3. The photosensitive resin composition according to claim 1,
wherein a1 in Formula (R-1) is an integer of 1 or more, and
A¹ is a radically polymerizable group.

4. The photosensitive resin composition according to claim 1,
wherein X¹ and Y¹ in Formula (1-1) each include a structure obtained by removing two or more hydrogen atoms from a structure represented by any of Formulae (V-1) to (V-10), 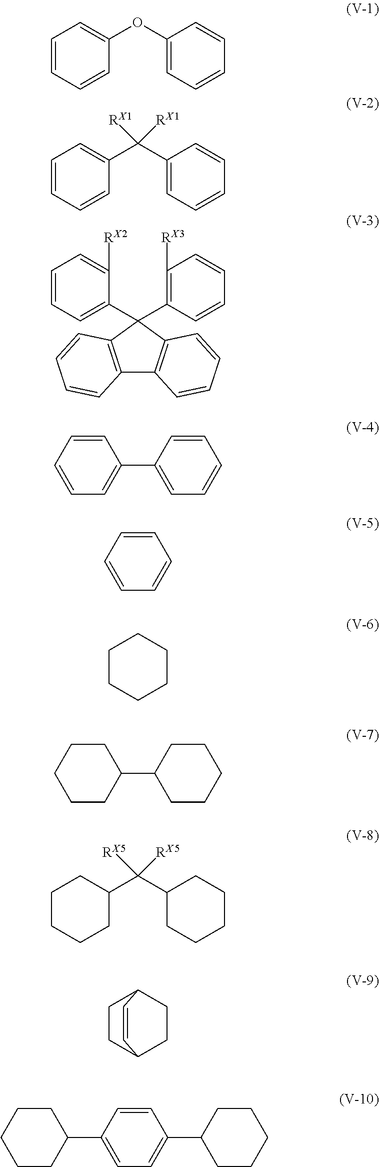
in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure,
in Formula (V-8), R^{X5}'s each independently represent a hydrogen atom, an alkyl group, or a halogenated alkyl group.

5. A photosensitive resin composition comprising:
a polyimide from which a structure having 2 or more carbon atoms is eliminated upon heating at 230° C. for 3 hours; and
a photosensitizer.

6. The photosensitive resin composition according to claim 5,
wherein the eliminated structure includes a polymerizable group.

7. The photosensitive resin composition according to claim 1,
wherein a polymerizable group equivalent of the polyimide is 0.2 to 5.0 mmol/g.

8. The photosensitive resin composition according to claim 1,
wherein in a case where a film-like cured substance having a film thickness of 10 m is formed using the photosensitive resin composition, a transmittance of the cured substance at a wavelength of 365 nm is 15% or more.

9. The photosensitive resin composition according to claim 1,
wherein the polyimide has a chain-like alkyl group having 6 or more carbon atoms.

10. The photosensitive resin composition according to claim 1, further comprising:
an azole compound.

11. The photosensitive resin composition according to claim 1, further comprising:
a silane coupling agent.

12. The photosensitive resin composition according to claim 1, further comprising:
an organic titanium compound.

13. The photosensitive resin composition according to claim 1, further comprising:
a solvent having a boiling point of 100° C. to 260° C. at 1 atm.

14. The photosensitive resin composition according to claim 13,
wherein a content of the solvent having the boiling point of 100° C. to 260° C. is 40% by mass or more with respect to a total mass of the composition.

15. The photosensitive resin composition according to claim 13, further comprising:
two or more kinds of the solvents having the boiling point of 100° C. to 260° C.

16. The photosensitive resin composition according to claim 1,
wherein the photosensitive resin composition is used for forming an interlayer insulating film for a re-distribution layer.

17. A cured substance obtained by curing the photosensitive resin composition according to claim 1.

18. A laminate comprising:
two or more layers consisting of the cured substance according to claim 17; and
a metal layer provided between any of the layers consisting of the cured substance.

19. A manufacturing method for a cured substance, comprising:
a film forming step of applying the photosensitive resin composition according to claim 1 onto a substrate to form a film;
an exposure step of selectively exposing the film; and
a development step of developing the film using a developer to form a pattern.

20. The manufacturing method for a cured substance according to claim 19, further comprising:
a heating step of heating the film at 50° C. to 450° C.

21. A manufacturing method for a laminate, comprising:
the manufacturing method for a cured substance according to claim 19.

22. A manufacturing method for a semiconductor device, comprising:
the manufacturing method for a cured substance according to claim 19.

23. A semiconductor device comprising:
the cured substance according to claim 17.

24. A polyimide comprising:
a repeating unit represented by Formula (1-1), 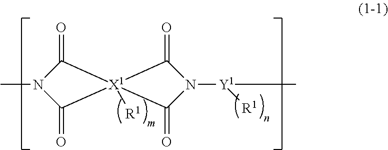
in Formula (1-1), X¹ represents an organic group having 4 or more carbon atoms, Y¹ represents an organic group having 4 or more carbon atoms, R¹'s each independently represent a structure represented by Formula (R-1), n and m each represent an integer of 0 to 4, and n+m represents an integer of 1 or more, 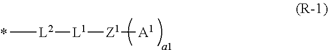
in Formula (R-1), L¹ represents an acetal group, L² represents a single bond or a divalent linking group, Z¹ represents a (1+a1)-valent linking group, A¹ represents a polymerizable group, a1 represents an integer of 0 or more and a maximum substituent number of Z¹ or less, and * represents a bonding site to X¹ or Y¹ in Formula (1-1).

25. The polyimide according to claim 24,
wherein a1 in Formula (R-1) is an integer of 1 or more, and
A¹ is a radically polymerizable group.

26. The polyimide according to claim 24,
wherein a polymerizable group equivalent is 0.2 to 5 mmol/g.

27. The polyimide according to claim 24,
wherein the polyimide has a chain-like alkyl group having 6 or more carbon atoms.

28. The polyimide according to claim 24,
wherein both of X¹ and Y¹ in Formula (1-1) include a ring structure.

29. The polyimide according to claim 24,
wherein X¹ and Y¹ in Formula (1-1) each include a structure obtained by removing two or more hydrogen atoms from a structure represented by any of Formulae (V-1) to (V-10), 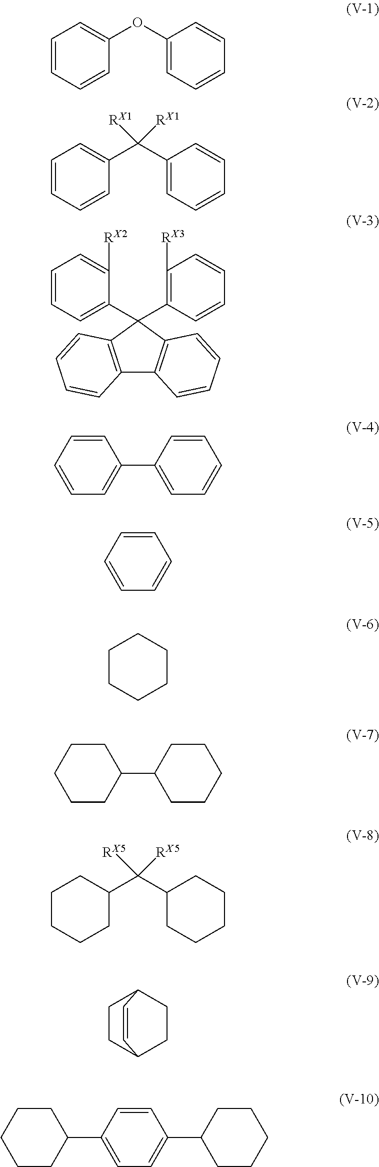
in Formula (V-2), R^{X1}'s each independently represent a hydrogen atom, an alkyl group, or a halogenated alkyl group,
in Formula (V-3), R^{X2} and R^{X3} each independently represent a hydrogen atom or a substituent, and R^{X2} and R^{X3} may be bonded to each other to form a ring structure,
in Formula (V-8), R^{X5}'s each independently represent a hydrogen atom, an alkyl group, or a halogenated alkyl group.

30. A manufacturing method for the polyimide according to claim 24, the method comprising:
a step of reacting a polyimide having an acid group or a precursor of the polyimide with a compound represented by Formula (B-1), 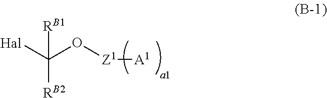
in Formula (B-1), Z¹ represents a (1+a1)-valent linking group, A¹ represents a polymerizable group, a1 represents an integer of 1 or more and a maximum substituent number of Z¹ or less, R^{B1} and R^{B2} each independently represent a hydrogen atom or a monovalent organic group, and Hal represents a halogen atom.

31. The manufacturing method for a polyimide according to claim 30, further comprising:
a step of obtaining the polyimide having the acid group from an acid dianhydride and a diamine.

32. A compound represented by Formula (A-1), 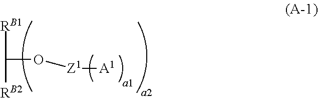
in Formula (A-1), Z¹'s each independently represent a (1+a1)-valent linking group, A¹'s each independently represent a polymerizable group, a1's each independently represent an integer of 1 or more and a maximum substituent number of Z¹ or less, R^{B1} and R^{B2} each independently represent a hydrogen atom or a monovalent organic group, and a2 represents 2.

33. A compound represented by Formula (B-1), 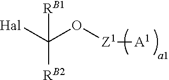
in Formula (B-1), Z¹ represents a (1+a1)-valent linking group, A¹ represents a polymerizable group, a1 represents an integer of 1 or more and a maximum substituent number of Z¹ or less, R^{B1} and R^{B2} each independently represent a hydrogen atom or a monovalent organic group, and Hal represents a halogen atom.

34. The compound according to claim 33,
wherein the polymerizable group in A¹ in Formula (B-1) is a radically polymerizable group.
